(19) United States
(12) Patent Application Publication
(10) Pub. No.: US 2011/0207214 A1
(43) Pub. Date: Aug. 25, 2011

(54) NOVEL TOOLS FOR THE PRODUCTION OF GLYCOSYLATED PROTEINS IN HOST CELLS

(75) Inventors: Jonne Helenius, Dresden (DE); Christine Neupert, Zuerich (CH); Markus Aebi, Wettingen (CH); Farnoush Parsaie Nasab, Zuerich (CH); Alexander-Daniel Frey, Olten (CH)

(73) Assignee: Lonza Ltd, Basel (CH)

(21) Appl. No.: 13/126,856

(22) PCT Filed: Oct. 31, 2009
(86) PCT No.: PCT/EP2009/007816

§ 371 Date: Apr. 29, 2011

Related U.S. Application Data
(60) Provisional application No. 61/198,023, filed on Oct. 31, 2008.

(30) Foreign Application Priority Data
Oct. 31, 2008 (EP) 08400044.7

(51) Int. Cl. C12N 5/10; C12N 1/15

(52) U.S. Cl. 435/348; 435/254.11; 435/325; 435/419

(57) ABSTRACT

The invention improves glycoprotein production and protein glycosylation engineering in eukaryotes, specifically the production of human-like complex or hybrid glycosylated proteins in lower eukaryotes such as yeasts. The invention provides glycosylation modified eukaryotic host cells capable of producing glycosylation optimized proteins useful as immunoglobulins and other therapeutic proteins, and provides cells capable of producing glycoproteins having glycan structures similar to glycoproteins produced in human cell. The invention further provides proteins with human-like glycan structures and novel compositions thereof producible by these cells.

### FIELD OF THE INVENTION

The invention relates to the field of glycoprotein production and protein glycosylation engineering in eukaryotes, specifically the production of human-like complex or hybrid glycosylated proteins in lower eukaryotes such as yeasts. The invention further relates to glycosylation modified eukaryotic host cells capable of producing glycosylation optimized proteins that are particularly useful as immunoglobulins and other therapeutic proteins for humans. The invention also relates to engineered eukaryotic, non-human cells capable of producing glycoproteins having glycan structures similar to glycoproteins produced in human cell. Accordingly, the invention further relates to proteins with human-like glycan structures and novel compositions thereof that are producible by said cells.

### BACKGROUND OF THE INVENTION

The majority of protein-based biopharmaceuticals bare some form of post-translational modification which can profoundly affect protein properties relevant to their therapeutic application. Protein glycosylation represents the most common modification (about 50% of human proteins are glycosylated). Glycosylation can introduce considerable heterogeneity into a protein composition through the generation of different glycan structures on the proteins within the composition. Such glycan structures are made by the action of diverse enzymes of the glycosylation machinery as the glycoprotein transits the Endoplasmatic Reticulum (ER) and the Golgi-Complex (glycosylation cascade). The nature of the glycan structure(s) of a protein has impact on the protein's folding, stability, life time, trafficking, pharmaco-dynamics, pharmacokinetics and immunogenicity. The glycan structure has great impact on the protein's primary functional activity. Glycosylation can affect local protein structure and may help to direct the folding of the polypeptide chain. One important kind of glycan structures are the so called N-glycans. They are generated by covalent linkage of an oligosaccharide to the amino (N)-group of asparagin residues in the consensus sequence NXS/T of the nascent polypeptide chain. N-glycans may further participate in the sorting or directing of a protein to its final target: the N-glycan of an antibody, for example, may interact with complement components. N-glycans also serve to stabilize a glycoprotein, for example, by enhancing its solubility, shielding hydrophobic patches on its surface, protecting from proteolysis, and directing intra-chain stabilizing interactions. Glycosylation may regulate protein half-life, for example, in humans the presence of terminal sialic acids in N-glycans may increase the half-life of proteins, circulating in the blood stream.

Synthesis of the oligosaccharide occurs on both sides of the ER membrane. The glycosylation cascade starts with the generation of a lipid-linked oligosaccharide (LLO) on the cytosolic surface of the ER membrane. At first a lipid-linked core oligosaccharide with a defined structure (Man3GlcNAc2) is synthezised. Further oligosaccharides are added onto the lipid dolichol-linked Man3GlcNAc2 on the cytosolic surface giving rise to the heptasaccharide Man5GlcNAc2 glycan structure. This LLO is then translocated (“flipped”) to the lumenal side of the ER. There further processing of the hepta-oligosaccharide chain to the branched oligosaccharide unit comprising three glucose, nine mannose, and two N-acetyl glucosamine residues (Glc3Man9GlcNAc2) structure takes place. The Glc3Man9GlcNAc2 structure is made by the action of several glycosyltransferases. Each individual glycosyltransferase displays strong preference towards a certain oligosaccharide substrate. This leads to a basically linear, stepwise biosynthesis of the branched oligosaccharides. The Glc3Man9GlcNAc2 structure is than transferred from the dolichol lipid to the nascent polypeptide. FIG. 1 depicts the LLO processing at the ER in wild type yeasts.

Two steps of this ER glycosylation pathway are not directly related to the action of glycosyltransferases: (1) the flipping of the Man5GlcNAc2-LLO from the cytosolic side of the ER membrane to the luminal side and (2) the oligosaccharyltransfer from the lipid-linker to the nascent polypeptide.

Flipping is catalyzed by an ATP-independent bi-directional flippase. In yeast, the flippase activity is supported or conferred by “Rft1”, a polytopic membrane protein comprising about ten transmembrane domains, which span through the ER membrane. Genes for homologous proteins occur in the genomes of other eukaryotes.

Without wishing to be bound to the theory, the complete oligosaccharide Glc3Man9GlcNAc2 is the optimal substrate for the oligosaccharyl transferase (OT or OST), which then transfers the oligosaccharide en bloc from the donor LLO onto the amino group of a selected asparagin residue within a Asn-X-Ser/Thr consensus sequences of a nascent protein or polypeptide. In most organisms the oligosaccharyl transferase is a multimeric complex containing seven or eight different proteins, one of which (Stt3p) is the catalytic subunit. Once the glycoproteins have been folded and oligomerized properly, they move to the Golgi complex. The N-linked glycans are then subject to further trimming and modification and new saccharides are added to generate e.g. hybrid or complex type glycans in human cells.

Glycosyl transferases and glycosidases line the inner (lumenal) surface of the ER and Golgi apparatus and thereby provide a “catalytic” surface that allows for the sequential processing of glycoproteins as they proceed through the ER and Golgi network. In fact, the multiple compartments of the cis, medial, and trans Golgi and the trans-Golgi Network (TGN), provide the different localities in which the ordered sequence of glycosylation reactions can take place. As a glycoprotein proceeds from synthesis in the ER to full maturation in the late Golgi or TGN, it is sequentially exposed to different glycosidases, mannosidases and glycosyl transferases such that a specific oligosaccharide glycan structure may be synthesized

Different organisms provide different glycosylation enzymes (glycosyltransferases and glycosidases). Thus, the final composition of a glycan structure of a protein may vary markedly depending upon the host. For example, lower eukaryotes such as yeast and filamentous fungi typically add high amounts of mannose residues within the Golgi to give rise to “high-mannose” type glycoproteins; whereas, in mammalian cells, glycan structures may be trimmed within the Golgi to remove several of the nine mannose residues and to be further elongated with additional sugar residues that typically do not occur in the N-glycans of lower eukaryotes, for example, sialic acid or fucose.

The possibility of producing recombinant proteins has revolutionized the treatment of patients with a variety of different diseases. Most therapeutic proteins need to be modified by the addition of glycan structures. This glycosylation may be necessary for correct folding, for long circulation and, in many cases, for optimal activity of the protein. Mammalian cells, like the commonly used Chinese hamster ovary cells (CHO cells) can produce complex glycan structures similar to human glycan structures, Nevertheless, glycan structures from e.g. CHO cells differ from glycan structures of human origin, as CHO cells a) sialylate at a lower degree, b) integrate additionally oligosaccharides to the common sialic acid (NeuAc) another non-human sialic acid (NeuGc) and c) contain terminally bound α-1-3 galactose which is absent in human cells. Disadvantages of the currently used mammalian expression systems for the production of recombinant proteins are (1) low productivity, (2) cost-intensive fermentation procedures, (3) complex strain design and (4) the risk of virus contamination.

In contrast to mammalian cells, yeast cells are robust organisms for industrial fermentation and can be cultivated to high densities in well-defined media. Although glycosylation in yeast and fungi is very different from that in mammals and humans, some common elements are shared. The first step, the transfer of the LLO to the nascent protein in the ER, is highly conserved in all eukaryotes including yeast, fungi, plants and humans. Subsequent processing of the N-glycan in the Golgi, however, differs significantly in yeast and in mammals. In yeast it involves the addition of several mannose sugars. These mannosylations are catalyzed by mannosyltransferases residing in the Golgi (e.g. Och1, Mnn1, Mnn2, etc.), which sequentially add mannose sugars to the N-glycan.

The manufacture of therapeutic proteins with a reproducible and consistent glycoform profile remains a considerable challenge to the biopharmaceutical industry. In particular, therapeutic glycoproteins produced in yeast may trigger an unwanted immune response in higher eukaryotes, in particular animals and humans, leading to a low therapeutic value of therapeutic proteins produced in yeast and the like. The impact of glycosylation on secretion, stability, immunogenicity and activity of several therapeutic proteins has been observed for several important therapeutic classes, including, blood factors, anticoagulants, thrombolytics, antibodies, hormones, stimulating factors and cytokines, for example, regulatory proteins of the TFN-family, EPO, gonadotropins, immunoglobulin G (IgG), granulocyte-macrophage colony-stimulating factor and interferons.

A number of yeasts, for example, Pichia pastoris, Yarrowia lipolytica and Saccharomyces cerevisiae are recently under development to use the advantages of such systems but to eliminate the disadvantages in respect to glycosylation. Several strains are under genetical development to produce defined, human-like glycan structures on a protein.

### SUMMARY OF THE INVENTION

It is the object of the present invention to provide means and methods for the production of glycosylated molecules such as lipids and proteins, in particular, recombinant glycoproteins, and as preferred examples immunoglobulins. It is a further object to provide a glycoprotein with a defined glycan structure, such as in particular a human-like or hybrid or complex glycan structure, and novel compositions thereof, that are producible by said means and methods. A particular object of the invention is the provision of N-glycosylated proteins and in particular immunoglobulins with a human-like glycan structure that are useable for therapy in humans with high therapeutic efficacy and without triggering unwanted side effects.

The technical problem underlying the present invention is primarily solved by the provision of a novel lipid-linked oligosaccharide (LLO) flippase activity (LLO flippase activity). The novel flippase activity is primarily characterized in that it is capable of efficiently flipping LLOs comprising glycan structures that comprise one mannose residue, in particular Man1GlcNAc2; is capable of efficiently flipping LLOs comprising glycan structures that comprise two mannose residue, in particular Man2GlcNAc2; and is is capable of efficiently flipping LLOs comprising glycan structures that comprise three mannose residues, in particular Man3GlcNAc2, and particularly with great activity.

The present invention provides a novel type of “LLO flippase activity” which, in contrast to known flippase activities, in particular a Rft1-type activity, exhibits a “relaxed” specifity in respect to the oligosaccharyl structure to be flipped. Without wishing to be bound to the theory, known flippase activities, e.g., of lower eukaryotes that have been characterized before, show high specifity to a certain glycan structure of the LLO to be flipped. More particular, the Rft1-type activity (synonymous name: YBL020W; Man5GlcNAc2-PP-Dol flippase) is primarily capable of flipping LLOs comprising 5 mannose residues, in particular a Man5GlcNAc2 glycan structure, but is basically unable to flip LLOs comprising a Man1GlcNAc2 glycan structure.

The term “efficiently” as used herein, primarily refers to enzymatic or transfer activity, that takes place in an amount or rate sufficient to pursue the technical purpose of the host cells with in the scope and objectives of the present invention as described herein. For example, an “efficient” transfer or synthesis is considered not to resemble or reflect the primary rate limiting step in the flux of compounds in the cascade of enzymatic synthesis steps provided with in the host cell in order to produce the glycoprotein according the invention.

The technical problem underlying the present invention is also solved by the provision of a modified or genetically engineered cell or host cell, particularly a eukaryotic cell, which comprises and expresses that novel LLO flippase activity.

The inventors surprisingly found that the provision of that novel type of “LLO flippase activity” with a relaxed specifity to the glycan structure of the LLO to be flipped is possible. This novel LLO flippase advantageously allows a genetic engineering of the glycosylation process that takes place at the membrane of an intracellular organelle, in particular at the ER membrane.

According to the first aspect of the invention there is provided a novel LLO flippase activity with relaxed specifity which is useful as a valuable tool for the modification and control of glycosylation in a host cell. In preferred embodiments, this modification of the host cell is combined with at least one or more genetic modifications of the building process of the LLO structures at the cytosolic side of the membrane and/or at the lumenal side of the organelle (see FIG. 1).

In more preferred embodiments these modifications are further combined with genetic modifications of the oligosaccharyl transferase activity in the organelle mediated the oligosaccharyltransfer to the nascent polypeptide at the end of the building process. These composite systems of modifications advantageously allows the provision of novel modified host cells, which in particular are specifically capable of synthesizing glycan structures consisting of 1, 2, or 3 mannose residues, in particular Man1GlcNAc2, Man2GlcNAc2, or Man3GlcNAc2, in intracellular organelles, and more particular the ER.

In a preferred aspect of the invention, the cell is further modified to lack or to have suppressed, diminished, or depleted one or more organelle- or ER-localized glycosyl transferase activities, in particular mannosyl transferase activities, and in particular to express instead heterologous glycosyltranferase activities and other enzymes necessary for hybrid or complex N-glycosylation of proteins.

In a second aspect of the invention there is provided a cell that is modified, alternatively or in addition, to comprise or express one or more organelle- or ER-localized modified, and in particular heterologous, oligosaccharyl transferase (OT) activities with a relaxed specifity for glycan structures to be transferred from LLO to the protein. In particular, the activity of such OT to transfer Man1GlcNAc2, Man2GlcNAc2, or Man3GlcNAc2 glycan structures is high. In particular, the activity of such OT to transfer Man1GlcNAc2, Man2GlcNAc2, or Man3GlcNAc2 glycan structures is high. In this context, the term “high” means that Man1GlcNAc2, Man2GlcNAc2, or Man3GlcNAc2 will be transferred to at least 20%, at least 40%, at least 60%, and preferably at least 80%, and most preferred at least 90% of the nascent proteins. The cell may be further characterized in that the cell comprises one or more nucleic acid molecules coding for oligosaccharyl transferase activity, characterized in that the activity not preferentially transfers Glc3Man9GlcNAc2 to a protein but is also capable of transferring oligosaccharides other than Glc3Man9GlcNAc2, preferably oligosaccharides having 1 to 9 mannose residues, most preferably, Man1GlcNAc2, Man2GlcNAc2, or Man3GlcNAc2 to a protein. More particular, the cell is characterized in that the activity not only transfers Glc3Man9GlcNAc2 to a protein but is also capable of efficiently transferring oligosaccharides other than Glc3Man9GlcNAc2, preferably oligosaccharides having 1 to 9 mannose residues (Man1GlcNAc2, Man2GlcNAc2, Man3GlcNAc2, Man4GlcNAc2 Man5GlcNAc2, Man6GlcNAc2, Man7GlcNAc2, Man8GlcNAc2 Man9GlcNAc2), most preferably, Man1GlcNAc2, Man2GlcNAc2, and/or Man3GlcNAc2 to a protein.

More particularly, the oligosaccharyl transferase (OT) activity is a single unit or protozoan-type OT which brings about OT activity in the form of a single protein unit. In a more particular embodiment the the derived from a protozoan organism, i.e. a protozoan OT (POT). The cell of this aspect is preferably further characterized in that protozoan oligosaccharyl transferase activity is derived from Toxoplasma gondii (Tg), Leishmania major (Lm); Leishmania infantum (Li), Leishmania braziliensis (Lb), Leishmania Mexicana (Lmx), Leishmania donovani (Ld), Leishmania guyanensis (Lg), Leishmania tropica (Lt), Trypanosoma cruzi (Tc), and Trypanosoma brucei (Tb). The invention also concerns homologous or artificial structures related to or derived from said POT which function to bring about POT activity in the cell.

In a particular aspect of the invention the cell is further modified to lack or to have suppressed, diminished, or depleted one or more Golgi-localized mannosyl transferase activities.

The cells of the invention preferably comprises one or more nucleic acid molecules that code for one or more, in particular heterologous and recombinant, glycoproteins and is capable of producing the glycoprotein or compositions of one or more thereof. The invention also provides the method or process to produce said glycoprotein or glycoprotein composition, wherein the method is primarily characterized in that the cell according the invention is provided and used to produce the glycoprotein. The invention also provides glycoproteins, and in particular novel glycoprotein compositions, that are producible or are produced by the cell of the invention.

The cells according to the invention exhibits an increased intralumenal concentration of Man1 to Man3 type LLO in comparison to an unmodified wild type strain of the host cell. In particular, intralumenal concentration is increased by at least 5%, 10%, 15%, 20%, 25%, 30%, 40%, 50%, 70%, or 90%, more particular by at least 100%, 200%, 500%, 700%, 1000%, 1500%, 2000% or more. The cell thus exhibits an increased glycosylation efficiency in comparison to an unmodified wild type strain of the host cell. In particular glycosylation is increased, in particular for Man3 based structures, by at least 5%, 10%, 15%, 20%, 25%, 30%, 40%, 50%, 70%, or 90%, more particular by at least 100%, 200%, 500%, 700%, 1000%, 1500%, 2000% or more.

In connection with ER knock out mutant strains, ie. strains having a modified glysosylation in the ER, in particular a alg modified pathway, such mutant strains if modified according to the invention exhibit an increased growth rate and/or a reduced temperature sensitivity in comparison to unmodified ER knock out mutant strains. In particular, growth rate in ER knock out mutant strains is increased by at least 5%, 10%, 15%, 20%, 25%, 30%, 40%, 50%, 70%, or 90%, more particular by at least 100%, 200%, 500%, 700%, 1000%, 1500%, 2000% or more.

A particular aspect of the invention relates to an isolated LLO flippase and to isolated nucleic acid molecules encoding said flippase. The flippase according to the invention is a protein which comprises at least one transmembrane-domain and at least one localization sequence for an intracellular membrane and is membrane bound. The flippase is further characterized in being capable of flipping a Man1GlcNAc2, Man2GlcNAc2, or Man3GlcNAc2 structure of a lipid-linked oligosaccharide across a membrane e.g. flipping said Man1GlcNAc2, Man2GlcNAc2, or Man3GlcNAc2 structure from the cytosolic into the lumenal side of said organelle. Said LLO flippase can be isolated according to the methods described further herein below. The invention further relates to an expression cassette and a vector for the expression of the flippase activity in a cell.

Further particular aspects of the invention relate to the use of said LLO flippase, preferably in combination with a oligosaccharyltransferase with relaxed specifity for glycan structures such as in particular a protozoan oligosaccharyl transferase (POT) or to use of any one of the cells according to the invention for the production of a glycoprotein or a composition comprising such glycoproteins. Other aspects of the invention relate to glycoproteins produced by and to kits comprising the cells of the invention and their use for the production of said glycoproteins.

More particular, in a first aspect the invention provides a cell or host cell modified to express LLO flippase activity that is capable of efficiently flipping lipid-linked all oligosaccharides comprising from 1 to 3 mannose residues from the cytosolic side to the lumenal side of an intracellular organelle.

In a particular aspect thereof the cell is further characterized in that said LLO flip-pase is active in efficiently flipping lipid-linked oligosaccharides selected from the group consisting of Man1GlcNAc2, Man2GlcNAc2, and Man3GlcNAc2.

In a preferred aspect thereof the cell is further characterized in that said LLO flip-pase activity is conferred by the expression of one or more of nucleic acid molecules, selected from the group consisting of:
a) nucleic acid molecules comprising or consisting of the sequence of one or more of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, and SEQ ID NO: 17; SEQ ID NO: 21, SEQ ID NO: 23, SEQ ID NO: 25, SEQ ID NO: 27, and SEQ ID NO: 29;
b) nucleic acid molecules, coding for a poly amino acid, comprising the sequence of one or more of: SEQ ID NO: 2, SEQ ID NO: 4, SEQ ID NO: 6, SEQ ID NO: 8, SEQ ID NO: 10, SEQ ID NO: 12, SEQ ID NO: 14; SEQ ID NO 16 and SEQ ID NO: 18; SEQ ID NO: 22, SEQ ID NO: 24, SEQ ID NO: 26, SEQ ID NO: 28, and SEQ ID NO: 30; and
c) fragments, variants, analogues or derivatives of the nucleic acid molecule of a) or b).

A cell of one of the preceding aspects may be further characterized in that the intra-cellular organelle is the endoplasmatic reticulum (ER).

A cell of one of the preceding claims may be further characterized in that the cell comprises at least one nucleic acid encoding a heterologous(glyco)protein and preferably expresses that (glyco)protein.

The cell may be further characterized in that the cell is lacking or is having suppressed, diminished or depleted Rft1-type LLO flippase activity. The cell of this aspect is preferably further characterized in that the Rft1-type LLO flippase is characterized in that its activity for flipping lipid-linked oligosaccharides having less than 5 mannose residues is less than its activity for flipping lipid-linked oligosaccharides with 5 mannose residues. More particular; a Rft1-type LLO flippase is characterized in that its activity for flipping lipid-linked oligosaccharides having less than 5 mannose residues is less than its activity for flipping lipid-linked oligosaccharides with 5 mannose residues, wherein “less” means less than 10%, 20%, 50%, 80% lipid-linked oligosaccharides having less than 5 mannose residues are being flipped when compared to the amount of lipid-linked oligosaccharides with 5 mannose residues.

The cell of this particular aspect is preferably further characterized in that the cell is a knock-out mutant of the gene rft1 or rft1 homologues.

The cell may be further characterized in that the cell is lacking or is having suppressed, diminished or depleted one or more of ER-localized glycosyl transferase activities. The cell of this aspect is preferably further characterized in that the ER-localized glycosyl transferase is a mannosyl transferase.

The cell may be further characterized in that the cell is lacking or is having suppressed, diminished or depleted one or more of ER-localized lipid-linked monosaccharide (LLM) flippase activities.

The cell may be further characterized in that the cell is lacking or is having suppressed, diminished or depleted Alg11-type activity. The cell of this aspect is preferably further characterized in that the cell is a knock-out mutant of the gene alg11 or alg11 homologues.

The cell may be further characterized in that the cell is lacking or is having suppressed, diminished or depleted Alg11-type activity and is further lacking or is having suppressed, diminished or depleted one or more lipid-linked monosaccharide (LLM) flippase type activities. The cell of this aspect is preferably further characterized in that the cell is a knock-out mutant of the gene alg11 or alg11 homologues and of one or more genes coding for lipid-linked monosaccharide (LLM) flippase activity.

The cell may be further characterized in that the cell is lacking or is having suppressed, diminished or depleted Alg11-type activity and is further lacking or is having a suppressed, diminished or depleted Alg3-type activity. The cell of this aspect is preferably further characterized in that the cell is a knock-out mutant of the gene alg11 or alg11 homologues and of alg3 or alg3 homologues.

The cell may be further characterized in that the cell is lacking or is having suppressed, diminished or depleted Alg11-type activity and is further lacking or is having suppressed, diminished or depleted beta-D-mannosyl transferase or DPMI-type activity. The cell of this aspect is preferably further characterized in that the cell is a knock-out mutant of the gene alg11 or alg11 homologues and of dpm1 or dpm1 homologues.

The cell may be further characterized in that the cell is lacking or is having suppressed, diminished or depleted Alg2-type activity. The cell of this aspect is preferably further characterized in that the cell is a knock-out mutant of alg2 or alg2 homologues.

The cell may be further characterized in that the cell comprises one or more nucleic acid molecules coding for oligosaccharyl transferase activity, characterized in that the activity not preferentially transfers Glc3Man9GlcNAc2 to a protein but is also capable of transferring oligosaccharides other than Glc3Man9GlcNAc2, preferably oligosaccharides having 1 to 9 mannose residues, most preferably, Man1GlcNAc2, Man2GlcNAc2, or Man3GlcNAc2 to a protein. More particular, the cell is characterized in that the activity not only transfers Glc3Man9GlcNAc2 to a protein but is also capable of efficiently transferring oligosaccharides other than Glc3Man9GlcNAc2, preferably oligosaccharides having 1 to 9 mannose residues (Man1GlcNAc2, Man2GlcNAc2, Man3GlcNAc2, Man4GlcNAc2 Man5GlcNAc2, Man6GlcNAc2, Man7GlcNAc2, Man8GlcNAc2 Man9GlcNAc2), most preferably, Man1GlcNAc2, Man2GlcNAc2, and/or Man3GlcNAc2 to a protein.

The cell of the preceding aspect is preferably further characterized in that the protozoan oligosaccharyl transferase activity is selected from the group consisting of: TbStt3Bp-type activity, TbStt3Cp-type activity, LmStt3Ap-type activity, LmStt3Bp-type activity, and LmStt3Dp-type activity.

The cell may be further characterized in that the cell is lacking or is having suppressed, diminished or depleted one or more Golgi-localized mannosyl transferase activity.

The cell of one or more of the preceding aspects is particularly characterized in that the Golgi-localized mannosyl transferase is selected from the group consisting of: Och1-type activity and the Mnn mannosyl transferase family, in particular Mnn1-type activity, Mnn2-type activity, Mnn4-type activity, Mnn5-type activity, Mnn9-type activity, Mnn10-type activity, and Mnn11-type activity. The cell of this aspect is preferably further characterized in that the cell is a knock-out mutant of at least one gene of: och1, mnn1, mnn2, mnn4, mnn5, mnn9, mnn10, mnn11 and/or the homologues thereof.

The cell of one or more of the preceding aspects is particularly characterized in that the Golgi-localized mannosyl transferase is selected from the group consisting of the Ktr mannosyl transferase family, in particular Ktr1-type activity, Ktr2-type activity, Ktr3-type activity, Ktr5-type activity, Ktr6-type activity, and Ktr7-type activity. The cell of this aspect is preferably further characterized in that the cell is a knock-out mutant of at least one gene of: ktr1, ktr2, ktr3, ktr4, ktr5, ktr6, ktr7 and/or the homologues thereof.

The cell of one or more of the preceding aspects is particularly characterized in that the Golgi-localized mannosyl transferase is selected from the group consisting of the Van mannosyl transferase family, in particular Van1-type activity and Vrg4-type activity. The cell of this aspect is preferably further characterized in that the cell is a knock-out mutant of at least one gene of: van1, vrg4 and/or the homologues thereof.

The cell of one preceding aspect is preferably further characterized in that the cell is lacking or is having a suppressed, diminished or depleted Mnn2-type activity and is further lacking or is having a suppressed, diminished or depleted Mnn5-type activity. The cell of the preceding aspect is preferably further characterized in that the cell is a knock-out mutant of the gene mnn2 or mnn2 homologues and of the gene mnn5 or mnn5 homologues.

The cell may be further characterized in that the cell is lacking or is having a suppressed, diminished or depleted Och1-type activity. The cell of this aspect is preferably further characterized in that the cell is a knock-out mutant of the gene och1 or och1 homologues.

The cell may be further characterized in that the cell expresses one or more Golgi-localized heterologous enzyme or catalytic domain thereof, preferably selected from the group consisting of:
mannosyl(alpha-1,3-)-glycoprotein beta-1,2-N-acetylglucosaminyl transferase (GnTI);
mannosyl(alpha-1,6-)-glycoprotein beta-1,2-N-acetylglucosaminyl transferase (GnTII);
beta-1,4-mannosyl-glycoprotein 4-beta-N-acetylglucosaminyl transferase (GnTIII);
mannosyl(alpha-1,3-)-glycoprotein beta-1,4-N-acetylglucosaminyl transferase (GnTIV);
mannosyl(alpha-1,6-)-glycoprotein beta-1,6-N-acetylglucosaminyl transferase (GnTV);
mannosyl(alpha-1,6-)-glycoprotein beta-1,4-N-acetylglucosaminyl transferase (GnTVI);
beta-N-acetylglucosaminyl glycopeptide beta-1,4-galactosyl transferase (GaIT);
alpha (1,6) fucosyl transferase (FucT);
beta-galactoside alpha-2,6-sialyl transferase (ST);
UDP-N-acetylglucosamine 2-epimerase (NeuC);
sialic acid synthase (NeuB);
CMP-Neu5Ac synthetase;
N-acylneuraminate-9-phosphate synthase;
N-acylneuraminate-9-phosphatase;
UDP-N-acetylglucosamine transporter;
UDP-galactose transporter;
GDP-fucose transporter;
CMP-sialic acid transporter;
nucleotide diphoshatases;
GDP-D-mannose 4,6-dehydratase; and
GDP-4-keto-6-deoxy-D-mannose-3,5-epimerase-4-reductase.

The cell may be further characterized in that the cell is selected from: lower eukaryotic cells including fungal cells and higher eukaryotic cells including mammalian cells, plant cells, and insect cells.

In a third aspect, the invention provides an isolated nucleic acid molecule or a plurality thereof, capable of coding for or conferring the LLO flippase activity as characterized in the first aspect of the invention. In a preferred aspect thereof, the nucleic acid molecule is characterized in that the molecule is selected from one or more of the nucleic acid molecules as characterized in one of the preceding aspects of the invention.

In a forth aspect, the invention provides an expression cassette for the expression in a eukaryotic host cell, comprising one or more copies of one of the nucleic acid molecules as characterized in one of the preceding aspects of the invention, in conjunction with at least one of: nucleic acid molecules coding for a promoter and nucleic acid molecules coding for a terminator.

In a preferred aspect thereof, the expression cassette, is further comprising one or more copies of a nucleic acid molecule coding for oligosaccharyl transferase activity as characterized in one of the preceding aspects of the invention.

In a fifth aspect, the invention provides a vector for the transformation of a eukaryotic host cell, the vector is comprising one or more selected from: copies of one of the nucleic acid molecules as characterized in one of the preceding aspects of the invention and one or more copies of the expression cassette as characterized in one of the preceding aspects of the invention.

In a sixth aspect, the invention provides a method for the production of a cell that is specifically capable of the synthesis of lipid-linked oligosaccharides having a Man1GlcNAc2, Man2GlcNAc2, or Man3GlcNAc2 glycan structure in the intracellular organelle endoplasmatic reticulum, the method comprising at least the step(s) of:

transforming the cell with at least one construct or structure coding for LLO flippase activity selected from the group of:
nucleic acid molecules as characterized in one of the preceding aspects of the invention;
expression cassettes as characterized in one of the preceding aspects of the invention; and
vectors as characterized in one of the preceding aspects of the invention,

such that the cell is able to express LLO flippase activity encoded by that construct or structure.

In a preferred aspect thereof, the construct further codes for oligosaccharyl transferase activity, such that the cell is able to express LLO flippase activity and oligosaccharyl transferase activity encoded by that structure.

In a preferred aspect of one or more of the preceding aspects the method is further comprising the step(s) of diminishing or depleting in the cell at least one enzyme activity selected from the group of:
Alg2-type activity;
Alg11-type activity;
Alg3-type activity;
DPM1-type activity; and
lipid-linked monosaccharide (LLM) flippase-type activity.

In a seventh aspect, the invention provides an isolated cell or a plurality thereof, that specifically capable of synthesizing lipid-linked oligosaccharides having a Man1GlcNAc2, Man2GlcNAc2, or Man3GlcNAc2 glycan structure in an intracellular organelle and transferring the glycan structure to a nascent protein expressed in that cell, characterized in that the cell is producible or actually produced according to the method of one of the preceding aspects of the invention.

In an eighth aspect, the invention provides a method for the production of a glycoprotein or a glycoprotein-composition, comprising the step(s) of:
providing a cell according to one of the preceding aspects of the invention;
culturing the cell in a culture medium under conditions that allow the production of the glycoprotein or glycoprotein-composition in said cell; and,
if necessary, isolating the glycoprotein or glycoprotein-composition from said cell and/or said culture medium.

In a ninth aspect, the invention provides a kit or kit-of-parts for producing glycoprotein, comprising:
the cell according to one of the preceding aspects of the invention and
culture medium for culturing the cell so as to confer the production of the glycoprotein.

In a tenth aspect, the invention provides glycoprotein or glycoprotein composition, characterized in that the glycan structures thereof are selected from:
GlcNAcMan3-5GlcNAc2,
GlcNAc2Man3GlcNAc2,
GlcNAc3Man3GlcNAc2-bisecting
Gal2GlcNAc2Man3GlcNAc2,
Gal2GlcNAc2Man3GlcNAc2Fuc,
Gal2GlcNAc3Man3GlcNAc2-bisecting,
Gal2GlcNAc3Man3GlcNAc2Fuc-bisecting,
NeuAc2Gal2GlcNAc2Man3GlcNAc2, NeuAc2Gal2GlcNAc2Man3GlcNAc2Fuc,
NeuAc2Gal2GlcNAc3Man3GlcNAc2-bisecting,
NeuAc2Gal2GlcNAc3Man3GlcNAc2Fuc-bisecting,
GlcNAc3Man3GlcNAc2,
Gal3GlcNAc3Man3GlcNAc2,
Gal3GlcNAc3Man3GlcNAc2Fuc,
NeuAc3Gal3GlcNAc3Man3GlcNAc2, and
NeuAc3Gal3GlcNAc3Man3GlcNAc2Fuc.

In an eleventh aspect, the invention provides a host cell, specifically capable of producing one or more of the glycoprotein or glycoprotein composition as characterized in the ninth aspect of the invention.

In a twelfth aspect, the invention provides a glycoprotein, selected from:
glycoproteins, producible by the cell according to one of the preceding aspects of the invention,
glycoproteins, producible by the method according to one of the preceding aspects of the invention; and
glycoproteins according to the tenth aspect of the invention.

A preferred aspect thereof is a glycoprotein composition, comprising two or more of the glycoproteins according to the tenth aspect.

A preferred aspect thereof is a recombinant protein or a plurality thereof. A preferred aspect thereof is a therapeutically active protein or a plurality thereof.

A preferred aspect thereof is an immunoglobulin or a plurality of immunoglobulins.

In a thirteenth aspect, the invention provides a pharmaceutical composition, comprising: one or more of the glycoprotein of one of the preceding aspects of the invention and preferably at least one pharmaceutically acceptable carrier or adjuvant.

In a fourteenth aspect, the invention provides a method of treating a disorder that is treatable by administration of one or more of the glycoproteins or compositions of one or more of the preceding aspects, comprising the step(s) of: administering to a subject the glycoprotein or composition as described above, wherein the subject is suffering from, or is suspected to, a disease treatable by administration of that glycoprotein or composition.

### DETAILED DESCRIPTION OF THE INVENTION

The present invention primarily relates to host cells having modified lipid-linked oligosaccharides which may be modified further by heterologous expression of a set of glycosyltransferases, sugar transporters and mannosidases to become host-strains for the production of mammalian, e.g., human therapeutic glycoproteins. The process provides an engineered host cell which can be used to express and target any desirable gene(s) involved in glycosylation. Host cells with modified lipid-linked oligosaccharides are created or selected. N-glycans made in the engineered host cells have a Man1GlcNAc2, Man2GlcNAc2, and/or Man3GlcNAc2 core structure which may then be modified further by heterologous expression of one or more enzymes, e.g., glycosyl-transferases, sugar transporters and mannosidases, to yield humanlike glycoproteins. For the production of therapeutic proteins, this method may be adapted to engineer cell lines in which any desired glycosylation structure may be obtained.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present invention are generally performed according to conventional methods well known in the art. Generally, nomenclatures used in connection with, and techniques of biochemistry, enzymology, molecular and cellular biology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane Antibodies: A Laboratory Manual Cold Spring Harbor Laboratory Press, Cold Spring Harbor, N.Y. (1990); Introduction to Glycobiology, Maureen E. Taylor, Kurt Drickamer, Oxford Univ. Press (2003); Worthington Enzyme Manual, Worthington Biochemical Corp. Freehold, N.J.; Handbook of Biochemistry: Section A Proteins Vol I 1976 CRC Press; Handbook of Biochemistry: Section A Proteins Vol II 1976 CRC Press; Essentials of Glycobiology, Cold Spring Harbor Laboratory Press (1999). The nomenclatures used in connection with, and the laboratory procedures and techniques of, biochemistry and molecular biology described herein are those well known and commonly used in the art.

Provision of Novel LLO Flippases

In the context of the present invention, a “LLO flippase activity” or “flippase” is defined as the function of translocating lipid-linked, particularly dolichol linked, oligosaccharides (LLO) that are bound to the membrane of an intracellular organelle, primarily at the cytosolic side of the membrane, from the cytosolic side through the membrane and to the lumenal side of the organelle. In particular, the intracellular organelle is the endoplasmatic reticulum (ER). This process of translocation of the LLO is being characterized as “flipping”. In a preferred embodiment, the flippase activity is targeted to the ER. Without whishing to be bound the theory, the terms “flippase” and “flipping” also refer to a supportive action for supporting another potential flippase protein to bring about flippase activity.

It has surprisingly been found that novel LLO flippases are isolatable and functional in a glycosylation cascade of a cell, and that they are able to compensate for a decrease or lack of endogenous LLO flippase activity such as for example the Rft1 type activity. Furthermore, it has surprisingly been found that a LLO flippase activity according to the invention is able to function in an altered glycosylation cascade. Said alterations comprise the generation and flipping across the ER membrane of lipid-linked oligosaccharides having less oligosaccharides such as for example LLO comprising less than 5 mannose residues. Such LLO structures are usually not predominantly produced or flipped in a wild-type cell. It has been surprisingly found that the novel LLO flippase is efficiently active in flipping lipid-linked oligosaccharides comprising less than 5 mannose residues, in particular Man1GlcNAc2, Man2GlcNAc2, Man3GlcNAc2, or Man4GlcNAc2, across the membrane of an intracellular organelle. The novel LLO flippase exhibits high actitivity in flipping lipid-linked oligosaccharides comprising Man5GlcNAc2; it exhibits high actitivity in flipping lipid-linked oligosaccharides comprising Man4GlcNAc2; it exhibits high actitivity in flipping lipid-linked oligosaccharides comprising Man3GlcNAc2; it exhibits still high actitivity in flipping lipid-linked oligosaccharides comprising Man3GlcNAc2; it exhibits still high actitivity in flipping lipid-linked oligosaccharides comprising Man2GlcNAc2; and exhibits still high actitivity in flipping lipid-linked oligosaccharides comprising Man1GlcNAc2. The novel LLO flippase is found to exhibit a “relaxed” specifity in respect to the oligosaccharyl structure to be flipped.

Without wishing to be bound to the theory, the term “activity” as used herein in particular for LLO flippase concerns the rate of transport, transfer or synthesis specific for a certain compound or molecule to be transported or synthesized. In connection with a trans-membrane transport of a molecule the transport activity as expressed rate of transport is assessed by assessing the net flux of the specific molecule or structure to be transporter over a biological barrier, and more particular is “flipped” over or through the membrane of an intracellular organelle. The net flux is calculated in particular from the intake rate and the outflow rate. It is found that the net flux may be dependent to a great extent on the molecular structure of the transported molecule. Net flux, and in turn, transport activity may be specific for each individual structure to be transportet or flipped. Without wishing to be bound to the theory, flippase activity may be calculated by determining the amount of incorporated labeled mannose into the LLO present on the cytoplasmatic side of the ER and dividing said number by the total amount of labelled mannose, preferably [3H]-mannose, incorporated into the LLO. Alternatively, the LLO flippase activity may be determined using “artificial” vesicles. For example, in LLO flippase of Rft1-type the activity to flip LLO with Man5GlcNAc2 structure is high, but is found to be low, if any, for LLO with Man1GlcNAc2 structure. LLO flippase of Rft1-type thus exhibits high specifity for flipping Man5GlcNAc2 structures. In contrast, in the novel LLO flippase according the invention the activity to flip LLO with Man1GlcNAc2 structure is high and the activity to flip LLO with Man2GlcNAc2 or Man3GlcNAc2 structure is also high. The novel LLO flippase according the invention exhibits activity which is less specific to a certain glycan structure, thus exhibits a “relaxed” or less specified flippase activity.

A gene or an “artificial” gene encoding LLO flippase activity according to the invention may be isolated, in a preferred example from yeast cells, by way of a high copy suppressor screen (HCSS) as outlined in detail in the enclosed examples. In short, a cell in which the endogenous LLO flippase has been inactivated such as for example, a yeast cell carrying a deletion of the rft1 gene, may be used in a HCSS. Said cells may then be transformed with a genomic DNA library, such as a genomic yeast DNA library, expressed from a high copy plasmid such as for example Yep352, also carrying a selectable marker. Cells having a defect in the glycosylation cascade will produce hypoglycosylated proteins, and have increased temperature as well as osmotic sensitivity. Accordingly, selected cells obtained in the HCSS are tested for their ability to grow in the absence of an osmotic stabilizer such as for example sorbitol. Positive colonies may then be further analyzed in respect to their temperature sensitivity and their ability to glycosylate expressed proteins.

The present invention also relates to an isolated nucleic acid or pluralty thereof encoding a novel LLO flippase polypeptide having a novel LLO flippase activity, a vector including the isolated nucleic acid, and a cell comprising this vector.

In a particular embodiment the invention provides an “artificial” novel LLO flippase activity, which is a transcript of flc2′. The “artificial” gene flc2′ is derived from the flc2 gene (synonymous name: YAL053W; located on yeast chromosome 1; bases 45900 to 48251). The Flc2-transcript is a putative FAD transporter, that is localized in the ER-membrane and functions to import FAD into the ER. The endogenous Flc2-protein does not function as a flippase and does not transport LLOs.

The “artificial” flc2′ is primarily a 3′ truncated version of flc2. The full sequence of flc2′ is listed SEQ ID NO: 1 (FIG. 5A) and represents yeast chromosome 1, bases 45900 to 47222. The transcript of flc2′ yields a protein of 452 amino acids which comprises four complete transmembrane domain and a fifth truncated transmembrane domain (SEQ ID NO 2; FIG. 5B). The C-terminal 11 amino acids from amino acids 442 to 452 originate from the cloning procedure. Unexpectedly Flc2′, i.e. the N-terminal fragment of Flc2, is able to compensate for lacking flippase activity in a Δrft1 mutant strain, whereas the full length Flc2 itself does not exhibit flip-pase activity at all. More particular, the Flc2′ flippase was found to exhibit a great affinity to the Man1 structure and flipps the Man1 structure at a high rate.

The invention provides several “artificial” genes or gene constructs that encode a novel LLO flippase according to the invention. These are all derived from the flc2 gene. In particular, fragments of “artificial” flc2′ and constructs of one or more of these fragments are provided. The invention is not limited to these sequences. The invention concerns particularly “artificial” genes or gene constructs that exhibit the novel LLO flippase type functionality as characterized and described herein. The inventors surprisingly found that “artificial” transmembrane proteins can be construed or are available which are localized in the membrane of an intracellular organelle and confer the flipping of LLOs into the organelle lumen. These proteins exhibit the novel LLO flippase activity that is primarily characterized in a relaxed specifity in respect to the glycan structure of the LLO as described herein.

After the pioneering “proof of principle” as provided herein, primarily in form of “artificial” genes or gene constructs derived from flc2, further “artificial” genes or gene constructs that code for LLO flippase activity of analogous functionality can be easily provided by the skilled person, simply by pursuing the screening method as described herein below.

The invention, alternatively or in addition, provides gene constructs that are based, and in particular include, the rft1 gene or an polynucleotide coding for Rft1 or Rft1-type activity to bring about the LLO flippase activity in a cell, in particular a genetically modified cell where Rft1 is present in high concentration by way of overexpression of rft1, and means to produces such cells.

In a preferred embodiment the LLO flippase activity is embodied in one or more protein or protein-like structures, such as multi-unit transporters.

According to the invention, there is provided an isolated or “substantially pure” nucleic acid molecule or a functional analog thereof, which is capable of encoding or conferring the flippase activity as characterized hereinabove. In preferred embodiments the nucleic acid molecule is selected from one or more of the nucleic acid molecules as characterized herein below.

The terms “polynucleotide” or “nucleic acid molecule” refer to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hair-pinned, circular, or in a padlocked conformation. The term includes single and double stranded forms of DNA.

An “isolated” or “substantially pure” nucleic acid or polynucleotide (e.g., an RNA, DNA or a mixed polymer) is one which is substantially separated from other cellular components that naturally accompany the native polynucleotide in its natural host cell, e.g., ribosomes, polymerases, and genomic sequences with which it is naturally associated. The term embraces a nucleic acid or polynucleotide that (1) has been removed from its naturally occurring environment, (2) is not associated with all or a portion of a polynucleotide in which the “isolated polynucleotide” is found in nature, (3) is operatively linked to a polynucleotide which it is not linked to in nature, or (4) does not occur in nature.

The term “isolated” also can be used in reference to recombinant or cloned DNA isolates, chemically synthesized polynucleotide analogs, or polynucleotide analogs that are biologically synthesized by heterologous systems. However, “isolated” does not necessarily require that the nucleic acid or polynucleotide so described has itself been physically removed from its native environment. For instance, an endogenous nucleic acid sequence in the genome of an organism is deemed “isolated” herein if a heterologous sequence (i.e., a sequence that is not naturally adjacent to this endogenous nucleic acid sequence) is placed adjacent to the endogenous nucleic acid sequence, such that the expression of this endogenous nucleic acid sequence is altered. By way of example, a non-native promoter sequence can be substituted (e.g., by homologous recombination) for the native promoter of a gene in the genome of a human cell, such that this gene has an altered expression pattern. This gene would now become “isolated” because it is separated from at least some of the sequences that naturally flank it. A nucleic acid is also considered “isolated” if it contains any modifications that do not naturally occur to the corresponding nucleic acid in a genome. For instance, an endogenous coding sequence is considered “isolated” if it contains an insertion, deletion or a point mutation introduced “artificially”, e.g., by human intervention. An “isolated nucleic acid” also includes a nucleic acid integrated into a host cell chromosome at a heterologous site, a nucleic acid construct present as an episome. Moreover, an “isolated nucleic acid” can be substantially free of other cellular material, or substantially free of culture medium when produced by recombinant techniques, or substantially free of chemical precursors or other chemicals when chemically synthesized.

In a primary aspect the invention concerns nucleic acid molecules derived from flc2 and coding for the LLO flippase activity. In the preferred embodiments of that aspect the nucleic acid molecule carry at least the sequences of the ER localization signal and of one or more of transmembrane regions.

In preferred embodiments said LLO flippase activity in the host cell is conferred by the expression of one or more of nucleic acid molecules, selected from:
nucleic acid molecules comprising or consisting of the sequence of one or more of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15; and SEQ ID NO: 17;
nucleic acid molecules that code for a poly amino acid comprising or consisting of the sequence of one or more of: SEQ ID NO: 2, SEQ ID NO: 4, SEQ ID NO: 6, SEQ ID NO: 8, SEQ ID NO: 10, SEQ ID NO: 12, SEQ ID NO: 14; SEQ ID NO: 16, and SEQ ID NO: 18;
nucleic acid molecules comprising or consisting of the sequence of one or more of: SEQ ID NO: 21, SEQ ID NO: 23, SEQ ID NO: 25, SEQ ID NO: 27, and SEQ ID NO: 29, particularly when fused to one or more nucleic acid molecules that code for an ER localization signal, preferably selected from one of SEQ ID NO: 19 and nucleotide sequences coding for poly amino acid sequences comprising the HDEL motif and/or the KKxx motif;
nucleic acid molecules coding for a poly amino acid comprising or consisting of the sequence of one or more of: SEQ ID NO: 22, SEQ ID NO: 24, SEQ ID NO: 26, SEQ ID NO: 28, and SEQ ID NO: 30, particularly further comprising one or more ER localization signals, preferably selected from one of SEQ ID NO: 20 and poly amino acid sequences comprising the HDEL motif and/or the KKxx motif; and
fragments, variants, analogues or derivatives of the above identified nucleic acid molecules, conferring the LLO flippase activity of the invention.

The term “fragment” as used herein, refers to a segment of a polynucleotide. Fragments can have terminal (5′- or 3′-ends) and/or internal deletions. Generally, fragments of a polynucleotide will be at least four, in particular at least five, at least six, at least seven, at least eight, at least nine, at least 10, at least 12, at least 15, at least 18, at least 25, at least 30, at least 35, at least 40, at least 50, at least 60, at least 65, at least 70, at least 75, at least 80, at least 85, at least 90, or at least 100 or more, nucleotides in length.

The term “deletion” as used herein refers to variants of nucleotide sequence where one, two, three, four, five, six, seven, eight, nine, ten, 11, 12, 13, 14, 15, 16, 17, 18, 19, or 20 polynucleotide segments (of two or more nucleotides) are missing or deleted from the nucleotide sequence.

The term “addition” as used herein refers to variants of nucleotide sequence where one, two, three, four, five, six, seven, eight, nine, ten, 11, 12, 13, 14, 15, 16, 17, 18, 19, or 20 polynucleotide segments (of two or more nucleotides) are added or fused to the nucleotide sequence. Addition variants also include fusion molecules.

It is understood that in the preferred variants of the above mentioned modifications, in particular by addition or deletion of one or more nucleotides a frameshift is avoided, by adding or deleting a number of nuceotides which is three or an integer multiple thereof.

The term “analogue” or “analog” as used herein, primarily refers to compounds that are structurally similar (analog) to naturally occurring RNA and DNA. Nucleic acids are chains of nucleotides, which are composed of three parts: a phosphate back-bone, a pucker-shaped pentose sugar, either ribose or deoxyribose, and one of four nucleobases. An analogue may have any of these altered, typically the analogue nucleobases confer, among other things, different base pairing and base stacking proprieties such as universal bases, which can pair with all four canon bases, while the phosphate-sugar backbone analogues affect the properties of the chain, such as PNA (Petersson B et al. Crystal structure of a partly self-complementary peptide nucleic acid (PNA) oligomer showing a duplex-triplex network. J Am Chem Soc. 2005 Feb. 9; 127(5):1424-30), the secondary structure of which differs significantly from DNA, and may form a triplex (a triple stranded helix).

A preferred embodiment is an isolated nucleic acid molecule or a plurality thereof that is selected from: (a) the nucleic acid molecules as characterized above and (b) nucleic acid molecules that hybridize under highly stringent conditions to the complement of the nucleic acid molecules of (a). Highly stringent conditions are commonly defined as equivalent to hybridization in 6× sodium chloride/sodium citrate (SSC) at 45° C., followed by a wash in 0.2×SSC, 0.1% SDS at 65° C.

Preferred variants of that embodiment are isolated nucleic acid molecules that comprise or consist of a sequence that is at least 80% identical to any of the nucleic acid sequences described herein.

An “ER localization signal” refers to a peptide sequence which directs a protein having such peptide sequence to be transported to and retained in the ER. Such ER localization sequences are often found in proteins that reside and function in the ER. ER localization or “retention” signals are available to those skilled in the art, for example, the first 21 amino acid residues of the S. cerevisiae ER protein MNS1 (Martinet et al. Biotechnology Letters 20: 1171-1177, 1998). A preferred ER localization signal for use in the present invention is peptide HDEL (SEQ ID NO: 31). The HDEL peptide sequence, found in the C-terminus of a number of yeast proteins, acts as a retention/retrieval signal for the ER (Pelham EMBO J. 7: 913-918, 1988). Proteins with an HDEL sequence are bound by a membrane-bound receptor (Erd2p) and then enter a retrograde transport pathway for return to the ER from the Golgi apparatus.

Alternatively, a KKxx sequence can provide ER localization (Jackson J. Cell Biol. 121:317). This motif is present on several endogenous ER membrane proteins. This sequence can be present either on the N- or C-terminus of the protein and is retrieved from a post-ER compartment.

The primary aspect of this invention is to provide tools and means for the modification or genetic engineering of suitable host cells (see below) and to confer altered and more suitable N-glycosylation in that cell.

Accordingly, there is also provided an expression cassette or a functional analog thereof for the expression of the novel LLO flippase activity as characterized above in a eukaryotic host cell, comprising one or more copies of one of the nucleic acid molecules as characterized above. The nucleic acid sequence in the vector can be operably linked to an expression control sequence. Preferably, one or more of said nucleic acid molecules are present in conjunction with at least one of: nucleic acid molecules encoding a promoter and nucleic acid molecules encoding a terminator.

As used herein, a “promoter” refers to a DNA sequence that enables a gene to be transcribed. The promoter is recognized by RNA polymerase, which then initiates transcription. A promoter contains a DNA sequence that is either bound directly by, or is involved in the recruitment, of RNA polymerase. A promoter sequence can also include “enhancer regions,” which are one or more regions of DNA that can be bound with proteins (namely, the trans-acting factors, much like a set of transcription factors) to enhance transcription levels of genes (hence the name) in a gene-cluster. The enhancer, while typically at the 5′ end of a coding region, can also be separate from a promoter sequence and can be, e.g., an intrinsic region of a gene or 3′ to the coding region of the gene.

According to the present invention the promoter is preferably the endogenous promoter of the gene. In a preferred embodiment the gene is on a high copy number plasmid which preferably leads to overexpression. In another preferred embodiment the gene is on a low copy number plasmid. The promoter may be a heterologous promoter. In a particular variant the promoter is a constitutive promoter. In another particular variant the promoter is an inducible promoter. A particular promoter according to the invention confers an overexpression of one or more copies of the nucleic acid molecule. In preferred embodiments, the molecule(s) is overexpressed two times, more preferred 5 times, 10 times, 20 times, 50 times, 100 times, 200 times, 500 times, 1000 times, and most preferred 2000 or more times when compared to expression from endogenous promoter. For example, where the host cell is Pichia pastoris, suitable promoters include, but are not limited to, aox1, aox2, das, gap, pex8, ypt1, fld1, and p40; where the host cell is Saccharomyces cerevisiae suitable promoters include, but are not limited to, gall, mating factor a, cyc-1, pgk1, adh2, adh, tef, gpd, met25, galL, galS, ctr1, ctr3, and cup1. Where the host cell, for example, is a mammalian cell, suitable promoters include, but are not limited to CMV, SV40, actin promoter, rps21, Rous sarcoma virus genome large genome long terminal repeats (RSV), metallothionein, thymidine kinase or interferon gene promoter.

A “terminator” or 3′ termination sequences are able to the stop codon of a structural gene which function to stabilize the mRNA transcription product of the gene to which the sequence is operably linked, such as sequences which elicit polyadenylation. 3′ termination sequences can be obtained from Pichia or other methylotrophic yeast or other yeasts or higher fungi or other eukaryotic organisms. Examples of Pichia pastoris 3′ termination sequences useful for the practice of the present invention include termination sequences from the aox1 gene, p40 gene, his4 gene and fld1 gene.

According to the invention, there is also provided a vector for the transformation of a eukaryotic host cell, comprising one or more copies of one of the nucleic acid molerules characterized above or one or more copies of the expression cassette as characterized above.

The term “vector” as used herein is intended to refer to a nucleic acid molecule capable of transporting another nucleic acid to which it has been linked. One type of vector is a “plasmid”, which refers to a circular double stranded DNA loop into which additional DNA segments may be ligated. Other vectors include cosmids, bacterial “artificial” chromosomes (BAC) and yeast “artificial” chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply, “expression vectors”).

The vectors of the present invention preferably contain a selectable marker gene. Examples of such systems include the Saccharomyces cerevisiae or Pichia pastoris his4 gene which may be used to complement his4 Pichia strains, or the S. cerevisiae or Pichia pastoris arg4 gene which may be used to complement Pichia pastoris arg mutants, or the Pichia pastoris ura3 and ade1 genes, which may be used to complement Pichia pastoris ura3 or ade1 mutants, respectively. Other selectable marker genes which function in Pichia pastoris include the zeo^{R} gene, the g418^{R} gene, blastisidin resistance gene, and the like.

The vectors of the present invention can also include an autonomous replication sequence (ARS). The vectors can also contain selectable marker genes which function in bacteria, as well as sequences responsible for replication and extrachromosomal maintenance in bacteria. In alternative embodiments the selection is conferred by auxothrophic markers. Examples of bacterial selectable marker genes include ampicillin resistance (amp^{r}), tetracycline resistance (tet^{r}), neomycin resistance, hygromycin resistance and zeocin resistance (zeo^{R}) genes.

The invention also provides respective means for direct genetic integration. The nucleotide sequence according to the invention, encoding the protein to be expressed in a cell may be placed either in an integrative vector or in a replicative vector (such as a replicating circular plasmid). Integrative vectors generally include serially arranged sequences of at least a first insertable DNA fragment, a selectable marker gene, and a second insertable DNA fragment. The first and second insertable DNA fragments are each about 200 nucleotides in length and have nucleotide sequences which are homologous to portions of the genomic DNA of the species to be transformed. A nucleotide sequence containing a structural gene of interest for expression is inserted in this vector between the first and second insertable DNA fragments whether before or after the marker gene. Integrative vectors can be linearized prior to yeast transformation to facilitate the integration of the nucleotide sequence of interest into the host cell genome.

The invention also provides a poly amino acid molecule, in particular a protein, or a plurality thereof, that is capable of flipping of lipid-linked, truncated or complete precursor oligosaccharides (LLO), in particular Man1GlcNAc2, Man2GlcNAc2 and/or Man3GlcNAc2. The terms “polyaminoacid molecule” “polypeptide” and “protein” are used interchangeably and mean any peptide-linked chain of amino acids, regardless of length or post-translational modification.

In a particular and preferred embodiment of the invention, the molecule comprises or substantially consists of a fragment that codes for transmembrane domain 4 (TM4) of Flc2′ or a homologous functional structure thereof. In a particular and preferred embodiment thereof, the molecule comprises or substantially consists of a fragment that codes for transmembrane domains 3 to 4 (TM3-4) of Flc2′ or a homologous functional structure thereof.

The molecule may comprise or substantially consist of a fragment that codes for transmembrane domain 1 (TM1) of Flc2′ or a homologous functional structure thereof. The molecule may also comprise or substantially consist of a fragment that codes for transmembrane domain 2 (TM3) of Flc2′ or a homologous functional structure thereof. In a particular and preferred embodiment thereof, the molecule comprises or substantially consists of a fragment that codes for transmembrane domains 1 to 2 (TM1-2) of Flc2′ or a homologous functional structure thereof. In another embodiment thereof, the molecule comprises or substantially consists of a fragment that codes for transmembrane domains 2 to 4 (TM2-4) of Flc2′ or a homologous functional structure thereof.

The molecule may comprise or substantially consist of a fragment that codes for transmembrane domain 3 (TM3) of Flc2′ or a homologous functional structure thereof. In a particular embodiment thereof, the molecule comprises or substantially consists of a fragment that codes for transmembrane domains 1 to 3 (TM1-3) of Flc2′ or a homologous functional structure thereof. In another embodiment thereof, the molecule comprises or substantially consists of a fragment that codes for transmembrane domains 2 to 3 (TM2-3) of Flc2′ or a homologous functional structure thereof.

In a primary aspect, the poly amino acid is a transcript of one or more of the above-identified “artificial” constructs derived from flc2′ and including flc2′. In a preferred embodiment the transcript is comprising or is consisting of the sequence of one or more of: SEQ ID NO: 2, SEQ ID NO: 4, SEQ ID NO: 6, SEQ ID NO: 8, SEQ ID NO: 10, SEQ ID NO: 12, SEQ ID NO: 14; SEQ ID NO: 16 and SEQ ID NO: 18.

In another preferred embodiment the transcript is comprising or is consisting of the sequence of one or more of: SEQ ID NO: 22, SEQ ID NO: 24, SEQ ID NO: 26, SEQ ID NO: 28, and SEQ ID NO: 30, fused to an ER localization signal, preferably selected from one of SEQ ID NO: 20 and poly amino acid sequences comprising the HDEL and KKxx motif.

In another preferred embodiment the poly amino acid molecule is a fragment, analog and derivative of one or more of the above-identified transcripts. As used herein “fragment”, “analog” and “derivative” of transcripts refer to biologically active variants that may contain additions, deletions, or substitutions.

Variants with substitutions preferably have not more than 50, in particular, not more than one, two, three, four, five, six, seven, eight, nine, ten, 12, 15, 20, 25, 30, 35, 40, or 45, conservative amino acid substitutions. A “conservative substitution” is understood as the substitution of one amino acid for another with similar characteristics. Conservative substitutions include substitutions within the following groups: valine, alanine and glycine; leucine, valine, and isoleucine; aspartic acid and glutamic acid;

asparagine and glutamine; serine, cysteine, and threonine; lysine and arginine; and phenylalanine and tyrosine. The non-polar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid, and glutamic acid. By contrast, a non-conservative substitution is a substitution of one amino acid for another with similar characteristics.

The poly amino acid molecule according to the invention exhibits or confers a LLO flippase activity as described herein. It is particularly characterized in being capable of flipping of lipid-linked, truncated or complete precursor oligosaccharides (LLO), in particular Man1GlcNAc2, Man2GlcNAc2 and/or Man3GlcNAc2.

Without wishing to be bound to the theory, the activity or specificity of a LLO flippase or fragments, variants, analogues or derivatives may be measured by methods known to the person skilled in the art. Without wishing to be bound to the theory, a preferred method for assessing the LLO flippase activity according to the invention may comprise the following steps: growing and culturing of a cell that is expressing a protein which is a putative LLO flippase; exposing the cell to a labeled mannose substrate, in particular radioactively labeled [3H]-mannose, for a certain period of time and at a certain temperature (labeling); and isolating the mannose labeled LLO; and assaying the oligosaccharide content of the [3H]-mannose labeled LLOs. [3H]-labeled LLO may be isolated as described in detail in the examples included herein. The oligosaccharide content of the [3H]-mannose labeled LLOs may be analyzed by appropiate detection methods such as for example mass spectrometry (e.g. MALDI-TOF-MS) or high-pressure liquid chromatography (HPLC). Flippase activity may then be calculated by determining the amount of incorporated [3H]-mannose into the LLO present on the cytoplasmatic side of the ER and dividing said number by the total amount of [3H]-mannose incorporated into the LLO. A cell not capable of flipping LLO of a certain glycan structure will accumulate cytoplasmic LLO. For example, a putative LLO flippase according to the invention is positively detected when LLO having a Man5GlcNAc2 structure is flipped and being further modified by mannosyltransferases in the ER lumen.

In wild-type cells a LLO having a Man5GlcNAc2 structure is the substrate of the LLO flippase such as for example the Rft1 flippase. Wild-type cells expressing a functional flippase will produce mainly lumenal LLO which are further processed to the final LLO having Glc3Man9GlcNAc2 structure. Whereas cells lacking or having a defect in the LLO flippase, such as for example a rft1 knock out cell, produce mainly LLO having a Man5GlcNAc2 structure being present and measurable on the cytoplasmic side of the ER, indicating a block of translocating the LLO into the lumen of the ER, i.e. a block of further processing of the LLO to the final, ER luminal LLO having a Glc3Man9GlcNAc2 structure.

Alternatively, the LLO flippase activity or specificity may be determined using “artificial” vesicles. Such vesicles may be generated by extracting ER-membranes from cells. Reconstituting vesicles from such membranes depleted for endogenous LLO flippase such as for example Rft1 and equipping those vesicles with new LLO flippases allows to determine flippase activities of said new proteins. For labeling [3H]-Mannose is added and the cytoplasmic mannosyl transferases activities incorporate the [3H]-mannose into the LLO on the cytoplasmic side. The LLO may then be flipped into the ER lumen by way of an active LLO flippase. By treating the vesicles with an Endo H enzyme, LLO exposed on the surface of the vesicles are trimmed leaving only the terminal GlcNAc residue on the Dolichol lipid and thereby removing the radioactive label from the surface of the vesicle. By quantifying the amount of radioactivity present in Endo H treated [3H]-mannose in the lumen of the vesicles versus not Endo H treated vesicles the amount of flipping can then be calculated; wherein the less radioactivity is determined, the less active or specific a LLO flippase is for a certain LLO.

The specificity or activity of a LLO flippase for certain types of LLO varying in their oligosaccharide structure may be determined by using cells lacking or having a defect in at least one of the cytoplasmic mannosyl transferases. For example, cells having a defect in the Alg2-type activity will produce LLO having Man1GlcNAc2 or Man2GlcNAc2 structures; whereas cells lacking or having a defect in Alg11- and optionally Alg3-type activity will generate LLO having a Man3GlcNAc2 structure. Such mutant cells or reconstituted ER-membrane vesicles thereof may be used for measuring and determining the activity and specificity of a newly isolated LLO flippase.

For a flippase further described in detail herein, a flippase activity or specificity is measured having flippase activity that is less specific or basically un-specific for flipping lipid-linked low-mannose oligosaccharides, i.e. having a Man1-3GlcNAc2 structure, wherein a Man1-3GlcNAc2 structure is a Man1GlcNAc2, Man2GlcNAc2 or Man3GlcNAc2 structure. By contrast, the endogenous LLO flippase activity, in particular the Rft1-type activity, has the highest flippase activity or specificity for LLO having Man5GlcNAc2, followed by Man3GlcNAc2. More particularly, the flippase of the invention displays relative to the endogenous Rft1 an inverted specificity for LLO, being highest for small LLO such as Man1GlcNAc2 and smallest for Man5GlcNAc2.

Suitable Host Cells

The transfer of the LLO to the nascent protein in the ER is highly conserved in all eukaryotes including yeast, fungi, plants, animals, and humans. Therefore, the cells of the invention as describes in detail above may in principle be any type of eukaryotic cell including lower eukaryotic cells, fungal cells, but also plant cells, insect cells or mammalian cells.

A “host cell” according to the invention, is intended to relate to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism. The term “cell” or “host cell” used for the production of a heterologous glycoprotein refers to a cell into which a nucleic acid, e.g. encoding a heterologous glycoprotein, can be or is introduced/transfected. Such cells include both prokaryotic cells, which are used for propagation of vectors/plasmids, and eukaryotic cells.

In preferred embodiments, the host cell is a mammalian cell. Preferably, the cell is selected from, preferably immortalized, cell lines of hybridoma cells, myeloma cells, preferably rat myeloma cells and mouse myeloma cells, or human cells.

In more preferred variants thereof the cell is selected from, but not limited to, CHO cells, in particular CHO K-1 and CHO DG44, BHK cells, NSO cells, SP2/0 cells, HEK293 cells, HEK293EBNA cells, PER.C6 cells, COS cells, 3T3 cells, YB2 cells, HeLa cells, and Vero cells. In preferred variants the cell is selected from DHFR-deficient CHO cells, such as dhfr⁻CHO (Proc. Natl. Acad. Sci. USA, Vol. 77, p. 4216-4220, 1980) and CHO K-1 (Proc. Natl. Acad. Sci. USA, Vol. 60, p. 1275, 1968).

In other preferred embodiments, the host cell is an amphibian cell. Preferably, the cell is selected from, but not limited to, Xenopus laevis oocytes (Nature, Vol. 291, p. 358-360, 1981).

In other preferred embodiments, the host cell is an insect cell. Preferably, the cell is selected from, but not limited to, Sf9, Sf21, and Tn5.

In other preferred embodiments, the host cell is a plant cell. Preferably, the cell is selected from, but not limited to, cells derived from Nicotiana tabacum, the acquatic plant Lemna minor or the moss Physcomitrella patens. These cells are known as a system for producing polypeptides, and may be cultured also as calli.

In currently most preferred embodiments, the host cell is a lower eukaryotic cell. Lower eukaryotic cells according to the invention include, but are not limited to, unicellular, multicellular, and filamentous fungi, preferably selected from: Pichia sp. Candida sp. Saccharomyces sp., Saccharomycodes sp., Saccharomycopsis sp., Schizosaccharomyces sp., Zygosaccharomyces sp. Yarrowia sp., Hansenula sp., Kluyveromyces sp., Trichoderma sp, Aspergillus sp., and Fusarium sp. and Myceteae, preferably selected from Ascomycetes, in particular Chysosporium lucknowense, and Basidiomycetes, in particular Coniphora sp. as well as Arxula sp.

In more preferred variants thereof the cell is selected from, but not limited to, P. pastoris, P. stiptis, P. methanolica, P. bovis, P. canadensis, P. fermentans, P. membranaefaciens, P. pseudopolymorpha, P. quercuum, P. robertsii, P. saitoi, P. silvestrisi, P. strasburgensis; P. finlandica, P. trehalophila, P. koclamae, P. opuntiae, P. thermotolerans, P. salictaria, P. guercuum, P. pijperi; C. albicans, C. amphixiae, C. atlantica, C. corydalis, C. dosseyi, C. fructus, C. glabrata, C. fermentati, C. krusei, C. lusitaniae, C. maltosa, C. membranifaciens, C. utilis; S. bayanus, S. cerevisiae, S. bisporus, S. delbrueckii, S. fermentati, S. fragilis, S. mellis, S. rosei; Saccharomycodes ludwigii, Saccharomycopsis capsularis; Schizosaccharomyces pombe, Schizosaccharomyces octosporus, Zygosaccharomyces bisporus, Zygosaccharomyces mellis, Zygosaccharomyces rouxii; Yarrowia fipolytica, Hansenula polymorpha, Kluyveromyces sp., Trichoderma reseei., A. nidulans, A. candidus, A. carneus, A. clavatus, A. fumigatus , A. niger, A. oryzae, A. versicolor, Fusarium gramineum, Fusarium venenatum, and Neurospora crassa as well as Arxula adeninivorans.

Host Cells Lacking Rft1-Type Flippase Activity

All enzyme activities and genes described herein and referred to in Tables 1 and 2 are named according to their respective gene locus in the yeast S. cerevisiae. The skilled person is able to provide respective activities present in other organisms, including prokaryotes. Examples of alternative sources are strains of Saccharomyces, Pichia, Aspergillus, Candida, and similar. Based on homologies amongst known enzymatic activates, one may either design PCR primers or use genes or gene fragments encoding such enzymes as probes to identify homologues in DNA libraries of the target organism. Alternatively, one may be able to complement particular phenotypes in related organisms.

Alternatively, if the entire genomic sequence of a particular fungus of interest is known, one may identify such genes simply by searching publicly available DNA databases, which are available from several sources such as NCBI, Swissprot etc. For example, by searching a given genomic sequence or data base with a known gene from S. cerevisiae, one can identify genes of high homology in such a genome, which with a high degree of certainty encodes a gene that has a similar or identical activity. For example, homologues to known mannosyl transferases from S. cerevisiae in P. pastoris have been identified using either one of these approaches; these genes have similar functions to genes involved in the mannosylation of proteins in S. cerevisiae and thus their deletion may be used to manipulate the glycosylation pattern in P. pastoris or any other fungus with similar glycosylation pathways.

In preferred variants of the above-characterized embodiments, the host cell is further modified or genetically engineered to lack or to be diminished or depleted in an (endogenous) LLO flippase activity, in particular of the Rft1 type, by e.g. way of knocking-out rft1 and/or rft1 homologues. More particular, the cell is a knock-out mutant to the gene rft1. The invention also concerns methods for producing this cell.

The present invention therefore relates to genetically engineered cells where at least one endogenous enzyme activity is lacking or is being ineffective due one or more means, selected from suppression by inversion, suppression by antisense constructs, suppression by deletion, suppression on the level of transcription, suppression on the level of translation and other means. These are well known to a person skilled in molecular biology.

In the context of the present invention by the term “knock-out” or “knock-out mutant” refers to both, full knock-out systems wherein the gene or transcript is not present at all, and partial knock-out mutants wherein the gene or transcript is still present but is silent or of little concentration, respectively, so that no considerable effect is exerted by the transcript in the cell.

The creation of gene knock-outs, once a given target gene sequence has been determined, is a well-established technique in the yeast and fungal molecular biology community, and can be carried out by anyone of ordinary skill in the art (e.g. see: R. Rothsteins, (1991) Methods in Enzymology, vol. 194, p. 281). In fact, the choice of a host organism may be influenced by the availability of good transformation and gene disruption techniques for such a host. If several transferases have to be knocked out, methods have been developed that allow for the repeated use of markers, for example, the URA3 markers to sequentially eliminate all undesirable endogenous transferase or other enzyme activity referred to herein. This technique has been refined by others but basically involves the use of two repeated DNA sequences, . flanking a counter selectable marker. The presence of the marker is useful in the subsequent selection of transformants; for example, in yeast the ura3, his4, suc2, g418, b/a, or shb/e genes may be used. For example, ura3 may be used as a marker to ensure the selection of a transformants that have integrated a construct. By flanking the ura3 marker with direct repeats one may first select for transformants that have integrated the construct and have thus disrupted the target gene. After isolation of the transformants, and their characterization, one may counter select in a second round for those that are resistant to 5′FOA. Colonies that are able to survive on plates containing 5′FOA have lost the ura3 marker again through a crossover event involving the repeats mentioned earlier. This approach thus allows for the repeated use of the same marker and facilitates the disruption of multiple genes without requiring additional markers.

As used herein, the term “wild-type” as applied to a nucleic acid or polypeptide refers to a nucleic acid or a polypeptide that occurs in, or is produced by, respectively, a biological organism as that biological organism exists in nature.

The term “heterologous” as applied herein to a nucleic acid in a host cell or a polypeptide produced by a host cell refers to any nucleic acid or polypeptide (e.g., a protein having N-glycosylation activity) that is not derived from a cell of the same species as the host cell. Accordingly, as used herein, “homologous” nucleic acids, or proteins, are those that occur in, or are produced by, a cell of the same species as the host cell.

More particular, the term “heterologous” as used herein with reference to nucleic acid and a particular host cell refers to any nucleic acid that does not occur in (and cannot be obtained from) that particular cell as found in nature. Thus, a non-naturally-occurring nucleic acid is considered to be heterologous to a host cell once introduced into the host cell. It is important to note that non-naturally-occurring nucleic acids can contain nucleic acid subsequences or fragments of nucleic acid sequences that are found in nature provided that the nucleic acid as a whole does not exist in nature. For example, a nucleic acid molecule containing a genomic DNA sequence within an expression vector is non-naturally-occurring nucleic acid, and thus is heterologous to a host cell once introduced into the host cell, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature.

It also follows that any nucleic acid containing a promoter sequence and polypeptide-encoding sequence (e.g., cDNA or genomic DNA) in an arrangement not found in nature is non-naturally-occurring nucleic acid. A nucleic acid that is naturally-occurring can be heterologous to a particular cell. For example, an entire chromosome isolated from a cell of yeast x is an heterologous nucleic acid with respect to a cell of yeast y once that chromosome is introduced into a cell of yeast y.

Host Cells Further Lacking ER-Localized Mannosyl Transferase Activity

The flippase according to the invention supports growth and stability when expressed in mutant cells lacking one or more enzyme activities of the ER-located glycan synthesis pathway e.g. by way of genetic engineering, in particular one or more enzymes having mannosyl transferase activity, that confer transfer of mannose residues to glycan structures such as for example a LLO having a Man1-3GlcNAc2 structure.

In a preferred embodiment the cell is specifically designed or selected to synthesize a nascent glycoprotein with a Man1GlcNAc2 structure suitable for further glycosylation processing at the Golgi.

In another preferred embodiment the cell is specifically designed or selected to synthesize a nascent glycoprotein with a Man2GlcNAc2 structure suitable for further glycosylation processing at the Golgi.

In another preferred embodiment the cell is specifically designed or selected to synthesize a nascent glycoprotein with a Man3GlcNAc2 structure suitable for further glycosylation processing at the Golgi.

In a preferred aspect the host cell of the invention which is modified to express the above-identified novel LLO flippase activity is further modified or genetically engineered to lacking one or more glycosyl transferase activity localized at the intracellular organelle. The principal idea behind these preferred embodiments is to diminish and control glycosylation, and in particular mannosylation, of the LLO at and/or in the intracellular organelle. The provision of the host cell of the invention which is modified to express the above-identified novel LLO flippase activity with relaxed specifity and thus capable of flipping low-mannose, in particular Man1-3, glycan structures to the lumen, enables the selective control of glycosylation and makes it possible to provide particularly the following improved embodiments.

The ER-localized glycosyl transferase activity to be knocked-out, diminished or depleted in the host cell preferably is a mannosyl transferase (see Table 1). In preferred embodiments of the host cell one or more of Alg2-, Alg3-, and Alg11-type activity is knocked-out, diminished or depleted. In more preferred variants these embodiments are further lacking or are diminished or depleted of one or more of beta-D-mannosyl transferase (Dpm1)-type activity and lipid-linked monosaccharide (LLM) flippase activity.

TABLE 1
ER-localized glycosyl transferase activity

| Name | Function | EC Number | Synonymous name |
|---|---|---|---|
| DPM1 | dolichyl-phosphate beta-D- | 2.4.1.83 | dolichol-phosphate mannose |
| | mannosyl transferase | | synthase, |
| | | | dolichol-phosphate mannosyl |
| | | | transferase, |
| | | | mannosylphosphodolichol synthase, |
| | | | mannosylphosphoryldolichol |
| | | | synthase |
| Alg2 | alpha-1,3-mannosyl transferase | 2.4.1.— | YGL065C |
| Alg11 | alpha-1,2-mannosyl transferase | 2.4.1.— | YNL048L |
| Alg3 | dolichyl-phosphate-mannose- | 2.4.1.130 | AlgC |
| | glycolipid alpha-mannosyl | | |
| | transferase | | |

In a particular embodiment, the host cell is a mutant that is lacking Alg2-type activity. More particularly, the cell is a knock-out mutant of the gene alg2 and/or alg2 homologues. The host cell is specifically capable of synthesizing LLOs with Man1GlcNAc2 and Man2GlcNAc2 structure. The invention also concerns methods for producing this cell.

In another particular embodiment, the host cell is a mutant that is lacking Alg11-type activity. More particularly, the cell is a knock-out mutant of the gene alg11 and/or homologues thereof. The cell is specifically capable of synthesizing LLO with Man3GlcNAc, Man6GlcNAc2 and Man7GlcNAc2 structure. In a preferred variant thereof, the host cell is a mutant that is lacking both, Alg11-type activity and a lipid-linked monosaccharide (LLM) flippase activity. More particularly, the cell is a knock-out mutant of both, alg11 and/or homologues thereof and the one or more genes encoding a lipid-linked monosaccharide (LLM) flippase activity. The cell is specifically capable of synthesizing primarily LLO with a Man3GlcNAc2 structure. The invention also concerns methods for producing this cell.

In another preferred variant thereof, the host cell is a mutant that is lacking both, an Alg11-type activity and a beta-D-mannosyl transferase (DPMI)-type activity. More particularly, the cell is a knock-out mutant to both genes, alg11 and/or homologues and dpm1 and/or homologues thereof. The cell is specifically capable of synthesizing primarily LLO with a Man3GlcNAc2 structure. The invention also concerns methods for producing this cell.

In another particular embodiment, the host cell is a mutant that is lacking Alg3 type activity. More particularly, the cell is a knock-out mutant of gene alg3 and/or homologues thereof. In a more preferred embodiment the host cell is a mutant that is lacking both, Alg3-type activity and Alg11-type activity. More particularly, the cell is a knock-out mutant of both genes, alg3 and alg11, and/or any homologues thereof. This cell is specifically capable of synthesizing LLO with a Man3GlcNAc2 structure. The invention also concerns methods for producing this cell.

In preferred variants of the above-characterized embodiments, the host cell is further modified or genetically engineered to lack or is diminished or depleted in at least one Golgi-localized mannosyl transferase activity. The invention also concerns methods for producing this cell.

Host Cells Expressing POT Activity-Composite Systems

A particularly preferred embodiment of the invention relates to the expression of a, preferably heterologous, and/or modified oligosaccharyl transferase, (OST or OT). The oligosaccharyl transferase is a glycosyl transferase. It is a membrane protein or protein complex that transfers the oligosaccharides of the LLO to the nascent protein. In wild-type cells the Glc3Man9GlcNAc2 structure of a LLO will be transferred and attached to an asparagine (Asn) residue of the protein which will be glycosylated. The reaction catalyzed by OT is the central step in the N-linked glycosylation pathway.

The yeast and vertebrate OTs are complex hetero-oligomeric proteins consisting of seven or eight subunits (Ost1p, Ost2p, Ost3p/Ost6p, Ost4p, Ost5p, Stt3p, Wbp1p, and Swp1p in yeast; ribophorin I, DAD1, N33/IAP, OST4, Stt3A/Stt3B, Ost48, and ribophorin II in mammalian cells). In contrast to the multi-protein complex of yeast or vertebrates the genome of protozoan organisms posses 2 to 4 subunits, except for Trypanosoma sp. and Leishmania sp which comprise only the catalytic Stt3 subunit, of which three or four complete paralogues are encoded. The protozoan oligosaccharyl transferase (POT) differs from the yeast and vertebrate OT in their specificity towards different lipid-linked oligosaccharide structures.

Without whishing to be bound to the theory, an endogenous oligosaccharyl transferase may be highly specified to transfer a LLO with a high-mannose glycan structure that is typical to the ER of the wild-type cell. An endogenous oligosaccharyl transferase may thus be highly specified to transfer a LLO having a Glc3Man9GlcNAc2 structure. In the host cell according to the invention mannosylation is suppressed in the ER and the modified cell predominantly produces LLO having Man1-3 GlcNAc2 structures. An endogenous oligosaccharyl transferase, such as yeast dolichyl-diphosphooligosaccharide-protein glycotransferase (subunits: Wbp1, Ost1, Ost2, Ost3, Ost4, Ost5, Ost6, Swp1, Stt3p), may have low activity for such low-mannose LLO. For example, yeast OT (see FIG. 1) is expected to have low activity for LLO having Man1GlcNAc2, Man2GlcNAc2 Man3GlcNAc2, Man4GlcNAc2 or Man5GlcNAc2 structures. Without wishing to be bound to the theory, the presence of endogenous oligosaccharyl transferase activity may impose a rate limiting step and may cause a “bottle neck” in the glycosylation cascade, since the transfer of low-mannose glycans to nascent proteins take place at very limited rates, if at all.

In a further aspect, the invention thus further provides one or more, modified or preferably heterologous, oligosaccharyl transferases, and in particular cells expressing or overexpressing one or more of these modified or preferably heterologous oligosaccharyl transferases. There is provided a host cell according to the invention which, alternatively or in addition, is modified or genetically engineered to express or comprise one or more, modified or preferably heterologous, oligosaccharyl transferase activity, which is characterized in that the activity does not preferentially transfer Glc3Man9GlcNAc2 to a protein but also is capable of transferring oligosaccharides other than Glc3Man9GlcNAc2, preferably oligosaccharides having 1 to 9 mannose residues, most preferably, Man1GlcNAc2, Man2GlcNAc2, and/or Man3GlcNAc2 to a proteins. In other words, the invention provides a host cell with at least one ER-localized oligosaccharyl transferase activity that exhibits a “relaxed” specifity towards different types of glycan structures to be transferred to the protein. In particular, such activity is referred to herein as “POT-like activity” or “POT activity”, single unit OT

In a particular embodiment, a protozoan oligosaccharyl transferase (POT) is provided for use in the host cell of the invention, that exhibits considerable activity for transferring low-mannose structures, in particular Man1GlcNAc2, Man2GlcNAc2 or Man3GlcNAc2.

In more preferred variants, the POT is a homologue of the Stt3 subunit of yeast oligosaccharyl transferase of a protozoan, in particular of a protozoan selected from, but not limited to: Toxoplasma sp., Leishmania sp., and Trypanosoma sp. The protozoan is preferably selected from, but not limited to: Toxoplasma gondii (Tg), Leishmania major (Lm); Leishmania infantum (Li), Leishmania braziliensis (Lb), Leishmania mexicana (Lmx), Leishmania donovani (Ld), Leishmania guyanensis (Lg), Leishmania tropica (Lt), Trypanosoma cruzi (Tc), and Trypanosoma brucei (Tb). In particular embodiments the POT is selected from one or more of the paralogues: TbStt3Bp and TbStt3Cp of Trypanosoma brucei; LiStt3-1, LiStt3-2, and LiStt3-3 of Leishmania infantum; LbStt3-1, LbStt3-2, and LbStt3-3 of Leishmania braziliensis; and LmStt3A, LmStt3B, LmStt3C, and LmStt3D of Leishmania major, and of homologous structures thereof. In another embodiment the POT is selected from one or more of: TbStt3Bp and TbStt3Cp of Trypanosoma brucei, and LmStt3Ap, LmStt3Bp, and LmStt3Dp of Leishmania major.

The invention thus also concerns a host cell according to the invention that comprises one or more nucleic acids encoding, one or more of POT. The promoter for expressing the POT or POT-like activity may be an endogenous promoter, endogenous in respect to the cell in which the activity shall be expressed in. The promoter may confer an overexpression of one or more copies of the nucleic acid molecule.

Promoters such as ADH, Tef or GPD may be used for the expression of POT- or POT-like activity in yeast. In a preferred embodiment the gene encoding the POT- or POT-like activity is on a high copy number plasmid which preferably leads to over-experssion. In preferred embodiments, the molecule(s) is overexpressed two times, more preferred 5 times, 10 times, 20 times, 50 times, 100 times, 200 times, 500 times, 1000 times, and most preferred 2000 or more times when compared to expression from a low copy number plasmid or from single copy chromosomal integration. The promoter for expressing the POT or POT-like activity may be a adh, Tef or gpd, for example, on a high copy number plasmid.

The invention also concerns methods for producing these cells.

LLM Knock Out—POT Composite System

The invention provides a modified or genetically engineered host cell which is termed in the following a “composite system”. The composite system of the invention refers to a host cell, which is specifically capable of synthesizing LLOs having low-mannose glycan structures and transfer the low-mannose glycans to one or more nascent proteins expressed in this cell; the cell is:
(i) modified to synthesize in an intracellular organelle LLOs having low-mannose glycan structures, in particular Man1GlcNAc2, Man2GlcNAc2 or Man3GlcNAc2; accomplished in particular by way of knocking out at least one organelle-localized mannosyl transferase and optionally a lipid-linked monosaccharide (LLM) flippase as described herein in more detail; and
(ii) further modified to express an exogenous/heterologous oligosaccharyl transferase, which exhibits a relaxed substrate specificity towards low-mannose glycan structures to be transferred to the nascent protein, in particular as compared to the substrate specificity of an endogenous OT, wherein the exogenous/heterologous oligosaccharyl transferase is a protozoan oligosaccharyl transferase (POT).

In particular embodiment, the oligosaccharyl transferase, which exhibits a relaxed substrate specificity towards low-mannose glycan structures to be transferred to the nascent protein is a protozoan oligosaccharyl transferase (POT). In a paticular embodiment, the POT to be expressed or over-expressed in the host cell according to the invention is the paralogue LmStt3A of Leishmania major or a homologous structure thereof. In another particular embodiment, the POT to be expressed or over-expressed in the host cell according to the invention is the paralogue LmStt3B of Leishmania major or a homologous structure thereof. In another particular embodiment, the POT to be expressed or over-expressed in the host cell according to the invention is the paralogue LmStt3C of Leishmania major or a homologous structure thereof. In another particular embodiment, the POT to be expressed or over-expressed in the host cell according to the invention is the paralogue LmStt3D of Leishmania major or a homologous structure thereof.

In another particular embodiment, the POT to be expressed or over-expressed in the host cell according to the invention is the paralogue LbStt3-1 of Leishmania brazilensis or a homologous structure thereof. The POT expressed or over-expressed in the host cell may also be the paralogue LbStt3-2 of Leishmania braziliensis or a homologous structure thereof. In another particular embodiment, the POT to be expressed or over-expressed in the host cell according to the invention is the paralogue LbStt3-3 of Leishmania braziliensis or a homologous structure thereof.

In another particular embodiment, the POT to be expressed or over-expressed in the host cell according to the invention is the paralogue LiStt3-1 of Leishmania infantum or a homologous structure thereof. In another particular embodiment, the POT to be expressed or over-expressed in the host cell according to the invention is the paralogue LiStt3-2 of Leishmania infantum or a homologous structure thereof. The POT expressed or over-expressed in the host cell may also be the paralogue LiStt3-3 of Leishmania infantum or a homologous structure thereof.

In yet another particular embodiment, the POT to be expressed or over-expressed in the host cell according to the invention is the paralogue TbStt3A of Trypanosoma brucei or a homologous structure thereof. In another particular embodiment, the POT to be expressed or over-expressed in the host cell according to the invention is the paralogue TbStt3B of Trypanosoma brucei or a homologous structure thereof. In another particular embodiment, the POT to be expressed or over-expressed in the host cell according to the invention is the paralogue TbStt3C of Trypanosoma brucei or a homologous structure thereof.

In particular embodiments of the invention, there is provided an expression cassette or a functional analog thereof for the expression of one or more POT having a relaxed substrate specificity towards low-mannose glycan structures such as in particular one or more of the above-characterized POT. The expression cassette is comprising one or more copies of one of the nucleic acid molecules coding for an oligosaccharyl transferase having relaxed substrate specificity towards low-mannose glycan structures, selected from the above-identifed POT.

In a particular variant thereof, there is also provided a vector for the transformation of a eukaryotic host cell, comprising one or more copies of a the nucleic acid molecule coding for one or more of the POT as characterized above. The nucleic acid sequences in the vector can be operably linked to an expression control sequence. Preferably, one or more of said nucleic acid molecules are present in conjunction with at least one of: nucleic acid molecules encoding a promoter and nucleic acid molecules encoding a terminator. The promoter for expressing the POT activity may be ADH, Tef or GPD, for example, on a high copy number plasmid.

In more preferred embodiments, the present invention provides a transgenic mutant cell expressing the paralogue LmStt3D of Leishmania major or a homologous structure thereof. In a particular variant thereof LmStt3D is expressed in the cell in a low copy vector. In another particular variant thereof LmStt3D is expressed in the cell in a high copy vector.

In another preferred embodiment, the cell provided expresses the paralogue LbStt3-3 of Leishmania braziliensis or a homologous structure thereof. In a particular variant thereof LbStt3-3 is expressed in the cell in a low copy vector. In another particular variant thereof LbStt3-3 is expressed in the cell in a high copy vector.

In another preferred embodiment, the cell provided expresses the paralogue LbStt3-1 of Leishmania braziliensis or a homologous structure thereof. In a particular variant thereof LbStt3-1 is expressed in the cell in a high copy vector.

In another preferred embodiment, the cell provided expresses the paralogue LiStt3-2 of Leishmania infantum or a homologous structure thereof. In a particular variant thereof LiStt3-2 is expressed in the cell in a low copy vector.

In yet another preferred embodiment, the cell provided expresses the paralogue TbStt3B of Trypanosome brucei or a homologous structure thereof. In a particular variant thereof TbStt3B is expressed in the cell in a high copy vector.

In yet another preferred embodiment, the cell provided expresses the paralogue TbStt3C of Trypanosoma brucei or a homologous structure thereof. In a particular variant thereof TbStt3C is expressed in the cell in a high copy vector.

In a particular embodiment of the composite system, the cell is a mutant that (i) is lacking at least Alg2-type activity, and (ii) expresses or over-expresses POT activity. More particularly, the cell (i) is a knock-out mutant of alg2 and/or alg2 homologues, and (ii) expresses one or more of the above-identified POT activities. The invention also concerns methods for producing this cell.

In a particular embodiment of the composite system, the cell is a mutant that (i) is lacking at least Alg11-type activity, and (ii) expresses or over-expresses POT activity. More particularly, the cell (i) is a knock-out mutant of alg11 and/or alg11 homologues, and (ii) expresses one or more of the above-identified POT activities. In a preferred embodiment, the invention provides a knock-out mutant of alg11 and/or alg11 homologues expressing the paralogue LmStt3D of Leishmania major. In a particular variant thereof LmStt3D is expressed in a low copy vector. In another preferred embodiment, this mutant cell expresses the paralogue LbStt3-3 of Leishmania braziliensis. In a particular variant thereof LbStt3-3 is expressed in a low copy vector. In a particular variant thereof LbStt3-3 is expressed in a low copy vector. The invention also concerns methods for producing these cells.

In another particular embodiment of the composite system, the cell is a mutant that (i) is lacking at least both, Alg3-type activity and Alg11-type activity, and (ii) expresses or over-expresses POT activity. More particularly, the cell (I) is a knock-out mutant of both, alg3 and alg11 and/or any homologues thereof, and (ii) expresses one or more of the above-identified POT activities. In a preferred embodiment, the invention provides a knock-out mutant of both, alg3 and alg11 and/or any homologues thereof, expressing the paralogue LmStt3D of Leishmania major. In a particular variant thereof LmStt3D is expressed in a low copy vector. In another preferred embodiment, this mutant cell expresses the paralogue LbStt3-3 of Leishmania braziliensis. In a particular variant thereof LbStt3-3 is expressed in a low copy vector. In yet another preferred embodiment, this mutant cell expresses the paralogue TbStt3B or TbStt3C of Trypanosoma brucei. In particular variants thereof TbStt3B or TbStt3C is expressed in a high copy vector. The invention also concerns methods for producing these cells.

In another particular embodiment of the composite system, the cell is a mutant that (i) is lacking at least both, Alg11-type activity and a lipid-linked monosaccharide (LLM) flippase activity, and (ii) expresses or over-expresses POT activity. More particularly, the cell (i) is a knock-out mutant of both, alg11 and/or alg11 homologues thereof and the homologues of one or more genes encoding a lipid-linked monosaccharide (LLM) flippase activity, and (ii) expresses one or more of the above-identified POT activities. The invention also concerns methods for producing these cells.

In yet another particular embodiment of the composite system, the cell is a mutant that (i) is lacking at least both, Alg11-type activity and a beta-D-mannosyl transferase (DPMI)-type activity, and (ii) expresses or over-expresses POT activity. More particularly, the cell (i) is a knock-out mutant of both, alg11 and/or dpm1 and/or homologues thereof, and (ii) expresses one or more of the above-identified POT activities. The invention also concerns methods for producing these cells.

Without whishing to be bound to the theory, in preferred variants, no knock-out mutation for the endogenous OT is required. In a preferred variant, however, endogenous OT is not present or suppressed in the cell. Accordingly, a cell is provided where one or more of the genes encoding endogenous OT subunits are knocked-out. In preferred variants comprising yeast cells said at least one subunit of the endogenous oligosaccharyl transferase is selected from the group consisting of: Wbp1 p, Ost1 p, Ost2p, Ost3p, Ost4p, Ost5p, Ost6p, Swp1 p, and Stt3p. In a preferred embodiment the cell is a knock out mutant of genes wbp1 and stt3. In another preferred embodiment the cell is a knock out mutant of the genes ost1 and ost2.

In a particular variant, the host cell is a mutant for Stt3p, more particular the host cell is yeast strain YG543, which has a temperature-sensitive phenotype of the stt3-7 allele (Spirig et al. Mol. Gen. Genet. 256, p. 628-637, 1997).

LLM Knock Out-LLO Flippase-POT Composite System

According to another aspect, the invention provides a host cell, which is specifically capable of synthesizing LLOs having low-mannose glycan structures and transfer the low-mannose glycans to one or more nascent proteins expressed in this cell; the cell is:
(i) modified to synthesize in an intracellular organelle LLOs having low-mannose glycan structures, in particular Man1GlcNAc2, Man2GlcNAc2 or Man3GlcNAc2; accomplished in particular by way knocking out at least one organelle-localized mannosyl transferase and optionally a lipid-linked monosaccharide (LLM) flippase as described herein in more detail;
(ii) modified to express a novel LLO flippase activity with relaxed specificity towards low-mannose LLOs as described herein in more detail; and
(iii) further modified to express an oligosaccharyl transferase, which exhibits a relaxed substrate specificity towards low-mannose glycan structures to be transferred to the nascent protein which preferably is a protozoan oligosaccharyl transferase (POT), more particular, selected from the above-identifed POT.

, there is provided an expression cassette or a functional analog thereof for the expression of both, the novel LLO flippase activity as characterized above, and an oligosaccharyl transferase having relaxed substrate specificity towards low-mannose glycan structures such as POT. The expression cassette is comprising one or more copies of one of the nucleic acid molecules coding for the novel LLO flippase activity as characterized above, and one or more copies of one of the nucleic acid molecules coding for a oligosaccharyl transferase having relaxed substrate specificity towards low-mannose glycan structures such as POT as characterized above.

In a particular variant thereof, there is also provided a vector for the transformation of a eukaryotic host cell, comprising one or more copies of one of the nucleic acid molecules characterized above or one or more copies of the expression cassette as characterized above. The nucleic acid sequences in the vector can be operably linked to an expression control sequence. Preferably, one or more of said nucleic acid molecules are present in conjunction with at least one of: nucleic acid molecules encoding a promoter and nucleic acid molecules encoding a terminator. The promoter for expressing the POT activity may be ADH, Tef or GPD, for example, on a high copy number plasmid.

A preferred embodiment for a vector conferring novel LLO flippase activity and POT activity to a host cell is depicted in FIG. 14. The nucleotide sequence is provided in SEQ ID NO: 32.

As used herein, the term “derived from flc2′” also encompasses molecules comprising the complete sequence of flc2′ (SEQ ID NO: 1) and in preferred further variants encompasses molecules comprising or more fragments of flc2′ which code for one or more transmembrane domains of the Flc2 molecule. In a particular and preferred embodiment of the invention, the molecule comprises or substantially consists of a fragment that codes for transmembrane domain 4 (TM4) of Flc2′ or a homologous functional structure thereof. In a particular and preferred embodiment thereof, the molecule comprises or substantially consists of a fragment that codes for transmembrane domains 3 to 4 (TM3-4) of Flc2′ or a homologous functional structure thereof.

The molecule may comprise or substantially consist of a fragment that codes for transmembrane domain 1 (TM1) of Flc2′ or a homologous functional structure thereof. The molecule may also comprise or substantially consist of a fragment that codes for transmembrane domain 2 (TM3) of Flc2′ or a homologous functional structure thereof. In a particular and preferred embodiment thereof, the molecule comprises or substantially consists of a fragment that codes for transmembrane domains 1 to 2 (TM1-2) of Flc2′ or a homologous functional structure thereof. In another embodiment thereof, the molecule comprises or substantially consists of a fragment that codes for transmembrane domains 2 to 4 (TM2-4) of Flc2′ or a homologous functional structure thereof.

The molecule may comprise or substantially consist of a fragment that codes for transmembrane domain 3 (TM3) of Flc2′ or a homologous functional structure thereof. In a particular embodiment thereof, the molecule comprises or substantially consists of a fragment that codes for transmembrane domains 1 to 3 (TM1-3) of Flc2′ or a homologous functional structure thereof. In another embodiment thereof, the molecule comprises or substantially consists of a fragment that codes for transmembrane domains 2 to 3 (TM2-3) of Flc2′ or a homologous functional structure thereof.

In a particular embodiment of the composite system, the cell is a mutant that (i) is lacking at least Alg2-type activity; (ii) expresses novel LLO flippase activity according to the invention; and (iii) expresses POT activity. More particularly, the cell (i) is a knock-out mutant of alg2 and/or alg2 homologues; (ii) expresses one or more nucleic acid molecules conferring LLO flippase activity; and (iii) expresses one or more of the above-identified POT activity. In a more particular embodiment, the cell expresses one or more nucleic acid molecules derived from flc2′, as described above in more detail, conferring a novel LLO flippase activity. In another variant, the cell expresses one or more nucleic acid molecules derived from rft1, as described above, conferring LLO flippase activity. This cell is specifically capable of synthesizing LLO with Man1GlcNAc2 and Man2GlcNAc2 structures and transferring said structure to a nascent protein. The invention also concerns methods for producing this cell.

In another preferred embodiment, the cell is a mutant that (i) is lacking at least Alg11-type activity; (ii) expresses novel LLO flippase activity according to the invention; and (iii) expresses POT activity. More particularly, the cell (i) is a knock-out mutant of alg11 and/or alg11 homologues; (ii) expresses one or more nucleic acid molecules conferring LLO flippase activity; and (iii) expresses one or more of the above-identified POT activity. In a more particular embodiment, the cell expresses one or more nucleic acid molecules derived from flc2′, as described above in more detail, conferring a novel LLO flippase activity. In another variant, the cell expresses one or more nucleic acid molecules derived from rft1, as described above, conferring LLO flippase activity. This cell is specifically capable of synthesizing LLO with Man3GlcNAc, Man6GlcNAc2, Man7GlcNAc2 and/or Man8GlcNAc2 structure and transferring said structure to a nascent protein. The invention also concerns methods for producing this cell.

In a most preferred embodiment, the cell is a mutant that (i) is lacking at least both, Alg3-type activity and Alg11-type activity; (ii) expresses novel LLO flippase activity according to the invention; and (iii) expresses POT activity. More particularly, the cell (i) is a knock-out mutant of both, alg3 and alg11, or any homologues thereof; (ii) expresses one or more nucleic acid molecules conferring LLO flippase activity; and (iii) expresses one or more of the above-identified POT activity. In a more particular embodiment, the cell expresses one or more nucleic acid molecules derived from flc2′, as described above in more detail, conferring a novel LLO flippase activity. In another variant, the cell expresses one or more nucleic acid molecules derived from rft1, as described above, conferring LLO flippase activity. This cell is specifically capable of synthesizing LLO with a Man3GlcNAc2 structure and transferring said structure to a nascent protein. A preferred mutant cell according to this invention expresses the paralogue LmStt3D of Leishmania major. In a particular variant thereof LmStt3D is expressed in a low copy vector. In another preferred embodiment, this mutant cell expresses the paralogue LbStt3-3 of Leishmania braziliensis. In a particular variant thereof LbStt3-3 is expressed in a low copy vector. In yet another preferred embodiment, this mutant cell expresses the paralogue TbStt3B or TbStt3C of Trypanosome brucei. In particular variants thereof TbStt3B or TbStt3C is expressed in a high copy vector. The invention also concerns methods for producing these cells.

In another preferred embodiment, the cell is a mutant that (i) is lacking at least both, Alg11-type activity and a lipid-linked monosaccharide (LLM) flippase activity; (ii) expresses novel LLO flippase activity according to the invention; and (iii) expresses POT activity. More particularly, the cell (i) is a knock-out mutant of both, alg11 and/or homologues thereof and the one or more genes encoding a lipid-linked monosaccharide (LLM) flippase activity; (ii) expresses one or more nucleic acid molecules conferring LLO flippase activity; and (iii) expresses one or more of the above-identified POT activity. In a more particular embodiment, the cell expresses one or more nucleic acid molecules derived from flc2′, as described above in more detail, conferring a novel LLO flippase activity. Alternatively or in addition, the cell expresses one or more nucleic acid molecules derived from rft1, as described above, conferring LLO flippase activity. A preferred mutant cell according to this invention expresses the paralogue LmStt3D of Leishmania major. In a particular variant thereof LmStt3D is expressed in a low copy vector. In another preferred embodiment, this mutant cell expresses the paralogue LbStt3-3 of Leishmania braziliensis. In a particular variant thereof LbStt3-3 is expressed in a low copy vector. In yet another preferred embodiment, this mutant cell expresses the paralogue TbStt3B or TbStt3C of Trypanosome brucei. In particular variants thereof TbStt3B or TbStt3C is expressed in a high copy vector. The invention also concerns methods for producing these cells. This cell is specifically capable of synthesizing primarily LLO with a Man3GlcNAc2 structure and transferring said structure to a nascent protein. The invention also concerns methods for producing this cell.

In yet another preferred embodiment, the cell is a mutant that (i) is lacking at least both, Alg11-type activity and a beta-D-mannosyl transferase (DPM1)-type activity; (ii) expresses novel LLO flippase activity according to the invention; and (iii) expresses POT activity. More particularly, the cell (i) is a knock-out mutant of both, alg11 and/or dpm1 and/or homologues thereof; (ii) expresses one or more nucleic acid molecules conferring LLO flippase activity; and (iii) expresses one or more of the above-identified POT activity. In a more particular embodiment, the cell expresses one or more nucleic acid molecules derived from flc2′, as described above in more detail, conferring a novel LLO flippase activity. Alternatively or in addition, the cell expresses one or more nucleic acid molecules derived from rft1, as described above, conferring LLO flippase activity. A preferred mutant cell according to this invention expresses the paralogue LmStt3D of Leishmania major. In a particular variant thereof LmStt3D is expressed in a low copy vector. In another preferred embodiment, this mutant cell expresses the paralogue LbStt3-3 of Leishmania braziliensis. In a particular variant thereof LbStt3-3 is expressed in a low copy vector. In yet another preferred embodiment, this mutant cell expresses the paralogue TbStt3B or TbStt3C of Trypanosome brucei. In particular variants thereof TbStt3B or TbStt3C is expressed in a high copy vector. The invention also concerns methods for producing these cells. This cell is specifically capable of synthesizing primarily LLO with a Man3GlcNAc2 structure and transferring said structure to a nascent protein. The invention also concerns methods for producing this cell.

In particular, acell is provided where one or more of the genes encoding endogenous OT subunits are knocked-out. In preferred variants comprising yeast cells said at least one subunit of the endogenous oligosaccharyl transferase is selected from the group consisting of: Wbp1 p, Ost1p, Ost2p, Ost3p, Ost4p, Ost5p, Ost6p, Swp1p, and Stt3p. In a preferred embodiment the cell is a knock out mutant of genes wbp1 and stt3. In another preferred embodiment the cell is a knock out mutant of the genes ost1 and ost2.

In further embodiments of the invention, any one of the cells described above may further comprise at least one nucleic acid encoding a heterologous glycoprotein. The promoter for expressing a heterologous glycoprotein may be an endogenous promoter, endogenous in respect to the cell in which the activity shall be expressed in. In another preferred embodiment the promoter is a heterologous promoter, an inducible or constitutive promoter that confers an overexpression of one or more copies of the nucleic acid molecule. These cells are specifically capable of synthesizing primarily LLO with a Man1-3GlcNAc2 structure and transferring said structure to said heterologous protein.

Without whishing to be bound to the theory, the above-specified knock-out deletion strains should only enable to produce low-mannose LLO, in particular Man3GlcNAc2, on or in the ER which are then attached to the protein in the ER. In some conditions it may be found that additional mannose residues are added afterwards in the Golgi apparatus by mannosyl transferases, which may result in Man4GlcNAc2 and Man5GlcNAc2 structures on the protein. In order to reduce the amount of the undesired Man4GlcNAc2 and Man5GlcNAc2 structures, the invention provides measures to avoid this. A preferred measure is the deletion of one or more of the genes encoding Golgi-localized mannosyl transferases in any one of the cells of the invention as described in detail above.

The present invention is in clear contrast to previous teachings of the prior art, wherein desired hypomannosylated glycans are obtained by trimming/cleavage of high-mannose (e.g. Man8GlcNAc2 or Man 9GlcNAc2) or hypermannosylated glycoforms using homologous or heterologous mannosidase activities. In a preferred embodiment the present invention thus concerns cells that do not exhibit an effective mannosidase activity or no mannosidase activity at all.

Host Cells with Modified Golgi-Glycosylation

The primary glycoprotein resulting from oligosaccharyl transferase activity at the ER may be subject to further glycosylation at the Golgi as described below in more detail. The further major aspect of the present invention is the provision of means and methods for the modification of the Golgi-based glycosylation in the host cell of the invention. Modification of ER-based glycosylation as described in more detail hereinabove and modification of the Golgi-based glycosylation as described in more detail herein below, go hand in hand. This invention advantageously provides primary glycoproteins with low-mannose glycan structure which form the ideal substrate for the subsequent modified glycosylation in the Golgi.

Host Cells Further Lacking Golgi-Localized Mannosyl Transferase Activity

In preferred embodiments the host cell of the invention is further modified or genetically engineered to lack or be diminished or depleted in one or more, at least two, preferably at least three, at least four or at least five of Golgi-localized mannosyl transferases. The mannosyl transferases are preferably selected from: Och1p, Mnn1p, Mnn2p, Mnn4p, Mnn5p, Mnn9p, Mnn10p, and Mnn11p, and homologues thereof (see Table 2). The cell is preferably a knock-out mutant of at least one of the genes selected from the group consisting of och1, mnn1, mnn2, mnn4, mnn5, mnn9, mnn10, and mnn11 gene and homologues thereof. Homologues also include other members of the same or a related gene family.

TABLE 2
Golgi-localized mannosyl transferases

| | | EC | |
|---|---|---|---|
| Name | Function | Number | Synonymous names |
| Och1 | alpha-1,6-mannosyl transferase | 2.4.1.232 | YGL048C |
| Mnn1 | alpha-1,3-mannosyl transferase | 2.4.1.— | YER001W |
| Mnn2 | alpha1,2-mannosyl transferase | 2.4.1.— | YBR015C, TTP1, CRV4, |
| | | | LDB8 |
| Mnn4 | regulator of mannosylphosphate | 2.4.1.— | YKL201C |
| | transferase | | |
| Mnn5 | alpha1,2-mannosyl transferase | 2.4.1.— | YJL186W |
| Mnn6 | mannosylphosphate transferase | 2.4.1.— | KTR6, YPL053C |
| Mnn8 | alpha-1,6 mannosyl transferase | 2.4.1.— | ANP1 |
| | complex | | |
| Mnn9 | Subunit of a Golgi mannosyl | 2.4.1.— | YPL050C |
| | transferase complex | | |
| Mnn10 | Subunit of a Golgi mannosyl | 2.4.1.— | YDR245W, BED1, SLC2, |
| | transferase complex | | REC41 |
| Mnn11 | Subunit of a Golgi mannosyl | 2.4.1.— | YJL183W |
| | transferase complex | | |
| Ktr1 | Alpha-1,2-mannosyltransferase | 2.4.1.— | YOR099W |
| Ktr2 | Mannosyltransferase | 2.4.1.— | YKR061W |
| Ktr3 | Putative alpha-1,2- | 2.4.1.— | YBR205W |
| | mannosyltransferase | | |
| Ktr4 | Putative mannosyltransferase | 2.4.1.— | YBR199W |
| Ktr5 | Putative mannosyltransferase | 2.4.1.— | YNL029C |
| Ktr6 | Probable mannosylphosphate | 2.4.1.— | YPL053C (Mnn6) |
| | transferase | | |
| Ktr7 | Putative mannosyltransferase | 2.4.1.— | YIL085C |
| Van1 | Component of the mannan | | YML115C |
| | polymerase I | | |
| Vrg4 | Golgi GDP-mannose | | YGL225W |
| | transporter | | |

The cell may be a knock-out mutant of at least one gene of: och1, or mnn1, mnn2, mnn4, mnn5, mnn9, mnn10, mnn11 and/or the homologues thereof. The cell may also be a knock-out mutant of at least one gene of: ktr1, ktr2, ktr3, ktr4, ktr5, ktr6, ktr7 and/or the homologues thereof. The cell may also be a knock-out mutant of at least one gene of: van1, vrg4 and/or the homologues thereof.

In a preferred embodiment, the cell of the invention, and in particular the above-identified composite system, is further lacking at least an Och1-type activity, more particular an alpha-1,6-mannosyl transferase. More particularly, the cell further is a knock-out mutant to och1. For example, the composite system of the invention can be engineered based on hypermannosylation-minus (Och1) mutant strains of Pichia pastoris.

In a preferred embodiment, the cell of the invention, and in particular the above-identified composite system, is lacking at least alpha-1,3-mannosyl transferase activity conferred by the mnn1 gene or the homologues thereof. more particular a knock-out mutant to at least mnn1 or its homologues. This cell may also lack one or more of the above characterized mannosyl transferase activities, and in particular is a knock-out mutant of one or more of these genes coding for this mannosyl transferase activities, in particular selected from one or more of mnn9, mnn5, van1 and its homologues.

In a preferred embodiment the cell is a mutant that is lacking at least Alg11-type activity, and Mnn1-type activity. More particularly, said cell is a knock-out mutant of at least: alg11 and mnn1. A preferred embodiment thereof is a mutant cell, preferably a yeast cell, that is a composite system, which is
(i) modified to express at least one of the novel LLO flippase activities, in particular encoded by one or more of the nucleic acid molecules as identified herein, and which is a knock-out mutant to alg11 or its homologues,
(ii) a knock-out mutant to at least mnn1 or its homologues, and
(iiia) further expresses or overexpresses at least one of the above characterized POT activity, and, alternatively or in addition,
(iiib) further expresses or overexpresses at least one of the above characterized LLO activity

This cell is specifically capable of synthesizing primarily LLO with a Man3GlcNAc2 structure and transferring said structure to a nascent protein. The invention also concerns methods for producing this cell.

In a preferred embodiment the cell is a mutant that is lacking at least Alg3-type activity, Alg11-type activity, and Mnn1-type activity. More particularly, said cell is a knock-out mutant of at least: alg11, alg3 and mnn1. A preferred embodiment thereof is a mutant cell, preferably a yeast cell, that is a composite system, which is
(i) modified to express at least one of the novel LLO flippase activities, in particular encoded by one or more of the nucleic acid molecules as identified herein, and which is a knock-out mutant to alg3 and alg11 or their homologues,
(ii) a knock-out mutant to at least mnn1 or its homologues, and
(iiia) further expresses or overexpresses at least one of the above characterized POT activity, and, alternatively or in addition,
(iiib) further expresses or overexpresses at least one of the above characterized LLO activity

This cell is specifically capable of synthesizing primarily LLO with a Man3GlcNAc2, Man6GlcNAc2, Man7GlcNAc2, or Man8GlcNAc2 structure and transferring said structure to a nascent protein. The invention also concerns methods for producing this cell.

In another preferred embodiment the cell is a mutant that is lacking at least Alg11-type activity, DPM1-type activity, and Mnn1-type activity. More particularly, said cell is a knock-out mutant of at least: alg11, dpm1, and mnn1. A preferred embodiment thereof is a mutant cell, preferably a yeast cell, that is a composite system, which is
(i) modified to express at least one of the novel LLO flippase activities, in particular encoded by one or more of the nucleic acid molecules as identified herein, and which is a knock-out mutant to dpm1 and alg11 or their homologues,
(ii) a knock-out mutant to at least mnn1 or its homologues, and
(iiia) further expresses or overexpresses at least one of the above characterized POT activity, and, alternatively or in addition,
(iiib) further expresses or overexpresses at least one of the above characterized LLO activity

In particular embodiments, these cells express or overexpress one or more nucleic acid molecules derived from flc2′, as described above in more detail, conferring a novel LLO flippase activity. Alternatively or in addition, the cell expresses one or more nucleic acid molecules derived from rft1, as described above, conferring LLO flippase activity.

In particular embodiments thereof, these cells express or overexpress the paralogue LmStt3D of Leishmania major. In a particular variant thereof LmStt3D is expressed in a low copy vector. In another preferred embodiment, this mutant cell expresses the paralogue LbStt3-3 of Leishmania braziliensis. In a particular variant thereof LbStt3-3 is expressed in a low copy vector. In yet another preferred embodiment, this mutant cell expresses the paralogue TbStt3B or TbStt3C of Trypanosoma brucei. In particular variants thereof TbStt3B or TbStt3C is expressed in a high copy vector. The invention also concerns methods for producing these cells.

In particular embodiments thereof, these cells are also a knock-out mutant of endogenous OT activity, in particular by knock-out of ost1 and ost2 and/or wbp1 and stt3 and/or the respective homologues thereof.

Specific Control of Golqi-Based Glycosylation by Expression of Heteroloqous Glycosyl Transferases

As described in more detail herein below preferred embodiments of the nucleic acid molecule or the poly amino acid molecule of the invention is used to produce modified host cell specified to produce glycoproteins or glycoprotein compositions as characterized in the following.

The cell of the invention may be further genetically engineered to alter the glycosylation cascade within the Golgi, which differs significantly between different eukaryotes and thus, the glycoproteins differ in their glycan structure depending on the cell type they have been expressed in and isolated from. For example, lower eukaryotes ordinarily produce high-mannose containing N-glycans. Accordingly, another object of the invention is to provide a cell useful for and method able to produce a glycoprotein having a certain type of N-glycan structure such as e.g. a human glycan structures in a cell other than a human cell. Accordingly, such cell will further be genetically modified in the Golgi glycosylation pathway that allow the cell to carry out a sequence of enzymatic reactions, which mimic the processing of glycoproteins in e.g. humans. Recombinant proteins expressed in these engineered cells yield glycoproteins more similar, if not substantially identical, to their human counterparts. If lower eukaryotic cells are used as exemplified above, which ordinarily produce high-mannose containing N-glycans, said cells are modified to produce N-glycans such as Man3GlcNAc2 or Man5GlcNAc2 or other structures along human glycosylation pathways. Preferred embodiments include, but are not limited to, recombinant glycoproteins comprising one or more of glycan structure selected from:
GlcNAcMan3-5GlcNAc2,
GlcNAc2Man3GlcNAc2,
GlcNAc3Man3GlcNAc2-bisecting
Gal2GlcNAc2Man3GlcNAc2,
Gal2GlcNAc2Man3GlcNAc2Fuc,
Gal2GlcNAc3Man3GlcNAc2-bisecting,
Gal2GlcNAc3Man3GlcNAc2Fuc-bisecting,
NeuAc2Gal2GlcNAc2Man3GlcNAc2,
NeuAc2Gal2GlcNAc2Man3GlcNAc2Fuc,
NeuAc2Gal2GlcNAc3Man3GlcNAc2-bisecting,
euAc2Gal2GlcNAc3Man3GlcNAc2Fuc-bisecting,
GlcNAc3Man3GlcNAc2,
Gal3GlcNAc3Man3GlcNAc2,
Gal3GlcNAc3Man3GlcNAc2Fuc,
NeuAc3Gal3GlcNAc3Man3GlcNAc2, and
NeuAc3Gal3GlcNAc3Man3GlcNAc2Fuc.

As used herein GlcNAc is N-acetylglucosamine, Gal is galactose, Fuc is fucose, and NeuAc is N-acetylneuraminic acid or sialic acid. As used herein, in preferred embodiments all glycan structures lack fucose in their glycan structures unless the presence of fucose (Fuc) is specifically exemplified.

According to the present invention this is preferably achieved by engineering and/or selection of strains which lack certain enzyme activities that create undesirable high mannose type structures characteristic of glycoproteins of lower eukaryotes, in particular fungal cells such as yeasts. This is preferably achieved by engineering host cells which express heterologous activities which generate glycan structures which are not recognized by enzymes creating the high mannose type, which are selected either to have optimal activity under the conditions present in the lower eukaryotic cell such as a fungi where activity is desired, or which are targeted to an organelle where optimal activity is achieved, and combinations thereof wherein the genetically engineered eukaryote expresses multiple heterologous enzymes required to produce “human-like” glycoproteins.

In preferred embodiments the present invention also concerns the integration of one or more heterologous enzyme activities in the Golgi that are capable of producing “human-like” N-glycans. In preferred embodiments, the invention provides genetically engineered cells which comprise in the Golgi at least one heterologous glycosyl transferase activity and/or one or more glycosyl transferase activity associated activity selected from the group of activities listed in Tables 3, 4, and 5.

Human-like glycosylation is primarily characterized by “complex” N-glycan structures containing N-acetylglusosamine, galactose, fucose and/or N-acetylneuraminic acid. Other sialic acids like N-glycolylneuraminic acid present in N-glycans from other mammals like hamster are absent in humans. Also special oligosaccharyl linkages like terminally bound alpha-1-3 galactose is typical for rodents but absent in human cells.

TABLE 3
Heterologous glycosyl transferases, transporters and associated enzymes

| | Function/enzymatic | | EC | Synonymous | Gene, |
|---|---|---|---|---|---|
| Name | activity | Location | Number | name(s) | exemplary |
| GnTI | mannosyl (alpha-1,3-)- | Golgi | 2.4.1.101 | GlcNAc transferase 1, | Mgat1 |
| | glycoprotein beta-1,2-N- | | | alpha-1,3-mannosyl- | |
| | acetylglucosaminyl | | | glycoprotein beta-1,2- | |
| | transferase | | | N-acetylglucosaminyl | |
| | | | | transferase | |
| GnTII | mannosyl (alpha-1,6-)- | Golgi | 2.4.1.143 | GlcNAc transferase 2, | Mgat2 |
| | glycoprotein beta-1,2-N- | | | N-acetylglucosaminyl | |
| | acetylglucosaminyl | | | transferase II, UDP- | |
| | transferase | | | GlcNAc: mannoside | |
| | | | | alpha-1-6 | |
| | | | | acetylglucosaminyl | |
| | | | | transferase, Alpha- | |
| | | | | 1,6-mannosyl- | |
| | | | | glycoprotein 2-beta-N- | |
| | | | | acetylglucosaminyl | |
| | | | | transferase | |
| GnTIII | beta-1,4-mannosyl- | Golgi | 2.4.1.144 | GlcNAc transferase 3, | Mgat3 |
| | glycoprotein 4-beta-N- | | | N-acetylglucosaminyl | |
| | acetylglucosaminyl | | | transferase III | |
| | transferase | | | | |
| GnTIV | mannosyl (alpha-1,3-)- | Golgi | 2.4.1.145 | GlcNAc transferase 4, | Mgat4 |
| | glycoprotein beta-1,4-N- | | | N-acetylglucosaminyl | |
| | acetylglucosaminyl | | | transferase IV, Alpha- | |
| | transferase | | | 1,3-mannosyl- | |
| | | | | glycoprotein 4-beta-N- | |
| | | | | acetylglucosaminyl | |
| | | | | transferase, isozymes | |
| | | | | A and B | |
| GnTV | mannosyl (alpha-1,6-)- | Golgi | 2.4.1.155 | GlcNAc transferase 5, | Mgat5 |
| | glycoprotein beta-1,6-N- | | | N-acetylglucosaminyl | |
| | acetyl-glucosaminyl | | | transferase V, Alpha- | |
| | transferase | | | 1,6-mannosyl- | |
| | | | | glycoprotein 6-beta-N- | |
| | | | | acetylglucosaminyl | |
| | | | | transferase | |
| GnTVI | alpha-1,6-mannosyl- | Golgi | 2.4.1.201 | GlcNAc transferase 6, | Mgat6 |
| | glycoprotein 4-beta-N- | | | N-acetylglucosaminyl | |
| | acetylglucosaminyl | | | transferase VI | |
| | transferase | | | | |
| GalT | beta-N- | Golgi | 2.4.1.38 | Gal-Transferase 8, | B4galT1 |
| | acetylglucosaminylglyco | | | UDP-Gal transferase | |
| | peptide beta-1,4- | | | | |
| | galactosyl transferase | | | | |
| FucT | alpha (1,6) fucosyltransferase | Golgi | 2.4.1.68 | Fuc-transferase 8, | Fut8 |
| | | | | GDP-Fuc transferase | |
| ST | beta-galactoside alpha- | Golgi | 2.4.99.1 | Sialyltransferase, | ST6gal1 |
| | 2,6-sialyl transferase | | | CMP-N- | |
| | | | | acetylneuraminate- | |
| | | | | beta-galactosamide- | |
| | | | | alpha-2,6-sialyl transferase, | |
| | UDP-N- | Cytosol | 5.1.3.14 | UDP-GlcNAc-2- | NeuC |
| | acetylglucosamine 2- | | | epimerase | |
| | epimerase | | | | |
| | sialic acid synthase | Cytosol | | | NeuB |
| | CMP-NeuNAc | Cytosol | 2.7.7.43 | | Cmas |
| | synthetase | | | | NeuA |
| | N-acylneuraminate-9- | | 2.5.1.57 | | |
| | phosphate synthase | | | | |
| | N-acylneuraminate-9- | | 3.1.3.29 | | |
| | phosphatase | | | | |
| | UDP-GlcNac transporter | Golgi | | | Slc35A3 |
| | UDP-Gal-transporter | Golgi | | | Slc35A2 |
| | GDP-fucose transporter | Golgi | | | Slc35C1 |
| | CMP-sialic acid | Golgi | | | Slc35A1 |
| | transporter | | | | |
| | nucleotide | Golgi | | | |
| | diphoshatases | | | | |
| | GDP-D-mannose 4,6- | Cytosol | 4.2.1.47 | | Gmds |
| | dehydratase | | | | |
| | GDP-4-keto-6-deoxy-D- | Cytosol | 1.1.1.271 | GDP L-fucose | Tsta3 |
| | mannose-3,5-epimerase- | | | synthase, FX protein | |
| | 4-reductase | | | | |

TABLE 4
Heterologous enzymes for Golgi-based synthesis of prefered biantennary glycans

| | | |
|---|---|---|
| N-acetylglucosaminylation | bisecting GlcNAc | alactosylation |

GlcNAcMan3-5GlcNAc2

mannosyl(alpha-1,3-)-glycoprotein beta-1,2-N-
acetylglucosaminyl transferase (GnTI)
UDP-N-acetylglucosamine transporter

GlcNAc2Man3GlcNAc2

mannosyl(alpha-1,3-)-glycoprotein beta-1,2-N-
acetylglucosaminyl transferase (GnTI)
UDP-N-acetylglucosamine transporter
mannosyl(alpha-1,6-)-glycoprotein beta-1,2-N-
acetylglucosaminyl transferase (GnTII)

GlcNAc3Man3GlcNAc2-bisecting

| | |
|---|---|
| mannosyl(alpha-1,3-)-glycoprotein beta-1,2-N- | beta-1,4-mannosyl- |
| acetylglucosaminyl transferase (GnTI) | glycoprotein 4-beta-N- |
| UDP-N-acetylglucosamine transporter | acetylglucosaminyl transferase |
| mannosyl(alpha-1,6-)-glycoprotein beta-1,2-N- | (GnTIII) |
| acetylglucosaminyl transferase (GnTII) | |

Gal2GlcNAc2Man3GlcNAc2

| | |
|---|---|
| mannosyl(alpha-1,3-)-glycoprotein beta-1,2-N- | beta-N-acetylglucosaminyl |
| acetylglucosaminyl transferase (GnTI) | glycopeptide beta-1,4- |
| UDP-N-acetylglucosamine transporter | galactosyl transferase |
| mannosyl(alpha-1,6-)-glycoprotein beta-1,2-N- | (GalT) |
| acetylglucosaminyl transferase (GnTII) | UDP-galactose transporter |

Gal2GlcNAc2Man3GlcNAc2Fuc

| | |
|---|---|
| mannosyl(alpha-1,3-)-glycoprotein beta-1,2-N- | beta-N-acetylglucosaminyl |
| acetylglucosaminyl transferase (GnTI) | glycopeptide beta-1,4- |
| UDP-N-acetylglucosamine transporter | galactosyl transferase |
| mannosyl(alpha-1,6-)-glycoprotein beta-1,2-N- | (GalT) |
| acetylglucosaminyl transferase (GnTII) | UDP-galactose transporter |

Gal2GlcNAc3Man3GlcNAc2-bisecting

| | | |
|---|---|---|
| mannosyl(alpha-1,3-)-glycoprotein beta-1,2-N- | beta-1,4-mannosyl- | beta-N-acetylglucosaminyl |
| acetylglucosaminyl transferase (GnTI) | glycoprotein 4-beta-N- | glycopeptide beta-1,4- |
| UDP-N-acetylglucosamine transporter | acetylglucosaminyl transferase | galactosyl transferase |
| mannosyl(alpha-1,6-)-glycoprotein beta-1,2-N- | (GnTIII) | (GalT) |
| acetylglucosaminyl transferase (GnTII) | | UDP-galactose transporter |

Gal2GlcNAc3Man3GlcNAc2Fuc-bisecting

| | | |
|---|---|---|
| mannosyl(alpha-1,3-)-glycoprotein beta-1,2-N- | beta-1,4-mannosyl- | beta-N-acetylglucosaminyl |
| acetylglucosaminyl transferase (GnTI) | glycoprotein 4-beta-N- | glycopeptide beta-1,4- |
| UDP-N-acetylglucosamine transporter | acetylglucosaminyl transferase | galactosyl transferase |
| mannosyl(alpha-1,6-)-glycoprotein beta-1,2-N- | (GnTIII) | (GalT) |
| acetylglucosaminyl transferase (GnTII) | | UDP-galactose transporter |

NeuAc2Gal2GlcNAc2Man3GlcNAc2

| | |
|---|---|
| mannosyl(alpha-1,3-)-glycoprotein beta-1,2-N- | beta-N-acetylglucosaminyl |
| acetylglucosaminyl transferase (GnTI) | glycopeptide beta-1,4- |
| UDP-N-acetylglucosamine transporter | galactosyl transferase |
| mannosyl(alpha-1,6-)-glycoprotein beta-1,2-N- | (GalT) |
| acetylglucosaminyl transferase (GnTII) | UDP-galactose transporter |

NeuAc2Gal2GlcNAc2Man3GlcNAc2Fuc

| | |
|---|---|
| mannosyl(alpha-1,3-)-glycoprotein beta-1,2-N- | beta-N-acetylglucosaminyl |
| acetylglucosaminyl transferase (GnTI) | glycopeptide beta-1,4- |
| UDP-N-acetylglucosamine transporter | galactosyl transferase |
| mannosyl(alpha-1,6-)-glycoprotein beta-1,2-N- | (GalT) |
| acetylglucosaminyl transferase (GnTII) | UDP-galactose transporter |

NeuAc2Gal2GlcNAc3Man3GlcNAc2-bisecting

| | | |
|---|---|---|
| mannosyl(alpha-1,3-)-glycoprotein beta-1,2-N- | beta-1,4-mannosyl- | beta-N-acetylglucosaminyl |
| acetylglucosaminyl transferase (GnTI) | glycoprotein 4-beta-N- | glycopeptide beta-1,4- |
| UDP-N-acetylglucosamine transporter | acetylglucosaminyl transferase | galactosyl transferase |
| mannosyl(alpha-1,6-)-glycoprotein beta-1,2-N- | (GnTIII) | (GalT) |
| acetylglucosaminyl transferase (GnTII) | | UDP-galactose transporter |

NeuAc2Gal2GlcNAc3Man3GlcNAc2Fuc-bisecting

| mannosyl(alpha-1,3-)-glycoprotein beta-1,2-N- | beta-1,4-mannosyl- | beta-N-acetylglucosaminyl |
|---|---|---|
| acetylglucosaminyl transferase (GnTI) | glycoprotein 4-beta-N- | glycopeptide beta-1,4- |
| UDP-N-acetylglucosamine transporter | acetylglucosaminyl transferase | galactosyl transferase |
| mannosyl(alpha-1,6-)-glycoprotein beta-1,2-N- | (GnTIII) | (GalT) |
| acetylglucosaminyl transferase (GnTII) | | UDP-galactose transporter |
| N-acetylglucosaminylation | fucosylation | sialylation |

GlcNAcMan3-5GlcNAc2

mannosyl(alpha-1,3-)-glycoprotein beta-1,2-N-
acetylglucosaminyl transferase (GnTI)
UDP-N-acetylglucosamine transporter

GlcNAc2Man3GlcNAc2

mannosyl(alpha-1,3-)-glycoprotein beta-1,2-N-
acetylglucosaminyl transferase (GnTI)
UDP-N-acetylglucosamine transporter
mannosyl(alpha-1,6-)-glycoprotein beta-1,2-N-
acetylglucosaminyl transferase (GnTII)

GlcNAc3Man3GlcNAc2-bisecting

mannosyl(alpha-1,3-)-glycoprotein beta-1,2-N-
acetylglucosaminyl transferase (GnTI)
UDP-N-acetylglucosamine transporter
mannosyl(alpha-1,6-)-glycoprotein beta-1,2-N-
acetylglucosaminyl transferase (GnTII)

Gal2GlcNAc2Man3GlcNAc2

mannosyl(alpha-1,3-)-glycoprotein beta-1,2-N-
acetylglucosaminyl transferase (GnTI)
UDP-N-acetylglucosamine transporter
mannosyl(alpha-1,6-)-glycoprotein beta-1,2-N-
acetylglucosaminyl transferase (GnTII)

Gal2GlcNAc2Man3GlcNAc2Fuc

| | |
|---|---|
| mannosyl(alpha-1,3-)-glycoprotein beta-1,2-N- | GDP-D-mannose 4,6- |
| acetylglucosaminyl transferase (GnTI) | dehydratase |
| UDP-N-acetylglucosamine transporter | GDP-4-keto-6-deoxy-D- |
| mannosyl(alpha-1,6-)-glycoprotein beta-1,2-N- | mannose-3,5-epimerase- |
| acetylglucosaminyl transferase (GnTII) | 4-reductase |
| | GDP-fucose transporter |
| | alpha (1,6) fucosyl transferase |
| | (FucT) |

Gal2GlcNAc3Man3GlcNAc2-bisecting

mannosyl(alpha-1,3-)-glycoprotein beta-1,2-N-
acetylglucosaminyl transferase (GnTI)
UDP-N-acetylglucosamine transporter
mannosyl(alpha-1,6-)-glycoprotein beta-1,2-N-
acetylglucosaminyl transferase (GnTII)

Gal2GlcNAc3Man3GlcNAc2Fuc-bisecting

| | |
|---|---|
| mannosyl(alpha-1,3-)-glycoprotein beta-1,2-N- | GDP-D-mannose 4,6- |
| acetylglucosaminyl transferase (GnTI) | dehydratase |
| UDP-N-acetylglucosamine transporter | GDP-4-keto-6-deoxy-D- |
| mannosyl(alpha-1,6-)-glycoprotein beta-1,2-N- | mannose-3,5-epimerase- |
| acetylglucosaminyl transferase (GnTII) | 4-reductase |
| | GDP-fucose transporter |
| | alpha (1,6) fucosyl transferase |
| | (FucT) |

NeuAc2Gal2GlcNAc2Man3GlcNAc2

| | |
|---|---|
| mannosyl(alpha-1,3-)-glycoprotein beta-1,2-N- | beta-galactoside alpha- |
| acetylglucosaminyl transferase (GnTI) | 2,6-sialyl transferase (ST) |
| UDP-N-acetylglucosamine transporter | UDP-N-acetylglucosamine |
| mannosyl(alpha-1,6-)-glycoprotein beta-1,2-N- | 2-epimerase (NeuC) |
| acetylglucosaminyl transferase (GnTII) | sialic acid synthase |
| | (NeuB) or: N- |
| | acylneuraminate-9- |
| | phosphate synthase |
| | N-acylneuraminate-9- |
| | phosphatase |
| | CMP-Neu5Ac synthetase |
| | CMP-sialic acid transporter |

NeuAc2Gal2GlcNAc2Man3GlcNAc2Fuc

| | | |
|---|---|---|
| mannosyl(alpha-1,3-)-glycoprotein beta-1,2-N- | GDP-D-mannose 4,6- | beta-galactoside alpha- |
| acetylglucosaminyl transferase (GnTI) | dehydratase | 2,6-sialyl transferase (ST) |
| UDP-N-acetylglucosamine transporter | GDP-4-keto-6-deoxy-D- | UDP-N-acetylglucosamine |
| mannosyl(alpha-1,6-)-glycoprotein beta-1,2-N- | mannose-3,5-epimerase- | 2-epimerase (NeuC) |
| acetylglucosaminyl transferase (GnTII) | 4-reductase | sialic acid synthase |
| | GDP-fucose transporter | (NeuB) or: N- |
| | alpha (1,6) fucosyl transferase | acylneuraminate-9- |
| | (FucT) | phosphate synthase + |
| | | N-acylneuraminate-9- |
| | | phosphatase |
| | | CMP-Neu5Ac synthetase |
| | | CMP-sialic acid transporter |

NeuAc2Gal2GlcNAc3Man3GlcNAc2-bisecting

| | |
|---|---|
| mannosyl(alpha-1,3-)-glycoprotein beta-1,2-N- | beta-galactoside alpha- |
| acetylglucosaminyl transferase (GnTI) | 2,6-sialyl transferase (ST) |
| UDP-N-acetylglucosamine transporter | UDP-N-acetylglucosamine |
| mannosyl(alpha-1,6-)-glycoprotein beta-1,2-N- | 2-epimerase (NeuC) |
| acetylglucosaminyl transferase (GnTII) | sialic acid synthase |
| | (NeuB) or: N- |
| | acylneuraminate-9- |
| | phosphate synthase + |
| | N-acylneuraminate-9- |
| | phosphatase |
| | CMP-Neu5Ac synthetase |
| | CMP-sialic acid transporter |

NeuAc2Gal2GlcNAc3Man3GlcNAc2Fuc-bisecting

| | | |
|---|---|---|
| mannosyl(alpha-1,3-)-glycoprotein beta-1,2-N- | GDP-D-mannose 4,6- | beta-galactoside alpha- |
| acetylglucosaminyl transferase (GnTI) | dehydratase | 2,6-sialyl transferase (ST) |
| UDP-N-acetylglucosamine transporter | GDP-4-keto-6-deoxy-D- | UDP-N-acetylglucosamine |
| mannosyl(alpha-1,6-)-glycoprotein beta-1,2-N- | mannose-3,5-epimerase- | 2-epimerase (NeuC) |
| acetylglucosaminyl transferase (GnTII) | 4-reductase | sialic acid synthase (NeuB |
| | GDP-fucose transporter | or: N-acylneuraminate-9- |
| | alpha (1,6) fucosyl transferase | phosphate synthase + |
| | (FucT) | N-acylneuraminate-9- |
| | | phosphatase |
| | | CMP-Neu5Ac synthetase |
| | | CMP-sialic acid transporter |

TABLE 5
Heterologous enzymes for Golgi-based synthesis of preferred triantennary glycans

| | | | |
|---|---|---|---|
| N-acetylglucosaminylation | galactosylation | fucosylation | sialylation |

GlcNAc3Man3GlcNAc2

mannosyl(alpha-1,3-)-glycoprotein beta-1,2-N-
acetylglucosaminyl transferase (GnTI)
UDP-N-acetylglucosamine transporter
mannosyl(alpha-1,6-)-glycoprotein beta-1,2-N-
acetylglucosaminyl transferase (GnTII)
mannosyl(alpha-1,3-)-glycoprotein beta-1,4-N-
acetylglucosaminyl transferase(GnTIV)

Gal3GlcNAc3Man3GlcNAc2

| | |
|---|---|
| mannosyl(alpha-1,3-)-glycoprotein beta-1,2-N- | beta-N-acetylglucosaminyl |
| acetylglucosaminyl transferase (GnTI) | glycopeptide beta-1,4-galactosyl |
| UDP-N-acetylglucosamine transporter | transferase (GalT) |
| mannosyl(alpha-1,6-)-glycoprotein beta-1,2-N- | UDP-galactose transporter |
| acetylglucosaminyl transferase (GnTII) | |
| mannosyl(alpha-1,3-)-glycoprotein beta-1,4-N- | |
| acetylglucosaminyl transferase(GnTIV) | |

Gal3GlcNAc3Man3GlcNAc2Fuc

| | | |
|---|---|---|
| mannosyl(alpha-1,3-)-glycoprotein beta-1,2-N- | beta-N-acetylglucosaminyl | GDP-D-mannose 4,6-dehydratase |
| acetylglucosaminyl transferase (GnTI) | glycopeptide beta-1,4-galactosyl | GDP-4-keto-6-deoxy-D-mannose- |
| UDP-N-acetylglucosamine transporter | transferase (GalT) | 3,5-epimerase-4-reductase |
| mannosyl(alpha-1,6-)-glycoprotein beta-1,2-N- | UDP-galactose transporter | GDP-fucose transporter |
| acetylglucosaminyl transferase (GnTII) | | alpha (1,6) fucosyl transferase |
| mannosyl(alpha-1,3-)-glycoprotein beta-1,4-N- | | (FucT) |
| acetylglucosaminyl transferase(GnTIV) | | |

NeuAc3Gal3GlcNAc3Man3GlcNAc

| | | |
|---|---|---|
| mannosyl(alpha-1,3-)-glycoprotein beta- | beta-N-acetylglucosaminyl | 2,6-sialyl transferase (ST) |
| 1,2-N-acetylglucosaminyl transferase | glycopeptide beta-1,4- | UDP-N-acetylglucosamine 2- |
| (GnTI) | galactosyl transferase | epimerase (NeuC) |
| UDP-N-acetylglucosamine transporter | (GalT) | sialic acid synthase (NeuB) |
| mannosyl(alpha-1,6-)-glycoprotein beta- | UDP-galactose transporter | or: N-acylneuraminate-9- |
| 1,2-N-acetylglucosaminyl transferase | | phosphate synthase + N- |
| (GnTII) | | acylneuraminate-9-phosphatase |
| mannosyl(alpha-1,3-)-glycoprotein beta- | | CMP-Neu5Ac synthetase |
| 1,4-N-acetylglucosaminyl transferase | | CMP-sialic acid transporter |
| (GnTIV) | | |

NeuAc3Gal3GlcNAc3Man3GlcNAcFuc

| | | | |
|---|---|---|---|
| mannosyl(alpha-1,3-)-glycoprotein beta- | beta-N-acetylglucosaminyl | GDP-D-mannose 4,6- | 2,6-sialyl transferase (ST) |
| 1,2-N-acetylglucosaminyl transferase | glycopeptide beta-1,4- | dehydratase | UDP-N-acetylglucosamine 2- |
| (GnTI) | galactosyl transferase | GDP-4-keto-6-deoxy-D- | epimerase (NeuC) |
| UDP-N-acetylglucosamine transporter | (GalT) | mannose-3,5-epimerase-4- | sialic acid synthase (NeuB) |
| mannosyl(alpha-1,6-)-glycoprotein beta- | UDP-galactose transporter | reductase | or: N-acylneuraminate-9- |
| 1,2-N-acetylglucosaminyl transferase | | GDP-fucose transporter | phosphate synthase + N- |
| (GnTII) | | alpha (1,6) fucosyl transferase | acylneuraminate-9-phosphatase |
| mannosyl(alpha-1,3-)-glycoprotein beta- | | (FucT) | CMP-Neu5Ac synthetase |
| 1,4-N-acetylglucosaminyl transferase | | | CMP-sialic acid transporter |
| (GnTIV) | | | |

The primary goal of this genetic engineering effort is to produces robust protein production strains that are able to perform proteins with defined, human-like glycan structures in an industrial fermentation process. The integration of multiple genes into the host (e.g., fungal) chromosome involves careful planning. The engineered strain will most likely have to be transformed with a range of different genes, and these genes will have to be transformed in a stable fashion to ensure that the desired activity is maintained throughout the fermentation process. Any combination of the enzyme activities will have to be engineered into the protein expression host cell.

With DNA sequence information, the skilled worker can clone DNA molecules encoding GnT activities Using standard techniques well-known to those of skill in the art, nucleic acid molecules encoding one or more GnT (or encoding catalytically active fragments thereof) may be inserted into appropriate expression vectors under the transcriptional control of promoters and other expression control sequences capable of driving transcription in a selected host cell of the invention, e.g., a fungal host such as Pichia sp., Kluyveromyces sp., Saccharomyces sp., Yarrowia sp. and Aspergillus sp., as described herein, such that one or more of these mammalian GnT enzymes may be actively expressed in a host cell of choice for production of a human-like complex glycoprotein.

The engineered strains will be stably transformed with different glycosylation related genes to ensure that the desired activity is maintained throughout the fermentation process. Any combination of the following enzyme activities will have to be engineered into the expression host. In parallel a number of host genes involved in undesired glycosylation reactions will have to be deleted.

In preferred embodiments a subset of genes, at least two genes (also named library), encoding heterologous glycosylation enzymes are transformed into the host organism, causing at first a genetically mixed population. Transformants having the desired glycosylation phenotypes are then selected from the mixed population. In a preferred embodiment, the host organism is a lower eukaryote and the host glycosylation pathway is modified by the stable expression of one or more human or animal glycosylation enzymes, yielding N-glycans similar or identical to human glycan structures. In an especially preferred embodiment, the subset of genes or “DNA library” include genetic constructs encoding fusions of glycosylation enzymes with targeting sequences for various cellular loci involved in glycosylation especially the ER, cis Golgi, medial Golgi, or trans Golgi.

In some cases the DNA library may be assembled directly from existing or wild-type genes. In a preferred embodiment however the DNA library is assembled from the fusion of two or more sub-libraries. By the in-frame ligation of the sub-libraries, it is possible to create a large number of novel genetic constructs encoding useful targeted glycosylation activities. For example, one useful sub-library includes DNA sequences encoding any combination of the enzymes and enzymatic activities set forth hereinafter.

Preferably, the enzymes are of human origin, although other eukaryotic or also procaryotic enzymes, more particular mammalian, protozoan, plant, bacterial or fungal enzymes are also useful. In a preferred embodiment, genes are truncated to give fragments encoding the catalytic domains of the enzymes. By removing endogenous targeting sequences, the enzymes may then be redirected and expressed in other cellular loci. The choice of such catalytic domains may be guided by the knowledge of the particular environment in which the catalytic domain is subsequently to be active. Another useful sub-library includes DNA sequences encoding signal peptides that result in localization of a protein to a particular locus within the ER, Golgi, or trans Golgi network. These signal sequences may be selected from the host organism as well as from other related or unrelated organisms. Membrane-bound proteins of the ER or Golgi typically may include, for example, N-terminal sequences encoding a cytosolic tail (ct), a transmembrane domain (tmd), and a stem region (sr). The ct, tmd, and sr sequences are sufficient individually or in combination to anchor proteins to the inner (lumenal) membrane of the organelle. Accordingly, a preferred embodiment of the sub-library of signal sequences includes ct, tmd, and/or sr sequences from these proteins. In some cases it is desirable to provide the sub-library with varying lengths of sr sequence. This may be accomplished by PCR using primers that bind to the 5′ end of the DNA encoding the cytosolic region and employing a series of opposing primers that bind to various parts of the stem region. Still other useful sources of signal sequences include retrieval signal peptides.

In addition to the open reading frame sequences, it is generally preferable to provide each library construct with such promoters, transcription terminators, enhancers, ribosome binding sites, and other functional sequences as may be necessary to ensure effective transcription and translation of the genes upon transformation into the host organism.

According to this, the invention thus further concerns the host cell according to the invention as described herein with is further genetically engineered or modified to express at least one preferably heterologous enzyme or catalytic domain thereof, said enzyme or catalytic domain thereof is represented in tables 3, 4 , and 5 and is preferably selected from the group of Golgi-based heterologous enzymes consisting of:

mannosyl(alpha-1,3-)-glycoprotein beta-1,2-N-acetylglucosaminyl transferase or N-acetylglucosaminyl transferase I (GnTI);

mannosyl(alpha-1,6-)-glycoprotein beta-1,2-N-acetylglucosaminyl transferase or N-acetylglucosaminyl transferase II (GnTII);

beta-1,4-mannosyl-glycoprotein 4-beta-N-acetylglucosaminyl transferase or N-acetylglucosaminyl transferase III (GnTIII);

mannosyl(alpha-1,3-)-glycoprotein beta-1,4-N-acetylglucosaminyl transferase or N-acetylglucosaminyl transferase IV (GnTIV);

mannosyl(alpha-1,6-)-glycoprotein beta-1,6-N-acetyl-glucosaminyl transferase or N-acetylglucosaminyl transferase V (GnTV); alpha-1,6-mannosyl-glycoprotein 4-beta-N-acetylglucosaminyl transferase or N-acetylglucosaminyl transferase VI (GnTVI);

beta-N-acetylglucosaminyl glycopeptide beta-1,4-galactosyl transferase or galactosyl transferase (GalT);

alpha(1,6)fucosyl transferase or fucosyl transferase (FucT); beta-galactoside alpha-2,6-sialyl transferase or sialyl transferase (ST)

These enzyme activities may be further supported by the activity of one or more of the following: UDP-GlcNAc transferase; UDP-GlcNac transporter; UDP-galactosyl transferase, UDP-galactose transporter; GDP-fucosyl transferase; GDP-fucose transporter; CMP-sialyl transferase CMP-sialic acid transporter; and nucleotide di-phoshatases.

It goes without saying that said at least one enzyme or catalytic domain described herein preferably comprises at least a localization sequence for an intracellular membrane or organelle. In the preferred embodiments the intracellular membrane or organelle is the Golgi.

In preferred variants thereof, N-acetylglucosaminyl transferase V (GnTV) and/or N-acetylglucosaminyl transferase VI (GnTVI) are not present or are lacking in the modified cell. In these variants the modifications catalyzed by one or both of these two enzyme activities are not required or excluded from the Golgi-based modification.

### Embodiments for the Synthesis of GlcNAcMan3-5GlcNAc2 Structures

In a preferred embodiment the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript.

This cell may also comprise a, preferably heterologous, enzyme activity that is selected from:
UDP-N-acetylglucosamine transporter type activity, in particular a Slc35A3-type transcript.

In a most preferred embodiment, this cell comprises at least both of or exclusively these Golgi processing associated enzyme activities.

In a preferred variant of this embodiment the cell expresses one or more of one of the following genes:
mgat1 and slc35A3

and/or homologues thereof.

This cell is particularly capable of producing N-glycan with GlcNAcMan3-5GlcNAc2 structures. The invention thus also concerns a host cell or a plurality thereof, that is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

### Embodiments for the Synthesis of a GlcNAc2Man3GlcNAc2 Structure

In another preferred embodiment the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript;
UDP-N-acetylglucosamine transporter type activity, in particular a Slc35A3-type transcript; and
mannosyl(alpha-1,6-)-glycoprotein beta-1,2-N-acetylglucosaminyl transferase (GnTII), in particular a Mgat2-type transcript.

In a most preferred embodiment, this cell comprises at least all of or exclusively these Golgi processing associated enzyme activities.

In a preferred variant of this embodiment the cell expresses one or more of one of the following genes:
mgat1, mgat2, and slc35A3

and/or homologues thereof.

This cell is particularly capable of producing N-glycan with GlcNAc2Man3GlcNAc2 structure. The invention thus also concerns a host cell or a plurality thereof, that is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

### Embodiments for the Synthesis of a GlcNAc3Man3GlcNAc2-Bisecting

In another preferred embodiment the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript;
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript; and
beta-1,4-mannosyl-glycoprotein 4-beta-N-acetylglucosaminyl transferase (GnTIII), in particular a Mgat3-type transcript.

In a most preferred embodiment, this cell comprises at least all of or exclusively these Golgi processing associated enzyme activities.

In a preferred variant of this embodiment the cell expresses one or more of one of the following genes:
mgat1, mgat2, mgat3, and slc35A3

and/or homologues thereof.

This cell is particularly capable of producing N-glycan with GlcNAc2Man3GlcNAc2-bisecting structure. The invention thus also concerns a host cell or a plurality thereof, that is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

### Embodiments for the Synthesis of a Gal2GlcNAc2Man3GlcNAc2 Structure

In another preferred embodiment the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript;
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-N-acetylglucosaminyl glycopeptide beta-1,4-galactosyl transferase (GalT), in particular a B4galt1-type transcript; and
UDP-galactose transporter type activity, in particular a Slc35A2-type transcript.

In a most preferred embodiment, this cell comprises at least all of or exclusively these Golgi processing associated enzyme activities.

In a preferred variant of this embodiment the cell expresses one or more of one of the following genes:
mgat1, mgat2, mgat3, b4galt1, and slc35a2

and/or homologues thereof.

This cell is particularly capable of producing N-glycan with Gal2GlcNAc2Man3GlcNAc2 structure. The invention thus also concerns a host cell or a plurality thereof, that is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

### Embodiments for the Synthesis of a Gal2GlcNAc2Man3GlcNAc2Fuc Structure

In another preferred embodiment the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript;
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript; and
beta-N-acetylglucosaminyl glycopeptide beta-1,4-galactosyl transferase (GalT), in particular a B4galt1-type transcript;
UDP-galactose transporter type activity, in particular a Slc35A2-type transcript;
GDP-D-mannose 4,6-dehydratase type activity, in particular a Gmds-type transcript;
GDP-4-keto-6-deoxy-D-mannose-3,5-epimerase-4-reductase type activity, in particular a Tsta3-type transcript;
GDP-fucose transporter type activity, in particular a Slc35C1-type transcript; and
alpha(1,6)fucosyl transferase (FucT) type activity, in particular a Fut8-type transcript.

In a most preferred embodiment, this cell comprises at least all of or exclusively these Golgi processing associated enzyme activities.

In a preferred variant of this embodiment the cell expresses one or more of one of the following genes:
mgat1, mgat2, slc35a3, mgat3, b4galt1, slc35a2, gmds, tsta3, slc35c1 and fut8

and/or homologues thereof.

This cell is particularly capable of producing N-glycan with Gal2GlcNAc2Man3GlcNAc2Fuc structure. The invention thus also concerns a host cell or a plurality thereof, that is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

### Embodiments for the Synthesis of a Gal2GlcNAc3Man3GlcNAc2-Bisecting Structure

In another preferred embodiment the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript;
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-1,4-mannosyl-glycoprotein 4-beta-N-acetylglucosaminyl transferase (GnTIII), in particular a Mgat3-type transcript.
beta-N-acetylglucosaminyl glycopeptide beta-1,4-galactosyl transferase (GalT), in particular a B4galt1-type transcript; and
UDP-galactose transporter type activity, in particular a Slc35A2-type transcript.

In a most preferred embodiment, this cell comprises at least all of or exclusively these Golgi processing associated enzyme activities.

In a preferred variant of this embodiment the cell expresses one or more of one of the following genes:
mgat1, mgat2, mgat3, slc35a3, b4gat1, and slc35a2

and/or homologues thereof.

This cell is particularly capable of producing N-glycan with Gal2GlcNAc3Man3GlcNAc2-bisecting structure. The invention thus also concerns a host cell or a plurality thereof, that is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

### Embodiments for the Synthesis of a Gal2GlcNAc3Man3GlcNAc2Fuc-Bisecting Structure

In another preferred embodiment the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript;
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-1,4-mannosyl-glycoprotein 4-beta-N-acetylglucosaminyl transferase (GnTIII), in particular a Mgat3-type transcript.
beta-N-acetylglucosaminyl glycopeptide beta-1,4-galactosyl transferase (GalT), in particular a B4galt1-type transcript;
UDP-galactose transporter type activity, in particular a Slc35A2-type transcript;
GDP-D-mannose 4,6-dehydratase type activity, in particular a Gmds-type transcript;
GDP-4-keto-6-deoxy-D-mannose-3,5-epimerase-4-reductase type activity, in particular a Tsta3-type transcript;
GDP-fucose transporter type activity, in particular a Slc35C1-type transcript; and
alpha(1,6)fucosyl transferase (FucT) type activity, in particular a Fut8-type transcript.

In a most preferred embodiment, this cell comprises at least all of or exclusively these Golgi processing associated enzyme activities.

In a preferred variant of this embodiment the cell expresses one or more of one of the following genes:
mgat1, mgat2, mgat3, slc3533, b4galt1, slc35a2, gmds, tsta3, slc35c1 and fut8

and/or homologues thereof.

This cell is particularly capable of producing N-glycan with Gal2GlcNAc3Man3GlcNAc2Fuc-bisecting structure. The invention thus also concerns a host cell or a plurality thereof, that is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

### Embodiments for the Synthesis of a NeuAc2Gal2GlcNAc2Man3GlcNAc2 Structure

In another preferred embodiment the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript;
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
sialic acid synthase (NeuB), in particular a NeuB-type transcript;
CMP-Neu5Ac synthetase, in particular a Slc35A1-type transcript; and
CMP-sialic acid transporter, in particular a NeuA/Cmas-type transcript.

In a most preferred embodiment, this cell comprises at least all of or exclusively these Golgi processing associated enzyme activities.

In an alternative variant thereof, the modified host cell exhibits N-acylneuraminate-9-phosphate synthase and N-acylneuraminate-9-phosphatase activity instead of sialic acid synthase activity, more particular the the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript;
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
N-acylneuraminate-9-phosphate synthase;
N-acylneuraminate-9-phosphatase;
CMP-Neu5Ac synthetase, in particular a Slc35A1-type transcript; and
CMP-sialic acid transporter, in particular a NeuA/Cmas-type transcript.

In a most preferred embodiment, this cell comprises at least all of or exclusively these Golgi processing associated enzyme activities.

In a preferred variant of these embodiments the cell expresses one or more of one of the following genes:
mgat1, mgat2, slc35a3, b4galt1, slc35a2, st6gal1, neuC, neuB, slc35a1, and neuC/cmas

and/or homologues thereof.

This cell is particularly capable of producing N-glycan with NeuAc2Gal2GlcNAc2Man3GlcNAc2 structure. The invention thus also concerns a host cell or a plurality thereof, that is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

### Embodiments for the Synthesis of a NeuAc2Gal2GlcNAc3Man3GlcNAc2-Bisecting Structure

In another preferred embodiment the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript;
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-1,4-mannosyl-glycoprotein 4-beta-N-acetylglucosaminyl transferase (GnTIII), in particular a Mgat3-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
sialic acid synthase (NeuB), in particular a NeuB-type transcript;
CMP-Neu5Ac synthetase, in particular a Slc35A1-type transcript; and
CMP-sialic acid transporter, in particular a NeuA/Cmas-type transcript.

In a most preferred embodiment, this cell comprises at least all of or exclusively these Golgi processing associated enzyme activities.

In an alternative variant thereof, the modified host cell exhibits N-acylneuraminate-9-phosphate synthase and N-acylneuraminate-9-phosphatase activity instead of sialic acid synthase activity, more particular the the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript;
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-1,4-mannosyl-glycoprotein 4-beta-N-acetylglucosaminyl transferase (GnTIII), in particular a Mgat3-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
N-acylneuraminate-9-phosphate synthase;
N-acylneuraminate-9-phosphatase;
CMP-Neu5Ac synthetase, in particular a Slc35A1-type transcript; and
CMP-sialic acid transporter, in particular a NeuA/Cmas-type transcript.

In a most preferred embodiment, this cell comprises at least all of or exclusively these Golgi processing associated enzyme activities.

In a preferred variant of these embodiments the cell expresses one or more of one of the following genes:
mgat1, mgat2, slc35a3, mgat3, b4galt1, slc35a2, st6gal1, neuC, neuB, slc35a1, and neuC/cmas

and/or homologues thereof.

This cell is particularly capable of producing N-glycan with NeuAc2Gal2GlcNAc2Man3GlcNAc2-bisecting structure. The invention thus also concerns a host cell or a plurality thereof, that is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

### Embodiments for the Synthesis of a NeuAc2Gal2GlcNAc2Man3GlcNAc2Fuc Structure

In another preferred embodiment the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript;
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
GDP-D-mannose 4,6-dehydratase type activity, in particular a Gmds-type transcript;
GDP-4-keto-6-deoxy-D-mannose-3,5-epimerase-4-reductase type activity, in particular a Tsta3-type transcript;
GDP-fucose transporter type activity, in particular a Slc35C1-type transcript;
alpha(1,6)fucosyl transferase (FucT) type activity, in particular a Fut8-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
sialic acid synthase (NeuB), in particular a NeuB-type transcript;
CMP-Neu5Ac synthetase, in particular a Slc35A1-type transcript; and
CMP-sialic acid transporter, in particular a NeuA/Cmas-type transcript.

In a most preferred embodiment, this cell comprises at least all of or exclusively these Golgi processing associated enzyme activities.

In an alternative variant thereof, the modified host cell exhibits N-acylneuraminate-9-phosphate synthase and N-acylneuraminate-9-phosphatase activity instead of sialic acid synthase activity, more particular the the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript;
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
GDP-D-mannose 4,6-dehydratase type activity, in particular a Gmds-type transcript;
GDP-4-keto-6-deoxy-D-mannose-3,5-epimerase-4-reductase type activity, in particular a Tsta3-type transcript;
GDP-fucose transporter type activity, in particular a Slc35C1-type transcript;
alpha(1,6)fucosyl transferase (FucT) type activity, in particular a Fut8-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
N-acylneuraminate-9-phosphate synthase;
N-acylneuraminate-9-phosphatase;
CMP-Neu5Ac synthetase, in particular a Slc35A1-type transcript; and
CMP-sialic acid transporter, in particular a NeuA/Cmas-type transcript.

In a most preferred embodiment, this cell comprises at least all of or exclusively these Golgi processing associated enzyme activities.

In a preferred variant of these embodiments the cell expresses one or more of one of the following genes:
mgat1, mgat2, slc35a3, b4galt1, slc35a2, gmds, tsta3, slc35c1, fut8, st6gal1, neuC, neuB, slc35a1, and neuC/cmas

and/or homologues thereof.

This cell is particularly capable of producing N-glycan with NeuAc2Gal2GlcNAc2Man3GlcNAc2Fuc structure. The invention thus also concerns a host cell or a plurality thereof, that is specifically designed to produce glycoproteins with this glycan structure. The invention thus also concerns a, preferably isolated, glycoprotein having this structure, which is preferably producible or actuality produced by this cell. The invention also provides a method or process for making that glycoprotein by using this cell.

### Embodiments for the Synthesis of a NeuAc2Gal2GlcNAc3Man3GlcNAc2Fuc-Bisecting Structure

In another preferred embodiment the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript;
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-1,4-mannosyl-glycoprotein 4-beta-N-acetylglucosaminyl transferase (GnTIII), in particular a Mgat3-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
GDP-D-mannose 4,6-dehydratase type activity, in particular a Gmds-type transcript;
GDP-4-keto-6-deoxy-D-mannose-3,5-epimerase-4-reductase type activity, in particular a Tsta3-type transcript;
GDP-fucose transporter type activity, in particular a Slc35C1-type transcript;
alpha(1,6)fucosyl transferase (FucT) type activity, in particular a Fut8-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
sialic acid synthase (NeuB), in particular a NeuB-type transcript;
CMP-Neu5Ac synthetase, in particular a Slc35A1-type transcript; and
CMP-sialic acid transporter, in particular a NeuA/Cmas-type transcript.

In a most preferred embodiment, this cell comprises at least all of or exclusively these Golgi processing associated enzyme activities.

In an alternative variant thereof, the modified host cell exhibits N-acylneuraminate-9-phosphate synthase and N-acylneuraminate-9-phosphatase activity instead of sialic acid synthase activity, more particular the the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript;
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-1,4-mannosyl-glycoprotein 4-beta-N-acetylglucosaminyl transferase (GnTIII), in particular a Mgat3-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
GDP-D-mannose 4,6-dehydratase type activity, in particular a Gmds-type transcript;
GDP-4-keto-6-deoxy-D-mannose-3,5-epimerase-4-reductase type activity, in particular a Tsta3-type transcript;
GDP-fucose transporter type activity, in particular a Slc35C1-type transcript;
alpha(1,6)fucosyl transferase (FucT) type activity, in particular a Fut8-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
N-acylneuraminate-9-phosphate synthase;
N-acylneuraminate-9-phosphatase;
CMP-Neu5Ac synthetase, in particular a Slc35A1-type transcript; and
CMP-sialic acid transporter, in particular a NeuA/Cmas-type transcript.

In a most preferred embodiment, this cell comprises at least all of or exclusively these Golgi processing associated enzyme activities.

In a preferred variant of these embodiments the cell expresses one or more of one of the following genes:
mgat1, mgat2, slc35a3, b4galt1, mgat3, slc35a2, gmds, tsta3, slc35c1, fut8, st6gal1, neuC, neuB, slc35a1, and neuC/cmas

and/or homologues thereof.

This cell is particularly capable of producing N-glycan with NeuAc2Gal2GlcNAc2Man3GlcNAc2Fuc-bisecting structure. The invention thus also concerns a host cell or a plurality thereof, that is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

### Embodiments for the Synthesis of a GlcNAc3Man3GlcNAc2 Structure

In another preferred embodiment the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript,
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript; and
mannosyl(alpha-1,3-)-glycoprotein beta-1,4-N-acetylglucosaminyl transferase(GnTIV), in particular a Mgat4-type transcript.

In a most preferred embodiment, this cell comprises at least all of or exclusively these Golgi processing associated enzyme activities.

In a preferred variant of this embodiment the cell expresses one or more of one of the following genes:
mgat1, mgat2, mgat4, and slc35A3

and/or homologues thereof.

This cell is particularly capable of producing N-glycan with GlcNAc3Man3GlcNAc2 structure. The invention thus also concerns a host cell or a plurality thereof, that is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

### Embodiments for the Synthesis of a Gal3GlcNAc3Man3GlcNAc2 Structure

In another preferred embodiment the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript,
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
mannosyl(alpha-1,3-)-glycoprotein beta-1,4-N-acetylglucosaminyl transferase(GnTIV), in particular a Mgat4-type transcript;
beta-N-acetylglucosaminyl glycopeptide beta-1,4-galactosyl transferase (GalT), in particular a B4galt1-type transcript; and
UDP-galactose transporter type activity, in particular a Slc35A2-type transcript.

In a most preferred embodiment, this cell comprises at least all of or exclusively these Golgi processing associated enzyme activities.

In a preferred variant of this embodiment the cell expresses one or more of one of the following genes:
mgat1, mgat2, maga4, slc35a3, b4galt1 and slc35a2

and/or homologues thereof.

This cell is particularly capable of producing N-glycan with Gal3-GlcNAc3Man3GlcNAc2 structure. The invention thus also concerns a host cell or a plurality thereof, that is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

### Embodiments for the Synthesis of a Gal3GlcNAc3Man3GlcNAc2Fuc Structure

In another preferred embodiment the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript,
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
mannosyl(alpha-1,3-)-glycoprotein beta-1,4-N-acetylglucosaminyl transferase(GnTIV), in particular a Mgat4-type transcript;
beta-N-acetylglucosaminyl glycopeptide beta-1,4-galactosyl transferase (GalT), in particular a B4galt1-type transcript; and
UDP-galactose transporter type activity, in particular a Slc35A2-type transcript;
GDP-D-mannose 4,6-dehydratase type activity, in particular a Gmds-type transcript;
GDP-4-keto-6-deoxy-D-mannose-3,5-epimerase-4-reductase type activity, in particular a Tsta3-type transcript;
GDP-fucose transporter type activity, in particular a Slc35C1-type transcript; and
alpha (1,6) fucosyl transferase (FucT) type activity, in particular a Fut8-type transcript.

In a most preferred embodiment, this cell comprises at least all of or exclusively these Golgi processing associated enzyme activities.

In a preferred variant of this embodiment the cell expresses one or more of one of the following genes:
mgat1, mgat2, maga4, slc35a3, b4galt1, slc35a2, gmds, tsta3, slc35c1 and fut8

and/or homologues thereof.

This cell is particularly capable of producing N-glycan with Gal3-GlcNAc3Man3GlcNAc2Fuc structure. The invention thus also concerns a host cell or a plurality thereof, that is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

### Embodiments for the Synthesis of a NeuAc3Gal3GlcNAc3Man3GlcNAc2 Structure

In another preferred embodiment the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript;
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
mannosyl(alpha-1,3-)-glycoprotein beta-1,4-N-acetylglucosaminyl transferase(GnTIV), in particular a Mgat4-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
sialic acid synthase (NeuB), in particular a NeuB-type transcript;
CMP-Neu5Ac synthetase, in particular a Slc35A1-type transcript; and
CMP-sialic acid transporter, in particular a NeuA/Cmas-type transcript.

In a most preferred embodiment, this cell comprises at least all of or exclusively these Golgi processing associated enzyme activities.

In an alternative variant thereof, the modified host cell exhibits N-acylneuraminate-9-phosphate synthase and N-acylneuraminate-9-phosphatase activity instead of sialic acid synthase activity, more particular the the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript;
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
mannosyl(alpha-1,3-)-glycoprotein beta-1,4-N-acetylglucosaminyl transferase(GnTIV), in particular a Mgat4-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
N-acylneuraminate-9-phosphate synthase;
N-acylneuraminate-9-phosphatase;
CMP-Neu5Ac synthetase, in particular a Slc35A1-type transcript; and
CMP-sialic acid transporter, in particular a NeuA/Cmas-type transcript.

In a most preferred embodiment, this cell comprises at least all of or exclusively these Golgi processing associated enzyme activities.

In a preferred variant of these embodiments the cell expresses one or more of one of the following genes:
mgat1, mgat2, slc35a3, b4galt1, mgat4, slc35a2, st6gal1, neuC, neuB, slc35a1, and neuC/cmas

and/or homologues thereof.

This cell is particularly capable of producing N-glycan with NeuAc3Gal3GlcNAc3Man3GlcNAc2 structure. The invention thus also concerns a host cell or a plurality thereof, that is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

### Embodiments for the Synthesis of a NeuAc3Gal3GlcNAc3Man3GlcNAc2Fuc Structure

In another preferred embodiment the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript;
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
mannosyl(alpha-1,3-)-glycoprotein beta-1,4-N-acetylglucosaminyl transferase(GnTIV), in particular a Mgat4-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
GDP-D-mannose 4,6-dehydratase type activity, in particular a Gmds-type transcript;
GDP-4-keto-6-deoxy-D-mannose-3,5-epimerase-4-reductase type activity, in particular a Tsta3-type transcript;
GDP-fucose transporter type activity, in particular a Slc35C1-type transcript;
alpha (1,6) fucosyl transferase (FucT) type activity, in particular a Fut8-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
sialic acid synthase (NeuB), in particular a NeuB-type transcript;
CMP-Neu5Ac synthetase, in particular a Slc35A1-type transcript; and
CMP-sialic acid transporter, in particular a NeuA/Cmas-type transcript.

In a most preferred embodiment, this cell comprises at least all of or exclusively these Golgi processing associated enzyme activities.

In an alternative variant thereof, the modified host cell exhibits N-acylneuraminate-9-phosphate synthase and N-acylneuraminate-9-phosphatase activity instead of sialic acid synthase activity, more particular the the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript;
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
mannosyl(alpha-1,3-)-glycoprotein beta-1,4-N-acetylglucosaminyl transferase(GnTIV), in particular a Mgat4-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
GDP-D-mannose 4,6-dehydratase type activity, in particular a Gmds-type transcript;
GDP-4-keto-6-deoxy-D-mannose-3,5-epimerase-4-reductase type activity, in particular a Tsta3-type transcript;
GDP-fucose transporter type activity, in particular a Slc35C1-type transcript;
alpha (1,6) fucosyl transferase (FucT) type activity, in particular a Fut8-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
N-acylneuraminate-9-phosphate synthase;
N-acylneuraminate-9-phosphatase;
CMP-Neu5Ac synthetase, in particular a Slc35A1-type transcript; and
CMP-sialic acid transporter, in particular a NeuA/Cmas-type transcript.

In a most preferred embodiment, this cell comprises at least all of or exclusively these Golgi processing associated enzyme activities.

In a preferred variant of these embodiments the cell expresses one or more of one of the following genes:
mgat1, mgat2, slc35a3, b4galt1, mgat4, slc35a2, gmds, tsta3, slc35c1, fut8, st6gal1, neuC, neuB, slc35a1, and neuC/cmas

and/or homologues thereof.

This cell is particularly capable of producing N-glycan with NeuAc3Gal2GlcNAc2Man3GlcNAc2Fuc structure. The invention thus also concerns a host cell or a plurality thereof, that is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

Method or Process for Making a Glycoprotein

The invention also provides a method or process for making a glycoprotein by using any one of the host cell according to the invention. Without wishing to be bound to the theory, a cell according to the invention is capable of producing high amounts of a N-Glycan with a Man1GlcNac2, Man2GlcNac2 or Man3GlcNac2 structure on said glycoprotein. The glycoprotein may be a homologous or a heterologous protein. Accordingly, any one of the host cells as outlined above preferably comprise at least one nucleic acid encoding a heterologous glycoprotein. Homologous proteins primarily refers to proteins from the host cell itself, whereas proteins encoded by “foreign”, cloned genes are heterologous proteins of the host cell. More particular, any nucleic acid encoding a heterologous protein according to the invention can be codon-optimized for expression in the host cell of interest. For example, a nucleic acid encoding a POT activity of Trypanosoma brucei can be codon-optimized for expression in a yeast cell such as Saccharomyces cerevisiae.

The host cell according to the invention is capable of producing complex N-linked oligosaccharides and hybrid oligosaccharides. Branched complex N-glycans have been implicated in the physiological activity of therapeutic proteins, such as human erythropoietin (hEPO). Human EPO having bi-antennary structures has been shown to have a low activity, whereas hEPO having tetra-antennary structures resulted in slower clearance from the bloodstream and thus in higher activity (Misaizu T et al. (1995) Blood December 1;86(1 1):4097-104).

A glycan structure means an oligosaccharide bound to a protein core. High mannose structures contain more than 5 mannoses whereas glycan structures consisting primarily of mannose but only to an extend less than 5 mannose moieties are low mannose glycan structures, e.g. Man3GlcNac2. More particular, as used herein, the term “glycan” or “glycoprotein” refers to an N-linked oligosaccharide, e.g., one that is attached by an asparagine-N-acetylglucosamine linkage to an asparagine residue of a polypeptide. N-glycans have a common pentasaccharide core of Man3 GlcNAc2 (“Man” refers to mannose; “Glc” refers to glucose; and “NAc” refers to N-acetyl; GlcNAc refers to N-acetylglucosamine). N-glycans differ with respect to the number of branches (antennae) comprising peripheral sugars (e.g., fucose and sialic acid) that are added to the Man3GlcNAc2 (“Man3”) core structure. N-glycans are classified according to their branched constituents (e.g., high mannose, complex or hybrid). A glycoform represents a glycosylated protein which carries a specific N-glycan. Therefore, glycoforms represent glycosylated proteins carrying different N-glycans. A “high mannose” type N-glycan has five or more mannose residues.

Common to all classes of N-glycans is the core structure Man3GlcNac2. The core structure is followed by an extension sequence on each branch, terminated by a cell-type specific hexose. Three general types of N-glycan structures could be defined: (1) High-mannose glycans, which contain mainly mannoses within their extension sequences and also as terminating moiety. (2) Complex glycans in contrast are composed of different hexoses. In humans they often contain N-acatylnauraminic acid as terminal hexose. And (3) hybrid glycans contain both, polymannosylic and complex extension sequences within one single glycan.

A “complex” type N-glycan typically has at least one GlcNAc attached to the 1,3 mannose arm and at least one GlcNAc attached to the 1,6 mannose arm of a “trimannose” core. The “trimannose core” is the pentasaccharide core having a Man3 structure. Complex N-glycans may also have galactose (“Gal”) residues that are optionally modified with sialic acid or derivatives (“NeuAc”, where “Neu” refers to neuraminic acid and “Ac” refers to acetyl). Complex N-glycans may also have intrachain substitutions comprising “bisecting” GlcNAc and core fucose (“Fuc”). A “hybrid” N-glycan has at least one GlcNAc on the terminal of the 1,3 mannose arm of the trimannose core and zero or more mannoses on the 1,6 mannose arm of the trimannose core.

A further aspect of the invention is a process for making a glycoprotein with a low mannose glycan structure or a glycoprotein-composition comprising one or more glycoproteins having low mannose glycan structure.

In a preferred embodiment the protein is an heterologous protein. In a preferred variant thereof the heterologous protein is a recombinant protein. A preferred embodiment of the invention is a composition that is comprising an heterologous and/or recombinant glycoprotein that is produced or producible by the cell of the invention, wherein the composition comprises a high yield of glycoprotein having a glycan structure of Man1-3GlcNAc2

“Recombinant protein”, “heterologous protein” and “heterologous protein” are used interchangeably to refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid.

In a preferred variant there is provided a process for making a glycoprotein with a Man3GlcNAc2 glycan structure or a glycoprotein-composition comprising at least one glycoprotein with a Man3GlcNAc2 glycan structure. In another preferred variant there is provided a process for making a glycoprotein with a Man2GlcNAc2 glycan structure or a glycoprotein-composition comprising at least one glycoprotein with a Man2GlcNAc2 glycan structure. In another preferred variant there is also provided a process for making a glycoprotein with a Man1GlcNAc2 glycan structure or a glycoprotein-composition comprising at least one glycoprotein with a Man1GlcNAc2 glycan structure. In another preferred variant there is also provided a process for making a human-like glycoprotein with a Man4GlcNAc2 glycan structure or a glycoprotein-composition comprising at least one glycoprotein with a Man4GlcNAc2 glycan structure. In another preferred variant there is also provided a process for making a human-like glycoprotein with a Man5GlcNAc2 glycan structure or a glycoprotein-composition comprising at least one glycoprotein with a Man5GlcNAc2 glycan structure.

The process comprises at least the following step: Provision of a mutant cell according to the invention. The cell is cultured in a preferably liquid culture medium and preferably under conditions that allow or most preferably support the production of said glycoprotein or glycoprotein composition in the cell. If necessary, required said glycoprotein or glycoprotein composition may be isolated from said cell and/or said culture medium. The isolation is preferably performed using methods and means known in the art.

The invention also provides new glycoproteins and compositions thereof, which are producible or are produced by the cells or methods according to the invention. Such compositions are further characterized in comprising glycan core structures selected from Man1GlcNAc2, Man2GlcNAc2, and Man3GlcNAc2, preferably a Man3GlcNAc2 structure. The invention may also provide compositions characterized in comprising glycan structures selected from Man4GlcNAc2 and Man5GlcNAc2, which may be produced due to further mannosylation of a Man1GlcNAc2, Man2GlcNAc2 or Man3GlcNAc2 core in the Golgi.

In preferred embodiments one or more said glycan structure is present in the composition in an amount of at least 40% or more, more preferred at least 50% or more, even more preferred 60% or more, even more preferred 70% or more, even more preferred 80% or more, even more preferred 90% or more, even more preferred 95% or more, most preferred to 99% or 100%. It goes without saying that other substances and by-products that are common to such protein compositions are excluded from that calculation. In a most preferred embodiment basically all glycan structures produced by the cell exhibit a Man3GlcNAc2 structure. In another preferred embodiment basically all glycoforms produced by the cell exhibit a Man4GlcNAc2 and/or a Man5GlcNAc2 structure.

As the result of the Golgi-modification, as described hereinabove in more detail, a glycoprotein carrying complex as well as hybrid N-glycans are obtainable. The glycoproteins comprise glycan structures selected from, but not limited to:
GlcNAcMan3-5GlcNAc2,
GlcNAc2Man3GlcNAc2,
GlcNAc3Man3GlcNAc2-bisecting
Gal2GlcNAc2Man3GlcNAc2,
Gal2GlcNAc2Man3GlcNAc2Fuc,
Gal2GlcNAc3Man3GlcNAc2-bisecting,
Gal2GlcNAc3Man3GlcNAc2Fuc-bisecting,
NeuAc2Gal2GlcNAc2Man3GlcNAc2, NeuAc2Gal2GlcNAc2Man3GlcNAc2Fuc,
NeuAc2Gal2GlcNAc3Man3GlcNAc2-bisecting,
NeuAc2Gal2GlcNAc3Man3GlcNAc2Fuc-bisecting,
GlcNAc3Man3GlcNAc2,
Gal3GlcNAc3Man3GlcNAc2,
Gal3GlcNAc3Man3GlcNAc2Fuc,
NeuAc3Gal3GlcNAc3Man3GlcNAc2, and
NeuAc3Gal3GlcNAc3Man3GlcNAc2Fuc.

In preferred embodiments one or more of the above-identified glycan structures is present in the glycoprotein or glycoprotein composition in an amount of at least about 40% or more, more preferred at least about 50% or more, even more preferred about 60% or more, even more preferred about 70% or more, even more preferred 80% or more, even more preferred about 90% or more, even more preferred about 95% or more, and most preferred 99% to all glycoproteins. It goes without saying that other substances and by-products that are common to such protein compositions are excluded from that calculation. In a most preferred embodiment basically all glycoproteins that are produced by the host cell of the invention exhibit one or more of the above-identified glycan structures.

In some embodiments, the N-glycosylation form of the glycoprotein according to the invention can be homogenous or substantially homogenous. In particular, the fraction of one particular glycan structure in the glycoprotein is at least about 20% or more, about 30% or more, about 40% or more, more preferred at least about 50% or more, even more preferred about 60% or more, even more preferred about 70% or more, even more preferred 80% or more, even more preferred about 90% or more, even more preferred about 95% or more, and most preferred 99% to all glycoproteins.

Preferred embodiments of the invention are novel glycoprotein compositions that are produced or are producible by the host cells exhibiting at two or more different glycoproteins of the above-identified glycan structures. Without wishing to be bound to the theory, in a preferred embodiment a particular host cell of the invention is capable of producing two or more different at the same time, which results in “mixtures” of glycoproteins of different structure. This also refers to intermediate forms of glycosylation. It must be noted that in most preferred variants of the invention the host cell provides to an essential extend, mainly or even purely (more than 90%, preferably more than 95%, most preferred 99% or more), one particular glycan structure.

In another preferred embodiment, two or more different host cells of the invention that preferably are co-cultivated to produce two or more different N-glycan structures, which results in “mixtures” of glycoproteins of different structure.

Instrumentation suitable for N-glycan analysis includes, e.g., the ABI PRISM® 377 DNA sequencer (Applied Biosystems). Data analysis can be performed using, e.g., GENESCAN® 3.1 software (Applied Biosystems). Additional methods of N-glycan analysis include, e.g., mass spectrometry (e.g., MALDI-TOF-MS), high-pressure liquid chromatography (HPLC) on normal phase, reversed phase and ion exchange chromatography (e.g., with pulsed amperometric detection when glycans are not labeled and with UV absorbance or fluorescence if glycans are appropriately labeled).

A preferred embodiment is a recombinant immunoglobulin such as an IgG, producible by the cell of the invention, comprising N-glycan of Gal2GlcNAc2Man3GlcNAc2 structure.

Another more preferred embodiment is a recombinant human Erythropoetin (rhuEPO), producible by the cell of the invention, comprising three N-glycans of NeuAc3Gal3GlcNAc3Man3GlcNAc2Fuc structure.

In preferred embodiments the glycoproteins or glycoprotein compositions can, but need not, be isolated from the host cells. In preferred embodiments the glycoproteins or glycoprotein compositions can, but need not, be further purified from the host cells. As used herein, the term “isolated” refers to a molecule, or a fragment thereof, that has been separated or purified from components, for example, proteins or other naturally-occurring biological or organic molecules, which naturally accompany it. Typically, an isolated glycoprotein or glycoprotein composition of the invention constitutes at least 60%, by weight, of the total molecules of the same type in a preparation, e.g., 60% of the total molecules of the same type in a sample. For example, an isolated glycoprotein constitutes at least 60%, by weight, of the total protein in a preparation or sample. In some embodiments, an isolated glycoprotein in the preparation consists of at least 75%, at least 90%, or at least 99%, by weight, of the total molecules of the same type in a preparation.

The genetically engineered host cells can be used in methods to produce novel glycoprotein or compositions thereof that are therapeutically active.

Preferred glycoproteins or glycoprotein compositions that are produced or are producible by the host cells according the above identified preferred embodiments include, but are not limited to, blood factors, anticoagulants, thrombolytics, antibodies, antigen-binding fragments thereof, hormones, growth factors, stimulating factors, chemokines, and cytokines, more particularly, regulatory proteins of the TFN-family, erythropoietin (EPO), gonadotropins, immunoglobulins, granulocyte-macrophage colony-stimulating factors, interferones, and enzymes. Most preferred glycoproteins or glycoprotein compositions are selected from: erythropoietin (EPO), interferon-[alpha], interferon-[beta], interferon-[gamma], interferon-[omega], and granulocyte-CSF, factor VIII, factor IX, human protein C, soluble IgE receptor [alpha]-chain, immunoglobuline-G (IgG), Fab of IgG, IgM, urokinase, chymase, urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteoprotegerin, glucocerebrosidase, galactocerebrosidase, alpha-L-iduronidase, beta-D-galactosidase, beta-glucosidase, beta-hexosaminidase, beta-D-mannosidase, alpha-L-fucosidase, arylsulfatase B, arylsulfatase A, alpha-N-acteylgalactosaminidase, aspartyiglucosaminidase, iduronate-2-sulfatase, alpha-glucosaminide-N-acetyltransferase, beta-D-glucoronidase, hyaluronidase, alpha-L-mannosidase, alpha-neuraminidase, phosphotransferase, acid lipase, acid ceramidase, sphinogmyelinase, thioesterase, cathepsin K, and lipoprotein lipase.

Another embodiment of the invention is a recombinant therapeutically active protein or a plurality of such proteins which is comprising one or more of the above-identified glycoproteins, in particular glycoproteins having an above-identified low-mannose glycan structure. The therapeutically active protein is preferably producible by the cell according to the present invention.

A preferred embodiment thereof is an immunoglobulin or a plurality of immunoglobulins. Another preferred embodiment thereof is an antibody or antibody-composition comprising one or more of the above-identified immunoglobulins. The term “immunoglobulin” refers to any molecule that has an amino acid sequence by virtue of which it specifically interacts with an antigen and wherein any chains of the molecule contain a functionally operating region of an antibody variable region including, without limitation, any naturally occurring or recombinant form of such a molecule such as chimeric or humanized antibodies. As used herein, “immunoglobulin” means a protein which consists of one or more polypeptides essentially encoded by an immunoglobulin gene. The immunoglobulin of the present invention preferably encompasses active fragments, preferably fragments comprising one or more glycosylation site. The active fragments mean fragments of antibody having an antigen-antibody reaction activity, and include F(ab′)2, Fab′, Fab, Fv, and recombinant Fv.

Yet another preferred embodiment is a pharmaceutical composition which is comprising one or more of the following: one or more of the above-identified glycoprotein or glycoprotein-composition according the invention, one or more of the above-identified recombinant therapeutic protein according the invention, one or more of the above-identified immunoglobulin according the invention, and one or more of the above-identified antibody according the invention. If necessary or applicable, the composition further comprises at least one pharmaceutically acceptable carrier or adjuvant.

The glycoproteins of the invention can be formulated in pharmaceutical compositions. These compositions may comprise, in addition to one of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal or patch routes.

Pharmaceutical compositions for oral administration may be in tablet, capsule, powder or liquid form. A tablet may include a solid carrier such as gelatine or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles. Preservatives, stabilizers, buffers, antioxidants and/or other additives may be included, as required.

Whether it is a polypeptide, peptide, or nucleic acid molecule, other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners.

In another aspect, the invention provides a method of treating a disorder treatable by administration of one or more of the above-identified glycoproteins or compositions thereof, the method comprising the step(s) of: administering to a subject the glycoprotein or composition as described above, wherein the subject is suffering from, or is suspected to, a disease treatable by administration of that glycoprotein or composition. In a preferred embodiment, the method also includes the steps of (a) providing a subject and/or (b) determining whether the subject is suffering from a disease treatable by administration of said glycoprotein or composition. The subject can be mammal such as a human. The disorder can be, for example, a cancer, an immunological disorder, an inflammatory condition or a metabolic disorder.

According to the invention, there is also provided a kit or kit-of-parts for producing a glycoprotein, the kit is comprising at least: one or more host cells according to the invention, that are capable of producing the recombinant protein, and preferably a culture medium for culturing the cell so as to produce the recombinant protein.

### DESCRIPTION OF THE DRAWINGS

FIG. 1 depicts a schematic representation of biosynthetic lipid-linked oligosaccharide (LLO) pathway in yeast. LLO synthesis is initiated at the outer membrane of the ER, upon generation of Man5GlcNAc2 (M5) structure, the LLO is flipped into the ER lumen and the LLO synthesis is completed. The oligosaccharide is transferred to the protein by the OT (OST).

FIG. 2 depicts HPLC traces of [3H]-mannose-labeled lipid-linked oligosaccharides from Δalg11 mutant strains (YG1365) (FIG. 2A), and Δalg3Δalg11 mutant strains (YG1363) (FIG. 2B), showing the generation of a Man3GlcNAc2 structure (M3) in YG1363.

FIG. 3 depicts HPLC traces of [3H]-mannose-labeled protein-linked oligosaccharides from Δalg11 mutant strains (YG1365) (FIG. 3A), and Δalg3Δalg11 mutant strains (YG1363) (FIG. 3B). The ER synthesized Man3GlcNAc2 LLO structure (M3) is further extended in the Golgi compartment to Man4GlcNAc2 (M4) and Man5GlcNAc2 (M5).

FIG. 4 depicts MALDI-TOF MS spectra of 2-AB-labeled N-glycans isolated from cell wall proteins from wild-type strains (WT) (FIG. 4A), Δalg11 mutant strains (YG1365) (FIG. 4B), and Δalg3Δalg11 mutant strains (YG1363) (FIG. 4C). The individual N-glycan peaks are annotated below the respective peaks, representing the Man3GlcNAc2 to Man12GlcNAc2 Glycan structures (M3 to M12). Each marked structure is composed of two N-acetylglucosamine (GlcNAc) residues and the respective indicated number of mannose; peaks at m/z 1053 represent M3, at m/z 1215 M4, and at m/z 1377 M5. The ER synthesized M3 LLO structure is further extended in the Golgi compartment to M4 and M5.

FIGS. 5A-K list the nucleotide sequences encoding Flc2′ or fragments thereof or the amino acid sequences of the transcripts thereof. (ER localization signal is printed underlined, the transmembrane domains are printed in bold letters):

FIG. 5A shows the nucleotide sequence encoding Flc2′ (SEQ ID NO: 1); FIG. 5B shows the amino acid sequence of the Flc2′ transcript (SEQ ID NO: 2);

FIG. 5C shows the nucleotide sequence coding for the ER localization signal and for the transmembrane domains (TM) 1 to 3 of the coding region of flc2′ (TM1-3) (SEQ ID NO: 3); FIG. 5D shows the amino acid sequence of the transcript of the nucleotide sequence of FIG. 5C (SEQ ID NO: 4);

FIG. 5E shows the nucleotide sequence coding for the ER localization signal and for the transmembrane domains (TM) 1 to 2 of the coding region of flc2′ (TM1-2) (SEQ ID NO: 5); FIG. 5F shows the amino acid sequence of the transcript of the nucleotide sequence of FIG. 5E (SEQ ID NO: 6);

FIG. 5G shows the nucleotide sequence coding for the ER localization signal and for the transmembrane domains (TM) 2 to 4 of the coding region of flc2′ (TM3-4) (SEQ ID NO: 7); FIG. 5H shows the amino acid sequence of the transcript of the nucleotide sequence of FIG. 5G (SEQ ID NO: 8);

FIG. 5I shows the nucleotide sequence coding for the ER localization signal and for the transmembrane domains (TM) 3 to 4 of the coding region of flc2′ (TM3-4) (SEQ ID NO: 9); FIG. 5K shows the amino acid sequence of the transcript of the nucleotide sequence of FIG. 5I (SEQ ID NO: 10);

FIG. 5L shows the nucleotide sequence representing the endogenous promoter of flc2′ (SEQ ID NO: 61), the underlined portion identifies the start codon.

FIG. 6A depicts a spotting assay of wild type strain compared to Δrft1 mutant strains carrying either the empty vector, Rft1 (oe RFT1) or Flc2′ expression plasmid (oe Flc2′). Each row consists of a serial dilution of the indicated strain. Plasmid borne Flc2′ can complement the Rft1 deletion.Δ; FIG. 6B depicts a respective spotting assay of wild type strain compared to Δalg11 mutant strains; FIG. 6C depicts a respective spotting assay of wild type strain compared to Δalg2-1 mutant strains.

FIGS. 7A and B depict spotting assays of Δrft1 mutant strains carrying either the empty vector, Rft1, Flc2′, Flc2′ fragments comprising transmembrane domains 3 (TM 3), transmembrane domains 1 and 3 (TM 1-3) or transmembrane domains 3 and 4 (TM 3-4) or Flc2 expression plasmid. Each row consists of a serial dilution of the indicated strain. Plasmid borne Flc2′ can complement the Rft1 deletion. In contrast, overexpression of full lenght Flc2 (oe Flc2) can not complement the growth defects, thus does bring about a compensation for the lack of endogenous flippase activity.

FIG. 7C depicts the N-Glycosylation of carboxypeptidase Y in wildtype yeast strain Δrft1 mutant strains carrying either an empty plasmid (YEp352), or plasmids for overexpression of Rft1, and Flc2′ flippase. Bands representing fully glycosylated (mCPY) and hypoglycosylated forms of CPY are indicated with −1, −2, −3 and −4. YEp26.2 represents the original clone identified in the HCSS.

FIG. 8 depicts HPLC traces of [3H]-mannose-labeled lipid-linked oligosaccharides from Δrft1 mutant strains: FIG. 8A: Δrft1 mutant strains carrying empty vector YEp352; FIG. 8B: Δrft1 mutant strains carrying Rft1 expression construct; FIG. 8C: Δrft1 mutant strains carrying Flc2′ expression construct.

FIG. 9 depicts the results of a N-Glycosylation of carboxypeptidase Y in wildtype or Δalg3Δalg11 mutant yeast strains carrying either an empty plasmid (YEp352), or plasmids for overexpression of Flc2′, or Rft1 flippase.

FIG. 10 depicts the Western blot results of a N-Glycosylation of carboxypeptidase Y (CPY) and beta-1,3-glucanosyltransferase (Gas1p) in wid-type yeast (YG1509) or mutant yeast strains YG1365 (Δalg11) and YG1363 (Δalg3Δalg11) expressing Flc2′ flippase, LmStt3D, or the combination of Flc2′ flippase and LmStt3D. Bands representing fully glycosylated (M CPY) and hypoglycosylated forms of CPY and Gas1p are indicated.

FIG. 11 depicts the N-Glycosylation of carboxypeptidase Y in Δalg11 mutant strains carrying either an empty vector (e.v., YEp352), or plasmids for overexpression of Flc2′, POT, or Flc2′ and POT. Bands representing fully glycosylated (mCPY) and hypoglycosylated forms of CPY are indicated with −1, −2, −3 and −4.

FIG. 12 depicts a schematic representation of a preferred composite system according to the invention for N-linked glycosylation in a lower eukaryote, as exemplified for yeast. In more detail, the synthesis of the lipid-linked oligosaccharides occurs on the cytoplasmic side of the ER; the synthesis is initiated by the transfer of phosphate residues to dolichol by Sec59p and the oligosaccharide donor is extended by the consecutive action of several monosaccharide transferases on the cytoplasmic and lumenal side of the ER finally leading to lipid-linked Glc3Man9GlcNAc2. Lipid-linked Glc3Man9GlcNAc2 serves as substrate for the endogenous multi-subunit yeast oligosaccharylcomplex (Ost complex); in the composite system alg3 and alg11 genes are deleted (Δalg11, Δalg3) resulting in the generation of lipid-linked Man3GlcNAc2. The remaining transferases are still present in the cell, however, are inactive on the lipid-linked GlcNAc2Man3 substrate. A novel LLO flippase according to the invention (Flc2′) and a protozoan oligosaccharyl transferase (POT Leishmania major Stt3D) are added. In an alternative embodiment the generation of lipid-linked Man3GlcNAc2 is conferred by the deletion of dpm1 gene, the product of which generates lipid-linked mannose on the cytoplasmic side of the ER membrane (DPM1). In an alternative embodiment the generation of lipid-linked Man3GlcNAc2 is conferred by the deletion of the monosaccharide flippase, which flipps the dolichol-linked mannose into the ER lumen (asterisk). Lipid-linked mannose serves a donor for the ER lumen located oligosaccharyltransferases. In combination with the alg11 mutation such a cell would also produce lipid-linked Man3GlcNAc2. Redundant non used transferases, flippase (Rft1), components of the yeast Ost complex and the non-synthesized structures are printed in grey.

FIG. 13 depicts the nucleotide sequence of a preferred embodient, a Flc2′ expression plasmid YEp352Flc2′ (SEQ ID NO: 31).

FIG. 14 depicts the nucleotide sequence of another preferred embodient, a LmStt3D and Flc2′ co-expression plasmid pAX306f (SEQ ID NO: 32).

FIG. 15A depicts a schematic representation of the truncated version Flc2′ (transmembrane domains 1 to 4) of the yeast Flc2 protein. FIG. 15B depicts a spotting assay of Δrft1 mutant strains carrying either the empty vector (v. c.), or the vectors for overexpression of Flc2′ (oe Flc2*) or of truncated elements (TMD1-2, TMD1-3, TMD3-4) or individual transmembrane domains 1, 3, or 4 (TMD1, TMD3, TMD4) of Flc2′. Truncated elements with transmembrane domains 3 and 4 (TMD3-4) and transmembrane domain 4 (TMD4) are shown to complement deletion of Rft1 to similar levels as full length Flc2′(=transmembrane domains 1 to 4). FIG. 15C depicts the results of N-Glycosylation of carboxypeptidase Y (CPY) in Δrft1 mutant yeast strains carrying either an empty plasmid (v. c.), or plasmids for overexpression of Flc2′ (oe Flc2*) or truncated version of Flc2′ comprising only transmembrane domain 4 of Flc2′ (Flc2*-TMD4). Bands representing fully glycosylated (mCPY) and hypoglycosylated forms of CPY are indicated. Overexpression of transmembrane domain 4(Flc2*-TMD4) alone can complement the glycosylation deficiency in Δrft1 mutant yeast strains.

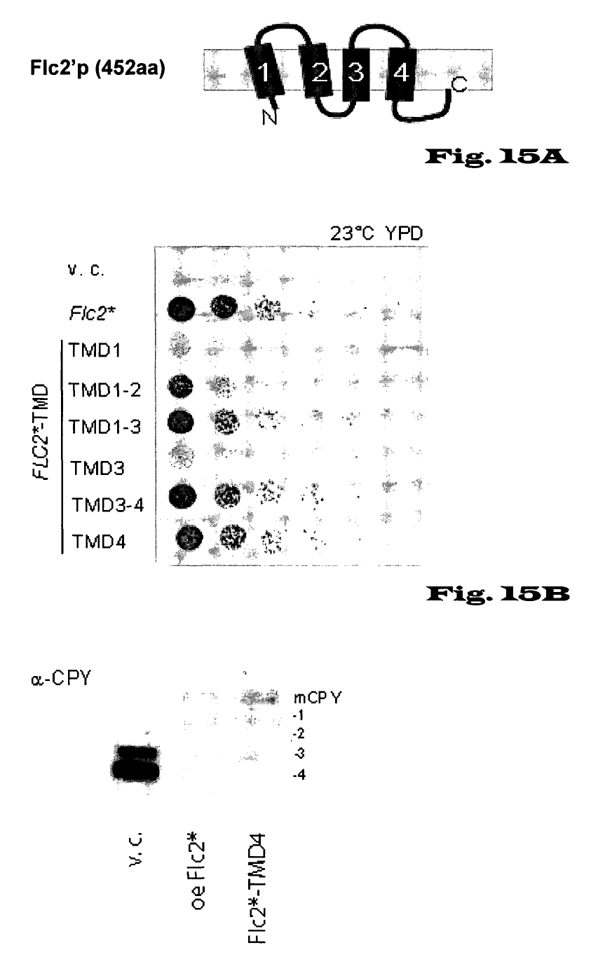

FIG. 16A depicts the results of a N-Glycosylation of carboxypeptidase Y (CPY) in Δrft1 mutant yeast strains carrying either an empty plasmid (v. c.), or plasmids for overexpression of Rft1 (oe Rft1), Flc2′ (oe Flc2*) or endogenous Flc2 (oe Flc2). Bands representing fully glycosylated (mCPY) and hypoglycosylated forms of CPY are indicated. Overexpression of Flc2 cannot complement the hypoglycosylation phenotype observed upon deletion of Rft1. FIG. 16B depicts a growth assay of Δrft1cells carrying the empty vector (v. c.), plasmids for overexpression of Rft1 (oe Rft1), Flc2* (oe Flc2*) or the Flc2. The growth assay confirms the capability of Flc2* and the inability of full length Flc2 to complement Rft1 defect.

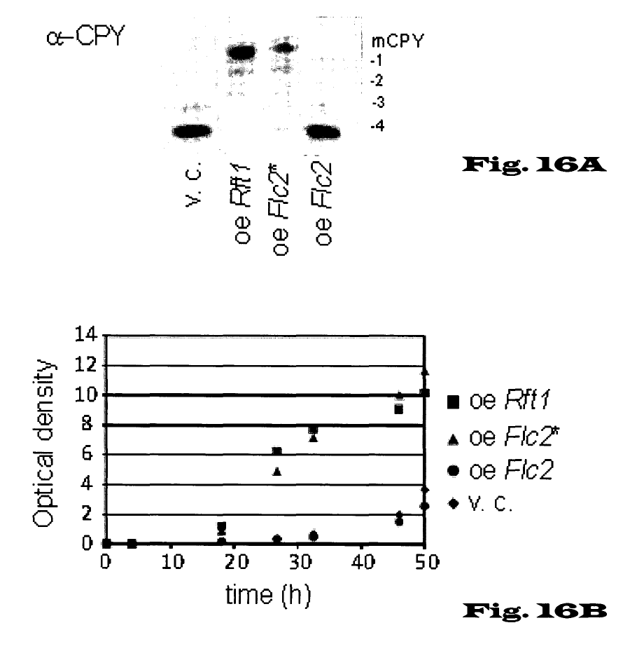

FIGS. 17ABC depict HPLC traces of [3H]-mannose-labeled lipid-linked oligosaccharides isolated from Δalg11 mutant strains (YG1365) carrying empty vector (v. c.) (FIG. 17A), the plasmid for overexpression of Rft1 (oe Rft1) (FIG. 17B), or Flc2′ (oe Flc21(FIG. 17C). The LLO species detected are Man2GlcNac2 (Man2, M2), Man3GlcNac2 (Man3, M3), Man5GlcNac2 (Man5, M5), Man6GlcNac2 (Man6, M6), and Man7GlcNac2 (Man7, M7). M2 and M3 oligosaccharides are located on the cytoplasmic side of the ER membrane (cytopl.), M5 to M7 oligosaccharides are located on the lumenal side of the ER membrane (lumenal). The relative amounts of cytoplasmic versus lumenal LLO species is indicative for flippase activities of the expressed proteins.

FIG. 18A depicts a growth assay of Δalg11Δalg3 mutant yeast strain carrying the empty vector (v. c.), plasmids for overexpression of Rft1 (oe Rft1) or Flc2′ (oe Flc2*). FIG. 18B depicts a spotting assay of the respective cells. The growth assay and spotting assay show the capability of the overexpression of either Flc2′ or Rft1 to improve growth of the Δalg11Δalg3 mutant yeast strain. FIG. 18C depicts the results of N-Glycosylation of carboxypeptidase Y (CPY) in Δalg11Δalg3 mutant yeast strain carrying either an empty plasmid (v. c.), or plasmids for overexpression of Rft1 (oe Rft1) or Flc2′ (oe Flc2*). Bands that represent fully glycosylated (mCPY) and hypoglycosylated forms of CPY are indicated. Overexpression of Rft1 or Flc2′ improves N-glycosylation of CPY.

FIG. 19A depicts a growth assay of Δalg11 mutant yeast strain carrying the empty vector (v. c.), plasmids for overexpression of Rft1 (oe Rft1) or Flc2′ (oe Flc2*). FIG. 19B depicts a spotting assay of the respective cells. The growth assay and spotting assay show the capability of the overexpression of either Flc2′ or Rft1 to improve growth of the Δalg11 mutant yeast strain. FIG. 19C depicts the results of N-Glycosylation of carboxypeptidase Y (CPY) in Δalg11 mutant yeast strain carrying either an empty plasmid (v. c.), or plasmids for overexpression of Rft1 (oe Rft1) or Flc2′ (oe Flc2*). Bands that represent fully glycosylated (mCPY) and hypoglycosylated forms of CPY are indicated. Overexpression of Rft1 or Flc2′ improves N-glycosylation of CPY.

FIG. 20A depicts a schematic representation of LLO synthesis in alg2-1 strain harboring a temperature sensitive Alg2 protein. Alg2 catalyzes two consecutive additions of Mannoses to the Man1GlcNAc2 (M1) structure generating Man2GlcNAc2 (M2) and Man3GlcNAc2 (M3). This mutation reduces the Alg2 activity, which in turn reduces the synthesis of LLO species bigger than M1. The residual activity of Alg2 is however sufficient to sustain regular LLO synthesis, leading to the generation of Glc3Man9GlcNAc2 structure. Flipping of M1 and M2 structures competes with elongation reactions catalyzed by Alg2. If M1 and M2 structures become flipped into the ER lumen, these structures do not represent a substrate for Mannosyltranferases in the ER lumen and are not elongated further. Finally the oligosaccharides from the different LLO donors are transferred onto the Asn residues in the N-glycosylation consensus sequence of proteins. FIG. 20B depicts a schematic representation of a MALDI-TOF spectra with the expected peaks being Man1GlcNAc2 (M1), Man2GlcNAc2 (M2) and the high-mannose structures Man8GlcNAc2 to Man12GlcNAc2 (M8-M12). Based on the peak intensities of NLO species relative abundances of the individual structures can be calculated. A relative increase in M1 species indicates that flipping of M1 dominates elongation reaction of Man1GlcNAc2 (M1) by Alg2.

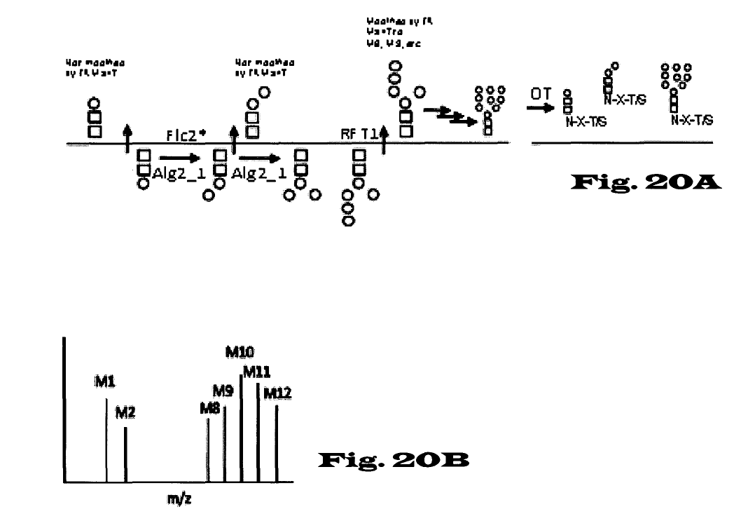

FIG. 21A depicts the results of N-Glycosylation of carboxypeptidase Y (CPY) in Δalg11 mutant yeast cells carrying vectors for overexpression of Flc2′ (oe Flc2*) or protozoan oligosaccharyl transferase POT (oe POT) and the combination of Flc2′ and POT (oe Flc2* & POT). Bands representing fully glycosylated (mCPY) and hypoglycosylated forms of CPY are indicated. FIG. 21B depicts the results of N-Glycosylation of carboxypeptidase Y (CPY) in Δalg11Δalg3 mutant yeast cells carrying vectors for overexpression of Flc2* (oe Flc2*), POT (oe POT) and the combination of Flc2′ and POT (oe Flc2* & POT). Bands representing fully glycosylated (mCPY) and hypoglycosylated forms of CPY are indicated. Coexpression of POT and Flc2′ suppresses the hypoglycosylation phenotype to a higher degree in both, Δalg11 and Δalg11Δalg3 yeast strains.

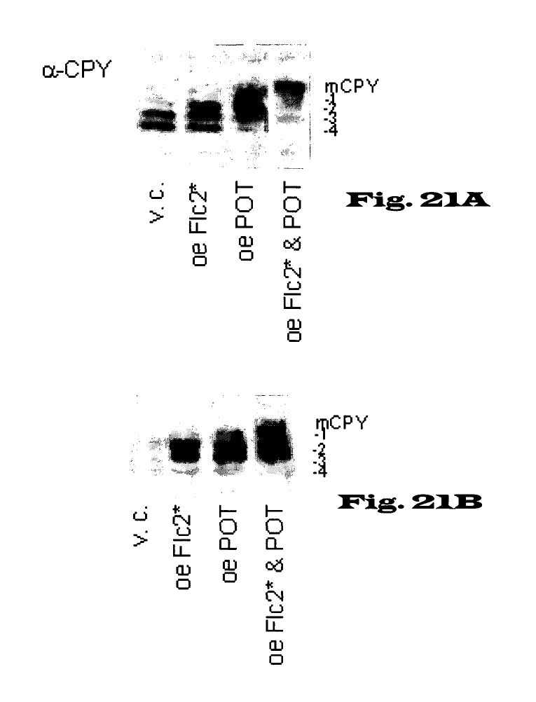

FIG. 22AB depict MALDI-TOF MS spectra of 2-AB-labeled N-glycans isolated from cell wall proteins from Δalg3Δalg11 yeast mutant strains (FIG. 22A) and cell wall proteins from Δalg11Δalg3Δmnn1 yeast mutant strains (FIG. 22B). The individual N-glycan peaks are annotated above the respective peaks, being Man3GlcNAc2 (M3) to Man6GlcNAc2 (M6). In addition to Mannose each indicated structure contains two additional Gn residues. The peaks at m/z 1053 represent M3, at m/z 1215 M4, at m/z 1377 M5 and at m/z 1539 M6. The ER synthesized Man3GlcNAc2 LLO structure is further extended in the Golgi compartment to Man4GlcNAc2, Man5GlcNAc2 and very small amounts of Man6GlcNAc2. Deletion of mnn1 partially abolishes processing of ER synthesized Man3GlcNAc2 structure as revealed by the strong reduction of Man5 peak in the Golgi compartment.

### SEQUENCE LISTING

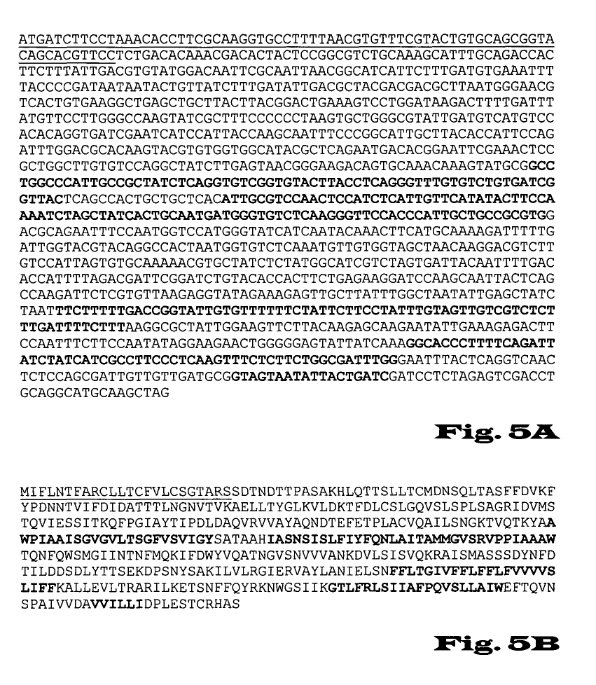

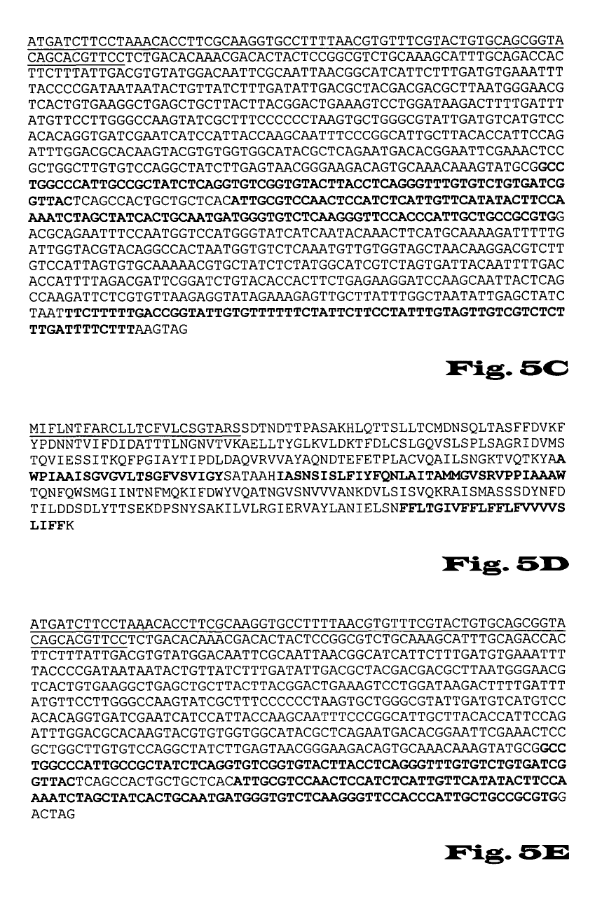

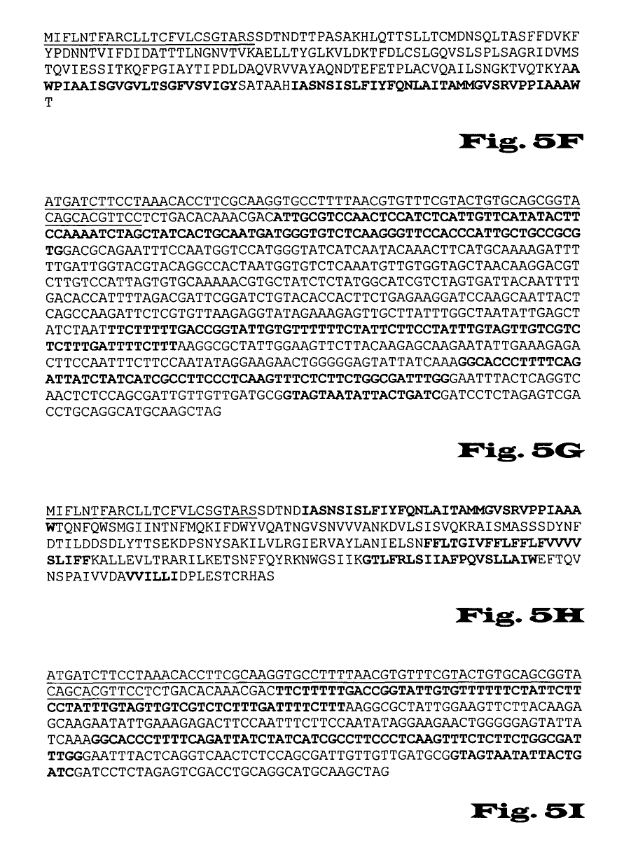

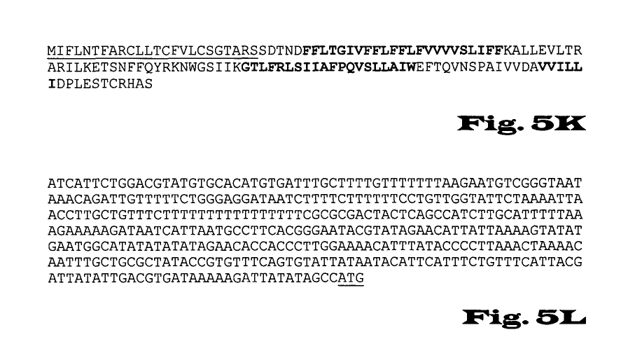

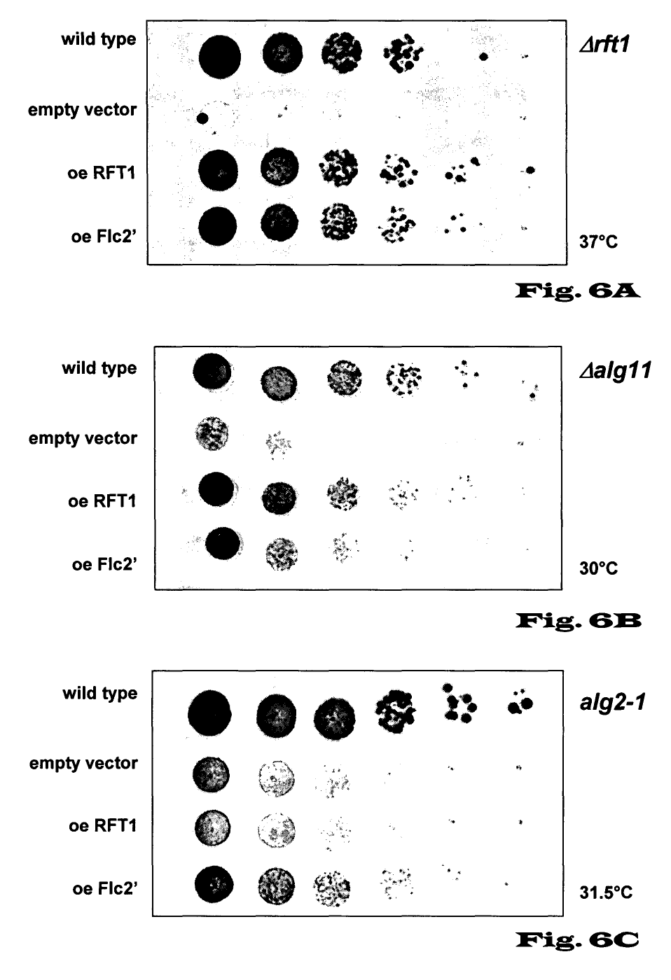

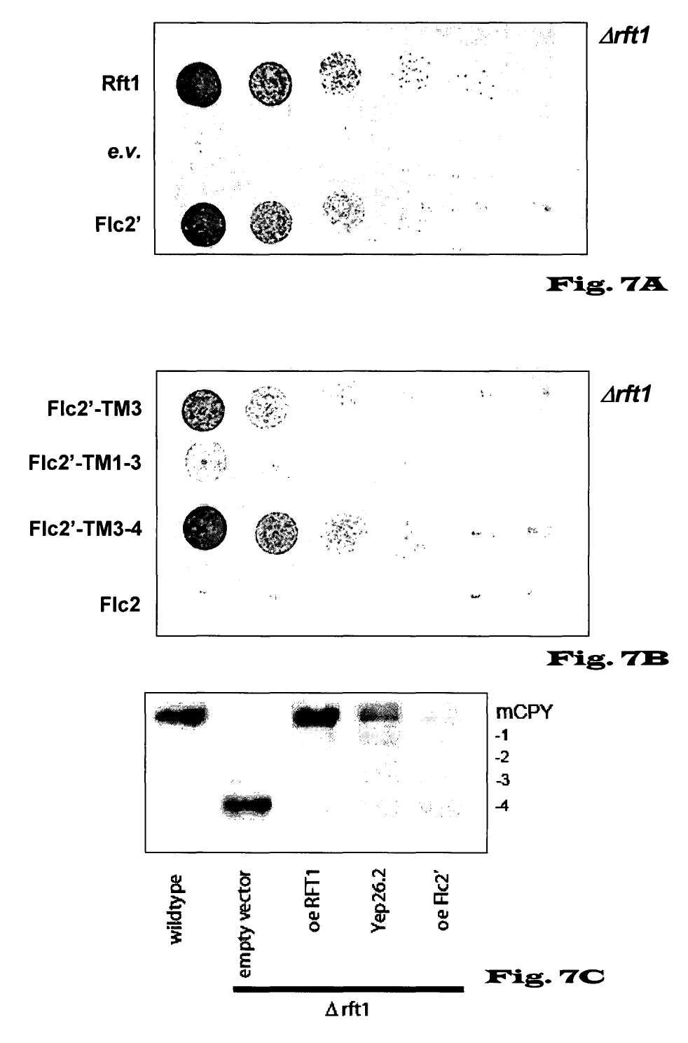

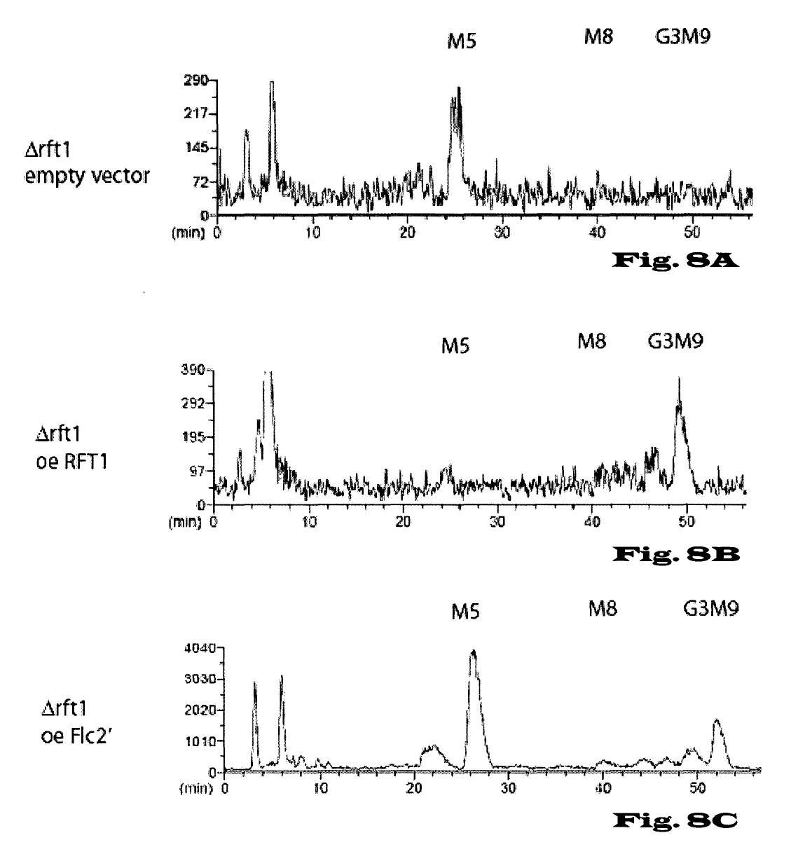

SEQ ID NO: 1 represents the nucleotide sequence
coding for flc2′, which is a truncated fragment
of the gene flc2 (FIG. 5A).
ATGATCTTCCTAAACACCTTCGCAAGGTGCCTTTTAACGTGTTTCGTACT
GTGCAGCGGTACAGCACGTTCCTCTGACACAAACGACACTACTCCGGCGT
CTGCAAAGCATTTGCAGACCACTTCTTTATTGACGTGTATGGACAATTCG
CAATTAACGGCATCATTCTTTGATGTGAAATTTTACCCCGATAATAATAC
TGTTATCTTTGATATTGACGCTACGACGACGCTTAATGGGAACGTCACTG
TGAAGGCTGAGCTGCTTACTTACGGACTGAAAGTCCTGGATAAGACTTTT
GATTTATGTTCCTTGGGCCAAGTATCGCTTTCCCCCCTAAGTGCTGGGCG
TATTGATGTCATGTCCACACAGGTGATCGAATCATCCATTACCAAGCAAT
TTCCCGGCATTGCTTACACCATTCCAGATTTGGACGCACAAGTACGTGTG
GTGGCATACGCTCAGAATGACACGGAATTCGAAACTCCGCTGGCTTGTGT
CCAGGCTATCTTGAGTAACGGGAAGACAGTGCAAACAAAGTATGCGGCCT
GGCCCATTGCCGCTATCTCAGGTGTCGGTGTACTTACCTCAGGGTTTGTG
TCTGTGATCGGTTACTCAGCCACTGCTGCTCACATTGCGTCCAACTCCAT
CTCATTGTTCATATACTTCCAAAATCTAGCTATCACTGCAATGATGGGTG
TCTCAAGGGTTCCACCCATTGCTGCCGCGTGGACGCAGAATTTCCAATGG
TCCATGGGTATCATCAATACAAACTTCATGCAAAAGATTTTTGATTGGTA
CGTACAGGCCACTAATGGTGTCTCAAATGTTGTGGTAGCTAACAAGGACG
TCTTGTCCATTAGTGTGCAAAAACGTGCTATCTCTATGGCATCGTCTAGT
GATTACAATTTTGACACCATTTTAGACGATTCGGATCTGTACACCACTTC
TGAGAAGGATCCAAGCAATTACTCAGCCAAGATTCTCGTGTTAAGAGGTA
TAGAAAGAGTTGCTTATTTGGCTAATATTGAGCTATCTAATTTCTTTTTG
ACCGGTATTGTGTTTTTTCTATTCTTCCTATTTGTAGTTGTCGTCTCTTT
GATTTTCTTTAAGGCGCTATTGGAAGTTCTTACAAGAGCAAGAATATTGA
AAGAGACTTCCAATTTCTTCCAATATAGGAAGAACTGGGGGAGTATTATC
AAAGGCACCCTTTTCAGATTATCTATCATCGCCTTCCCTCAAGTTTCTCT
TCTGGCGATTTGGGAATTTACTCAGGTCAACTCTCCAGCGATTGTTGTTG
ATGCGGTAGTAATATTACTGATCGATCCTCTAGAGTCGACCTGCAGGCAT
GCAAGCTAG
SEQ ID NO: 2 represents the amino acid
sequence of Flc2′ (FIG. 5B).
MIFLNTFARCLLTCFVLCSGTARSSDTNDTTPASAKHLQTTSLLTCMDNS
QLTASFFDVKFYPDNNTVIFDIDATTTLNGNVTVKAELLTYGLKVLDKTF
DLCSLGQVSLSPLSAGRIDVMSTQVIESSITKQFPGIAYTIPDLDAQVRV
VAYAQNDTEFETPLACVQAILSNGKTVQTKYAAWPIAAISGVGVLTSGFV
SVIGYSATAAHIASNSISLFIYFQNLAITAMMGVSRVPPIAAAWTQNFQW
SMGIINTNFMQKIFDWYVQATNGVSNVVVANKDVLSISVQKRAISMASSS
DYNFDTILDDSDLYTTSEKDPSNYSAKILVLRGIERVAYLANIELSNFFL
TGIVFFLFFLFVVVVSLIFFKALLEVLTRARILKETSNFFQYRKNWGSII
KGTLFRLSIIAFPQVSLLAIWEFTQVNSPAIVVDAVVILLIDPLESTCRH
AS
SEQ ID NO: 3 represents the nucleotide sequence
coding for the ER localization signal and for
the transmembrane domains (TM) 1 to 3 of the
coding region of flc2′ (TM1-3)
ATGATCTTCCTAAACACCTTCGCAAGGTGCCTTTTAACGTGTTTCGTACT
GTGCAGCGGTACAGCACGTTCCTCTGACACAAACGACACTACTCCGGCGT
CTGCAAAGCATTTGCAGACCACTTCTTTATTGACGTGTATGGACAATTCG
CAATTAACGGCATCATTCTTTGATGTGAAATTTTACCCCGATAATAATAC
TGTTATCTTTGATATTGACGCTACGACGACGCTTAATGGGAACGTCACTG
TGAAGGCTGAGCTGCTTACTTACGGACTGAAAGTCCTGGATAAGACTTTT
GATTTATGTTCCTTGGGCCAAGTATCGCTTTCCCCCCTAAGTGCTGGGCG
TATTGATGTCATGTCCACACAGGTGATCGAATCATCCATTACCAAGCAAT
TTCCCGGCATTGCTTACACCATTCCAGATTTGGACGCACAAGTACGTGTG
GTGGCATACGCTCAGAATGACACGGAATTCGAAACTCCGCTGGCTTGTGT
CCAGGCTATCTTGAGTAACGGGAAGACAGTGCAAACAAAGTATGCGGCCT
GGCCCATTGCCGCTATCTCAGGTGTCGGTGTACTTACCTCAGGGTTTGTG
TCTGTGATCGGTTACTCAGCCACTGCTGCTCACATTGCGTCCAACTCCAT
CTCATTGTTCATATACTTCCAAAATCTAGCTATCACTGCAATGATGGGTG
TCTCAAGGGTTCCACCCATTGCTGCCGCGTGGACGCAGAATTTCCAATGG
TCCATGGGTATCATCAATACAAACTTCATGCAAAAGATTTTTGATTGGTA
CGTACAGGCCACTAATGGTGTCTCAAATGTTGTGGTAGCTAACAAGGACG
TCTTGTCCATTAGTGTGCAAAAACGTGCTATCTCTATGGCATCGTCTAGT
GATTACAATTTTGACACCATTTTAGACGATTCGGATCTGTACACCACTTC
TGAGAAGGATCCAAGCAATTACTCAGCCAAGATTCTCGTGTTAAGAGGTA
TAGAAAGAGTTGCTTATTTGGCTAATATTGAGCTATCTAATTTCTTTTTG
ACCGGTATTGTGTTTTTTCTATTCTTCCTATTTGTAGTTGTCGTCTCTTT
GATTTTCTTTAAGTAG
SEQ ID NO: 4 represents the amino acid
sequence of the ER localization signal
and the transmembrane domains (TM) 1
to 3 of Flc2′ (TM1-3)
MIFLNTFARCLLTCFVLCSGTARSSDTNDTTPASAKHLQTTSLLTCMDNS
QLTASFFDVKFYPDNNTVIFDIDATTTLNGNVTVKAELLTYGLKVLDKTF
DLCSLGQVSLSPLSAGRIDVMSTQVIESSITKQFPGIAYTIPDLDAQVRV
VAYAQNDTEFETPLACVQAILSNGKTVQTKYAAWPIAAISGVGVLTSGFV
SVIGYSATAAHIASNSISLFIYFQNLAITAMMGVSRVPPIAAAWTQNFQW
SMGIINTNFMQKIFDWYVQATNGVSNVVVANKDVLSISVQKRAISMASSS
DYNFDTILDDSDLYTTSEKDPSNYSAKILVLRGIERVAYLANIELSNFFL
TGIVFFLFFLFVVVVSLIFFK
SEQ ID NO: 5 represents the nucleotide sequence
coding for the ER localization signal and for
the transmembrane domains (TM) 1 to 2 of
the coding region of flc2′ (TM1-2)
ATGATCTTCCTAAACACCTTCGCAAGGTGCCTTTTAACGTGTTTCGTACT
GTGCAGCGGTACAGCACGTTCCTCTGACACAAACGACACTACTCCGGCGT
CTGCAAAGCATTTGCAGACCACTTCTTTATTGACGTGTATGGACAATTCG
CAATTAACGGCATCATTCTTTGATGTGAAATTTTACCCCGATAATAATAC
TGTTATCTTTGATATTGACGCTACGACGACGCTTAATGGGAACGTCACTG
TGAAGGCTGAGCTGCTTACTTACGGACTGAAAGTCCTGGATAAGACTTTT
GATTTATGTTCCTTGGGCCAAGTATCGCTTTCCCCCCTAAGTGCTGGGCG
TATTGATGTCATGTCCACACAGGTGATCGAATCATCCATTACCAAGCAAT
TTCCCGGCATTGCTTACACCATTCCAGATTTGGACGCACAAGTACGTGTG
GTGGCATACGCTCAGAATGACACGGAATTCGAAACTCCGCTGGCTTGTGT
CCAGGCTATCTTGAGTAACGGGAAGACAGTGCAAACAAAGTATGCGGCCT
GGCCCATTGCCGCTATCTCAGGTGTCGGTGTACTTACCTCAGGGTTTGTG
TCTGTGATCGGTTACTCAGCCACTGCTGCTCACATTGCGTCCAACTCCAT
CTCATTGTTCATATACTTCCAAAATCTAGCTATCACTGCAATGATGGGTG
TCTCAAGGGTTCCACCCATTGCTGCCGCGTGGACTAG
SEQ ID NO: 6 represents the amino acid
sequence of the ER localization signal
and the transmembrane domains (TM)
1 to 2 of Flc2′ (TM1-2)
MIFLNTFARCLLTCFVLCSGTARSSDTNDTTPASAKHLQTTSLLTCMDNS
QLTASFFDVKFYPDNNTVIFDIDATTTLNGNVTVKAELLTYGLKVLDKTF
DLCSLGQVSLSPLSAGRIDVMSTQVIESSITKQFPGIAYTIPDLDAQVRV
VAYAQNDTEFETPLACVQAILSNGKTVQTKYAAWPIAAISGVGVLTSGFV
SVIGYSATAAHIASNSISLFIYFQNLAITAMMGVSRVPPIAAAWT
SEQ ID NO: 7 represents the nucleotide sequence
coding for the ER localization signal and
for the transmembrane domains (TM) 2 to 4
of the coding region of flc2′ (TM2-4)
ATGATCTTCCTAAACACCTTCGCAAGGTGCCTTTTAACGTGTTTCGTACT
GTGCAGCGGTACAGCACGTTCCTCTGACACAAACGACATTGCGTCCAACT
CCATCTCATTGTTCATATACTTCCAAAATCTAGCTATCACTGCAATGATG
GGTGTCTCAAGGGTTCCACCCATTGCTGCCGCGTGGACGCAGAATTTCCA
ATGGTCCATGGGTATCATCAATACAAACTTCATGCAAAAGATTTTTGATT
GGTACGTACAGGCCACTAATGGTGTCTCAAATGTTGTGGTAGCTAACAAG
GACGTCTTGTCCATTAGTGTGCAAAAACGTGCTATCTCTATGGCATCGTC
TAGTGATTACAATTTTGACACCATTTTAGACGATTCGGATCTGTACACCA
CTTCTGAGAAGGATCCAAGCAATTACTCAGCCAAGATTCTCGTGTTAAGA
GGTATAGAAAGAGTTGCTTATTTGGCTAATATTGAGCTATCTAATTTCTT
TTTGACCGGTATTGTGTTTTTTCTATTCTTCCTATTTGTAGTTGTCGTCT
CTTTGATTTTCTTTAAGGCGCTATTGGAAGTTCTTACAAGAGCAAGAATA
TTGAAAGAGACTTCCAATTTCTTCCAATATAGGAAGAACTGGGGGAGTAT
TATCAAAGGCACCCTTTTCAGATTATCTATCATCGCCTTCCCTCAAGTTT
CTCTTCTGGCGATTTGGGAATTTACTCAGGTCAACTCTCCAGCGATTGTT
GTTGATGCGGTAGTAATATTACTGATCGATCCTCTAGAGTCGACCTGCAG
GCATGCAAGCTAG
SEQ ID NO: 8 represents the amino acid
sequence of the ER localization signal
and the transmembrane domains (TM)
2 to 4 of Flc2′ (TM2-4)
MIFLNTFARCLLTCFVLCSGTARSSDTNDIASNSISLFIYFQNLAITAMM
GVSRVPPIAAAWTQNFQWSMGIINTNFMQKIFDWYVQATNGVSNVVVANK
DVLSISVQKRAISMASSSDYNFDTILDDSDLYTTSEKDPSNYSAKILVLR
GIERVAYLANIELSNFFLTGIVFFLFFLFVVVVSLIFFKALLEVLTRARI
LKETSNFFQYRKNWGSIIKGTLFRLSIIAFPQVSLLAIWEFTQVNSPAIV
VDAVVILLIDPLESTCRHAS
SEQ ID NO: 9 represents the nucleotide sequence
coding for the ER localization signal and for
the transmembrane domains (TM) 3 to 4 of
the coding region of flc2′ (TM3-4)
ATGATCTTCCTAAACACCTTCGCAAGGTGCCTTTTAACGTGTTTCGTACT
GTGCAGCGGTACAGCACGTTCCTCTGACACAAACGACTTCTTTTTGACCG
GTATTGTGTTTTTTCTATTCTTCCTATTTGTAGTTGTCGTCTCTTTGATT
TTCTTTAAGGCGCTATTGGAAGTTCTTACAAGAGCAAGAATATTGAAAGA
GACTTCCAATTTCTTCCAATATAGGAAGAACTGGGGGAGTATTATCAAAG
GCACCCTTTTCAGATTATCTATCATCGCCTTCCCTCAAGTTTCTCTTCTG
GCGATTTGGGAATTTACTCAGGTCAACTCTCCAGCGATTGTTGTTGATGC
GGTAGTAATATTACTGATCGATCCTCTAGAGTCGACCTGCAGGCATGCAA
GCTAG
SEQ ID NO: 10 represents the amino acid
sequence of the ER localization signal
and the transmembrane domains (TM)
3 to 4 of Flc2′ (TM3-4)
MIFLNTFARCLLTCFVLCSGTARSSDTNDFFLTGIVFFLFFLFVVVVSLI
FFKALLEVLTRARILKETSNFFQYRKNWGSIIKGTLFRLSIIAFPQVSLL
AIWEFTQVNSPAIVVDAVVILLIDPLESTCRHAS
SEQ ID NO: 11 represents the nucleotide
sequence coding for the ER localization
signal and for thetransmembrane domain
(TM) 1 of the coding region of flc2′ (TM1)
ATGATCTTCCTAAACACCTTCGCAAGGTGCCTTTTAACGTGTTTCGTACT
GTGCAGCGGTACAGCACGTTCCTCTGACACAAACGACACTACTCCGGCGT
CTGCAAAGCATTTGCAGACCACTTCTTTATTGACGTGTATGGACAATTCG
CAATTAACGGCATCATTCTTTGATGTGAAATTTTACCCCGATAATAATAC
TGTTATCTTTGATATTGACGCTACGACGACGCTTAATGGGAACGTCACTG
TGAAGGCTGAGCTGCTTACTTACGGACTGAAAGTCCTGGATAAGACTTTT
GATTTATGTTCCTTGGGCCAAGTATCGCTTTCCCCCCTAAGTGCTGGGCG
TATTGATGTCATGTCCACACAGGTGATCGAATCATCCATTACCAAGCAAT
TTCCCGGCATTGCTTACACCATTCCAGATTTGGACGCACAAGTACGTGTG
GTGGCATACGCTCAGAATGACACGGAATTCGAAACTCCGCTGGCTTGTGT
CCAGGCTATCTTGAGTAACGGGAAGACAGTGCAAACAAAGTATGCGGCCT
GGCCCATTGCCGCTATCTCAGGTGTCGGTGTACTTACCTCAGGGTTTGTG
TCTGTGATCGGTTACTCATAG
SEQ ID NO: 12 represents the amino acid
sequence of the ER localization signal
and the transmembrane domain (TM)
1 of Flc2′ (TM1)
MIFLNTFARCLLTCFVLCSGTARSSDTNDTTPASAKHLQTTSLLTCMDNS
QLTASFFDVKFYPDNNTVIFDIDATTTLNGNVTVKAELLTYGLKVLDKTF
DLCSLGQVSLSPLSAGRIDVMSTQVIESSITKQFPGIAYTIPDLDAQVRV
VAYAQNDTEFETPLACVQAILSNGKTVQTKYAAWPIAAISGVGVLTSGFV
SVIGYS
SEQ ID NO: 13 represents the nucleotide
sequence coding for the ER localization
signal and for the transmembrane domain
(TM) 2 of the coding region of flc2′ (TM2)
ATGATCTTCCTAAACACCTTCGCAAGGTGCCTTTTAACGTGTTTCGTACT
GTGCAGCGGTACAGCACGTTCCTCTGACACAAACGACATTGCGTCCAACT
CCATCTCATTGTTCATATACTTCCAAAATCTAGCTATCACTGCAATGATG
GGTGTCTCAAGGGTTCCACCCATTGCTGCCGCGTGGACTAG
SEQ ID NO: 14 represents the amino acid
sequence of the ER localization signal
and the transmembrane domain (TM)
2 of Flc2′ (TM2)
MIFLNTFARCLLTCFVLCSGTARSSDTNDIASNSISLFIYFQNLAITAMM
GVSRVPPIAAAWT
SEQ ID NO: 15 represents the nucleotide
sequence coding for the ER localization
signal and for the transmembrane domain
(TM) 3 of the coding region of flc2′ (TM3)
ATGATCTTCCTAAACACCTTCGCAAGGTGCCTTTTAACGTGTTTCGTACT
GTGCAGCGGTACAGCACGTTCCTCTGACACAAACGACTTCTTTTTGACCG
GTATTGTGTTTTTTCTATTCTTCCTATTTGTAGTTGTCGTCTCTTTGATT
TTCTTTAAGTAG
SEQ ID NO: 16 represents the amino acid
sequence of the ER localization signal and
the transmembrane domain (TM) 3 of Flc2′
(TM3)
MIFLNTFARCLLTCFVLCSGTARSSDTNDFFLTGIVFFLFFLFVVVVSLI
FFK
SEQ ID NO: 17 represents the nucleotide
sequence coding for the ER localization
signal and for the transmembrane domain
(TM) 4 of the coding region of flc2′ (TM4)
ATGATCTTCCTAAACACCTTCGCAAGGTGCCTTTTAACGTGTTTCGTACT
GTGCAGCGGTACAGCACGTTCCTCTGACACAAACGACGGCACCCTTTTCA
GATTATCTATCATCGCCTTCCCTCAAGTTTCTCTTCTGGCGATTTGGGAA
TTTACTCAGGTCAACTCTCCAGCGATTGTTGTTGATGCGGTAGTAATATT
ACTGATCGATCCTCTAGAGTCGACCTGCAGGCATGCAAGCTAG
SEQ ID NO: 18 represents the amino acid
sequence of the ER localization signal and
the transmembrane domain (TM) 4 of Flc2′ (
TM4)
MIFLNTFARCLLTCFVLCSGTARSSDTNDGTLFRLSIIAFPQVSLLAIWE
FTQVNSPAIVVDAVVILLIDPLESTCRHAS
SEQ ID NO: 19 represents the nucleotide
sequence coding for the ER localization
signal of the coding region of flc2′:
ATGATCTTCCTAAACACCTTCGCAAGGTGCCTTTTAACGTGTTTCGTA
CTGTGCAGCGGTACAGCACGTTCC
SEQ ID NO: 20 represents the amino acid
sequence of the ER localization signal
of flc2′:
MIFLNTFARCLLTCFVLCSGTARS
SEQ ID NO: 21 represents the nucleotide
sequence coding for a first transmembrane
domain of flc2′ (TM1)
GCCTGGCCCATTGCCGCTATCTCAGGTGTCGGTGTACTTACCTCAGGGTT
TGTGTCTGTGATCGGTTAC
SEQ ID NO: 22 represents the amino acid
sequence of a first transmembrane domain
of Flc2′ (TM1):
AWPIAAISGVGVLTSGFVSVIGY
SEQ ID NO: 23 represents the nucleotide
sequence coding for a second transmembrane
domain of flc2′ (TM2)
ATTGCGTCCAACTCCATCTCATTGTTCATATACTTCCAAAATCTAGCTAT
CACTGCAATGATGGGTGTCTCAAGGGTTCCACCCATTGCTGCCGCGTG
SEQ ID NO: 24 represents the amino acid
sequence of a second transmembrane domain
of Flc2′ (TM2)
IASNSISLFIYFQNLAITAMMGVSRVPPIAAAW
SEQ ID NO: 25 represents the nucleotide
sequence coding for a third transmembrane
domain of flc2′ (TM3)
TTCTTTTTGACCGGTATTGTGTTTTTTCTATTCTTCCTATTTGTAGTTGT
CGTCTCTTTGATTTTCTTT
SEQ ID NO: 26 represents the amino acid
sequence of a third transmembrane domain
of Flc2′ (TM3)
FFLTGIVFFLFFLFVVVVSLIFF
SEQ ID NO: 27 represents the nucleotide
sequence coding for a forth transmembrane
domain of flc2′ (TM4)
GGCACCCTTTTCAGATTATCTATCATCGCCTTCCCTCAAGTTTCTCTTCT
GGCGATTTGG
SEQ ID NO: 28 represents the amino acid
sequence of a forth transmembrane domain
of Flc2′ (TM4)
GTLFRLSIIAFPQVSLLAIW
SEQ ID NO: 29 represents the nucleotide
sequence coding for a fifth transmembrane
domain of flc2′ (TM5)
GTAGTAATATTACTGAT
SEQ ID NO: 30 represents the amino acid
sequence of a fifth transmembrane domain
of Flc2′ (TM5):
VVILLI

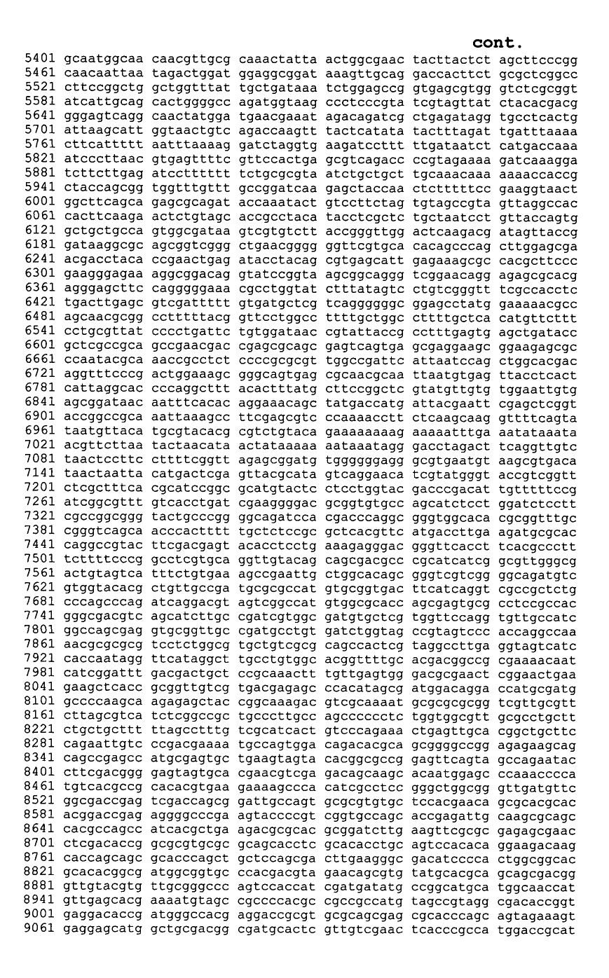

SEQ ID NO: 31 represents the nucleotide sequence of Flc2′ expression plasmid YEp352Flc2′ (FIG. 13) with a Flc2′ expression cassette comprising the ACS promoter (1 . . . 399), flc2′ (400 . . . 1722) with potential stop codon (1753 . . . 1758).

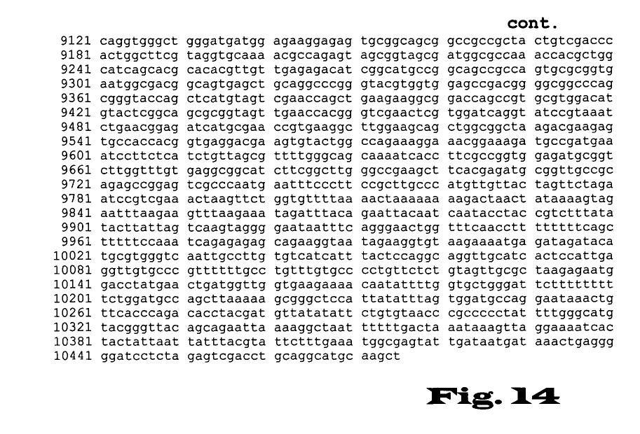

SEQ ID NO: 32 represents the nucleotide sequence of LmStt3D and Flc2′ co-expression plasmid pAX306f (FIG. 14) comprising a Flc2′ expression cassette comprising the ACS promoter (1 . . . 399), flc2′ORF (400 . . . 1722), after the STOP codon there is the CYC1 terminator (6904 . . . 7155) and further comprising a POT LmStt3D expression cassette comprising, in reverse direction, LmStt3D ORF (complement) (7192 . . . 9762) and the strong constitutive GPD promoter (complement) (9781 . . . 10435); ATG of LmStt3D is right after the GPD promoter.

SEQ ID NO: 33 represents the nucleotide sequence coding for the paralogue LbStt3-1 of Leishmania braziliensis.

SEQ ID NO: 34 represents the amino acid sequence of LbStt3-1.

SEQ ID NO: 35 represents the nucleotide sequence coding for the paralogue LbStt3-2 of Leishmania braziliensis.

SEQ ID NO: 36 represents the amino acid sequence of LbStt3-2.

SEQ ID NO: 37 represents the nucleotide sequence coding for the paralogue LbStt3-3 of Leishmania braziliensis.

SEQ ID NO: 38 represents the amino acid sequence of LbStt3-3.

SEQ ID NO: 39 represents the nucleotide sequence coding for the paralogue LiStt3-1 of Leishmania infantum.

SEQ ID NO: 40 represents the amino acid sequence of LiStt3-1.

SEQ ID NO: 41 represents the nucleotide sequence coding for the paralogue LiStt3-2 of Leishmania infantum.

SEQ ID NO: 42 represents the amino acid sequence of LiStt3-2.

SEQ ID NO: 43 represents the nucleotide sequence coding for the paralogue LiStt3-3 of Leishmania infantum.

SEQ ID NO: 44 represents the amino acid sequence of LiStt3-3.

SEQ ID NO: 45 represents the nucleotide sequence coding for the paralogue LmStt3A of Leishmania major.

SEQ ID NO: 46 represents the amino acid sequence of LmStt3A.

SEQ ID NO: 47 represents the nucleotide sequence coding for the paralogue LmStt3B of Leishmania major.

SEQ ID NO: 48 represents the amino acid sequence of LmStt3B.

SEQ ID NO: 49 represents the nucleotide sequence coding for the paralogue LmStt3C of Leishmania major.

SEQ ID NO: 50 represents the amino acid sequence of LmStt3C.

SEQ ID NO: 51 represents the nucleotide sequence coding for the paralogue LmStt3D of Leishmania major.

SEQ ID NO: 52 represents the amino acid sequence of LmStt3D.

SEQ ID NO: 53 represents the nucleotide sequence coding for the paralogue TbStt3A of Trypanosoma brucei.

SEQ ID NO: 54 represents the amino acid sequence of TbStt3A.

SEQ ID NO: 55 represents the nucleotide sequence coding for the paralogue TbStt3B of Trypanosoma brucei.

SEQ ID NO: 56 represents the amino acid sequence of TbStt3B.

SEQ ID NO: 57 represents the nucleotide sequence coding for the paralogue TbStt3C of Trypanosoma brucei.

SEQ ID NO: 58 represents the amino acid sequence of TbStt3C.

SEQ ID NO: 59 represents the nucleotide sequence coding for the paralogue TbStt3 of Trypanosoma cruzi.

SEQ ID NO: 60 represents the amino acid sequence of TbStt3.

SEQ ID NO: 61 represents the nucleotide sequence of the endogenous promoter element of flc2′.

### EXAMPLE 1

### Production of Glycoproteins with Man3GlcNAc2 Structure

1.1 Yeast Medium and Methods

All strains were grown on YPD medium unless otherwise stated. Strain YG1137 was maintained on YPGal. Strains YCN1 (Δrft1), YG1363 66 alg3Δalg11), YG1365 (Δalg11), and YG1830 (alg2-1) were grown in medium supplemented with 1M sorbitol unless otherwise stated.

1.2 Strain Construction

The entire Alg11 open reading frame was replaced in SS328XSS330 by integration of a PCR product containing the S. cerevisiae HIS3 locus. Transformed yeast strain YG1141 (MATa/α ade2-201/ade2-201 ura3-52/ura3-52 his3Δ200/his3Δ200 tyr1/+ lys2-801/+ Δalg11::HIS3/+) was sporulated and tetrads were dissected to obtain a Δalg11 haploid, YG1361 (MATα ade2-201 ura3-52 his3Δ200 Δalg11::HIS3), which was mated with YG248 (MATa Δalg3::HIS3 ade2-101 his3Δ200 lys2-801 ura3-52). The resulting diploid YG1362 (MATa/α ade2-201/ade2-201 ura3-52/ura3-52 his3Δ200/his3Δ200 lys2-801/+ Δalg3::HIS3 Δalg11::HIS3/+) was sporulated on YPD plates containing 1M sorbitol to obtain the haploid strains YG1365 (MATα ade2-101 ura3-52 his3Δ200 Δalg11::HIS3) and YG1363 (MATα ade2-101 ura3-52 his3Δ200 lys2-801 Δalg3::HIS3 Δalg11::HIS3). A Δrft1 strain was generated by replacing rft1 gene with a HIS3 cassette in a diploid strain, sporulation of the resulting diploid heterozygous strain and selection of the resulting haploid Δrft1::HIS3 strain (YCN1).

1.3 Protein Analysis

Protein extraction and western analysis were performed as described. The antibody against CPY was diluted 3,000-fold.

1.4 Lipid- and Protein-Linked Oligosaccharide Analysis

Lipid-linked oligosaccharides were labeled, extracted and analyzed as described. In brief, yeast cells (50 ml culture with an absorbance at 546 nm of 1) were grown in YPD and incubated in medium containing [3H]-mannose before lyses with organic solvents. Lipid-linked oligosaccharide was extracted using organic solvents and oligosaccharides were released by mild acid hydrolysis. The released oligosaccharides were analyzed by HPLC using an NH2-column with flow-through counting. The number of counts per minute divided by total counts in the run were conted. The percentage of total signal in a sample is the average using two measurements. N-linked oligosaccharide was purified from cell debris after lipid-linked oligosaccharide extraction. Protein of the debris pellet was solubilized (10 min at 100° C.) in 0.2 ml 1% SDS, 50 mmol/l Tris-HCl, 1% β-mercaptoethanol. After centrifugation (2 min at 15,000 g) supernatant was supplemented to 1% (v/v) NP40 in 0.25 ml and protein-linked oligosaccharides were digested off using PNGaseF (2 units, overnight at 37 C). Proteins were precipitated with 0.75 ml ethanol and samples were spun for 20 min at 15,000 g. The supernatant was dried and resuspended in 0.2 ml 70:30 acetonitrile:water, 0.1 ml of which was analyzed by HPLC as above.

1.5 MALDI-TOF-MS

For analysis of N-glycans from cell wall proteins, cells were broken in 10 mmol/l Tris using glass beads and the insoluble cell wall fractions was reduced in a buffer containing 2M thiourea, 7 mol/l Urea, 2% SDS 50 mmol/l Tris, pH 8.0 and 10 mmol/l DTT. Alkylation was performded in the identical buffer containing 25 mmol/l iodoacetamid for 1 hour at 37° C. under vigorous shaking. The cell wall fraction was collected by centrifugation and the resulting pellet washed in 50 mmol/l NH4CO3.

N-glycan were released overnight at 37° C. using 1 μl PNGase F in a buffer containing 1× denaturation buffer, 50 mmol/l phosphate buffer, pH7.5, and 1% NP-40. N-glycans were purified via C18 and Carbon columns and the eluate containing the N-glycans evaporated. N-glycans were labeled with 2-aminobenzamide and finally purified using carbon column. Mass spectra of purified N-glycan preparation were acquired using an Autoflex MALDI-TOF MS (Bruker Daltonics, Fällanden, Switzerland) in positive ion mode and operated in reflector mode. An m/z range of 800-3000 was measured.

1.6 High Copy Suppressor Screen

For a high copy suppressor screen 1 μg of a genomic library (Stagljar et al., 1994), containing partially digested yeast chromosomal DNA ligated into the vector YEp352 (Hill et al., 1986), was transformed via electroporation into 1×10⁹ YNC1 (Δrft1) cells and transformants were selected on minimal medium with 1M Sorbitol lacking uracil at 25° C. Grown transformants were tested for growth by replica-plating on YPD and YPDS at 33° C. The positive colonies (growing at 33° C. on YPD and YPDS) were tested for their ability to support growth of Δrft1 at 33, 35 and 37° C. The plasmid DNA of colonies showing full or partial suppression were isolated by extracting total yeast DNA and used for plasmid amplification in E. coli strain DH5α. Recovered plasmids were re-transformed and tested for their ability to support growth of strain Δrft1 at 33, 35 and 37° C. on YPD. 64 C.tes were further analyzed for their ability to improve the glycosylation in Δrft1 cells. Selected high copy suppressor plasmids were sequenced with M13 (GTA AAA CGA CGG CCA GT) and M13rev (GAG CGG ATA ACA ATT) primers.

1.7 Spotting Assay

To evaluate growth of yeast strains or yeast mutant strains such as e.g. Δrft1, Δalg11 or Δalg2 mutant strains expressing Rft1 or Flc2′ or fragments thereof spotting assays of such yeast strains were performed. Strains were grown overnight and cultures adjusted to equal cell densities. Serial dilutes were plated onto agar plates and the plates incubated for the indicated temperatures and 3 days.

Growth assays in liquid media were performed as follows, precultures, inoculated with a single colony, were grown for 48 hours in 5 ml SD media lacking Uracil for plasmid maintenance and supplemented with 1 mol/l sorbitol. Cell density was measured at 600 nm. For the growth assays 25 ml of the identical media were inoculated with equal amounts of cells reaching a starting cell density of 0.05. Cells were grown for 48 hours on a rotatory shaker at 200 rpm at 23° C. or 30° C. Cell density was measured at the indicated time points.

1.8 Generation of Man3GlcNAc2 Structure

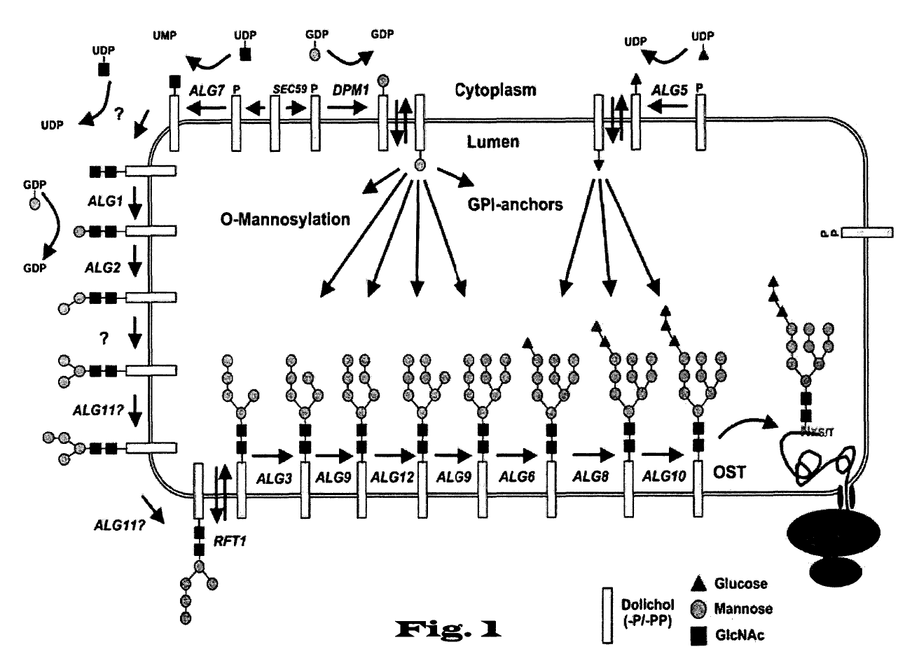

Lipid-linked oligosaccharides (LLO) represent the substrate for oligosaccharyl transferase in the endoplasmic reticulum (ER), transferring the assembled sugar to the asparagine residue of the N-glycosylation consensus sequence. The build up of the LLO is a sequential process, in which sugars from activated sugar donors are added to the growing LLO structure. The detailed pathway for the LLO synthesis is described in FIG. 1. By removing specific transferases from the cell tailored LLO structures can be generated.

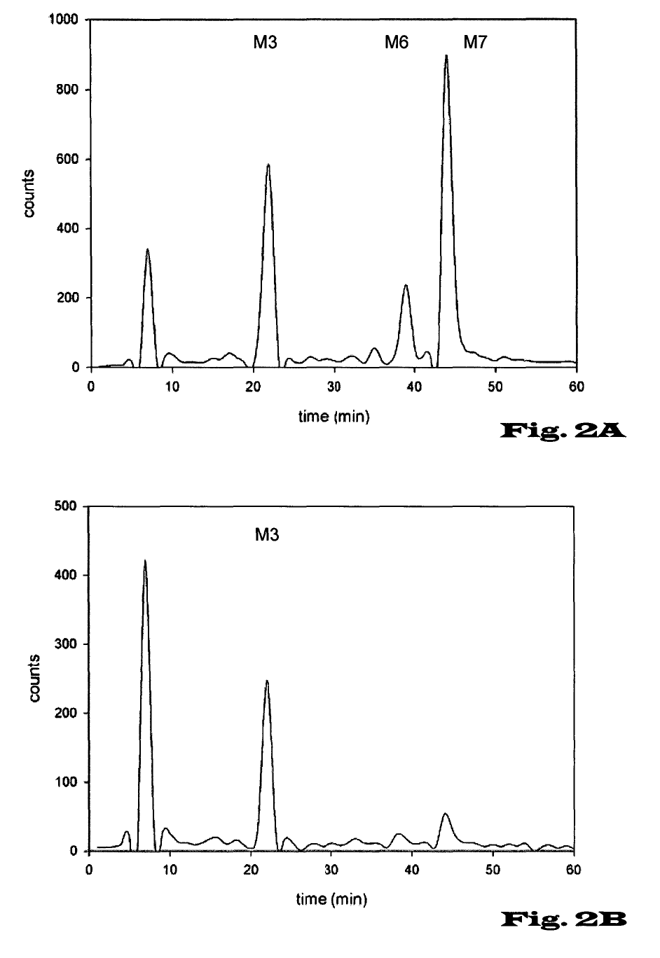

Inventors have found, without wishing to be bound to the theory, that in this process the proteins Alg3p and Alg11p play a major role in the build up of the LLO structure. By targeted removal of Alg11p the synthesis of the A-branch can successfully be prevented leading mainly to the generation of Man6GlcNAc2 and Man7GlcNAc2 structures (FIG. 2A). In a host cell of the invention a Man3GlcNAc2 structure can be synthesized on the cytosolic side of the ER and then flipped into the ER lumen, where it serves as substrate for the ER-lumen located transferases. Moreover Alg3p, the enzyme catalyzing the introduction of the α(1,3)-mannose initiating the B-branch is also identified to play a crucial role in the processing of the flipped LLO substrate. Elimination of Alg3p not only prevents the formation of the B-branch but the presence of the α(1,3)-mannose is a prerequisite for the formation of the C-branch. Therefore, a mutant yeast strain or similar is provided lacking both Alg3p-type and Alg11p-type of activities. The invented host cell thus produces mainly and preferably only low-mannose, and in particular Man3GlcNAc2 glycan structures, as revealed, for example, by [3H]-mannose labeling and HPLC profiling of the LLO structures produced (FIG. 2B).

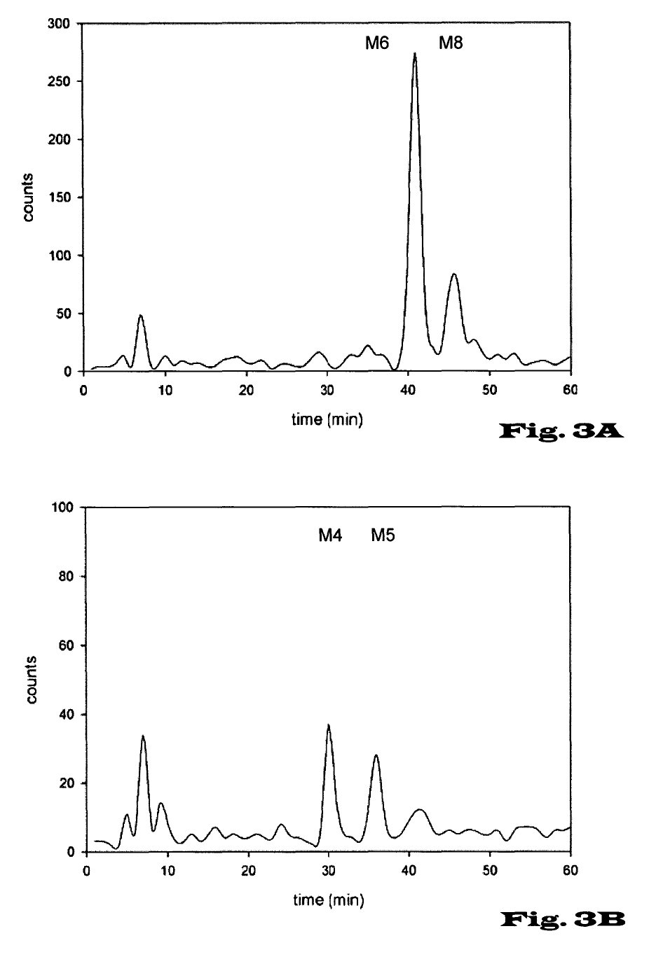

Protein linked oligosaccharides (NLO) analysis using [3H]-mannose labeling revealed in the Δalg3Δalg11 strain a structure bigger than Man3GlcNac2 but smaller than the N-glycans produced in the Δalg11 strain (FIG. 3B).

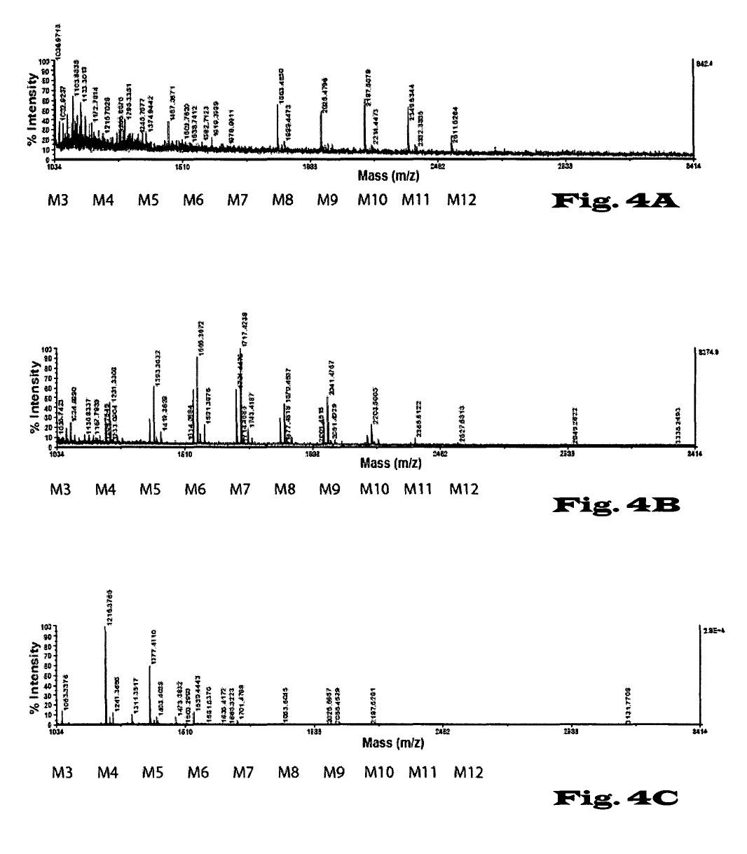

This structure was further characterized using MALDI-TOF MS of 2-AB labeled N-glycans isolated from cell wall proteins (FIG. 4). In contrast to wildtype yeast where an array of glycans comprising eight and more hexose residues in additions to the GlcNAc2 at the reducing end are present (FIG. 4A), in the Δalg11 strain mainly N-glycans comprising 5 to 9 hexoses in additions to the GlcNAc2 at the reducing end have been detected (FIG. 4B). In the Δalg3Δalg11 strain a small fraction of Man3GlcNAc2 (m/z 1053) and a bigger fraction of Man4GlcNAc2 (m/z 1215) and Man5GlcNAc2 (m/z 1377) structures was detected (FIG. 4C)

Overall the analysis of LLO and NLO show that in the Δalg3Δalg11 strain Man3GlcNAc2 is produced in the ER and transferred to protein, but that this structure is modified further in the Golgi apparatus.

1.9 High Copy Suppressor Screen—Identification of Novel Flippases

A high copy suppressor screen (HCSS) represents a preferred and efficient tool for the selection of gene providing a desired phenotype.

In order to identify genes able to compensate for the loss of the essential Rft1 function a HCSS was performedin a Δrft1 strain. A genomic yeast DNA library was expressed from the high copy plasmid Yep352 in the mutant strain.

Transformants were selected on minimal medium with 1 mol/l Sorbitol lacking uracil at 25° C. Grown transformants were tested for growth by replica-plating on YPD and YPDS at 33° C. The positive colonies (growing at 33° C. on YPD and YPDS) were tested for their ability to support growth of Δrft1 at 33, 35 and 37° C. 64 C.tes were further analyzed for their ability to improve the glycosylation in Δrft1 cells.

One of the clones contained a 3′ truncated version of the flc2 gene (flc2′). Flc2′ is encoded on the yeast chromosome 1. The truncated version identified in the HCSS screen comprises bases 43309 to 44631 of the full-length gene including its native promoter. The sequence of the Flc2′ expression plasmid (YEp352Flc2′) is given in FIG. 13 (SEQ ID NO: 33) or the coding sequence of the Flc2′ is depicted in FIG. 5A. Flc2′ encodes a protein of 452 amino acids comprising 4 complete and a fifth truncated transmembrane domain. The C-terminal 11 amino acids from amino acids 442 to 452 originate from the cloning procedure. (FIG. 5B). The flc2′ gene sequence and its promoter are presented in FIG. 5 (FIG. 5L).

1.10 Mutant Host Cells

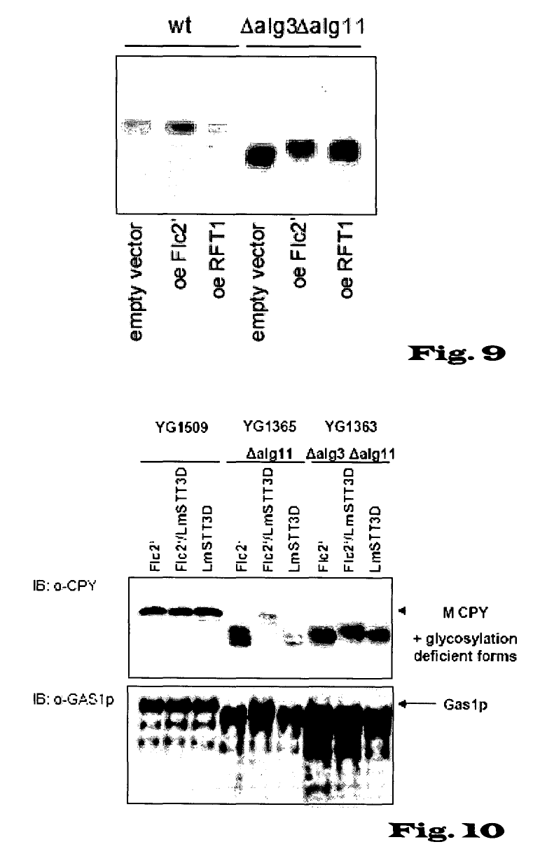

A spotting assay of Δrft1, Δalg11 or alg2-1 mutant strains carrying either Rft1 or Flc2′ expression plasmid was performed. Cells were spotted onto YPD plates. The plates were incubated for 3 days at 37 C, 30 C, or 31.5 C, respectively as indicated). Overexpression of Flc2′ results in improved growth of a Δrft1 or Δalg11 strain, displaying an identical or similar growth phenotype as the mutant strain expressing Rft1 (FIGS. 6A and 6B). Overexpression of Flc2′ also results in improved growth of a alg2-1 strain, while overexpression of Rft1 does not lead to inproved growth (FIG. 6C).

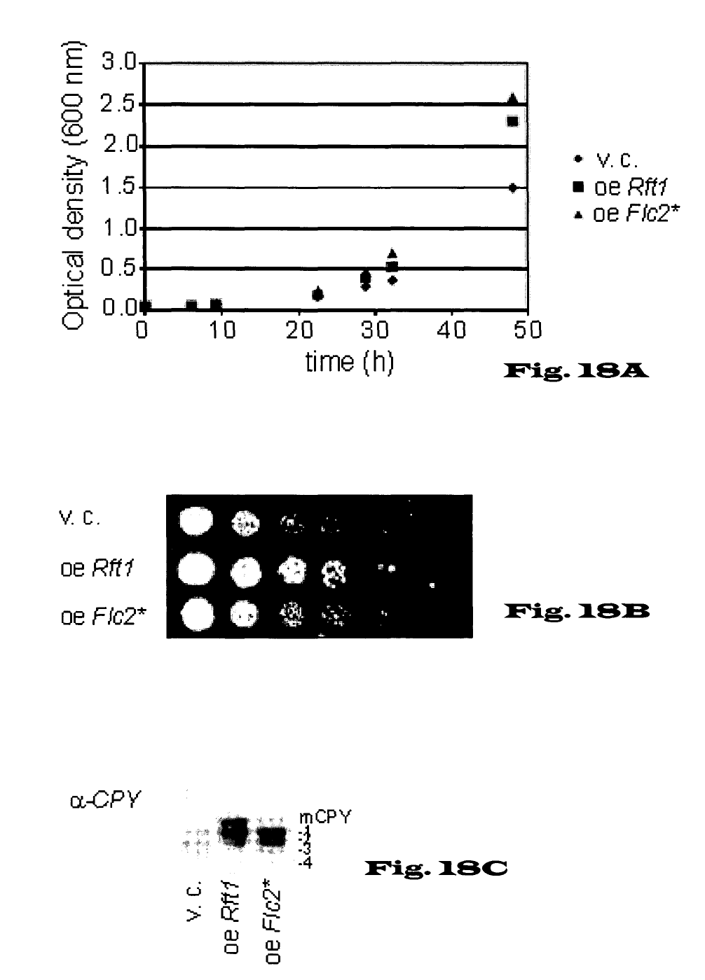

The Δalg3Δalg11 strain displays a highly temperature sensitive phenotype and growth defects. These defects can be strongly attenuated by expression of Flc2′. Expression of Flc2′ strongly improves the growth behavior of the strain and reduces the temperature sensitivity (FIG. 18B).

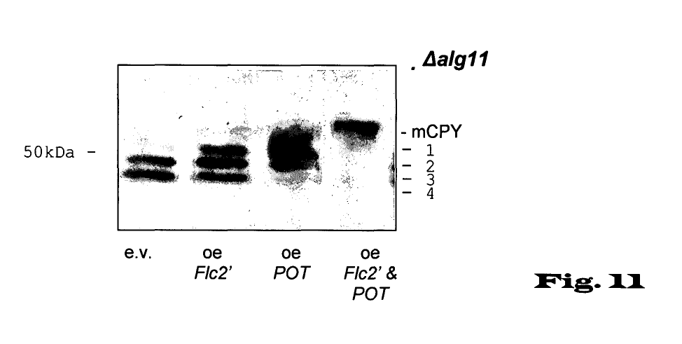

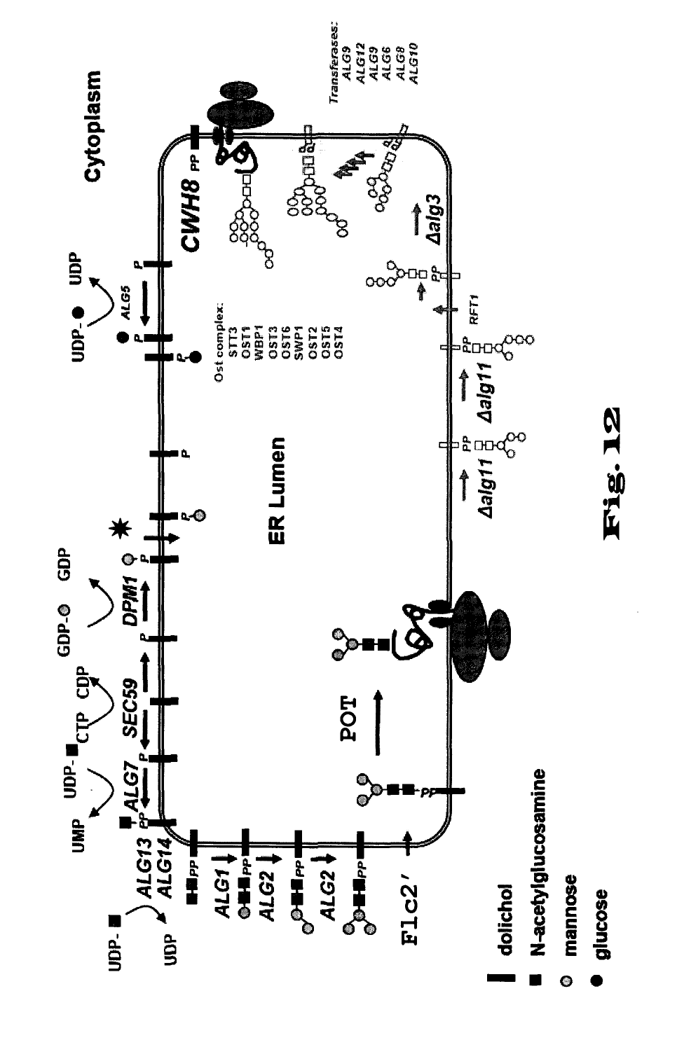

Further, a spotting assay of Δrft1 mutant strains carrying an expression plasmid encoding transmembrane domains 3 (SEQ ID NO: 16) or transmembrane domains 3 and 4 of Flc2′ (SEQ ID NO: 10) was performed. Cells were spotted as described above and incubated for 3 days at 37° C. Overexpression transmembrane domains 1-3 of Flc2′ or transmembrane domains 3-4 of Flc2′ results in increased growth, while cells expressing full length Flc2 do not show improved growth (FIGS. 7A and 7B).

Furthermore, Flc2′ was tested for its ability to restore glycosylation deficiency in a Δrft1 strain. Wildtype yeast strain and a Δrft1 carrying either an empty plasmid (YEp352), or plasmids for overexpression of Rft1, and Flc2′ were grown in SD-ura media (synthetic dextrose medium lacking uracil). Total soluble proteins were separated on SDS-PAGE gels and analyzed by immunoblotting using an anti-CPY antibody. Overexpression of Flc2′ restores N-glycosylation of carboxypeptidase CPY in a Δrft1 strain to similar levels as observed upon overexpression of Rft1 as revealed by immunoblotting (FIG. 7C).

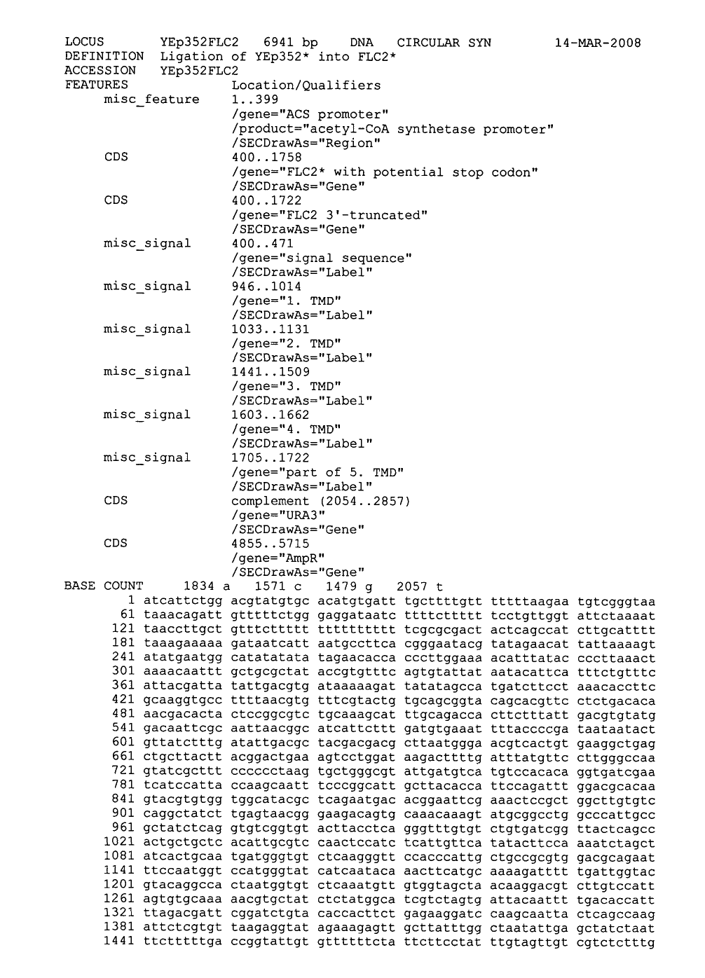

To investigate the effect of Flc2′ on the LLO synthesis, 3H-mannose labeling of Δrft1 cells carrying Flc2′ expression construct (FIG. 8C) was performed. As control Δrft1 cells carrying empty vector YEp352 (FIG. 8A) and Δrft1 cells carrying Rft1 expression construct (FIG. 8B) were used. Cells were labeled first with [3H]-mannose. Oligosaccharides were released from the lipid carrier by acid hydrolysis, purified and analyzed using HPLC. The HPLC profiles of [3H]-mannose labeled LLO show that in the absence of a functional flippase cells accumulate Man5GlcNAc2 (FIG. 8A). This indicates that the LLO synthesis is halted after the step catalyzed by Alg11p on the cytoplasmic side of the ER, since no molecule is present, which can flip the Man5GlcNAc2 into the ER lumen. Providing rft1 on plasmid restores LLO synthesis and leads to the accumulation of Glc3Man9GlcNAc2 (FIG. 8B). Upon expression of Flc2′ in Δrft1 cells flipping is restored and besides Man5GlcNAc2 also Glc3Man9GlcNAc2 accumulated in the cells (FIG. 8C). Overall this data indicates that Flc2′ functions as an flippase in Δrft1 yeast cells.

Expression of Flc2′ and/or Rft1 in a Δrft1 mutant strain improved final cell densities of the cultures after 48 to similar extents reaching approximately three times higher cell densities relative to the control strain (Table 6). In contrast to Flc2′, overexpression of full-length Flc2 did not compensate for flippase knock out in the Δrft1 strain: no growth improvement relative to the control of the Δrft1 strain was detectable. Endogenous full length Flc2 cannot complement the growth deficiencies of Δrft1 strain.

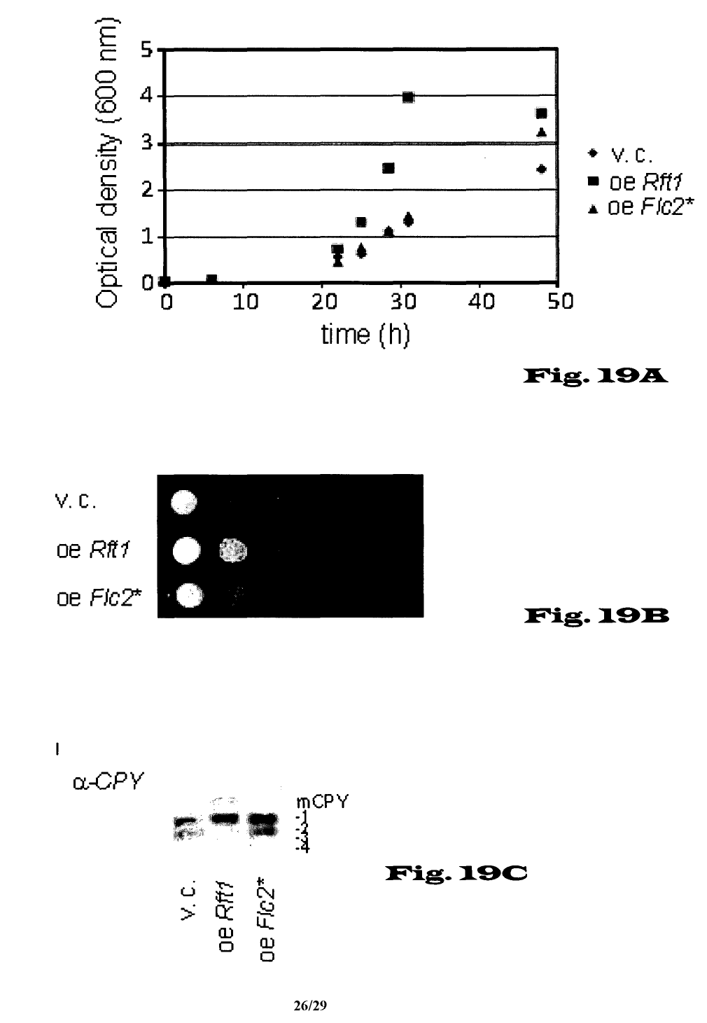

Expression of Rft1 or Flc2′ improved growth and led to higher final optical cell densities after 48 hours of Δalg11 (FIG. 19A) and Δalg3Δalg11 (FIG. 18A) mutant strains compared to the corresponding control carrying only empty plasmid. In the Δalg11 strain expression of Flc2* improved growth by 33% relative to the vector control, overexpression of Rft1 resulted in an increase of 49%. In the Δalg3Δalg11 mutant strain expression of Flc2* improved growth by 54% relative to the vector control, overexpression of Rft1 resulted in an increase of 74% in final cell density (Table 6).

Table 6 summarize the results of the growth assays of yeast strains overexpressing Rft1, Flc2′, full-length Flc2 or carrying and empty vector (control) (n.d.=not determined/measured).

TABLE 6

| | mutant strain | | |
|---|---|---|---|
| plasmid | Δrft1 | Δalg11 | Δalg11 Δalg3 |
| empty vector | 3.75 | 2.44 | 1.49 |
| Flc2* | 11.70 | 3.63 | 2.30 |
| Rft1 | 10.20 | 3.24 | 2.59 |
| Flc2 | 2.60 | n.d. | n.d. |

1.11 Flipping and Transfer of Man3GlcNAc2 Structure

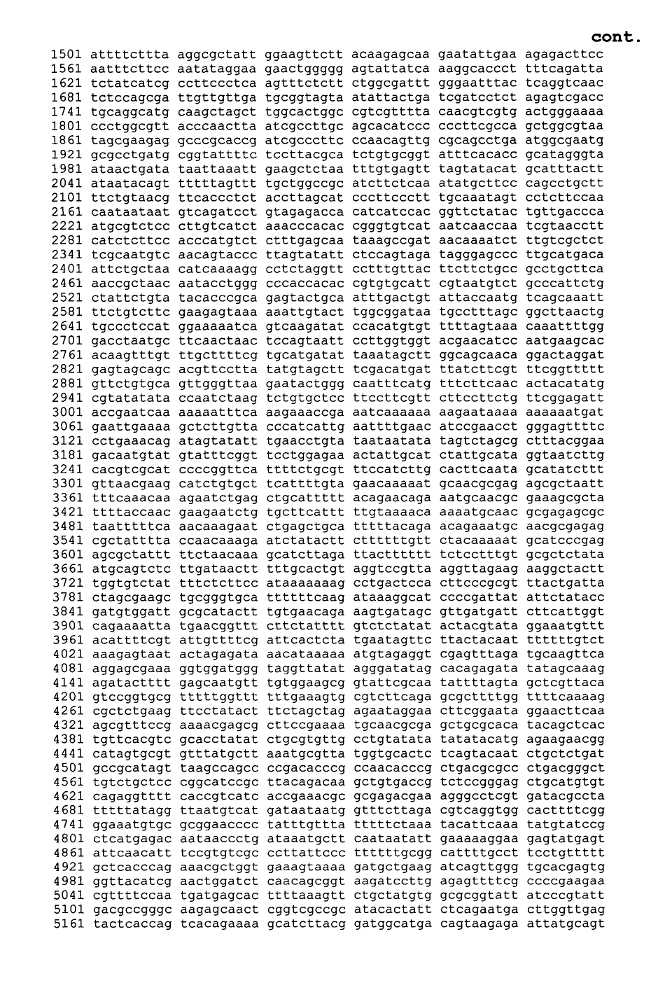

The effect of overexpression of Flc2′ on the N-glycosylation efficiency of carboxypeptidase Y (CPY) was analyzed in the Δalg3Δalg11 strain. Wildtype yeast strain and a Δalg3Δalg11 carrying either an empty plasmid (YEp352), or plasmids for overexpression of Flc2′, or Rft1 were grown in SD-ura media. Total soluble proteins were separated on SDS-PAGE gels and analyzed by immunoblotting using an anti-CPY antibody (FIG. 9). In wild type cells CPY is completely glycosylated independent on the overexpression of Rft1 or Flc2′. However, expression of Flc2′ or Rft1 in Δalg3Δalg11 strain improved the glycosylation of CPY as revealed by a shift of CPY to a higher molecular weight (FIG. 9).

1.12 Specificity Assay for Flippases in alg2-1 Strain

To establish activity and specificity of Flc2′ towards short LLO a yeast strain carrying a temperature sensitive Alg2 protein was selected. Due to lower Alg2 activity, this strain mainly accumulates Man1GlcNAc2 (M1) and Man2GlcNAc2 (M2) structures. However the residual enzyme activity leads to the generation of the regular yeast LLO being Glc3Man9GlcNAc2. If M1 or M2 are flipped into the ER lumen these two LLO species are no substrates for the lumenal Mannosyltransferases involved in the Alg pathway. M1 or M2 as well as Glc3Man9GlcNAc2 are transferred onto the protein. The Glc3Man9GlcNAc2 structures are further processed in the ER as well as in the Golgi apparatus giving rise to the NLO species comprising 8 to 14 Mannose residues. Alg2_{—}1 strain was transformed with Flc2′ and Rft1 expression vectors as well as with the empty vector control. The strains were grown to an A600 of 1 and the cells were harvested. Cell wall proteins were isolated, reduced, alkylated, and the N-glycans were liberated using PNGase F. N-glycans were purified, permethylated and analyzed by MALDI-TOF MS in the range from m/z of 700 to 4000.

Peaks of the expected sizes of M1, M2 and the high-mannose structures Man8GlcNAc2 to Man14GlcNAc2 (M8 to M14) were detected in MALDI-TOF spectra. Based on the peak intensities of NLO species relative abundances of the individual structures were calculated. A relative increase in M1 or M2 species indicates that flipping of these structures dominates elongation catalyzed by Alg2. Expression of Flc2′ led to the accumulation of 88.5% of M1 structures. In contrast M1 structures contributed only 74.7% and 78.7% to the total N-glycans in the alg2-1 strain expressing Rft1 or carrying the empty vector (Table 7).

Table 7 summarizes the relative abundance of N-glycans (%) in alg2-1 strain overexpressing Rft1 or Flc2* or carrying empty vector.

TABLE 7

| | mutant strain | | |
|---|---|---|---|
| N-Glycan species | empty vector | oeRft1 | oeFlc2′ |
| M1 | 78.7 | 74.7 | 88.5 |
| M2 | 19.1 | 21.7 | 10.9 |
| M8 to M14 | 2.1 | 3.5 | 0.6 |

1.13 Specificity Assay for Flippases in Δalg11 Strain

To establish activity and specificity of Flc2′ towards Man3GlcNac2 (M3) structures a Δalg11 yeast strain was selected. The use of this strain allows determining the relative abundances of LLO structures on the cytoplasmic and lumenal side of the ER membrane. Due to the inactivation of the alg11 gene LLO synthesis on the cytoplasmic side only proceeds to the level of M3. This structure, if flipped into the ER lumen, becomes further modified by Alg3 and the following mannosyltransferases leading to the generation of M7. Labeling of cells with 3H-mannose allows to quantify the relative abundances of the different LLO species using HPLC. If flipping is inefficient, cytoplasmic LLO species accumulate on the cytoplasmic side of the ER membrane, in contrast the relative amounts of lumenal LLO decreases.

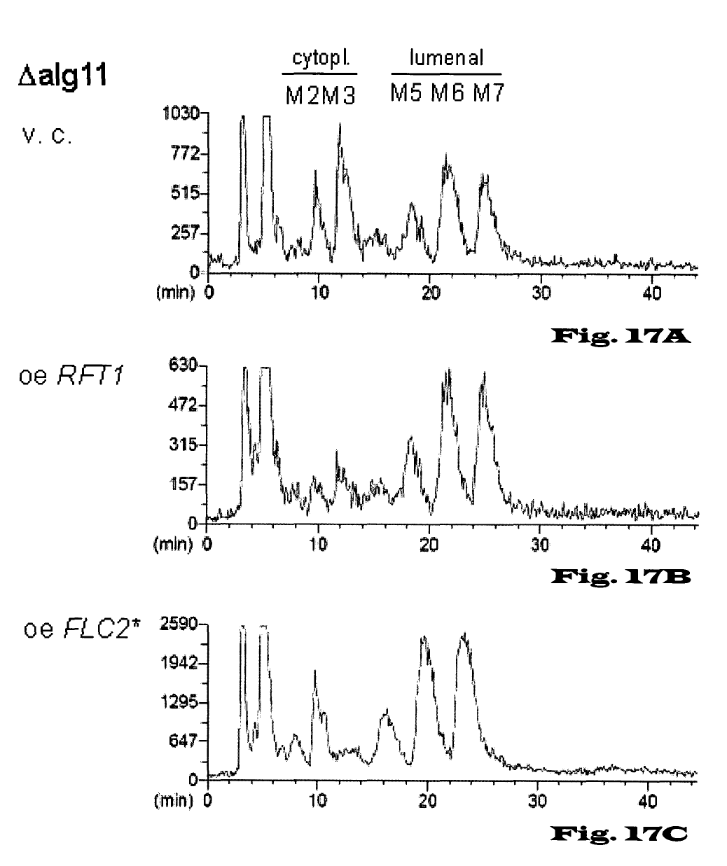

Expression of Flc2′ and Rft1 in Δalg11 strain decreases the relative contribution of cytoplamic LLO species to the total amount of LLO (FIGS. 17A, 17B, 17C), thereby increasing the lumenal LLO species from approximately 43% in the control strain to approximately 70% in both strains overexpressing Flc2′ or Rft1 (Table 8).

Table 8 summarizes the relative abundance (%) of different LLO species in Δalg11 strain overexpressing Rft1 or Flc2* or carrying empty vector. LLO species are assigned to either cytoplasmic or lumenal group.

TABLE 8

| | mutant strain | | |
|---|---|---|---|
| LLO species | empty vector | oeRft1 | oeFlc2′ |
| Cytoplasmic LLO | 43.5 | 28.5 | 31.0 |
| Lumenal LLO | 56.5 | 71.5 | 69.0 |

1.14 Generation of Δalg3 Δalg11 Δmnn1 Knock-Out Strain

A Δmnn1 deletion strain was crossed with a Δalg3 deletion strain. The diploid heterozygous Δalg3 Δmmn1 strain was sporulated and haploid spores tested for the absence of Δalg3 and mnn1 genes. Double knockout strains were tested by PCR analysis for the absence of alg3 and mnn1 genes. The selected Δalg3Δmmn1 strain was further crossed with a Δalg3Δalg11 strain, the resulting strains was sporulated and the tetrads were analyzed for strains lacking alg3, alg11 and mnn1 genes.

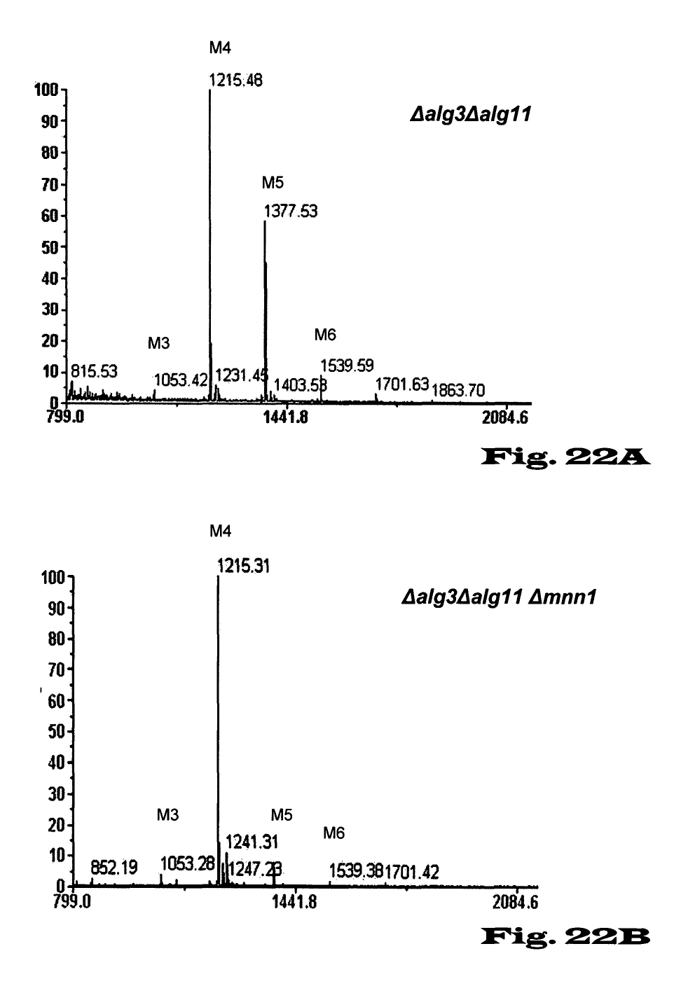

Glycoprofiles of double and triple mutants were analyzed as described. N-glycans were released from cell wall proteins by PNGase F, labeled with 2-AB and analyzed by MALDI-TOF MS. Comparison of an N-glycan spectra from a Δalg3Δalg11 and the triple mutant reveals the reduction of the peak at m/z=1377 representing a M5 structure. These data show that by elimination of mnn1 gene, the modification of the NLO in the Golgi apparatus can be abolished. FIG. 22 depicts the MALDI-TOF MS spectra of 2-AB-labeled N-glycans isolated from cell wall proteins from Δalg3Δalg11 yeast mutant strains (FIG. 22A) and cell wall proteins from Δalg11Δalg3Δmnn1 yeast mutant strains (FIG. 22B).

### EXAMPLE 2

### Composite System for Glycosylation

2.1 Expression of Novel LLO and Protozoan Oligosacharyl Transferase in Yeast Mutant Strains

In a preferred embodiment a composite system for glycosylation of proteins in particular in yeast, is provided which comprises at least three entities: (i) the generation of lipid-linked Man3GlcNAc2 as precursor for the oligosaccharyl transferase; (ii) a flippase e.g. (Flc2′), and (iii) the protozoan oligosaccharlytransferase (POT), which exhibits a relaxed substrate specificity.

In order to combine the two heterologous proteins, the flippase and POT a vector was constructed comprising both parts

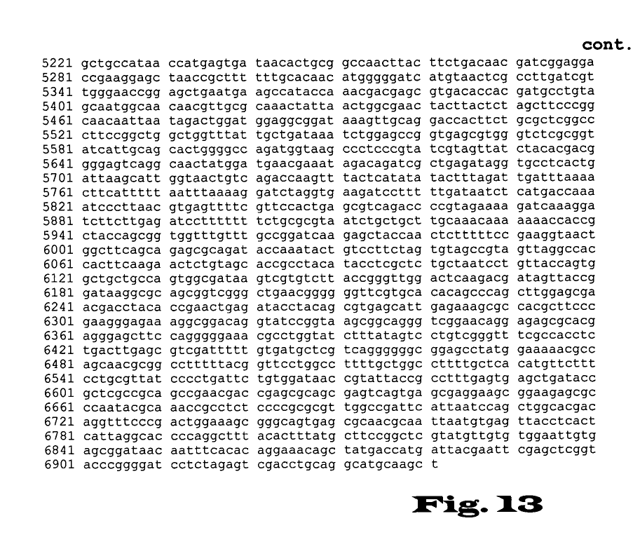

To that end, the protozoan oligosaccharyl transferase (LmStt3D) under the control of the GPD promoter and cyc1 terminator was inserted in the vector containing Flc2′ in such a manner that the genes are transcribed in opposite directions. Plasmid carrying either LmStt3D, Flc2′ or both enzymes were transformed into wild type yeast (YG1509) or yeast cells lacking either alg11 (YG1365) or alg11 and alg3 (YG1363), and the N-glycosylation of CPY and Gas1p was analyzed using Western blot (FIG. 10).

In the control strain without deletions of ER located oligosaccharyltranferase CPY mobility is identical upon expression of either Flc2′ or LmStt3D or both, Flc2′ and LmStt3D. In the yeast strain YG1365 which lacks alg11 and produces a lipid-linked GlcNAc2Man5 or YG1363 which lacks alg11 and alg3 and produces lipid-linked GlcNAc2Man3 coexpression of Flc2′ and LmStt3D shifts CPY to a higher molecular weight relative to cells expressing either Flc2′ or LmStt3D alone, indicating a more complete N-glycosylation of CPY in the presence of Flc2′ and LmStt3D. A similar change of mobility was observed on beta-1,3-glucanosyltransferase (Gas1p). This GPI-anchored protein is localized on the cell wall, and undergoes also the modifications occurring in the Golgi apparatus.

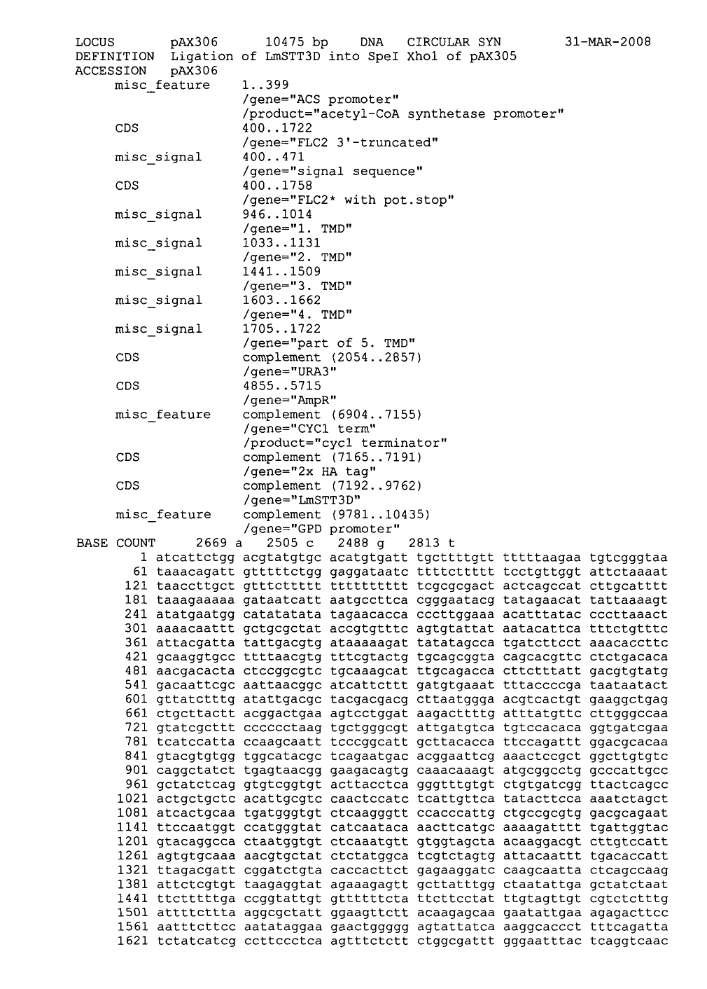

The effect of overexpression of Flc2′ and LmStt3D on the N-glycosylation efficiency of carboxypeptidase Y (CPY) was further analyzed in the Δalg11 strain carrying either an empty plasmid (YEp352), or plasmids for overexpression of Flc2′, or LmStt3D, or Flc2′ and LmStt3D were grown in SD-ura media at 23° C. Total soluble proteins were separated on SDS-PAGE gels and analyzed by immunoblotting using an anti-CPY antibody (FIG. 11). In Δalg11 cells overexpression of Flc2′ and LmStt3D CPY is completely glycosylated (mCPY), whereas cells either overexpressing only Flc2′ or POT LmStt3D strain reduce hypoglycosylation of CPY compared to the vector control but not to the same extent as the coepxression of Flc2′ and POT (FIG. 11).

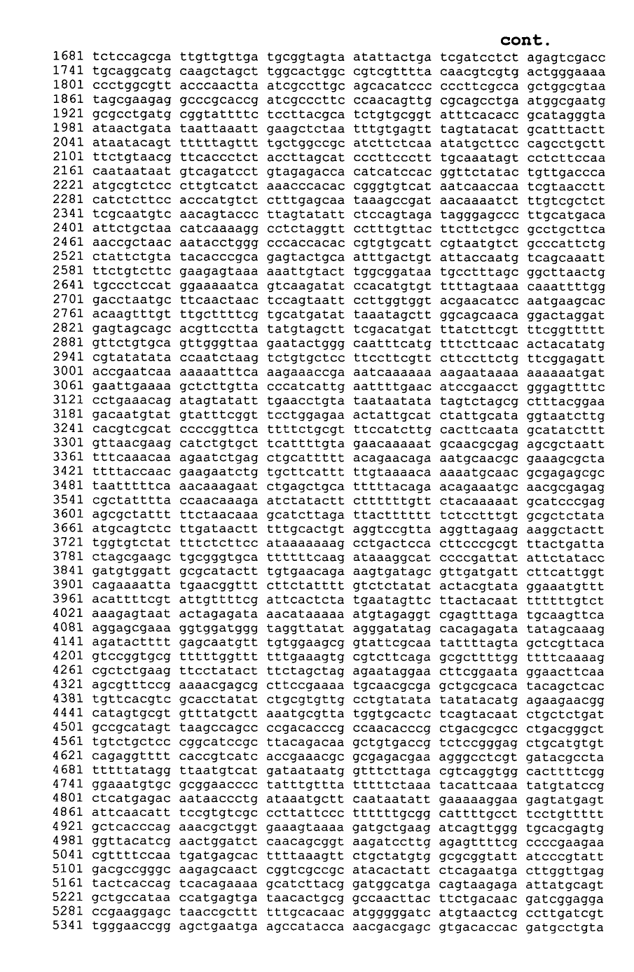

In the composite system which is schematically shown in FIG. 12, both, alg3 and alg11 genes are deleted resulting in the generation of lipid-linked Man3GlcNAc2. The remaining transferases are still present in the cell, but are inactive on a lipid-linked Man₃GlcNAc₂ substrate. In a first approach, a novel flippase (such as e.g. Flc2′) is added. Secondly a protozoan oligosaccharyltranferase (POT, such as Leishmania major Stt3D) is added. Alternatives for the generation of lipid-linked Man3GlcNAc2 would be the deletion of dpm1 gene, the product of which generates lipid-linked mannose on the cytoplasmic side of the ER membrane or the deletion of the monosaccharide flippase, which flipps the dolichol-linked mannose into the ER lumen. Lipid-linked mannose serves a donor for the ER lumen located oligosaccharyltransferases. In combination with the Δalg11 mutation such a cell would also produce lipid-linked Man3GlcNAc2. The redundant non used transferases, flippase (Rft1), components of the yeast Ost complex and the non-synthesized structures are depicted in grey.

2.2 Expression of Protozoan Oligosacharyl Transferase in Yeast Mutant Strains

A composite system for glycosylation of proteins in particular in yeast, is provided which comprises at least two entities: (i) the generation of lipid-linked Man3GlcNAc2 as precursor for the oligosaccharyl transferase and (ii) the expression of one or more paralogues of protozoan oligosaccharlytransferases (POT), which exhibit a relaxed substrate specificity.

A vector was constructed comprising POT. L. major possesses four Stt3 paralogous being LmStt3A to LmStt3D; L. braziliensis and L. infantum possess each three different Stt3 paralogues named Lb3_{—}1 to Lb3_{—}3, and Li3_{—}1 to Li3_{—}3, respectively. All respective POT genes were included on low copy number plasmids as well as on high copy number plasmids. In addition, POT genes for the paralogues TbStt3_B and TbStt3_C of Trypanosoma brucei were in cluded in high copy number plasmids.

The individual POT paralogues were expressed in modified Δalg11 mutant yeast strains and Δalg3Δalg11 mutant yeast strains in which the POT palsmids were introduced. Cell extracts of all strains were prepared and analyzed by a CPY specific antibody. Comparison of results of N-glycosylation efficiency reveals that effects of individual POT can differ in the different mutant strains, indicating different preferences of the different POT for the LLO substrate. Expression of POT from low copy number plasmids was more effective in improving N-glycosylation than the expression from high copy number plasmids, indicating that proper expression levels are crucial and can be optimized.

To establish a N-glycosylation score: a group of Western CPY blots (n=2 to 5) were analyzed and N-glycosylation efficiency scored from 0 (no additional effect) to 3 (large additional effect) in comparison to the unmodified Δalg11 and Δalg3Δalg11 backgrounds. N-glycosylation score calculated by summing points of the individual experiments and dividing the total trough the number of repetitions. The results are summarized in table 9.

TABLE 9

| | |
|---|---|
| POT plasmid | Glycosylation score |

| low copy plasmid | Δalg11 | Δalg3alg11 |
|---|---|---|
| LmStt3D | 2.25 | 1.33 |
| LbStt3-1 | 0 | 1 |
| LbStt3-2 | 0 | 0 |
| LbStt3-3 | 3 | 2.2 |
| LiStt3-1 | 0 | 1 |
| LiStt3-2 | 2.5 | 1 |
| LiStt3-3 | 0 | 0 |
| high copy plasmid | Δalg11 | Δalg3Δalg11 |
| LmStt3D | 2 | 1.75 |
| LbStt3-1 | 1 | 1 |
| LbStt3-2 | 0 | 0 |
| LbStt3-3 | 1.25 | 1 |
| LiStt3-1 | 0 | 0.5 |
| LiStt3-2 | 0 | 1 |
| LiStt3-3 | 0 | 0 |
| Tb3_B | 0.5 | 1 |
| Tb3_C | 0 | 1 |

1. A cell modified to express lipid-linked oligosaccharide (LLO) flippase activity that is capable of efficiently flipping LLO comprising 1 mannose residue, is capable of efficiently flipping LLO comprising 2 mannose residues and is capable of efficiently flipping LLO comprising 3 mannose residues, from the cytosolic side to the lumenal side of an intracellular organelle.

2-101. (canceled)


SEQUENCE LISTING (61 sequences)

SEQ ID NO: 1 - DNA, 1359 bp, Saccharomyces cerevisiae
atgatcttcctaaacaccttcgcaaggtgccttttaacgtgtttcgtactgtgcagcggtacagcacgttcctctgacacaaacgacactactccggcgtctgcaaagcatttgcagaccacttctttattgacgtgtatggacaattcgcaattaacggcatcattctttgatgtgaaattttaccccgataataatactgttatctttgatattgacgctacgacgacgcttaatgggaacgtcactgtgaaggctgagctgcttacttacggactgaaagtcctggataagacttttgatttatgttccttgggccaagtatcgctttcccccctaagtgctgggcgtattgatgtcatgtccacacaggtgatcgaatcatccattaccaagcaatttcccggcattgcttacaccattccagatttggacgcacaagtacgtgtggtggcatacgctcagaatgacacggaattcgaaactccgctggcttgtgtccaggctatcttgagtaacgggaagacagtgcaaacaaagtatgcggcctggcccattgccgctatctcaggtgtcggtgtacttacctcagggtttgtgtctgtgatcggttactcagccactgctgctcacattgcgtccaactccatctcattgttcatatacttccaaaatctagctatcactgcaatgatgggtgtctcaagggttccacccattgctgccgcgtggacgcagaatttccaatggtccatgggtatcatcaatacaaacttcatgcaaaagatttttgattggtacgtacaggccactaatggtgtctcaaatgttgtggtagctaacaaggacgtcttgtccattagtgtgcaaaaacgtgctatctctatggcatcgtctagtgattacaattttgacaccattttagacgattcggatctgtacaccacttctgagaaggatccaagcaattactcagccaagattctcgtgttaagaggtatagaaagagttgcttatttggctaatattgagctatctaatttctttttgaccggtattgtgttttttctattcttcctatttgtagttgtcgtctctttgattttctttaaggcgctattggaagttcttacaagagcaagaatattgaaagagacttccaatttcttccaatataggaagaactgggggagtattatcaaaggcacccttttcagattatctatcatcgccttccctcaagtttctcttctggcgatttgggaatttactcaggtcaactctccagcgattgttgttgatgcggtagtaatattactgatcgatcctctagagtcgacctgcaggcatgcaagctag

SEQ ID NO: 2 - protein, 452 aa, Saccharomyces cerevisiae
MIFLNTFARCLLTCFVLCSGTARSSDTNDTTPASAKHLQTTSLLTCMDNSQLTASFFDVKFYPDNNTVIFDIDATTTLNGNVTVKAELLTYGLKVLDKTFDLCSLGQVSLSPLSAGRIDVMSTQVIESSITKQFPGIAYTIPDLDAQVRVVAYAQNDTEFETPLACVQAILSNGKTVQTKYAAWPIAAISGVGVLTSGFVSVIGYSATAAHIASNSISLFIYFQNLAITAMMGVSRVPPIAAAWTQNFQWSMGIINTNFMQKIFDWYVQATNGVSNVVVANKDVLSISVQKRAISMASSSDYNFDTILDDSDLYTTSEKDPSNYSAKILVLRGIERVAYLANIELSNFFLTGIVFFLFFLFVVVVSLIFFKALLEVLTRARILKETSNFFQYRKNWGSIIKGTLFRLSIIAFPQVSLLAIWEFTQVNSPAIVVDAVVILLIDPLESTCRHAS

SEQ ID NO: 3 - DNA, 1116 bp, Saccharomyces cerevisiae
atgatcttcctaaacaccttcgcaaggtgccttttaacgtgtttcgtactgtgcagcggtacagcacgttcctctgacacaaacgacactactccggcgtctgcaaagcatttgcagaccacttctttattgacgtgtatggacaattcgcaattaacggcatcattctttgatgtgaaattttaccccgataataatactgttatctttgatattgacgctacgacgacgcttaatgggaacgtcactgtgaaggctgagctgcttacttacggactgaaagtcctggataagacttttgatttatgttccttgggccaagtatcgctttcccccctaagtgctgggcgtattgatgtcatgtccacacaggtgatcgaatcatccattaccaagcaatttcccggcattgcttacaccattccagatttggacgcacaagtacgtgtggtggcatacgctcagaatgacacggaattcgaaactccgctggcttgtgtccaggctatcttgagtaacgggaagacagtgcaaacaaagtatgcggcctggcccattgccgctatctcaggtgtcggtgtacttacctcagggtttgtgtctgtgatcggttactcagccactgctgctcacattgcgtccaactccatctcattgttcatatacttccaaaatctagctatcactgcaatgatgggtgtctcaagggttccacccattgctgccgcgtggacgcagaatttccaatggtccatgggtatcatcaatacaaacttcatgcaaaagatttttgattggtacgtacaggccactaatggtgtctcaaatgttgtggtagctaacaaggacgtcttgtccattagtgtgcaaaaacgtgctatctctatggcatcgtctagtgattacaattttgacaccattttagacgattcggatctgtacaccacttctgagaaggatccaagcaattactcagccaagattctcgtgttaagaggtatagaaagagttgcttatttggctaatattgagctatctaatttctttttgaccggtattgtgttttttctattcttcctatttgtagttgtcgtctctttgattttctttaagtag

SEQ ID NO: 4 - protein, 371 aa, Saccharomyces cerevisiae
MIFLNTFARCLLTCFVLCSGTARSSDTNDTTPASAKHLQTTSLLTCMDNSQLTASFFDVKFYPDNNTVIFDIDATTTLNGNVTVKAELLTYGLKVLDKTFDLCSLGQVSLSPLSAGRIDVMSTQVIESSITKQFPGIAYTIPDLDAQVRVVAYAQNDTEFETPLACVQAILSNGKTVQTKYAAWPIAAISGVGVLTSGFVSVIGYSATAAHIASNSISLFIYFQNLAITAMMGVSRVPPIAAAWTQNFQWSMGIINTNFMQKIFDWYVQATNGVSNVVVANKDVLSISVQKRAISMASSSDYNFDTILDDSDLYTTSEKDPSNYSAKILVLRGIERVAYLANIELSNFFLTGIVFFLFFLFVVVVSLIFFK

SEQ ID NO: 5 - DNA, 737 bp, Saccharomyces cerevisiae
atgatcttcctaaacaccttcgcaaggtgccttttaacgtgtttcgtactgtgcagcggtacagcacgttcctctgacacaaacgacactactccggcgtctgcaaagcatttgcagaccacttctttattgacgtgtatggacaattcgcaattaacggcatcattctttgatgtgaaattttaccccgataataatactgttatctttgatattgacgctacgacgacgcttaatgggaacgtcactgtgaaggctgagctgcttacttacggactgaaagtcctggataagacttttgatttatgttccttgggccaagtatcgctttcccccctaagtgctgggcgtattgatgtcatgtccacacaggtgatcgaatcatccattaccaagcaatttcccggcattgcttacaccattccagatttggacgcacaagtacgtgtggtggcatacgctcagaatgacacggaattcgaaactccgctggcttgtgtccaggctatcttgagtaacgggaagacagtgcaaacaaagtatgcggcctggcccattgccgctatctcaggtgtcggtgtacttacctcagggtttgtgtctgtgatcggttactcagccactgctgctcacattgcgtccaactccatctcattgttcatatacttccaaaatctagctatcactgcaatgatgggtgtctcaagggttccacccattgctgccgcgtggactag

SEQ ID NO: 6 - protein, 245 aa, Saccharomyces cerevisiae
MIFLNTFARCLLTCFVLCSGTARSSDTNDTTPASAKHLQTTSLLTCMDNSQLTASFFDVKFYPDNNTVIFDIDATTTLNGNVTVKAELLTYGLKVLDKTFDLCSLGQVSLSPLSAGRIDVMSTQVIESSITKQFPGIAYTIPDLDAQVRVVAYAQNDTEFETPLACVQAILSNGKTVQTKYAAWPIAAISGVGVLTSGFVSVIGYSATAAHIASNSISLFIYFQNLAITAMMGVSRVPPIAAAWT

SEQ ID NO: 7 - DNA, 813 bp, Saccharomyces cerevisiae
atgatcttcctaaacaccttcgcaaggtgccttttaacgtgtttcgtactgtgcagcggtacagcacgttcctctgacacaaacgacattgcgtccaactccatctcattgttcatatacttccaaaatctagctatcactgcaatgatgggtgtctcaagggttccacccattgctgccgcgtggacgcagaatttccaatggtccatgggtatcatcaatacaaacttcatgcaaaagatttttgattggtacgtacaggccactaatggtgtctcaaatgttgtggtagctaacaaggacgtcttgtccattagtgtgcaaaaacgtgctatctctatggcatcgtctagtgattacaattttgacaccattttagacgattcggatctgtacaccacttctgagaaggatccaagcaattactcagccaagattctcgtgttaagaggtatagaaagagttgcttatttggctaatattgagctatctaatttctttttgaccggtattgtgttttttctattcttcctatttgtagttgtcgtctctttgattttctttaaggcgctattggaagttcttacaagagcaagaatattgaaagagacttccaatttcttccaatataggaagaactgggggagtattatcaaaggcacccttttcagattatctatcatcgccttccctcaagtttctcttctggcgatttgggaatttactcaggtcaactctccagcgattgttgttgatgcggtagtaatattactgatcgatcctctagagtcgacctgcaggcatgcaagctag

SEQ ID NO: 8 - protein, 270 aa, Saccharomyces cerevisiae
MIFLNTFARCLLTCFVLCSGTARSSDTNDIASNSISLFIYFQNLAITAMMGVSRVPPIAAAWTQNFQWSMGIINTNFMQKIFDWYVQATNGVSNVVVANKDVLSISVQKRAISMASSSDYNFDTILDDSDLYTTSEKDPSNYSAKILVLRGIERVAYLANIELSNFFLTGIVFFLFFLFVVVVSLIFFKALLEVLTRARILKETSNFFQYRKNWGSIIKGTLFRLSIIAFPQVSLLAIWEFTQVNSPAIVVDAVVILLIDPLESTCRHAS

SEQ ID NO: 9 - DNA, 405 bp, Saccharomyces cerevisiae
atgatcttcctaaacaccttcgcaaggtgccttttaacgtgtttcgtactgtgcagcggtacagcacgttcctctgacacaaacgacttctttttgaccggtattgtgttttttctattcttcctatttgtagttgtcgtctctttgattttctttaaggcgctattggaagttcttacaagagcaagaatattgaaagagacttccaatttcttccaatataggaagaactgggggagtattatcaaaggcacccttttcagattatctatcatcgccttccctcaagtttctcttctggcgatttgggaatttactcaggtcaactctccagcgattgttgttgatgcggtagtaatattactgatcgatcctctagagtcgacctgcaggcatgcaagctag

SEQ ID NO: 10 - protein, 134 aa, Saccharomyces cerevisiae
MIFLNTFARCLLTCFVLCSGTARSSDTNDFFLTGIVFFLFFLFVVVVSLIFFKALLEVLTRARILKETSNFFQYRKNWGSIIKGTLFRLSIIAFPQVSLLAIWEFTQVNSPAIVVDAVVILLIDPLESTCRHAS

SEQ ID NO: 11 - DNA, 621 bp, Saccharomyces cerevisiae
atgatcttcctaaacaccttcgcaaggtgccttttaacgtgtttcgtactgtgcagcggtacagcacgttcctctgacacaaacgacactactccggcgtctgcaaagcatttgcagaccacttctttattgacgtgtatggacaattcgcaattaacggcatcattctttgatgtgaaattttaccccgataataatactgttatctttgatattgacgctacgacgacgcttaatgggaacgtcactgtgaaggctgagctgcttacttacggactgaaagtcctggataagacttttgatttatgttccttgggccaagtatcgctttcccccctaagtgctgggcgtattgatgtcatgtccacacaggtgatcgaatcatccattaccaagcaatttcccggcattgcttacaccattccagatttggacgcacaagtacgtgtggtggcatacgctcagaatgacacggaattcgaaactccgctggcttgtgtccaggctatcttgagtaacgggaagacagtgcaaacaaagtatgcggcctggcccattgccgctatctcaggtgtcggtgtacttacctcagggtttgtgtctgtgatcggttactcatag

SEQ ID NO: 12 - protein, 206 aa, Saccharomyces cerevisiae
MIFLNTFARCLLTCFVLCSGTARSSDTNDTTPASAKHLQTTSLLTCMDNSQLTASFFDVKFYPDNNTVIFDIDATTTLNGNVTVKAELLTYGLKVLDKTFDLCSLGQVSLSPLSAGRIDVMSTQVIESSITKQFPGIAYTIPDLDAQVRVVAYAQNDTEFETPLACVQAILSNGKTVQTKYAAWPIAAISGVGVLTSGFVSVIGYS

SEQ ID NO: 13 - DNA, 191 bp, Saccharomyces cerevisiae
atgatcttcctaaacaccttcgcaaggtgccttttaacgtgtttcgtactgtgcagcggtacagcacgttcctctgacacaaacgacattgcgtccaactccatctcattgttcatatacttccaaaatctagctatcactgcaatgatgggtgtctcaagggttccacccattgctgccgcgtggactag

SEQ ID NO: 14 - protein, 63 aa, Saccharomyces cerevisiae
MIFLNTFARCLLTCFVLCSGTARSSDTNDIASNSISLFIYFQNLAITAMMGVSRVPPIAAAWT

SEQ ID NO: 15 - DNA, 162 bp, Saccharomyces cerevisiae
atgatcttcctaaacaccttcgcaaggtgccttttaacgtgtttcgtactgtgcagcggtacagcacgttcctctgacacaaacgacttctttttgaccggtattgtgttttttctattcttcctatttgtagttgtcgtctctttgattttctttaagtag

SEQ ID NO: 16 - protein, 53 aa, Saccharomyces cerevisiae
MIFLNTFARCLLTCFVLCSGTARSSDTNDFFLTGIVFFLFFLFVVVVSLIFFK

SEQ ID NO: 17 - DNA, 243 bp, Saccharomyces cerevisiae
atgatcttcctaaacaccttcgcaaggtgccttttaacgtgtttcgtactgtgcagcggtacagcacgttcctctgacacaaacgacggcacccttttcagattatctatcatcgccttccctcaagtttctcttctggcgatttgggaatttactcaggtcaactctccagcgattgttgttgatgcggtagtaatattactgatcgatcctctagagtcgacctgcaggcatgcaagctag

SEQ ID NO: 18 - protein, 80 aa, Saccharomyces cerevisiae
MIFLNTFARCLLTCFVLCSGTARSSDTNDGTLFRLSIIAFPQVSLLAIWEFTQVNSPAIVVDAVVILLIDPLESTCRHAS

SEQ ID NO: 19 - DNA, 72 bp, Saccharomyces cerevisiae
atgatcttcctaaacaccttcgcaaggtgccttttaacgtgtttcgtactgtgcagcggtacagcacgttcc

SEQ ID NO: 20 - protein, 24 aa, Saccharomyces cerevisiae
MIFLNTFARCLLTCFVLCSGTARS

SEQ ID NO: 21 - DNA, 69 bp, Saccharomyces cerevisiae
gcctggcccattgccgctatctcaggtgtcggtgtacttacctcagggtttgtgtctgtgatcggttac

SEQ ID NO: 22 - protein, 23 aa, Saccharomyces cerevisiae
AWPIAAISGVGVLTSGFVSVIGY

SEQ ID NO: 23 - DNA, 98 bp, Saccharomyces cerevisiae
attgcgtccaactccatctcattgttcatatacttccaaaatctagctatcactgcaatgatgggtgtctcaagggttccacccattgctgccgcgtg

SEQ ID NO: 24 - protein, 33 aa, Saccharomyces cerevisiae
IASNSISLFIYFQNLAITAMMGVSRVPPIAAAW

SEQ ID NO: 25 - DNA, 69 bp, Saccharomyces cerevisiae
ttctttttgaccggtattgtgttttttctattcttcctatttgtagttgtcgtctctttgattttcttt

SEQ ID NO: 26 - protein, 23 aa, Saccharomyces cerevisiae
FFLTGIVFFLFFLFVVVVSLIFF

SEQ ID NO: 27 - DNA, 60 bp, Saccharomyces cerevisiae
ggcacccttttcagattatctatcatcgccttccctcaagtttctcttctggcgatttgg

SEQ ID NO: 28 - protein, 20 aa, Saccharomyces cerevisiae
GTLFRLSIIAFPQVSLLAIW

SEQ ID NO: 29 - DNA, 17 bp, Saccharomyces cerevisiae
gtagtaatattactgat

SEQ ID NO: 30 - protein, 6 aa, Saccharomyces cerevisiae
VVILLI

SEQ ID NO: 31 - DNA, 6941 bp, Saccharomyces cerevisiae
atcattctggacgtatgtgcacatgtgatttgcttttgtttttttaagaatgtcgggtaataaacagattgtttttctgggaggataatcttttcttttttcctgttggtattctaaaattaaccttgctgtttcttttttttttttttttcgcgcgactactcagccatcttgcatttttaaagaaaaagataatcattaatgccttcacgggaatacgtatagaacattattaaaagtatatgaatggcatatatatatagaacaccacccttggaaaacatttataccccttaaactaaaacaatttgctgcgctataccgtgtttcagtgtattataatacattcatttctgtttcattacgattatattgacgtgataaaaagattatatagccatgatcttcctaaacaccttcgcaaggtgccttttaacgtgtttcgtactgtgcagcggtacagcacgttcctctgacacaaacgacactactccggcgtctgcaaagcatttgcagaccacttctttattgacgtgtatggacaattcgcaattaacggcatcattctttgatgtgaaattttaccccgataataatactgttatctttgatattgacgctacgacgacgcttaatgggaacgtcactgtgaaggctgagctgcttacttacggactgaaagtcctggataagacttttgatttatgttccttgggccaagtatcgctttcccccctaagtgctgggcgtattgatgtcatgtccacacaggtgatcgaatcatccattaccaagcaatttcccggcattgcttacaccattccagatttggacgcacaagtacgtgtggtggcatacgctcagaatgacacggaattcgaaactccgctggcttgtgtccaggctatcttgagtaacgggaagacagtgcaaacaaagtatgcggcctggcccattgccgctatctcaggtgtcggtgtacttacctcagggtttgtgtctgtgatcggttactcagccactgctgctcacattgcgtccaactccatctcattgttcatatacttccaaaatctagctatcactgcaatgatgggtgtctcaagggttccacccattgctgccgcgtggacgcagaatttccaatggtccatgggtatcatcaatacaaacttcatgcaaaagatttttgattggtacgtacaggccactaatggtgtctcaaatgttgtggtagctaacaaggacgtcttgtccattagtgtgcaaaaacgtgctatctctatggcatcgtctagtgattacaattttgacaccattttagacgattcggatctgtacaccacttctgagaaggatccaagcaattactcagccaagattctcgtgttaagaggtatagaaagagttgcttatttggctaatattgagctatctaatttctttttgaccggtattgtgttttttctattcttcctatttgtagttgtcgtctctttgattttctttaaggcgctattggaagttcttacaagagcaagaatattgaaagagacttccaatttcttccaatataggaagaactgggggagtattatcaaaggcacccttttcagattatctatcatcgccttccctcaagtttctcttctggcgatttgggaatttactcaggtcaactctccagcgattgttgttgatgcggtagtaatattactgatcgatcctctagagtcgacctgcaggcatgcaagctagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaaaagctcttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatccagctggcacgacaggtttcccgactggaaagcgggcagtgagcgcaacgcaattaatgtgagttacctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagct

SEQ ID NO: 32 - DNA, 10475 bp, Saccharomyces cerevisiae
atcattctggacgtatgtgcacatgtgatttgcttttgtttttttaagaatgtcgggtaataaacagattgtttttctgggaggataatcttttcttttttcctgttggtattctaaaattaaccttgctgtttcttttttttttttttttcgcgcgactactcagccatcttgcatttttaaagaaaaagataatcattaatgccttcacgggaatacgtatagaacattattaaaagtatatgaatggcatatatatatagaacaccacccttggaaaacatttataccccttaaactaaaacaatttgctgcgctataccgtgtttcagtgtattataatacattcatttctgtttcattacgattatattgacgtgataaaaagattatatagccatgatcttcctaaacaccttcgcaaggtgccttttaacgtgtttcgtactgtgcagcggtacagcacgttcctctgacacaaacgacactactccggcgtctgcaaagcatttgcagaccacttctttattgacgtgtatggacaattcgcaattaacggcatcattctttgatgtgaaattttaccccgataataatactgttatctttgatattgacgctacgacgacgcttaatgggaacgtcactgtgaaggctgagctgcttacttacggactgaaagtcctggataagacttttgatttatgttccttgggccaagtatcgctttcccccctaagtgctgggcgtattgatgtcatgtccacacaggtgatcgaatcatccattaccaagcaatttcccggcattgcttacaccattccagatttggacgcacaagtacgtgtggtggcatacgctcagaatgacacggaattcgaaactccgctggcttgtgtccaggctatcttgagtaacgggaagacagtgcaaacaaagtatgcggcctggcccattgccgctatctcaggtgtcggtgtacttacctcagggtttgtgtctgtgatcggttactcagccactgctgctcacattgcgtccaactccatctcattgttcatatacttccaaaatctagctatcactgcaatgatgggtgtctcaagggttccacccattgctgccgcgtggacgcagaatttccaatggtccatgggtatcatcaatacaaacttcatgcaaaagatttttgattggtacgtacaggccactaatggtgtctcaaatgttgtggtagctaacaaggacgtcttgtccattagtgtgcaaaaacgtgctatctctatggcatcgtctagtgattacaattttgacaccattttagacgattcggatctgtacaccacttctgagaaggatccaagcaattactcagccaagattctcgtgttaagaggtatagaaagagttgcttatttggctaatattgagctatctaatttctttttgaccggtattgtgttttttctattcttcctatttgtagttgtcgtctctttgattttctttaaggcgctattggaagttcttacaagagcaagaatattgaaagagacttccaatttcttccaatataggaagaactgggggagtattatcaaaggcacccttttcagattatctatcatcgccttccctcaagtttctcttctggcgatttgggaatttactcaggtcaactctccagcgattgttgttgatgcggtagtaatattactgatcgatcctctagagtcgacctgcaggcatgcaagctagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaaaagctcttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatccagctggcacgacaggtttcccgactggaaagcgggcagtgagcgcaacgcaattaatgtgagttacctcactcattaggcaccccaggctttacactttatgcttccggctcgtatgttgtgtggaattgtgagcggataacaatttcacacaggaaacagctatgaccatgattacgaattcgagctcggtaccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgagttacgcatagtcaggaacatcgtatgggtaccgtcggttctcgctttcacgcatccggcgcatgtactcctcctggtacgacccgacattgtttttccgatcggcgtttgtcacctgatcgaaggggacgcggtgtgccagcatctcctggatctccttcgccggcgggtactgcccggggcagatccacgacccaggcgggtggcacacgcggtttgccgggtcagcaacccactttttgctctccgcgctcacgttcatgaccttgaagatgcgcaccaggccgtacttcgacgagtacacctcctgaaagagggacgggttcaccttcacgccctttcttttcccggcctcgtgcaggttgtacagcagcgacgcccgcatcatcggcgttgggcgactgtagtcatttctgtgaaagccgaattgctggcacagcgggtcgtcggggcagatgtcgtggtacacgctgttgccgatgcgcgccatgtgcggtgacttcatcaggtcgccgctctgcccagcccagatcaggacgtagtcggccatgtggcgcaccagcgagtgcgcctccgccacgggcgacgtcagcatcttgccgatcgtggcgatgtgctcgtggttccaggtgttgccatcggccagcgaggtgcggttgccgatgcctgtgatctggtagccgtagtcccaccaggccaaaacgcgcgcgtcctctggcgtgctgtcgcgcagccactcgtaggccttgaggtagtcatccaccaataggttcataggcttgcctgtggcacggttttgcacgacggccgcgaaaacaatcatcggatttgacgactgctccgcaaactttgttgagtgggacgcgaactcggaactgaagaagctcaccgcggttgtcgtgacgagagcccacatagcgatggacaggaccatgcgatggccccaagcaagagagctaccggcaaagacgtcgcaaaatgcgcgcgcggtcgttgcgttcttagcgtcatctcggccgctgcccttgccagcccccctctggtggcgttgcgcctgcttctgctgctttttagcctttgtcgcatcactgtcccagaaactgagttgcacggctgcttccagaattgtcccgacgaaaatgccagtggacagacacgcagcggggccggagagaagcagcagccgagccatgcgagtgctgaagtagtacacggcgccggagttcagtagccagaataccttcgacggggagtagtgcacgaacgtcgagacagcaagcacaatggagcccaaaccccatgtcacgccgcacacgtgaagaaaagcccacatcgcctccgggctggcgggttgatgttcggcgaccgagtcgaccagcggattgccagtgcgcgtgtgctccacgaacagcgcacgcacacggaccgagaggggcccgaagtaccccgtcggtgccagcaccgagattgcaagcgcagccacgccagccatcacgctgaagacgcgcacgcggatcttgaagttcgcgcgagagcgaacctcgacaccggcgcgtgcgcgcagcacctcgcacacctgcagtccacacaggaagacaagcaccagcagcgcacccagctgctccagcgacttgaagggcgacatccccactggcggcacgcacacggcgatggcggtgcccacgacgtagaacagcgtgtatgcacgcagcagcgacgggttgtacgtgttgcgggcccagtccaccatcgatgatatgccggcatgcatggcaaccatgttgagcacgaaaatgtagccgccccacgccgccgccatgtagccgtaggcgacaccggtgaggacaccgatgggccacgaggaccgcgtgcgcagcgagcgcacccagcagtagaaagtgaggagcatggctgcgacggcgatgcactcgttgtcgaactcacccgccatggaccgcatcaggtgggctgggatgatggagaaggagagtgcggcagcggccgccgctactgtcgacccactggcttcgtaggtgcaaaacgccagagtagcggtagcgatggcgccaaaccacgctggcatcagcacgcacacgttgttgagagacatcggcatgccggcagccgccagtgcgcggtgaatggcgacggcagtgagctgcaggcccgggtacgtggtggagccgacggggcggcccagcgggtaccagctcatgtagtcgaaccagctgaagaaggcggaccagccgtgcgtggacatgtactcggcagcgcggtagttgaaccacgggtcgaactcgtggatcaggtatccgtaaatctgaacggagatcatgcgaaccgtgaaggcttggaagcagctggcggctaagacgaagagtgccaccacggtgaggacgaagtgtactggccagaaaggaaacggaaagatgccgatgaaatccttctcatctgttagcgttttgggcagcaaaatcaccttcgccggtggagatgcggtcttggtttgtgaggcggcatcttcggcttgggccgaagcttcacgagatgcggttgccgcagagccggagtcgcccaatgaatttcccttccgcttgcccatgttgttactagttctagaatccgtcgaaactaagttctggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcgagtattgataatgataaactgagggggatcctctagagtcgacctgcaggcatgcaagct

SEQ ID NO: 33 - DNA, 2580 bp, Leishmania braziliensis
atgccgatcaagaaccagcgcaaaggatgcgaggagggtaaccccaacccctcctccacacccgcagcagagccactggcaaacgcagaaggcacgcagagggataccgctgaagggactcctatggagccacccagcgagacgtacctcttcaactgccgcgccgcaccgtactcgaagctgatatacgtctacaaaggtatcatgttcacattgattctctacgcgatccgcttagcgtaccagactcgcatgctatccgttcagacttatggctacatcatccacgagttcgacccgtggttcaactaccgcgccgccgagtacatgtccgcgcacggctggtccgccttcttcagctggttcgactacatgagctggtacccgctgggccgccctgttggcaccaccacgtacccgggcctgcagctcaccgccgttgccatccaccgcgcattggcagctgccggggtgccgatgtctctcaacaacgtgtgtgtgctgatccccgcgtggtatggtgccatcgctactgctatcatggccctcatggccttcgaaacgactggctcgatcgctgtttctgcatgggctgcactcctcttctccatcattccagcacacctgatgcggtccatggcgggcgagttcgacaacgagtgcatcgccgttgcagccatgctcctcaccttctacttgtgggtacgctcgctgcgcacgcggtgctcgtggcccatcggcatcctcaccggtatcgcctacggctacatggtggcggcgtggggcggatacatttttgtgctcaacatggttgccatgcacgccggcatatcatcgatggtcgactgggctcgcaacacgtacaacccgtcgctgctgcgcgcatacgcgctgttctacgttgtcggcaccgccatcgccacgcgcgtgccgcctgtggggatgtcgcccttcaggtcgctggagcagctgggtgcgctggtggtgctcctcttcctgtgcgggctgcaggcctgcgaggtgtttcgcgcacgggccgacgtcgaggttcgctcccgcgcgaacttcaagatccgcatgcgtgccttcagcgtgatggctggcgtgggtgcgcttgcaatcgcggtgctgtcgccgaccgggtactttggccccctcacggctcgtgtgcgtgcgctgttcatgaagcacacgcacactggcaatccgctggtcgactcggtcgctgagcaccaccccgcagacgcgctcgcctacctgcaatatttgaacattgtgtacgttttgtgggtatttagcatccctgtgcagctgatcctgcccacccctaacctgtatgcgattctctttctcctcgtgtacagttgcatggcgtactatttcagcactcgcatggtgcgcttgctcctgctggctggcccagtggcgtgccttagcgggagtttgatgagtggtacgctgacgaagtggtgctttcaacagctgttctgggacgacaacctgcgcaccgccgatatggcggcggctggtgatactccgttttcacaagaggaccaccccaacagcggtgcacgcgcccgacggaaccagcagaaacagaaggcgacccaggctcctgccagaggctcaagcacaggcgacgaagaacgacgttacacatcactaatcccttttgacttccgcaaggagatcaagatgaaccgctggccgaccggaaaaaagcaagccacgttcatcatctctgccaccatctgtaccgttcttccgcttgcctttgtctactacttctcatgcacttccatggcaaactccttgtcgagcccacagatcctgtaccaaacccgtatggggggcaagacgatcatggtggctgactatctcgagtcatacgagtggctgcgcgacaacacgccagcggacgcgcgcgtgctgtcctggtgggactacggctaccagatcacaggcatcggcaaccgcacctcgctggccgatggcaacacctggaaccacgagcacatcgccaccatcggcaagatgctgacgtcgcccgtggcggaggcgcactcactggtgcgccacatggcggactacgtcctcatctgggctgggcagggcggagacttgatgaagtcgccgcacatggcgcgcattggcaacagcgtgtaccacgacatctgccccaacgacccgctttgccagcatttcggcttttacgaagactacagtcgcccaaaaccgatgatgcgcgcgtcgctgctgtacaacctgcacgaggccggacgaagcgcgggtgtgaaggtggacccgtccctctttcaggaagtgtactcatccaagtacggcctggtgcgcatcttcaaggtcatgaacgtgagcgcggagagcaagaagtgggtggctgacccggcaaaccgcgtgtgccacccgcctgggtcgtggatctgccccgggcagtacccgccggcgaaggagatccaggagatgctggcgcaccgcgtcccctttgaccagatgggcaagaagcacgacgacacgcacaaggcgcgcatggcacgcagcagaacactgggcgaggcttga

SEQ ID NO: 34 - protein, 859 aa, Leishmania braziliensis
MPIKNQRKGCEEGNPNPSSTPAAEPLANAEGTQRDTAEGTPMEPPSETYLFNCRAAPYSKLIYVYKGIMFTLILYAIRLAYQTRMLSVQTYGYIIHEFDPWFNYRAAEYMSAHGWSAFFSWFDYMSWYPLGRPVGTTTYPGLQLTAVAIHRALAAAGVPMSLNNVCVLIPAWYGAIATAIMALMAFETTGSIAVSAWAALLFSIIPAHLMRSMAGEFDNECIAVAAMLLTFYLWVRSLRTRCSWPIGILTGIAYGYMVAAWGGYIFVLNMVAMHAGISSMVDWARNTYNPSLLRAYALFYVVGTAIATRVPPVGMSPFRSLEQLGALVVLLFLCGLQACEVFRARADVEVRSRANFKIRMRAFSVMAGVGALAIAVLSPTGYFGPLTARVRALFMKHTHTGNPLVDSVAEHHPADALAYLQYLNIVYVLWVFSIPVQLILPTPNLYAILFLLVYSCMAYYFSTRMVRLLLLAGPVACLSGSLMSGTLTKWCFQQLFWDDNLRTADMAAAGDTPFSQEDHPNSGARARRNQQKQKATQAPARGSSTGDEERRYTSLIPFDFRKEIKMNRWPTGKKQATFIISATICTVLPLAFVYYFSCTSMANSLSSPQILYQTRMGGKTIMVADYLESYEWLRDNTPADARVLSWWDYGYQITGIGNRTSLADGNTWNHEHIATIGKMLTSPVAEAHSLVRHMADYVLIWAGQGGDLMKSPHMARIGNSVYHDICPNDPLCQHFGFYEDYSRPKPMMRASLLYNLHEAGRSAGVKVDPSLFQEVYSSKYGLVRIFKVMNVSAESKKWVADPANRVCHPPGSWICPGQYPPAKEIQEMLAHRVPFDQMGKKHDDTHKARMARSRTLGEA

SEQ ID NO: 35 - DNA, 2319 bp, Leishmania braziliensis
atgtactgcctaaacaaggcctatcgcattcgcatgttttccgttcagctttatggctacatcatccacgagttcgacccgtggttcaactaccgcgccgccgagtacatgtccgcgcacggctggtccgccttcttcagctggttcgactacatgagctggtacccgctgggccgccccgttggcaccaccacgtacccgggcctgcagctcaccgccgttgccatccaccgcgcattggcagctgccggggtgccgatgtctctcaacaacgtgtgcgtgctgatccccgcgtggtatggtgccatcgctactgctctagaagcgctaatgatctatgagtgtaacggctccggaattaccgctgccatcggagcttttatctttatgattctccccgcacacctgatgcggtccatggcgggcgagttcgacaacgagtgcatcgccgttgcagccatgctcctcaccttctacttgtgggtacgctcgctgcgcacgcggtgctcgtggcccatcggcatcctcaccggtatcgcctacggctacatggtggcggcgtggggcggatacatttttgtgctcaacatggttgccatgcacgccggcatatcatcgatggtcgactgggctcgcaacacgtacaacccgtcgctgctgcgcgcatacgcgctgttctacgttgtcggcaccgccatcgccacgcgcgtgccgcctgtggggatgtcgcccttcaggtcgctggagcagctgggtgcgctggcggtgctcctcttcctgtgcgggctgcaggcctgcgaggtgtttcgcgcacgggccgacgtcgaggttcgctcccgcgcgaacttcaagatccgcatgcgtgccttcagcgtgatggctggcgtgggtgcgcttgcaatcgcggtgctgtcgccgaccgggtactttggccccctcacggctcgtgtgcgtgcgctgttcatggagcacacgcgcactggcaatccgctggtcgactcggtcgctgagcaccgcaaaacgaacccacaggcgtacgagtactttctggactttacctattcgatgtggatgctgggagcagtgttgcagttgctcggtgcagccgttggctcacgaaaggaggcgcggctgttcatggggctgtactcactcgccacctactacttctcagatcgcatgtcacggctgatggtacttgcggggcctgcggctgccgcgatagcagcggaaatcttgggcatcccatacgagtggtgttggacgcagctgacgggatgggcatctccgaacacctccgccagagagcgtaaaagcaaggaggacggtccctgcaagacaaaaagaaatcagagacagaccgtcgccacaaaactagatcatggggcgcgggctagggctacggccgctgtcaagttcatggagacggctctggagcgtgttcctctggtgtttcgagctgccatcgccataggcatcattggggccactgttggaacaccgtacgtctatcagttccaggctcgttgcattcaatcttcctattcttttgctgtcccgcgtatcatgttccacacgcagctgcgcaccggcgaaacagtgattgtaaaggactacgtggaagcatacgagtggctgcgcgacaacacgccagcggacgcgcgcgtgctgtcctggtgggactacggctaccagatcacaggtatcggcaaccgcacctcgctggccgatggcaacacctggaaccacgagcacatcgccaccatcggcaagatgctgacgtcgcccgtggcggaggcgcactcactggtgcgccacatggcggactacgtcctcatctgggctgggcagggcggagacttgatgaagtcgccgcacatggcgcgcattggcaacagcgtgtaccacgacatctgccccaacgacccgctttgccagcatttcggcttttacgaagactacagtcgcccaaaaccgatgatgcgcgcgtcgctgctgtacaacctgcacgaggccggacgaagcgcgggtgtgaaggtggacccgtccctctttcaggaagtgtactcatccaagtacggcctggtgcgcatcttcaaggtcatgaacgtgagcgcggagagcaagaagtgggtggctgacccggcaaaccgcgtgtgccacccgcctgggtcgtggatctgccccgggcagtacccgccggcgaaggagatccaggagatgctggcgcaccgcgtcccctttgaccagatgggcaagaagcacgacgacacgcacaaggcgcgcatggcacgcagcagaacactgggcgaggcttga

SEQ ID NO: 36 - protein, 772 aa, Leishmania braziliensis
MYCLNKAYRIRMFSVQLYGYIIHEFDPWFNYRAAEYMSAHGWSAFFSWFDYMSWYPLGRPVGTTTYPGLQLTAVAIHRALAAAGVPMSLNNVCVLIPAWYGAIATALEALMIYECNGSGITAAIGAFIFMILPAHLMRSMAGEFDNECIAVAAMLLTFYLWVRSLRTRCSWPIGILTGIAYGYMVAAWGGYIFVLNMVAMHAGISSMVDWARNTYNPSLLRAYALFYVVGTAIATRVPPVGMSPFRSLEQLGALAVLLFLCGLQACEVFRARADVEVRSRANFKIRMRAFSVMAGVGALAIAVLSPTGYFGPLTARVRALFMEHTRTGNPLVDSVAEHRKTNPQAYEYFLDFTYSMWMLGAVLQLLGAAVGSRKEARLFMGLYSLATYYFSDRMSRLMVLAGPAAAAIAAEILGIPYEWCWTQLTGWASPNTSARERKSKEDGPCKTKRNQRQTVATKLDHGARARATAAVKFMETALERVPLVFRAAIAIGIIGATVGTPYVYQFQARCIQSSYSFAVPRIMFHTQLRTGETVIVKDYVEAYEWLRDNTPADARVLSWWDYGYQITGIGNRTSLADGNTWNHEHIATIGKMLTSPVAEAHSLVRHMADYVLIWAGQGGDLMKSPHMARIGNSVYHDICPNDPLCQHFGFYEDYSRPKPMMRASLLYNLHEAGRSAGVKVDPSLFQEVYSSKYGLVRIFKVMNVSAESKKWVADPANRVCHPPGSWICPGQYPPAKEIQEMLAHRVPFDQMGKKHDDTHKARMARSRTLGEA

SEQ ID NO: 37 - DNA, 2565 bp, Leishmania braziliensis
atgggtaagaagaaagcaattccgtcgggcagcgtcggccctgcgacaaccacctcccgtgaagctccaggcaaagacgaaggtgcctcccaacccgccaagactgcagctctgccggtgaagccctttgtgttgcccaacacgctgacagacgaggaggagtttgttggcatctttccctgccctttctggccagtgcgatttgtcatcacagtgatggcactcgtcctcttgggtgccagctgtatccgcgccttcacgattcgcatgctatccgttcagctttatggctacatcatccacgagttcgacccgtggttcaactaccgcgccgccgagtacatgtccgcgcacggctggtccgccttcttcagctggttcgactacatgagctggtacccgctgggccgccccgttggcaccaccacgtacccgggcctgcagctcaccgccgttgccatccaccgcgcattggcggctgccggggtgccgatgtctctcaacaacgtgtgcgtgctgatccccgcgtggtatggtgccatcgctactgctatcctggccctttgcgcttacgaggtcagtaggtcaatggtagcggcggctgttgctgcactctcattctccatcattccagcacacctgatgcggtccatggcgggcgagttcgacaacgagtgcatcgccgttgcagccatgctcctcaccttctacttgtgggtacgctcgctgcgcacgcggtgctcgtggcccatcggcatcctcaccggtatcgcctacggctacatggtggcggcgtggggcggatacatttttgtgctcaacatggttgccatgcacgccggcatatcatcgatggtcgactgggctcgcaacacgtacaacccgtcgctgctgcgcgcatacgcgctgttctacgttgtcggcaccgccatcgccacgcgcgtgccgcctgtggggatgtcgcccttcaggtcgctggagcagctgggtgcgctggcggtgctcctcttcctgtgcgggctgcaggcctgcgaggtgtttcgcgcacgggccgacgtcgaggttcgctcccgcgcgaacttcaagatccgcatgcgtgccttcagcgtgatggctggcgtgggtgcgcttgcaatcgcggtgctgtcgccgaccgggtactttggccccctcacggctcgtgtgcgtgcgctgttcatggagcacacgcgcactggcaatccgctggtcgactcggtcgctgagcaccaccccgccagtcctgaggcgatgtggacatttcttcacgtgtgcggcgtgacttggggtttgggctccattgttcttcttgtgtcgttgctggtggactactcctcggcaaagctcttttggctgatgaactctggtgccgtgtactatttcagcacccgcatgtcacgactgctgcttctcacgggccccgctgcgtgtctgtccactggctgtttcgtggggacattactggaagcggcgatacagttcaccttctggtccagcgatgcaacaaaggccaaaaaacagcaagagacacaacttcaccaaaagggcgcgcgcaagcatagcgaccggagtaactctaagaatgcactgactgtgcgtacattgggcgacgtcttgaggagtacctctctggcatggggtcatcgcatggtgctctgcttcgctatgtgggctcttgttattacagtcgcggtgtgcctcttgggttccgatttcacttcccatgcaacgatgtttgcaaggcagacgtcgaacccgctgattgtctttgcaaccgtgctgcgagaccgcgctaccggcaagccaacacaggtattggtggatgactacctgcgcagctatctctggctgcgcgacaacacgcccagaaatgcgcgcgtgctgtcctggtgggactacggctaccagatcacaggtatcggcaaccgcacctcgctggccgatggcaacacctggaaccacgagcacatcgccaccatcggcaagatgctgacgtcgcccgtggcggaggcgcactcactggtgcgccacatggcggactacgtcctcatctgggctgggcagggcggagacttgatgaagtcgccgcacatggcgcgcattggcaacagcgtgtaccacgacatctgccccaacgacccgctttgccagcatttcggcttttacaagaacgatcgcaatcgcccaaaaccgatgatgcgcgcgtcgctgctgtacaacctgcacgaggccggacgaagcgcgggtgtgaaggtggacccgtccctctttcaggaagtgtactcatccaagtacggcctggtgcgcatcttcaaggtcatgaacgtgagcgcggagagcaagaagtgggtggctgacccggcaaaccgcgtgtgccacccgcctgggtcgtggatctgccccgggcagtacccgccggcgaaggagatccaggagatgctggcgcaccgcgtcccctttgaccatgtgaacagcttcagtcggaaaaaggccgggtcttatcatgaagaatacatgcgccggatgcgtgaagagcaggaccgatga

SEQ ID NO: 38 - protein, 854 aa, Leishmania brucei
MGKKKAIPSGSVGPATTTSREAPGKDEGASQPAKTAALPVKPFVLPNTLTDEEEFVGIFPCPFWPVRFVITVMALVLLGASCIRAFTIRMLSVQLYGYIIHEFDPWFNYRAAEYMSAHGWSAFFSWFDYMSWYPLGRPVGTTTYPGLQLTAVAIHRALAAAGVPMSLNNVCVLIPAWYGAIATAILALCAYEVSRSMVAAAVAALSFSIIPAHLMRSMAGEFDNECIAVAAMLLTFYLWVRSLRTRCSWPIGILTGIAYGYMVAAWGGYIFVLNMVAMHAGISSMVDWARNTYNPSLLRAYALFYVVGTAIATRVPPVGMSPFRSLEQLGALAVLLFLCGLQACEVFRARADVEVRSRANFKIRMRAFSVMAGVGALAIAVLSPTGYFGPLTARVRALFMEHTRTGNPLVDSVAEHHPASPEAMWTFLHVCGVTWGLGSIVLLVSLLVDYSSAKLFWLMNSGAVYYFSTRMSRLLLLTGPAACLSTGCFVGTLLEAAIQFTFWSSDATKAKKQQETQLHQKGARKHSDRSNSKNALTVRTLGDVLRSTSLAWGHRMVLCFAMWALVITVAVCLLGSDFTSHATMFARQTSNPLIVFATVLRDRATGKPTQVLVDDYLRSYLWLRDNTPRNARVLSWWDYGYQITGIGNRTSLADGNTWNHEHIATIGKMLTSPVAEAHSLVRHMADYVLIWAGQGGDLMKSPHMARIGNSVYHDICPNDPLCQHFGFYKNDRNRPKPMMRASLLYNLHEAGRSAGVKVDPSLFQEVYSSKYGLVRIFKVMNVSAESKKWVADPANRVCHPPGSWICPGQYPPAKEIQEMLAHRVPFDHVNSFSRKKAGSYHEEYMRRMREEQDR

SEQ ID NO: 39 - DNA, 2592 bp, Leishmania infantum
atgcccgccaagaatcaacacaaagggggcggagacggcaaccccgatcctacctccacacccgaagcggcgtcgacaaatgtgacaagcacaaacgacggtgccgccgtcgattcttccgtgccaccgtccggcgagacatacctctttcattgccgcgccgccccgtactcgaagctatcgtacgccttcaaaggtatcatggccgtcctgattctctgcgcccttcgctcggcgtaccaggttcgcctgctctccgttcagatttacggatacctgatccacgagttcgacccgtggttcaactaccgcgctgccgagtacatgtccacgcacggctggtccgccttcttcagctggttcgactacatgagctggtacccgctgggccgccctgttggctccaccacgtacccgggcctgcagctcactgccgtcgccattcaccgcgcgctggcggctgccggcatgccgatgtctctcaacaacgtgtgcgtgctgatgccggcgtggtttggcgccatcgccaccgctactctggctctcatagcattcgaagtgagcgaatccatctgtatggcggcgtgggccgcactctccttctctatcatcccggcccacctgatgcggtccatggcgggtgagttcgacaacgagtgcattgccgtcgcagccatgctcctgaccttctactgctgggtgcgctcgctgcgcacgcggtcctcgtggcccatcggtgtcctcaccggtgtcgcctacggctacatggtggcggcgtggggcggctacattttcgtgctcaacatggttgccatgcatgccggcatatcatcgatggtggactgggcccgcaacacgtacaacccgtcgctgctgcgtgcatacacgctgttctacgtcgtcggcaccgccatcgccgtgtgcgtgccgccagtggggatgtcgcccttcaagtcgctggagcagctgggtgcactgctggtgcttgtcttcctgtgtggactgcaggcgtgcgaggtgtttcgcgcacgcgccggtgtcgaggttcgctctcgcgcgaacttcaagatccgcgtgcgcgtcttcagcgtgatggctggcgtggctgcgcttgcgatcgcggtgctggcaccgacggggtacttcgggcccctttcggtccgtgtgcgtgcgctgttcgtggagcacacgcgcactggcaatccgctggtcgactcggtcgccgagcaccatcctgccgacgcgctcgcgtatctgaactatttgcacatcgtttattttatgtggatattcagcttcccggtgcagctcatcctgccgagccgaaaccagtacgcggttctctttgtctttgtctacagcttcatggcctactacttcagcacccgcatggtgcgcttgctcattctggctggcccggcggcgtgcctcggcgcaagtgaggtaggtggaacgctgatggagtggtgctttcaacagctgttctgggacgacggcatgcggaccgccgatatggtagcagccggtgacatgccttaccaaaaacaagaccatgccagtagaggtgcaggcgcccgacagaagcagcagaaacagaagccgcgccaggttttcgcgagggactccagcactagcagcgaggagcgtccttacaggacactgatccccgtcgacttccgcagggacgcgcaaatgaaccgctggtcagccggaaagacaaacgccgccctcatcgtggctctcacgatcggtgttcttttaccgattgcgtttgtcttccacttctcgtgcgtcagctcagcgtactcctttgctggcccgcgtatcgtgttccagacgcagctgcgcaccggcgagcaagtgatagtgaaggactacctcgaggcctacgagtggctgcgcgacaacacgccagaggacgcgcgcattttggcctggtgggactacggctaccagatcacaggcatcggcaaccgcacctcgctggccgatggcaacacctggaaccacgagcacatcgccaccatcggcaagatgctgacgtcgcccgtggcggaggcgcactcgctggtgcgccacatggccgactacgtcctaatctgggctgggcagagcggcgacctgatgaagtcaccgcacatggcgcgcatcggcaacagtgtgtaccacgacatctgcccccacgacccgctgtgccagcaatttggcttttacagaaatgattacagtcgcccaacaccgatgatgcgggcgtcgctgctgtacaacctgcacgaggtcgggaaaacaaagggcgtgaaggtggacccgtctctctttcaggaggtgtactcgtccaagtacggcctggtgcgcgtcttcaaggtcatgaacgtgagcgaggagagcaagaagtgggttgctgacccggcaaaccgcgtgtgccacccgcctgggtcgtggatctgccccgggcagtacccgccggcgaaggagatccaggagatgctggcacaccgcgtccccttcgatcaggtgggcaaggacaagaaggacaaggaggcgtaccacaaggcgtacatggaacgcagcagaacgctgggtgaagtttga

SEQ ID NO: 40 - protein, 863 aa, Leishmania infantum
MPAKNQHKGGGDGNPDPTSTPEAASTNVTSTNDGAAVDSSVPPSGETYLFHCRAAPYSKLSYAFKGIMAVLILCALRSAYQVRLLSVQIYGYLIHEFDPWFNYRAAEYMSTHGWSAFFSWFDYMSWYPLGRPVGSTTYPGLQLTAVAIHRALAAAGMPMSLNNVCVLMPAWFGAIATATLALIAFEVSESICMAAWAALSFSIIPAHLMRSMAGEFDNECIAVAAMLLTFYCWVRSLRTRSSWPIGVLTGVAYGYMVAAWGGYIFVLNMVAMHAGISSMVDWARNTYNPSLLRAYTLFYVVGTAIAVCVPPVGMSPFKSLEQLGALLVLVFLCGLQACEVFRARAGVEVRSRANFKIRVRVFSVMAGVAALAIAVLAPTGYFGPLSVRVRALFVEHTRTGNPLVDSVAEHHPADALAYLNYLHIVYFMWIFSFPVQLILPSRNQYAVLFVFVYSFMAYYFSTRMVRLLILAGPAACLGASEVGGTLMEWCFQQLFWDDGMRTADMVAAGDMPYQKQDHASRGAGARQKQQKQKPRQVFARDSSTSSEERPYRTLIPVDFRRDAQMNRWSAGKTNAALIVALTIGVLLPIAFVFHFSCVSSAYSFAGPRIVFQTQLRTGEQVIVKDYLEAYEWLRDNTPEDARILAWWDYGYQITGIGNRTSLADGNTWNHEHIATIGKMLTSPVAEAHSLVRHMADYVLIWAGQSGDLMKSPHMARIGNSVYHDICPHDPLCQQFGFYRNDYSRPTPMMRASLLYNLHEVGKTKGVKVDPSLFQEVYSSKYGLVRVFKVMNVSEESKKWVADPANRVCHPPGSWICPGQYPPAKEIQEMLAHRVPFDQVGKDKKDKEAYHKAYMERSRTLGEV

SEQ ID NO: 41 - DNA, 2355 bp, Leishmania infantum
atgctgctcttgttcttctcctttctgtactgcctgaaaaatgcatatggcgttcgcctgctctccgttcagatttacggatacctgatccacgagttcgacccgtggttcaactaccgcgctgccgagtacatgtccacgcacggctggtccgccttcttcagctggttcgactacatgagctggtacccgctgggccgccctgttggctccaccacgtacccgggcctgcagctcactgccgtcgccattcaccgcgcgctggcggctgccggcatgccgatgtctctcaacaacgtgtgcgtgctgatgccggcgtggtttggcgccatcgccaccgctactatggctggcatgacgtatgagatgagcggatcaggcattaccgctgccatcgcagcttttatctttatgatcctcccagcccacctgatgcggtccatggcgggtgagttcgacaacgagtgcattgccgtcgcagccatgctcctgaccttctactgctgggtgcgctcgctgcgcacgcggtcctcgtggcccatcggtgtcctcaccggtgtcgcctacggctacatggtggcggcgtggggcggctacattttcgtgctcaacatggttgccatgcatgccggcatatcatcgatggtggactgggcccgcaacacgtacaacccgtcgctgctgcgtgcatacacgctgttctacgtcgtcggcaccgccatcgccgtgtgcgtgccgccagtggggatgtcgcccttcaagtcgctggagcagctgggtgcactgctggtgcttgtcttcctgtgtggactgcaggcgtgcgaggtgtttcgcgcacgcgccggtgtcgaggttcgctctcgcgcgaacttcaagatccgcgtgcgcgtcttcagcgtgatggctggcgtggctgcgcttgcgatcgcggtgctggcaccgacggggtacttcgggcccctttcggtccgtgtgcgtgcgctgttcgtggagcacacgcgcactggcaatccgctggtcgactcggtcgccgagcaccgcaagacgagtccggaagcgtacgcattttttctggacttcacctacccgatgtggctgctcggcacagtattgcagctgttcggtgcagggatggggtcacggaaggaggcgcggttgtttatggggctgtactcactcgccacctactacttttcagatcgtatgtcacgcttgatggtgctggctggcccggcggctgccgctatggcggcaggaatcctgggcatcgtgtacgaatggtgttgggcgcagctgaccggctgggcatctccgagcctctctgctgttggcagcaaaggcacggacggctttgacaaccatataggaaaaactcagacgcagtccagcaccgcaaaccgtaaccgtggtgtgcgagctcatgctgttgccgctgtaaagtcgatgaaggcagctgtggaccttcttccgctggtgttgcgagctggcgtcgctgtggccatccttgctgtcaccgttggtacgccgtacgtctctcagttccaggttcgttgtattcagtctgcgtactcctttgctggcccgcgtatcgtgttccagacgcagctgcgcaccggcgagcaagtgatagtgaaggactacctcgaggcctacgagtggctgcgcgacaacacgccagaggacgcgcgcattttggcctggtgggactacggctaccagatcacaggcatcggcaaccgcacctcgctggccgatggcaacacctggaaccacgagcacatcgccaccatcggcaagatgctgacgtcgcccgtggcggaggcgcactcgctggtgcgccacatggccgactacgtcctaatctgggctgggcagagcggcgacctgatgaagtcaccgcacatggcgcgcatcggcaacagtgtgtaccacgacatctgcccccacgacccgctgtgccagcaatttggcttttacagaaatgattacagtcgcccaacaccgatgatgcgggcgtcgctgctgtacaacctgcacgaggtcgggaaaacaaagggcgtgaaggtggacccgtctctctttcaggaggtgtactcgtccaagtacggcctggtgcgcgtcttcaaggtcatgaacgtgagcgaggagagcaagaagtgggttgctgacccggcaaaccgcgtgtgccacccgcctgggtcgtggatctgccccgggcagtacccgccggcgaaggagatccaggagatgctggcacaccgcgtccccttcgatcagatgggcaaggacaagaaggacaaggaggcgtaccacaaggcgtacatggaaaagtcgaagaaggtagtcgagttctga

SEQ ID NO: 42 - protein, 784 aa, Leishmania infantum
MLLLFFSFLYCLKNAYGVRLLSVQIYGYLIHEFDPWFNYRAAEYMSTHGWSAFFSWFDYMSWYPLGRPVGSTTYPGLQLTAVAIHRALAAAGMPMSLNNVCVLMPAWFGAIATATMAGMTYEMSGSGITAAIAAFIFMILPAHLMRSMAGEFDNECIAVAAMLLTFYCWVRSLRTRSSWPIGVLTGVAYGYMVAAWGGYIFVLNMVAMHAGISSMVDWARNTYNPSLLRAYTLFYVVGTAIAVCVPPVGMSPFKSLEQLGALLVLVFLCGLQACEVFRARAGVEVRSRANFKIRVRVFSVMAGVAALAIAVLAPTGYFGPLSVRVRALFVEHTRTGNPLVDSVAEHRKTSPEAYAFFLDFTYPMWLLGTVLQLFGAGMGSRKEARLFMGLYSLATYYFSDRMSRLMVLAGPAAAAMAAGILGIVYEWCWAQLTGWASPSLSAVGSKGTDGFDNHIGKTQTQSSTANRNRGVRAHAVAAVKSMKAAVDLLPLVLRAGVAVAILAVTVGTPYVSQFQVRCIQSAYSFAGPRIVFQTQLRTGEQVIVKDYLEAYEWLRDNTPEDARILAWWDYGYQITGIGNRTSLADGNTWNHEHIATIGKMLTSPVAEAHSLVRHMADYVLIWAGQSGDLMKSPHMARIGNSVYHDICPHDPLCQQFGFYRNDYSRPTPMMRASLLYNLHEVGKTKGVKVDPSLFQEVYSSKYGLVRVFKVMNVSEESKKWVADPANRVCHPPGSWICPGQYPPAKEIQEMLAHRVPFDQMGKDKKDKEAYHKAYMEKSKKVVEF

SEQ ID NO: 43 - DNA, 2382 bp, Leishmania infantum
atgtcctcgcagactcgtagcatcatctactcctcctgctttgcagtggccatggccattgccctccctatcgcgtacgacatgcgcgtccgctccatcggcgtgtacgggtacctcttccacagaagtgacccgtggttcaactaccgcgctgccgagtacatgtccacgcacggctggtccgccttcttcagctggttcgactacatgagctggtacccgctgggccgccctgttggctccaccacgtacccgggcctgcagctcactgccgtcgccattcaccgcgcgctggcggctgccggcatgccgatgtctctcaacaacgtgtgcgtgctgatgccggcatggttttcacttgtctcttcagcgatggtggctctgctggcgcatgagttgagcggcaatatggcggtggccagcatctcgtctattttgtttagtgtgattccagcccacctgatgcggtccatggcgggtgagttcgacaacgagtgcattgccgtcgcagccatgctcctgaccttctactgctgggtgcgctcgctgcgcacgcggtcctcgtggcccatcggtgtcctcaccggtgtcgcctacggctacatggtggcggcgtggggcggctacattttcgtgctcaacatggttgccatgcatgccggcatatcatcgatggtggactgggcccgcaacacgtacaacccgtcgctgctgcgtgcatacacgctgttctacgtcgtcggcaccgccatcgccgtgtgcgtgccgccagtggggatgtcgcccttcaagtcgctggagcagctgggtgcactgctggtgcttctcttcattttcggtcagtctgtgtgtgaggcccagcgcagacgattggaaatcgcgcgcttttcaaaggagggcgtggcgctgctcatccgcatctacgcagccttcttcgttggtatcgttgccgtggccaccattgccccggccggattcttcaagccgctctccctgcaagcgagcgcgataatcactggcgtatctcgtaccggaaacacactcgtagacactctgattgcgcaagacgcgtccaacctactcatagtgtggcagctttttctctttcccgtctttggttgggtggccggcatgagcgccttccttacagagttggtccggaattacacctacacaaagagtttcatgctgatgtacggcgtggttggtctgtacttcgccagccaatctgtccgaatgatggtgatgatggcccccgtggcgtgcatcttcactgcccttttgttccgctgggcactggactacctcctcgggtcgttgttttgggctgagatgccaccttgctttgacaccgacgcgcagcgcgggcggcagcaacagaccgcggaggaggcggaggcagagaccaagcgtaaggaggaagagtacaacaccatgcaggtcaagaagatgacgacgcgcatgttgcccttcatgtttttgctcttactgtttcgtctttcggggttcatcgaagatgtggcggcgatatcgcgcgagatggaggcaccgggtatagtttttcccagtggacaggtgcagggcgtgtcggagaaaaaggtggacgactactattcggggtacctgtacctgcgcgacaacacgccagaggacgcgcgcattttggcctggtgggactacggctaccagatcacaggcatcggcaaccgcacctcgctggccgatggcaacacctggaaccacgagcacatcgccaccatcggcaagatgctgacgtcgcccgtggcggaggcgcactcgctggtgcgccacatggccgactatgttctgatttttgccggagacacgtacttttccgacctgaatcgctcaccgcacatggcgcgcatcggcaacagtgtgtaccgcgacatctgcccccacgacccgctgtgtagtcggttcgtgttacagaaaagaccgaaagctgctgcagcgaagcgcagtcgacacgtcagcgttgacgaactagaggaggaggacaatgcagagcacgtggtatacgagccgtcatcactcatggccaagtccctcatatatcatctgcactcagcaggggtggtgaagggggtcacgctgaatgagacgctcttccagcacgtcttcacctcggcgcaaggtctcatacgcatcttcaaggtcatgaacgtgagcgaggagagcaagaagtgggttgctgacccggcaaaccgcgtgtgccacccgcctgggtcgtggatctgccccgggcagtacccgccggcgaaggagatccaggagatgctggcacaccaacacaccaacttcaaggaccttcttgatgccatgaacgacttggagcgggagcaggcgctgaacaaggag

SEQ ID NO: 44 - protein, 794 aa, Leishmania infantum
MSSQTRSIIYSSCFAVAMAIALPIAYDMRVRSIGVYGYLFHRSDPWFNYRAAEYMSTHGWSAFFSWFDYMSWYPLGRPVGSTTYPGLQLTAVAIHRALAAAGMPMSLNNVCVLMPAWFSLVSSAMVALLAHELSGNMAVASISSILFSVIPAHLMRSMAGEFDNECIAVAAMLLTFYCWVRSLRTRSSWPIGVLTGVAYGYMVAAWGGYIFVLNMVAMHAGISSMVDWARNTYNPSLLRAYTLFYVVGTAIAVCVPPVGMSPFKSLEQLGALLVLLFIFGQSVCEAQRRRLEIARFSKEGVALLIRIYAAFFVGIVAVATIAPAGFFKPLSLQASAIITGVSRTGNTLVDTLIAQDASNLLIVWQLFLFPVFGWVAGMSAFLTELVRNYTYTKSFMLMYGVVGLYFASQSVRMMVMMAPVACIFTALLFRWALDYLLGSLFWAEMPPCFDTDAQRGRQQQTAEEAEAETKRKEEEYNTMQVKKMTTRMLPFMFLLLLFRLSGFIEDVAAISREMEAPGIVFPSGQVQGVSEKKVDDYYSGYLYLRDNTPEDARILAWWDYGYQITGIGNRTSLADGNTWNHEHIATIGKMLTSPVAEAHSLVRHMADYVLIFAGDTYFSDLNRSPHMARIGNSVYRDICPHDPLCSRFVLQKRPKAAAAKRSRHVSVDELEEEDNAEHVVYEPSSLMAKSLIYHLHSAGVVKGVTLNETLFQHVFTSAQGLIRIFKVMNVSEESKKWVADPANRVCHPPGSWICPGQYPPAKEIQEMLAHQHTNFKDLLDAMNDLEREQALNKE

SEQ ID NO: 45 - DNA, 2559 bp, Leishmania infantum
atgccggccaagaaccagcacaaaggaggcggagacggcgaccccgaccctacctccacacctgcagcggagtcgacaaaagtgacgaacacaagcgacggtgccgccgtcgattccaccctgccaccgtccgacgagacatacctcttccattgccgcgccgccccgtactcgaagctgtcgtacgccttcaaaggtatcatgaccgtcctgattctgtgcgccattcgctcggcgtaccaggttcgcctgatctccgttcagatttacggatacctgatccacgagttcgacccgtggttcaactaccgcgctgccgagtacatgtccacgcacggctggtccgccttcttcagctggttcgactacatgagctggtacccgctgggccgccccgtcggctccaccacgtacccgggcctgcagctcactgccgtcgccattcaccgcgcactggcagctgccggcatgccgatgtctctcaacaacgtgtgcgtgctgatgccagcgtggtttggcgccatcgctaccgctactctggctctcatagcattcgaagtgagcgaatcgatctgtatggctgcgtgggccgcactctccttctccatcatcccagcccacctgatgcggtccatggcgggtgagttcgacaacgagtgcatcgccgtcgcagccatgctcctcactttctactgctgggtgcgctcgctgcgcacgcggtcctcgtggcccatcggtgtcctcaccggtgtcgcctacggctacatggcggcggcgtggggcggctacattttcgtgctcaacatggttgccatgcatgccggcatatcatcgatggtggactgggcccgcaacacgtacaacccgtcgctgctgcgtgcatacacgctgttctacgtcgtgggcaccgccatcgccgtgtgcgtgccgccagtggggatgtcgcccttcaagtcgctggagcagctgggtgcgctgctggtgcttgtcttcctgtgtggactgcaggtgtgcgaggtgctgcgcgcacgcgccggtgtcgaggttcgctctcgcgcgaacttcaagatccgcgtgcgcgtcttcagcgtgatggctggcgtggctgcgcttgcaatctcggtgctggcaccgacggggtacttcgggcccctctcggtccgtgtgcgtgcgctgttcgtggagcacacgcgcactggcaatccgctggtcgactcggtcgccgagcaccaccctgcggacgcgctcgcgtatctgaactatttgcacattgttcacttgatgtggatatgcagcttgccggtgcagctcatccttccaagccgaaaccagtacgcggttctctttgtcttggtctacagctttatggcctactacttcagcacccgcatggtgcgcttgctcattctggctggccctgtggcgtgcctcggcgcaagcgaggtaggtggaacgctgatggagtggtgctttcaacagctgttctgggacaacggcatgcggaccgccgatatggtagcagccggtgacatgccttaccaaaaagacgaccacaccagcagaggtgcaggcgcccgacagaagcagcagaaacagaagccgggccaggtttccgcgaggggctccagcactagcagcgaggagcgtccttacaggacactgatccccgtcgacttccgcagggacgcccaaatgaaccgctggtcggccggaaagacaaacgccgccctcatcgtggctctcacgatcggtgttcttttaccgcttgcgtttgtcttccacctctcatgcatcagctcagcgtactccttcgctggcccgcgtatcgtgttccagacgcagctgcacaccggcgagcaagtgatagtgaaggactacctcgaggcctacgagtggctgcgcgacagcacgccagaggacgcgcgcgttttggcctggtgggactacggctaccagatcacaggcatcggcaaccgcacctcgctggccgatggcaacacctggaaccacgagcacatcgccacgatcggcaagatgctgacgtcgcccgtggcggaggcgcactcgctggtgcgccacatggccgactacgtcctgatctgggctgggcagagcggcgacctgatgaagtcaccgcacatggcgcgcatcggcaacagcgtgtaccacgacatctgccccgacgacccgctgtgccagcaattcggctttcacagaaatgactacagtcgcccaacgccgatgatgcgggcgtcgctgctgtacaacctgcacgaggccgggaaaacaaagggcgtgaaggtgaacccgtccctctttcaggaggtgtactcgtcgaagtacggcctggtgcgcatcttcaaggtcatgaacgtgagcgcggagagcaaaaagtgggttgctgacccggcaaaccgcgtgtgccacccgcctgggtcgtggatctgccccgggcagtacccgccggcgaaggagatccaggagatgctggcacaccgcgtccccttcgatcagatggacaagcacaagcagcacaaggagacgcaccacaaggcgtag

SEQ ID NO: 46 - protein, 852 aa, Leishmania infantum
MPAKNQHKGGGDGDPDPTSTPAAESTKVTNTSDGAAVDSTLPPSDETYLFHCRAAPYSKLSYAFKGIMTVLILCAIRSAYQVRLISVQIYGYLIHEFDPWFNYRAAEYMSTHGWSAFFSWFDYMSWYPLGRPVGSTTYPGLQLTAVAIHRALAAAGMPMSLNNVCVLMPAWFGAIATATLALIAFEVSESICMAAWAALSFSIIPAHLMRSMAGEFDNECIAVAAMLLTFYCWVRSLRTRSSWPIGVLTGVAYGYMAAAWGGYIFVLNMVAMHAGISSMVDWARNTYNPSLLRAYTLFYVVGTAIAVCVPPVGMSPFKSLEQLGALLVLVFLCGLQVCEVLRARAGVEVRSRANFKIRVRVFSVMAGVAALAISVLAPTGYFGPLSVRVRALFVEHTRTGNPLVDSVAEHHPADALAYLNYLHIVHLMWICSLPVQLILPSRNQYAVLFVLVYSFMAYYFSTRMVRLLILAGPVACLGASEVGGTLMEWCFQQLFWDNGMRTADMVAAGDMPYQKDDHTSRGAGARQKQQKQKPGQVSARGSSTSSEERPYRTLIPVDFRRDAQMNRWSAGKTNAALIVALTIGVLLPLAFVFHLSCISSAYSFAGPRIVFQTQLHTGEQVIVKDYLEAYEWLRDSTPEDARVLAWWDYGYQITGIGNRTSLADGNTWNHEHIATIGKMLTSPVAEAHSLVRHMADYVLIWAGQSGDLMKSPHMARIGNSVYHDICPDDPLCQQFGFHRNDYSRPTPMMRASLLYNLHEAGKTKGVKVNPSLFQEVYSSKYGLVRIFKVMNVSAESKKWVADPANRVCHPPGSWICPGQYPPAKEIQEMLAHRVPFDQMDKHKQHKETHHKA

SEQ ID NO: 47 - DNA, 2322 bp, Leishmania infantum
atgctgctcttgttcttctcctttctgtactgcctaaaaaatgcgtatggccttcgcatgatctccgttcagatttacggatacctgatccacgagttcgacccgtggttcaactaccgcgctgccgagtacatgtccacgcacggctggtccgccttcttcagctggttcgactacatgagctggtacccgctgggccgccccgtcggctccaccacgtacccgggcctgcagctcactgccgtcgccattcaccgcgcactggcggctgccggcatgccgatgtctctcaacaacgtgtgcgtgctgatgccagcgtggtttggcgccatcgctaccgctactctggctctcatgacgtatgagatgagcggatcaggcattgccgctgccattgcagcttttatcttctccatcatcccagcccacctgatgcggtccatggcgggtgagttcgacaacgagtgcatcgccgtcgcagccatgctcctcactttctactgctgggtgcgctcgctgcgcacgcggtcctcgtggcccatcggtgtcctcaccggtgtcgcctacggctacatggcggcggcgtggggcggctacattttcgtgctcaacatggttgccatgcatgccggcatatcatcgatggtggactgggcccgcaacacgtacaacccgtcgctgctgcgtgcatacacgctgttctacgtcgtgggcaccgccatcgccgtgtgcgtgccgccagtggggatgtcgcccttcaagtcgctggagcagctgggtgcgctgctggtgcttgtcttcctgtgtggactgcaggtgtgcgaggtgctgcgcgcacgcgccggtgtcgaggttcgctctcgcgcgaacttcaagatccgcgtgcgcgtcttcagcgtgatggctggcgtggctgcgcttgcaatctcggtgctggcaccgacggggtacttcgggcccctctcggtccgtgtgcgtgcgctgttcgtggagcacacgcgcactggcaatccgctggtcgactcggtcgccgagcaccgcatgacgagcccgaaggcgtacgcattttttctggacttcacctacccggtgtggctgctcggcacagtattgcagttgttaggtgcattcatggggtcgcgaaaggaggcgcggttgtttatgggtctgcactcactcgccacctactactttgcagatcgtatgtcacgcttgatagtgctggcagggcccgcggctgccgctatgacggcaggaatcctgggccttgtgtatgagtggtgttgggcgcagctgactggctgggcgtctccgggcctctctgctgctggcagcggaggcatggacgactttgacaacaagcgaggccaaactcagatacagtccagcaccgcaaaccgcaaccgtggggtgcgtgctcatgctattgccgctgtaaagtcgatcaaggcaggtgtgaaccttcttcctctggtgttgcgagtcggcgtcgctgtggctatccttgctgtcaccgttggtacgccgtacgtctcgcagttccaggctcgttgtattcagtctgcgtactccttcgctggcccgcgcatcgtgttccaggcgcagctgcacaccggcgagcaagtgatagtgaaggactacctcgaggcctacgagtggctgcgcgacagcacgccagaggacgcgcgcgttttggcctggtgggactacggctaccagatcacaggcatcggcaaccgcacctcgctggccgatggcaacacctggaaccacgagcacatcgccacgatcggcaagatgctgacgtcgcccgtggcggaggcgcactcgctggtgcgccacatggccgactacgtcctgatctgggctgggcagagcggcgacctgatgaagtcaccgcacatggcgcgcatcggcaacagcgtgtaccacgacatctgccccgacgacccgctgtgccagcaattcggctttcacagaaatgactacagtcgcccaacgccgatgatgcgggcgtcgctgctgtacaacctgcacgaggccgggaaaacaaagggcgtgaaggtgaacccgtccctctttcaggaggtgtactcgtcgaagtacggcctggtgcgcatcttcaaggtcatgaacgtgagcgcggagagcaaaaagtgggttgctgacccggcaaaccgcgtgtgccacccgcctgggtcgtggatctgccccgggcagtacccgccggcgaaggagatccaggagatgctggcacaccgcgtccccttcgatcagatggacaagcacaagcagcacaaggagacgcaccacaaggcgtag

SEQ ID NO: 48 - protein, 773 aa, Leishmania infantum
MLLLFFSFLYCLKNAYGLRMISVQIYGYLIHEFDPWFNYRAAEYMSTHGWSAFFSWFDYMSWYPLGRPVGSTTYPGLQLTAVAIHRALAAAGMPMSLNNVCVLMPAWFGAIATATLALMTYEMSGSGIAAAIAAFIFSIIPAHLMRSMAGEFDNECIAVAAMLLTFYCWVRSLRTRSSWPIGVLTGVAYGYMAAAWGGYIFVLNMVAMHAGISSMVDWARNTYNPSLLRAYTLFYVVGTAIAVCVPPVGMSPFKSLEQLGALLVLVFLCGLQVCEVLRARAGVEVRSRANFKIRVRVFSVMAGVAALAISVLAPTGYFGPLSVRVRALFVEHTRTGNPLVDSVAEHRMTSPKAYAFFLDFTYPVWLLGTVLQLLGAFMGSRKEARLFMGLHSLATYYFADRMSRLIVLAGPAAAAMTAGILGLVYEWCWAQLTGWASPGLSAAGSGGMDDFDNKRGQTQIQSSTANRNRGVRAHAIAAVKSIKAGVNLLPLVLRVGVAVAILAVTVGTPYVSQFQARCIQSAYSFAGPRIVFQAQLHTGEQVIVKDYLEAYEWLRDSTPEDARVLAWWDYGYQITGIGNRTSLADGNTWNHEHIATIGKMLTSPVAEAHSLVRHMADYVLIWAGQSGDLMKSPHMARIGNSVYHDICPDDPLCQQFGFHRNDYSRPTPMMRASLLYNLHEAGKTKGVKVNPSLFQEVYSSKYGLVRIFKVMNVSAESKKWVADPANRVCHPPGSWICPGQYPPAKEIQEMLAHRVPFDQMDKHKQHKETHHKA

SEQ ID NO: 49 - DNA, 2373 bp, Leishmania infantum
atgccctcgcaaactcgtagcctcatctactcctcctgctttgcggtggccatggccattgccctccctatcgcgtacgacatgcgtgtccgctccatcggcgtgtacgggtacctcttccacagcagtgacccgtggttcaactaccgcgctgccgagtacatgtccacgcacggctggtccgccttcttcagctggttcgactacatgagctggtacccgctgggccgccccgtcggctccaccacgtacccgggcctgcagctcactgccgtcgccattcaccgcgcactggcggctgccggcatgccgatgtctctcaacaacgtgtgcgtgctgatgccagcgtggttttcacttgtctcttcagcgatggcggcactgctggcgcatgagatgagcggcaatatggcggtagccagcatctcgtctatcttattcagtgtggttccagcccacctgatgcggtccatggcgggtgagttcgacaacgagtgtatcgccgtcgcagccatgctcctcaccttctactgctgggtgcgctcgctgcgcacgcggtcctcgtggcccatcggtgtcctcaccggtgtcgcctacggctacatggcggcggcgtggggcggctacattttcgtgctcaacatggttgccatgcatgccggcatatcatcgatggtggactgggcccgcaacacgtacaacccgtcgctgctgcgtgcatacacgctgttctacgtcgtgggcaccgccatcgccgtgtgcgtgccgccagtggggatgtcgcccttcaagtcgctggagcagctgggtgcgctgctggtgcttgtcttcattttcggtcagtctgtgtgtgaggcccagcgcagacgattgggaatcgcgcgcctttcaaaggagggcgtggcgctgctcatccgcatcgacgcagccttcttcgtcggtatcgttgccgtggccaccattgccccggctggattcttcaagccgctctccctgcaagcgaacgcgataatcactggcgtatctcgtaccggaaacacactcgtagacattctgcttgcgcaagacgcgtccaacctactcatggtgtggcagctttttctctttcccttcttaggttgggtggcgggcatgagcgccttccttagagagttgatccggaactacacctacgcgaagagtttcatcctgatgtacggcgtggtcggtatgtacttcgccagccagtctgtccgaatgatggtgatgatggcccccgtggcgtgcatctttactgccctcttgttccgctgggcactggactacctcctcgggtctttgttttgggctgagatgccaccttcctttgacaccgacgcacagcgtgggcggcagcaacagaccgccgaggagtcggaggcagagaccaagcgtaaggaggaagagtacaacaccatgcaggtcaagaagatgtcggtgcgcatgttgcccttcatgctgttgctcttactgtttcgtctttcggggttcatcgaagatgtggcggcgatatcgcgcaagatggaggcgccgggtatagtttttcccagtgaacaggtgcaaggcgtgtcggagaaaaaggtcgacgactactatgcggggtacctgtatctgcgcgacagcacgccagaggacgcgcgcgttttggcctggtgggactacggctaccagatcacaggcatcggcaaccgcacctcgctggccgatggcaacacctggaaccacgagcacatcgccacgatcggcaagatgctgacgtcgcccgtggcggaggcgcactcgctggtgcgccacatggccgactatgttctgatttctgctggagacacatatttttccgacctgaatcgctcaccgatgatggcgcgcatcggcaacagcgtgtaccacgacatctgccccgacgacccactttgtagtcagttcgtgttgcagaaaagaccgaaagctgctgcagcgaagcgcagtcggcacgtcagcgttgacgcactagaggaggatgacactgcagagcatatggtatacgagccgtcatcactcatagccaagtcgctcatatatcacctgcactccacaggggtggtgacgggggtcacgctgaatgagacgctcttccagcacgtcttcacctcaccgcagggtctcatgcgcatcttcaaggtcatgaacgtgagcacggagagcaaaaagtgggttgctgactcggcaaaccgcgtgtgccacccgcctgggtcgtggatctgccccgggcagtacccgccggcgaaggagatccaggagatgctggcacaccaacacaccaacttcaaggaccttcttgatcccagaacgacttggagcgggagcaggcgctga

SEQ ID NO: 50 - protein, 790 aa, Leishmania infantum
MPSQTRSLIYSSCFAVAMAIALPIAYDMRVRSIGVYGYLFHSSDPWFNYRAAEYMSTHGWSAFFSWFDYMSWYPLGRPVGSTTYPGLQLTAVAIHRALAAAGMPMSLNNVCVLMPAWFSLVSSAMAALLAHEMSGNMAVASISSILFSVVPAHLMRSMAGEFDNECIAVAAMLLTFYCWVRSLRTRSSWPIGVLTGVAYGYMAAAWGGYIFVLNMVAMHAGISSMVDWARNTYNPSLLRAYTLFYVVGTAIAVCVPPVGMSPFKSLEQLGALLVLVFIFGQSVCEAQRRRLGIARLSKEGVALLIRIDAAFFVGIVAVATIAPAGFFKPLSLQANAIITGVSRTGNTLVDILLAQDASNLLMVWQLFLFPFLGWVAGMSAFLRELIRNYTYAKSFILMYGVVGMYFASQSVRMMVMMAPVACIFTALLFRWALDYLLGSLFWAEMPPSFDTDAQRGRQQQTAEESEAETKRKEEEYNTMQVKKMSVRMLPFMLLLLLFRLSGFIEDVAAISRKMEAPGIVFPSEQVQGVSEKKVDDYYAGYLYLRDSTPEDARVLAWWDYGYQITGIGNRTSLADGNTWNHEHIATIGKMLTSPVAEAHSLVRHMADYVLISAGDTYFSDLNRSPMMARIGNSVYHDICPDDPLCSQFVLQKRPKAAAAKRSRHVSVDALEEDDTAEHMVYEPSSLIAKSLIYHLHSTGVVTGVTLNETLFQHVFTSPQGLMRIFKVMNVSTESKKWVADSANRVCHPPGSWICPGQYPPAKEIQEMLAHQHTNFKDLLDPRTTWSGSRR

SEQ ID NO: 51 - DNA, 2574 bp, Leishmania infantum
atgggcaagcggaagggaaattcattgggcgactccggctctgcggcaaccgcatctcgtgaagcttcggcccaagccgaagatgccgcctcacaaaccaagaccgcatctccaccggcgaaggtgattttgctgcccaaaacgctaacagatgagaaggatttcatcggcatctttccgtttcctttctggccagtacacttcgtcctcaccgtggtggcactcttcgtcttagccgccagctgcttccaagccttcacggttcgcatgatctccgttcagatttacggatacctgatccacgagttcgacccgtggttcaactaccgcgctgccgagtacatgtccacgcacggctggtccgccttcttcagctggttcgactacatgagctggtacccgctgggccgccccgtcggctccaccacgtacccgggcctgcagctcactgccgtcgccattcaccgcgcactggcggctgccggcatgccgatgtctctcaacaacgtgtgcgtgctgatgccagcgtggtttggcgccatcgctaccgctactctggcgttttgcacctacgaagccagtgggtcgacagtagcggcggccgctgccgcactctccttctccatcatcccagcccacctgatgcggtccatggcgggtgagttcgacaacgagtgcatcgccgtcgcagccatgctcctcactttctactgctgggtgcgctcgctgcgcacgcggtcctcgtggcccatcggtgtcctcaccggtgtcgcctacggctacatggcggcggcgtggggcggctacattttcgtgctcaacatggttgccatgcatgccggcatatcatcgatggtggactgggcccgcaacacgtacaacccgtcgctgctgcgtgcatacacgctgttctacgtcgtgggcaccgccatcgccgtgtgcgtgccgccagtggggatgtcgcccttcaagtcgctggagcagctgggtgcgctgctggtgcttgtcttcctgtgtggactgcaggtgtgcgaggtgctgcgcgcacgcgccggtgtcgaggttcgctctcgcgcgaacttcaagatccgcgtgcgcgtcttcagcgtgatggctggcgtggctgcgcttgcaatctcggtgctggcaccgacggggtacttcgggcccctctcggtccgtgtgcgtgcgctgttcgtggagcacacgcgcactggcaatccgctggtcgactcggtcgccgaacatcaacccgccagcccggaggcgatgtgggcttttcttcacgtgtgcggcgtgacatggggtttgggctccattgtgcttgctgtctcgacgttcgtgcactactccccgtcgaaggtattctggctactgaactccggcgccgtgtactacttcagcactcgcatggctcggctgctgcttctctccggccccgctgcgtgtctgtccactggcattttcgtcgggacaattctggaagcagccgtgcaactcagtttctgggacagtgatgcgacaaaggctaaaaagcagcagaagcaggcgcaacgccaccagaggggggctggcaagggcagcggccgagatgacgctaagaacgcaacgaccgcgcgcgcattttgcgacgtctttgccggtagctctcttgcttggggccatcgcatggtcctgtccatcgctatgtgggctctcgtcacgacaaccgcggtgagcttcttcagttccgagttcgcgtcccactcaacaaagtttgcggagcagtcgtcaaatccgatgattgttttcgcggccgtcgtgcaaaaccgtgccacaggcaagcctatgaacctattggtggatgactacctcaaggcctacgagtggctgcgcgacagcacgccagaggacgcgcgcgttttggcctggtgggactacggctaccagatcacaggcatcggcaaccgcacctcgctggccgatggcaacacctggaaccacgagcacatcgccacgatcggcaagatgctgacgtcgcccgtggcggaggcgcactcgctggtgcgccacatggccgactacgtcctgatctgggctgggcagagcggcgacctgatgaagtcaccgcacatggcgcgcatcggcaacagcgtgtaccacgacatctgccccgacgacccgctgtgccagcaattcggctttcacagaaatgactacagtcgcccaacgccgatgatgcgggcgtcgctgctgtacaacctgcacgaggccgggaaaagaaagggcgtgaaggtgaacccgtccctctttcaggaggtgtactcgtcgaagtacggcctggtgcgcatcttcaaggtcatgaacgtgagcgcggagagcaaaaagtgggttgctgacccggcaaaccgcgtgtgccacccgcctgggtcgtggatctgccccgggcagtacccgccggcgaaggagatccaggagatgctggcacaccgcgtccccttcgatcaggtgacaaacgccgatcggaaaaacaatgtcgggtcgtaccaggaggagtacatgcgccggatgcgtgaaagcgagaaccgacggtaa

SEQ ID NO: 52 - protein, 857 aa, Leishmania infantum
MGKRKGNSLGDSGSAATASREASAQAEDAASQTKTASPPAKVILLPKTLTDEKDFIGIFPFPFWPVHFVLTVVALFVLAASCFQAFTVRMISVQIYGYLIHEFDPWFNYRAAEYMSTHGWSAFFSWFDYMSWYPLGRPVGSTTYPGLQLTAVAIHRALAAAGMPMSLNNVCVLMPAWFGAIATATLAFCTYEASGSTVAAAAAALSFSIIPAHLMRSMAGEFDNECIAVAAMLLTFYCWVRSLRTRSSWPIGVLTGVAYGYMAAAWGGYIFVLNMVAMHAGISSMVDWARNTYNPSLLRAYTLFYVVGTAIAVCVPPVGMSPFKSLEQLGALLVLVFLCGLQVCEVLRARAGVEVRSRANFKIRVRVFSVMAGVAALAISVLAPTGYFGPLSVRVRALFVEHTRTGNPLVDSVAEHQPASPEAMWAFLHVCGVTWGLGSIVLAVSTFVHYSPSKVFWLLNSGAVYYFSTRMARLLLLSGPAACLSTGIFVGTILEAAVQLSFWDSDATKAKKQQKQAQRHQRGAGKGSGRDDAKNATTARAFCDVFAGSSLAWGHRMVLSIAMWALVTTTAVSFFSSEFASHSTKFAEQSSNPMIVFAAVVQNRATGKPMNLLVDDYLKAYEWLRDSTPEDARVLAWWDYGYQITGIGNRTSLADGNTWNHEHIATIGKMLTSPVAEAHSLVRHMADYVLIWAGQSGDLMKSPHMARIGNSVYHDICPDDPLCQQFGFHRNDYSRPTPMMRASLLYNLHEAGKRKGVKVNPSLFQEVYSSKYGLVRIFKVMNVSAESKKWVADPANRVCHPPGSWICPGQYPPAKEIQEMLAHRVPFDQVTNADRKNNVGSYQEEYMRRMRESENRR

SEQ ID NO: 53 - DNA, 2406 bp, Trypanosoma brucei
atgacgaaaggtgggaaagtagctgtgactaagggctcagcacagagtgatggtgctggtgagggagggatgagtaaggccaagtcatccactacgttcgtcgccactggcggtggttctttgcctgcctgggcgctaaaggctgtaagcacgattgtgagtgcagtgattcttatatactctgtccatcgtgcttacgatatacgacttacttctgtccgtctttatggtgagcttattcacgagttcgacccttggttcaattaccgtgcaacgcagtacctcagcgacaacgggtggcgtgcttttttccaatggtacgactacatgagctggtacccgcttggccgaccggtgggcacaaccatcttccccggaatgcagcttaccggtgtagccattcatcgtgtgctggaaatgctcgggcgaggtatgtccatcaacaatatctgtgtgtacattcctgcatggttcggtagtattgccactgtgttggctgctctcattgcgtacgaatcatctaattcgctcagtgtcatggcgtttactgcgtactttttttccatcgtacctgcacacctgatgcgatcaatggctggtgaatttgacaatgagtgtgttgcaatggcggcgatgcttctgacgttctacatgtgggtacgatcgttacgcagctcaagttcgtggcccattggcgctttagctggtgtggcatacgggtacatggtgtccacgtggggtggttatattttcgtgctgaacatggtagccttccacgcttctgtatgtgtactgcttgattgggctcgtgggatatacagcgtcagtttgctgagggcgtattcactgttttttgtcattggcactgcccttgcgatttgcgtaccgccagtggagtggacgcccttccggtcgctggagcaactgacagcattgtttgtcttcgttttcatgtgggcactccactactcggaatacctgcgtgagcgtgcccgagcgcccattcactcttctaaagcacttcagatccgtgcccgcattttcatgggcaccctctccttgctgttgattgtggcaagtttgcttgccccgttcggattcttcaaacctacagcgtaccgcgtccgtgcgttgttcgtgaaacatacgcgtaccggaaatcccctcgtggattctgtggctgagcatcggccgacgactgccggggcgtatctgcgctactttcatgtttgttaccctctttgggggtgcggtgggctctctatgttggtattcatgaaaaaggaccgctggcgcgccattgtttttcttgcttcactttccactgttacgatgtatttcagcgcccgtatgtcgcgattacttctgttagcgggtcccgcagcaacggcttgcgccggcatgttcataggggggctttttgatctggcgctgtcacagtttggtgatttgcatagcccaaaagatgcctccggcgattccgatcccgcgggagggtcgaagcgggcaaagggcaaagttgttaatgagccttccaaaagagccatctttagtcaccgctggtttcaacgtttagtgcaatcgttgcccgtcccgctacgacgtggtatcgcggttgtggtgctcgtatgtctcttcgccaatcccatgagacactcattcgaaaagtcctgcgagaaaatggcacatgcactttcatctccaaggatcattgccgtgactgatctacccaatggagagagagtcctcgccgatgattactacgtgtcgtacttgtggctgcgaaacaatacgcctgaagatgcccgtattctctcatggtgggactacgggtatcaaatcactggaattggcaatcgcacaacccttgcggacggtaacacatggagtcacaagcacatagcaactattgggaagatgcttacatcccctgtgaaggagtcacatgctcttatacgccatctcgctgattatgtgctgatatgggccggtgaggatcggggcgatttacttaaatcgccacacatggctcggataggcaacagtgtatatcgcgatatgtgttcagaagacgatcctagatgcaggcagttcggctttgagggaggtgacctcaataagcctacgcctatgatgcagcggtccctattatacaatctgcacaggtttggtacggatggcgggaagacacaactggataagaacatgtttcagctcgcctacgtgtcaaagtatggtttggtgaagatctacaaggtggtgaatgtgagtgaagagagcaaggcgtgggtcgcagacccaaagaaccgcgtatgcgacccgcccggatcttggatatgcgccggccagtacccgccagcgaaggagatccaagacatgttagcgaagcggttccattacgaatga

SEQ ID NO: 54 - protein, 801 aa, Trypanosoma brucei
MTKGGKVAVTKGSAQSDGAGEGGMSKAKSSTTFVATGGGSLPAWALKAVSTIVSAVILIYSVHRAYDIRLTSVRLYGELIHEFDPWFNYRATQYLSDNGWRAFFQWYDYMSWYPLGRPVGTTIFPGMQLTGVAIHRVLEMLGRGMSINNICVYIPAWFGSIATVLAALIAYESSNSLSVMAFTAYFFSIVPAHLMRSMAGEFDNECVAMAAMLLTFYMWVRSLRSSSSWPIGALAGVAYGYMVSTWGGYIFVLNMVAFHASVCVLLDWARGIYSVSLLRAYSLFFVIGTALAICVPPVEWTPFRSLEQLTALFVFVFMWALHYSEYLRERARAPIHSSKALQIRARIFMGTLSLLLIVASLLAPFGFFKPTAYRVRALFVKHTRTGNPLVDSVAEHRPTTAGAYLRYFHVCYPLWGCGGLSMLVFMKKDRWRAIVFLASLSTVTMYFSARMSRLLLLAGPAATACAGMFIGGLFDLALSQFGDLHSPKDASGDSDPAGGSKRAKGKVVNEPSKRAIFSHRWFQRLVQSLPVPLRRGIAVVVLVCLFANPMRHSFEKSCEKMAHALSSPRIIAVTDLPNGERVLADDYYVSYLWLRNNTPEDARILSWWDYGYQITGIGNRTTLADGNTWSHKHIATIGKMLTSPVKESHALIRHLADYVLIWAGEDRGDLLKSPHMARIGNSVYRDMCSEDDPRCRQFGFEGGDLNKPTPMMQRSLLYNLHRFGTDGGKTQLDKNMFQLAYVSKYGLVKIYKVVNVSEESKAWVADPKNRVCDPPGSWICAGQYPPAKEIQDMLAKRFHYE

SEQ ID NO: 55 - DNA, 2466 bp, Trypanosoma brucei
atgacgaaaggtgggaaagtagctgtgactaagggctcagcacagagtgatggtgctggtgagggagggatgagtaaggccaagtcatccactacgttcgtcgccactggcggtggttctttgcctgcctgggcgctaaaggctgtaagcacggttgtgagtgcagtgattcttatatactctgtccatcgtgcttacgatatacgacttacttctgtccgtctttatggtgagcttattcacgagttcgacccttggttcaattaccgtgcaacgcagtacctcagcgacaacgggtggcgtgcttttttccaatggtacgactacatgagctggtacccgcttggccgaccggtgggcacaaccatcttccccggaatgcagcttaccggtgtagccattcatcgtgtgctggaaatgctcgggcgaggtatgtccatcaacaatatctgtgtgtacattcctgcatggttcggtagtattgccactgtgttggctgctctcattgcgtacgaatcatctaattcgctcagtgtcatggcgtttactgcgtactttttttccatcgtacctgcacacctgatgcgatcaatggctggtgaatttgacaatgagtgtgttgcaatggcggcgatgcttctgacgttctacatgtgggtacgatcgttacgcagctcaagttcgtggcccattggcgctttagctggtgtggcatacgggtacatggtgtccacgtggggtggttatattttcgtgctgaacatggtagccttccacgcttctgtatgtgtactgcttgattgggctcgtgggacatacagcgtcagtttgctgagggcgtattcactgttttttgtcattggcactgcccttgcgatttgcgtaccaccagtggagtggacgcccttccggtcgctggagcaactgacagcattgtttgtcttcgttttcatgtgggcactccactactcggaatacctgcgtgagcgtgcccgagcgcccattcactcttctaaagcacttcagatccgtgcccgcattttcatgggcaccctctccttgctgttgattgtagctatctacctattttcgacaggatacttcaggccgttttcttctcgtgtccgtgcgttgttcgtgaaacatacgcgtaccggaaatcccctcgtggattctgtggctgagcaccatccggcgtcgaatgatgatttctttggttaccttcatgtatgttacaacggctggataattggctttttcttcatgtccgtgtcgtgctttttccattgtacaccgggaatgtcgttcctgctgttgtactctatacttgcgtactacttctctctcaagatgagtcgtctgctgttactttctgcacctgtggcttccatactcactggctatgttgtgggatctattgttgacctcgcagcagattgtttcgccgcttccggaacagagcacgccgacagtaaggagcatcaagggaaagcccgtggtaagggacagaaggaacaaatcactgtcgagtgtgggtgccataatcccttctacaaattatggtgcaattcattttcctcccgcctggtagttggtaagttttttgtcgttgttgtcctttccatctgtggacccacatttcttgggtctaacttccggatatattctgagcaattcgcagacagcatgtcgagcccccagattataatgagggcaactgtcggtggacgacgagttatcttggatgattactacgtgtcgtacttgtggctgcgaaacaatacgcctgaagatgcccgtattctctcatggtgggactacgggtatcaaatcactggaattggcaatcgcacaacccttgcggatggtaacacatggaatcacgagcacatagcaactattgggaagatgcttacatcccctgtgaaggagtcacatgctcttatacgccatctcgctgattatgtgctgatatgggccggttatgatggcagcgatttacttaaatcgccacacatggctcggataggcaacagtgtatatcgcgatatatgctcagaggatgatccgctgtgtacgcagttcgggttttatagtggtgacttcagtaaacctacgcctatgatgcagcggtccctattatacaatctgcacaggtttggtacggatggcgggaagacacaactggataagaacatgtttcagctcgcctacgtgtcaaagtatggtttggtgaagatctacaaggtgatgaatgtgagtgaagagagcaaggcgtgggttgcagacccaaagaaccgtaagtgcgatgcacctggatcttggatatgcaccggccagtacccgccagcgaaggagatccaagacatgttagcgaagaggattgactacgaacaactcgaggatttcaaccgccgcaatcgaagtgacgcttattatcgtgcgtatatgcgtcagatgggttag

SEQ ID NO: 56 - protein, 821 aa, Trypanosoma brucei
MTKGGKVAVTKGSAQSDGAGEGGMSKAKSSTTFVATGGGSLPAWALKAVSTVVSAVILIYSVHRAYDIRLTSVRLYGELIHEFDPWFNYRATQYLSDNGWRAFFQWYDYMSWYPLGRPVGTTIFPGMQLTGVAIHRVLEMLGRGMSINNICVYIPAWFGSIATVLAALIAYESSNSLSVMAFTAYFFSIVPAHLMRSMAGEFDNECVAMAAMLLTFYMWVRSLRSSSSWPIGALAGVAYGYMVSTWGGYIFVLNMVAFHASVCVLLDWARGTYSVSLLRAYSLFFVIGTALAICVPPVEWTPFRSLEQLTALFVFVFMWALHYSEYLRERARAPIHSSKALQIRARIFMGTLSLLLIVAIYLFSTGYFRPFSSRVRALFVKHTRTGNPLVDSVAEHHPASNDDFFGYLHVCYNGWIIGFFFMSVSCFFHCTPGMSFLLLYSILAYYFSLKMSRLLLLSAPVASILTGYVVGSIVDLAADCFAASGTEHADSKEHQGKARGKGQKEQITVECGCHNPFYKLWCNSFSSRLVVGKFFVVVVLSICGPTFLGSNFRIYSEQFADSMSSPQIIMRATVGGRRVILDDYYVSYLWLRNNTPEDARILSWWDYGYQITGIGNRTTLADGNTWNHEHIATIGKMLTSPVKESHALIRHLADYVLIWAGYDGSDLLKSPHMARIGNSVYRDICSEDDPLCTQFGFYSGDFSKPTPMMQRSLLYNLHRFGTDGGKTQLDKNMFQLAYVSKYGLVKIYKVMNVSEESKAWVADPKNRKCDAPGSWICTGQYPPAKEIQDMLAKRIDYEQLEDFNRRNRSDAYYRAYMRQMG

SEQ ID NO: 57 - DNA, 2466 bp, Trypanosoma brucei
atgacgaaaggtgggaaagtagctgtgactaagggctcagcacagagtgatggtgctggtgagggagggatgagtaaggccaagtcatccactacgttcgtcgccactggcggtggttctttgcctgcctgggcgctaaaggctgtaagcacggttgtgagtgcagtgattcttatatactctgtccatcgtgcttacgatatacgacttacttctgtccgtctttatggtgagcttattcacgagttcgacccttggttcaattaccgtgcaacgcagtacctcagcgacaacgggtggcgtgcttttttccaatggtacgactacatgagctggtacccgcttggccgaccggtgggcacaaccatcttccccggaatgcagcttaccggtgtagccattcatcgtgtgctggaaatgctcgggcgaggtatgtccatcaacaatatctgtgtgtacattcctgcatggttcggtagtattgccactgtgttggctgctctcattgcgtacgagtcatctaattcgctcagtgtcatggcgtttactgcgtactttttttccatcgtacctgcacacctgatgcgatcaatggctggtgaatttgacaatgagtgtgttgcaatggcggcgatgcttctgacgttctacatgtgggtacgatcgttacgcagctcaagttcgtggcccattggcgctttagctggtgtggcatacgggtacatggtgtccacgtggggtggttatattttcgtgctgaacatggtagccttccacgcttctgtatgtgtactgcttgattgggctcgtgggacatacagcgtcagtttgctgagggcgtattcactgttttttgtcattggcactgcccttgcgatttgcgtaccaccagtggagtggacgcccttccggtcgctggagcaactgacagcattgtttgtcttcgttttcatgtgggcactccactactcggaatacctgcgtgagcgtgcccgagcgcccattcactcttctaaagcacttcagatccgtgcccgcattttcatgggcaccctctccttgctgttgattgtggctatctacctattttcgacaggatacttcaggtcgttttcttctcgtgtccgtgcgttgttcgtgaaacatacgcgtaccggaaatcccctcgtggattctgtggctgagcatcggccgacgactgccggggccttcctacgtcatcttcatgtgtgttacaacggctggataattggctttttcttcatgtccgtgtcgtgctttttccattgtacaccgggaatgtcgttcctgctgttgtactctatacttgcgtactacttctctctcaagatgagtcgtctgctgttactttctgcacctgtggcttccatactcactggctatgttgtgggatctattgttgacctcgcagcagattgtttcgccgcttccggaacagagcacgccgacagtaaggagcatcaagggaaagcccgtggtaagggacaaaagagacaaatcactgtcgagtgtgggtgccataatcccttctacaaattatggtgcaattcattttcctcccgcctggtagttggtaagttttttgtcgttgttgtcctttccatctgtggacccacatttcttgggtctgagtttagggctcattgcgaacgtttctccgtaagtgttgctaatccgcgtattatatcgagtattaggcactccggtaagttggttcttgccgatgattactacgtgtcgtacttgtggctgcgaaacaatacgcctgaagatgcccgtattctctcatggtgggactacgggtatcaaatcactggaattggcaatcgcacaacccttgcggacggtaacacatggaatcacgagcacatagcaactattgggaagatgcttacatcccctgtgaaggagtcacatgctcttatacgccatctcgctgattatgtgctgatatgggccggtgaggatcggggcgatttacgtaagtcacggcatatggctcggataggcaacagtgtatatcgcgatatgtgttcagaagacgatccgctgtgtacgcagttcgggttttatagtggtgacttcaataaacctacgcctatgatgcagcggtccctattatacaatctgcacaggtttggtacggatggcgggaagacacaactggataagaacatgtttcagctcgcctacgtgtcaaagtatggtttggtgaagatctacaaggtgatgaatgtgagtgaagagagcaaggcgtgggttgcagacccaaagaaccgtaagtgcgatgcacctggatcttggatatgcgccggccagtacccgccagcgaaggagatccaagacatgttagcgaagaggattgactacgaacaactcgaggatttcaatcgccgcaatcgaagtgacgcttattatcgtgcgtatatgcgtcagatgggttag

SEQ ID NO: 58 - protein, 821 aa, Trypanosoma brucei
MTKGGKVAVTKGSAQSDGAGEGGMSKAKSSTTFVATGGGSLPAWALKAVSTVVSAVILIYSVHRAYDIRLTSVRLYGELIHEFDPWFNYRATQYLSDNGWRAFFQWYDYMSWYPLGRPVGTTIFPGMQLTGVAIHRVLEMLGRGMSINNICVYIPAWFGSIATVLAALIAYESSNSLSVMAFTAYFFSIVPAHLMRSMAGEFDNECVAMAAMLLTFYMWVRSLRSSSSWPIGALAGVAYGYMVSTWGGYIFVLNMVAFHASVCVLLDWARGTYSVSLLRAYSLFFVIGTALAICVPPVEWTPFRSLEQLTALFVFVFMWALHYSEYLRERARAPIHSSKALQIRARIFMGTLSLLLIVAIYLFSTGYFRSFSSRVRALFVKHTRTGNPLVDSVAEHRPTTAGAFLRHLHVCYNGWIIGFFFMSVSCFFHCTPGMSFLLLYSILAYYFSLKMSRLLLLSAPVASILTGYVVGSIVDLAADCFAASGTEHADSKEHQGKARGKGQKRQITVECGCHNPFYKLWCNSFSSRLVVGKFFVVVVLSICGPTFLGSEFRAHCERFSVSVANPRIISSIRHSGKLVLADDYYVSYLWLRNNTPEDARILSWWDYGYQITGIGNRTTLADGNTWNHEHIATIGKMLTSPVKESHALIRHLADYVLIWAGEDRGDLRKSRHMARIGNSVYRDMCSEDDPLCTQFGFYSGDFNKPTPMMQRSLLYNLHRFGTDGGKTQLDKNMFQLAYVSKYGLVKIYKVMNVSEESKAWVADPKNRKCDAPGSWICAGQYPPAKEIQDMLAKRIDYEQLEDFNRRNRSDAYYRAYMRQMG

SEQ ID NO: 59 - DNA, 2397 bp, Trypanosoma cruzi
atggacacagcacaattaacactgtgtggaaaatatccactcgattattctacagcaaggcgggtcatctccatactcaatgtatttattattgctcttgccatttaccgcgcctacagcattcgtatgatttccattcgtgtgtacggcaaggtcatccacgaatttgatccgtggttcaacttccgagcgtcggagtacctcgacgagcacgggtgggatgcctttttccactggtacgactacatgagttggtatccgcttggtcggcccgtgggcacaaccatctttcctggactacagatcacaagcgtccttattcgcagggccctttccatgctgggaatgagcatgacgatgaacgatgtgtgttgcttgattccggcatggtttggctcagtggcgactgtattggctgcgcttctggcgtacgagacgtggggctcgttttcaggggctgccatgacggcgggtctttttgccatcctacccgcccacctaatgcgctccatggctggcgaatacgacaatgagtgtatagcaatggcggcgatgctgctcacattctacctgtgggtgcgctcgttgcgcaatgcaggctcatggcccattggtgtgcttacggggctggcgtacggctacatggtgtcgacctggggaggcttcatctttgtcctgaacatggtggcgctgcacgccgcggtgtgcgtttttgctgactggatgcgcggcaggtacgatgccagtctgctgtgggcgtactccctgttctttttggtgggcacggcgattgcgacgtgtgtgccgcccgtggggtggaccccattcaagtccttggagcagttgatggcgttgctggtgttcattttcatgtgggcattgcacttctccgagatattgcggcgccgcgctgatgtcccgattcgctccaccaaggcactgcggatccgtgcacgggtatttatgatcacctgcggagtgcttgtgctggctgcagcgctgctggcaccacagggatacttcgggcccctctcctcccgcgtccgtgctttgtttgtgcagcacacccgcaccggcaaccctctcgtagattccgtcgcggagcaccgtccatctagtgggggcgctttgtggaggcttcttcatctgtgttgtccgctgtggttgatcggaatgatttcgcaaatactgtcgggagaaaacgaaaatttaagggcaactacttttatgatttggtactccattatggtattttatttcggctgccgcatgtcacgcttgattttgctaactggaccagttgcagcatcatactccggaagagtaattggaggccttatggactgggcggtcaggcttcttttttggacgaatgtagagtcgatgaaaagcaaaggttccccaacgattcgaagcaaaaaacttgaaaaaaaggggcatctatctaataacaatgagcgttctttacaaaaccgttttcaagacgctgccaatttgtggccccacggaatacgtgtcacaatcgcaatgcttgtgtttgcagcacttcttttcaatccgatggcccgatcgtataacgaagattcaataaagatggcacacacactatctaatccacggattatgtggtattcgatgaccgagcagaacacccctgtacttgtagacgactattacgtctcgtacctgtggctgcgcaacaacacaccggcggacgcccgcattcttgcatggtgggactacggctaccagatcacggggatcgggaatcgcacgagccttgcggacggcaacacgtggaatcacgagcacattgccacaattggaaagctgcttacgtcgcccgtggcgaaggcccacttgctcattcggcaccttgccgactatgtactgatatggaccggcagccgggcggaggacttgatgaagtcgccgcacatggcacgcattggcaacagtgtgtaccgcgacatctgccccgaggacgacccgttgtgctccaactttgggtttgaggactacgacctaagtcgtcccacgccgatgatgcggatgtcgttgctgtacaacctgcatgtctctggggagagccccagtccggcgatcgacaatatgttcaggcttgcctacaggtcgcgccacggcctggtgaagatctacaaggtgatgaatgtgagcgcggagagcaaggcgtgggtggcggacccgaagaaccgcaagtgcgacgcgccagggtcgtggctgtgcactgggcagtacccgccagcgaaggagatccaggagatgctggcgaggcgcatcgactacggccagctggaggacttcaaccgcggcaaacgagatgacgcgtactaccgtgcgtacatgcgtcgcatccgcaatgaagggcgtggctag

SEQ ID NO: 60 - protein, 798 aa, Trypanosoma cruzi
MDTAQLTLCGKYPLDYSTARRVISILNVFIIALAIYRAYSIRMISIRVYGKVIHEFDPWFNFRASEYLDEHGWDAFFHWYDYMSWYPLGRPVGTTIFPGLQITSVLIRRALSMLGMSMTMNDVCCLIPAWFGSVATVLAALLAYETWGSFSGAAMTAGLFAILPAHLMRSMAGEYDNECIAMAAMLLTFYLWVRSLRNAGSWPIGVLTGLAYGYMVSTWGGFIFVLNMVALHAAVCVFADWMRGRYDASLLWAYSLFFLVGTAIATCVPPVGWTPFKSLEQLMALLVFIFMWALHFSEILRRRADVPIRSTKALRIRARVFMITCGVLVLAAALLAPQGYFGPLSSRVRALFVQHTRTGNPLVDSVAEHRPSSGGALWRLLHLCCPLWLIGMISQILSGENENLRATTFMIWYSIMVFYFGCRMSRLILLTGPVAASYSGRVIGGLMDWAVRLLFWTNVESMKSKGSPTIRSKKLEKKGHLSNNNERSLQNRFQDAANLWPHGIRVTIAMLVFAALLFNPMARSYNEDSIKMAHTLSNPRIMWYSMTEQNTPVLVDDYYVSYLWLRNNTPADARILAWWDYGYQITGIGNRTSLADGNTWNHEHIATIGKLLTSPVAKAHLLIRHLADYVLIWTGSRAEDLMKSPHMARIGNSVYRDICPEDDPLCSNFGFEDYDLSRPTPMMRMSLLYNLHVSGESPSPAIDNMFRLAYRSRHGLVKIYKVMNVSAESKAWVADPKNRKCDAPGSWLCTGQYPPAKEIQEMLARRIDYGQLEDFNRGKRDDAYYRAYMRRIRNEGRG

SEQ ID NO: 61 - DNA, 402 bp, Saccharomyces cerevisiae
atcattctggacgtatgtgcacatgtgatttgcttttgtttttttaagaatgtcgggtaataaacagattgtttttctgggaggataatcttttcttttttcctgttggtattctaaaattaaccttgctgtttcttttttttttttttttcgcgcgactactcagccatcttgcatttttaaagaaaaagataatcattaatgccttcacgggaatacgtatagaacattattaaaagtatatgaatggcatatatatatagaacaccacccttggaaaacatttataccccttaaactaaaacaatttgctgcgctataccgtgtttcagtgtattataatacattcatttctgtttcattacgattatattgacgtgataaaaagattatatagccatg